SEQUENCE LISTING (105 sequences)

SEQ ID NO: 1 - DNA, 357 bp, Artificial Sequence
  Synthetic Polynucleotide
caggttcagctgcagcagtctggagctgagctgatgaagcctggggcctcagtgaagatatcctgcaaggcaactggctacacattcagtaaccactggatagagtgggttaaggaaaggcctggacatggcctggagtggattggagagattttacttggaagtggtagagctcattataatgagaagttcaagggcaaggccacattcactgcagatacatcctccaacacagcctacatgcaactcagcagcctgacatctgaggactctgccgtctattactgtgcaagacactataggtacgacgggtggtttgcttactggggccaagggactccggtcactgtctctgca

SEQ ID NO: 2 - DNA, 24 bp, Artificial Sequence
  Synthetic Polynucleotide
ggctacacattcagtaaccactgg

SEQ ID NO: 3 - DNA, 24 bp, Artificial Sequence
  Synthetic Polynucleotide
attttacttggaagtggtagagct

SEQ ID NO: 4 - DNA, 36 bp, Artificial Sequence
  Synthetic Polynucleotide
gcaagacactataggtacgacgggtggtttgcttac

SEQ ID NO: 5 - DNA, 333 bp, Artificial Sequence
  Synthetic Polynucleotide
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaa

SEQ ID NO: 6 - DNA, 30 bp, Artificial Sequence
  Synthetic Polynucleotide
aaaagtgtcagtacatctggctatagttat

SEQ ID NO: 7 - DNA, 9 bp, Artificial Sequence
  Synthetic Polynucleotide
cttgcatcc

SEQ ID NO: 8 - DNA, 27 bp, Artificial Sequence
  Synthetic Polynucleotide
cagcacagtagggagcttccgtacacg

SEQ ID NO: 9 - DNA, 354 bp, Artificial Sequence
  Synthetic Polynucleotide
caggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccacgtactataggtacgacgggtggtttgcttactggggccaagggactctggtcactgtctctgca

SEQ ID NO: 10 - DNA, 24 bp, Artificial Sequence
  Synthetic Polynucleotide
ggtttctcattaactagctatggt

SEQ ID NO: 11 - DNA, 21 bp, Artificial Sequence
  Synthetic Polynucleotide
atatggagtggtggaagcaca

SEQ ID NO: 12 - DNA, 36 bp, Artificial Sequence
  Synthetic Polynucleotide
gccacgtactataggtacgacgggtggtttgcttac

SEQ ID NO: 13 - DNA, 333 bp, Artificial Sequence
  Synthetic Polynucleotide
gacattgtgctgacacagtctcctgcttccttagctgtatctctggggcagagggccaccatctcatgcagggccagcaaaagtgtcagtacatctggctatagttatatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattcctgtcagcacagtagggagcttccgtggacgttcggtggaggcaccaagctggaaatcaaa

SEQ ID NO: 14 - DNA, 30 bp, Artificial Sequence; identical to SEQ ID NO: 6
  Synthetic Polynucleotide

SEQ ID NO: 15 - DNA, 9 bp, Artificial Sequence
  Synthetic Polynucleotide
cttgcatcc

SEQ ID NO: 16 - DNA, 27 bp, Artificial Sequence
  Synthetic Polynucleotide
cagcacagtagggagcttccgtggacg

SEQ ID NO: 17 - DNA, 351 bp, Artificial Sequence
  Synthetic Polynucleotide
gaggttcagctgcagcagtctggggcagagcttgtgaagccaggggcctcagtcaagttgtcctgcacagcttctggcttcaacattaaagacagctatatgcactgggtgaagcagaggcctgaacagggcctggagtggattggaaggattgatcctgcgaatgctaatcctaaatatgacccgaagttccagggcaaggccactataacaacagacacatcctccaacacagcctacctgcagctcagcagcctgacatctgaggacactgccgtctattactgtgctagagactacggagggtacttcgatgtctggggcgcagggaccacggtcaccgtctcctca

SEQ ID NO: 18 - DNA, 24 bp, Artificial Sequence
  Synthetic Polynucleotide
ggcttcaacattaaagacagctat

SEQ ID NO: 19 - DNA, 27 bp, Artificial Sequence
  Synthetic Polynucleotide
attgatcctgcgaatgctaatcctaaa

SEQ ID NO: 20 - DNA, 30 bp, Artificial Sequence
  Synthetic Polynucleotide
gctagagactacggagggtacttcgatgtc

SEQ ID NO: 21 - DNA, 322 bp, Artificial Sequence
  Synthetic Polynucleotide
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgccacttattactgtcaacagtattggagtactccgtggacgttcggtggaggcaccaagctggaaatcaaac

SEQ ID NO: 22 - DNA, 18 bp, Artificial Sequence
  Synthetic Polynucleotide
gaccacattaataattgg

SEQ ID NO: 23 - DNA, 9 bp, Artificial Sequence
  Synthetic Polynucleotide
ggtgcaacc

SEQ ID NO: 24 - DNA, 27 bp, Artificial Sequence
  Synthetic Polynucleotide
caacagtattggagtactccgtggacg

SEQ ID NO: 25 - DNA, 735 bp, Artificial Sequence
  Synthetic Polynucleotide
caggttcagctgcagcagtctggagctgagctgatgaagcctggggcctcagtgaagatatcctgcaaggcaactggctacacattcagtaaccactggatagagtgggttaaggaaaggcctggacatggcctggagtggattggagagattttacttggaagtggtagagctcattataatgagaagttcaagggcaaggccacattcactgcagatacatcctccaacacagcctacatgcaactcagcagcctgacatctgaggactctgccgtctattactgtgcaagacactataggtacgacgggtggtttgcttactggggccaagggactccggtcactgtctctgcaggtggaggcggttcaggcggaggtggctctggcggtggcgctagcgacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaa

SEQ ID NO: 26 - DNA, 735 bp, Artificial Sequence
  Synthetic Polynucleotide
gacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaaggtggaggcggttcaggcggaggtggctctggcggtggcgctagccaggttcagctgcagcagtctggagctgagctgatgaagcctggggcctcagtgaagatatcctgcaaggcaactggctacacattcagtaaccactggatagagtgggttaaggaaaggcctggacatggcctggagtggattggagagattttacttggaagtggtagagctcattataatgagaagttcaagggcaaggccacattcactgcagatacatcctccaacacagcctacatgcaactcagcagcctgacatctgaggactctgccgtctattactgtgcaagacactataggtacgacgggtggtttgcttactggggccaagggactccggtcactgtctctgca

SEQ ID NO: 27 - DNA, 732 bp, Artificial Sequence
  Synthetic Polynucleotide
caggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccacgtactataggtacgacgggtggtttgcttactggggccaagggactctggtcactgtctctgcaggtggaggcggttcaggcggaggtggctctggcggtggcgctagcgacattgtgctgacacagtctcctgcttccttagctgtatctctggggcagagggccaccatctcatgcagggccagcaaaagtgtcagtacatctggctatagttatatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattcctgtcagcacagtagggagcttccgtggacgttcggtggaggcaccaagctggaaatcaaa

SEQ ID NO: 28 - DNA, 732 bp, Artificial Sequence
  Synthetic Polynucleotide
gacattgtgctgacacagtctcctgcttccttagctgtatctctggggcagagggccaccatctcatgcagggccagcaaaagtgtcagtacatctggctatagttatatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattcctgtcagcacagtagggagcttccgtggacgttcggtggaggcaccaagctggaaatcaaaggtggaggcggttcaggcggaggtggctctggcggtggcgctagccaggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccacgtactataggtacgacgggtggtttgcttactggggccaagggactctggtcactgtctctgca

SEQ ID NO: 29 - DNA, 718 bp, Artificial Sequence
  Synthetic Polynucleotide
gaggttcagctgcagcagtctggggcagagcttgtgaagccaggggcctcagtcaagttgtcctgcacagcttctggcttcaacattaaagacagctatatgcactgggtgaagcagaggcctgaacagggcctggagtggattggaaggattgatcctgcgaatgctaatcctaaatatgacccgaagttccagggcaaggccactataacaacagacacatcctccaacacagcctacctgcagctcagcagcctgacatctgaggacactgccgtctattactgtgctagagactacggagggtacttcgatgtctggggcgcagggaccacggtcaccgtctcctcaggtggaggcggttcaggcggaggtggctctggcggtggcgctagcgacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgccacttattactgtcaacagtattggagtactccgtggacgttcggtggaggcaccaagctggaaatcaaac

SEQ ID NO: 30 - DNA, 718 bp, Artificial Sequence
  Synthetic Polynucleotide
gacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgccacttattactgtcaacagtattggagtactccgtggacgttcggtggaggcaccaagctggaaatcaaacggtggaggcggttcaggcggaggtggctctggcggtggcgctagcgaggttcagctgcagcagtctggggcagagcttgtgaagccaggggcctcagtcaagttgtcctgcacagcttctggcttcaacattaaagacagctatatgcactgggtgaagcagaggcctgaacagggcctggagtggattggaaggattgatcctgcgaatgctaatcctaaatatgacccgaagttccagggcaaggccactataacaacagacacatcctccaacacagcctacctgcagctcagcagcctgacatctgaggacactgccgtctattactgtgctagagactacggagggtacttcgatgtctggggcgcagggaccacggtcaccgtctcctca

SEQ ID NO: 31 - DNA, 45 bp, Artificial Sequence
  Synthetic Polynucleotide
ggtggaggcggttcaggcggaggtggctctggcggtggcgctagc

SEQ ID NO: 32 - DNA, 2640 bp, Artificial Sequence
  Synthetic Polynucleotide
atggggatggccctgcctgtgacagctctgctcctccctctggccctgctgctccatgccgccagaccccaggttcagctgcagcagtctggagctgagctgatgaagcctggggcctcagtgaagatatcctgcaaggcaactggctacacattcagtaaccactggatagagtgggttaaggaaaggcctggacatggcctggagtggattggagagattttacttggaagtggtagagctcattataatgagaagttcaagggcaaggccacattcactgcagatacatcctccaacacagcctacatgcaactcagcagcctgacatctgaggactctgccgtctattactgtgcaagacactataggtacgacgggtggtttgcttactggggccaagggactccggtcactgtctctgcaggtggaggcggttcaggcggaggtggctctggcggtggcgctagcgacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 33 - DNA, 2640 bp, Artificial Sequence
  Synthetic Polynucleotide
atggggatggccctgcctgtgacagctctgctcctccctctggccctgctgctccatgccgccagacccgacattgtgctgacacagtctcctgcttccttagctgtatctctggggcagagggccaccatctcatgcagggccagcaaaagtgtcagtacatctggctatagttatatgcactggtaccaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattactgtcagcacagtagggagcttccgtacacgttcggaggggggaccaagctggaaataaaaggtggaggcggttcaggcggaggtggctctggcggtggcgctagccaggttcagctgcagcagtctggagctgagctgatgaagcctggggcctcagtgaagatatcctgcaaggcaactggctacacattcagtaaccactggatagagtgggttaaggaaaggcctggacatggcctggagtggattggagagattttacttggaagtggtagagctcattataatgagaagttcaagggcaaggccacattcactgcagatacatcctccaacacagcctacatgcaactcagcagcctgacatctgaggactctgccgtctattactgtgcaagacactataggtacgacgggtggtttgcttactggggccaagggactccggtcactgtctctgcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 34 - DNA, 2637 bp, Artificial Sequence
  Synthetic Polynucleotide
atggggatggccctgcctgtgacagctctgctcctccctctggccctgctgctccatgccgccagaccccaggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccacgtactataggtacgacgggtggtttgcttactggggccaagggactctggtcactgtctctgcaggtggaggcggttcaggcggaggtggctctggcggtggcgctagcgacattgtgctgacacagtctcctgcttccttagctgtatctctggggcagagggccaccatctcatgcagggccagcaaaagtgtcagtacatctggctatagttatatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattcctgtcagcacagtagggagcttccgtggacgttcggtggaggcaccaagctggaaatcaaacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 35 - DNA, 2637 bp, Artificial Sequence
  Synthetic Polynucleotide
atggggatggccctgcctgtgacagctctgctcctccctctggccctgctgctccatgccgccagacccgacattgtgctgacacagtctcctgcttccttagctgtatctctggggcagagggccaccatctcatgcagggccagcaaaagtgtcagtacatctggctatagttatatgcactggtatcaacagaaaccaggacagccacccaaactcctcatctatcttgcatccaacctagaatctggggtccctgccaggttcagtggcagtgggtctgggacagacttcaccctcaacatccatcctgtggaggaggaggatgctgcaacctattcctgtcagcacagtagggagcttccgtggacgttcggtggaggcaccaagctggaaatcaaaggtggaggcggttcaggcggaggtggctctggcggtggcgctagccaggtgcagctgaagcagtcaggacctggcctagtgcagccctcacagagcctgtccatcacctgcacagtctctggtttctcattaactagctatggtgtacactgggttcgccagtctccaggaaagggtctggagtggctgggagtgatatggagtggtggaagcacagactataatgcagctttcatatccagactgagcatcagcaaggacaattccaagagccaagttttctttaaaatgaacagtctgcaagctaatgacacagccatatattactgtgccacgtactataggtacgacgggtggtttgcttactggggccaagggactctggtcactgtctctgcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 36 - DNA, 2625 bp, Artificial Sequence
  Synthetic Polynucleotide
atggggatggccctgcctgtgacagctctgctcctccctctggccctgctgctccatgccgccagacccgaggttcagctgcagcagtctggggcagagcttgtgaagccaggggcctcagtcaagttgtcctgcacagcttctggcttcaacattaaagacagctatatgcactgggtgaagcagaggcctgaacagggcctggagtggattggaaggattgatcctgcgaatgctaatcctaaatatgacccgaagttccagggcaaggccactataacaacagacacatcctccaacacagcctacctgcagctcagcagcctgacatctgaggacactgccgtctattactgtgctagagactacggagggtacttcgatgtctggggcgcagggaccacggtcaccgtctcctcaggtggaggcggttcaggcggaggtggctctggcggtggcgctagcgacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgccacttattactgtcaacagtattggagtactccgtggacgttcggtggaggcaccaagctggaaatcaaaccccgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 37 - DNA, 2625 bp, Artificial Sequence
  Synthetic Polypeptide
atggggatggccctgcctgtgacagctctgctcctccctctggccctgctgctccatgccgccagacccgacatccagatgacacaatcttcatcctacttgtctgtatctctaggaggcagagtcaccattacttgcaaggcaagtgaccacattaataattggttagcctggtatcagcagaaaccaggaaatgctcctaggctcttaatatctggtgcaaccagtttggaaactggggttccttcaagattcagtggcagtggatctggaaaggattacactctcagcattaccagtcttcagactgaagatgttgccacttattactgtcaacagtattggagtactccgtggacgttcggtggaggcaccaagctggaaatcaaacccggtggaggcggttcaggcggaggtggctctggcggtggcgctagcgaggttcagctgcagcagtctggggcagagcttgtgaagccaggggcctcagtcaagttgtcctgcacagcttctggcttcaacattaaagacagctatatgcactgggtgaagcagaggcctgaacagggcctggagtggattggaaggattgatcctgcgaatgctaatcctaaatatgacccgaagttccagggcaaggccactataacaacagacacatcctccaacacagcctacctgcagctcagcagcctgacatctgaggacactgccgtctattactgtgctagagactacggagggtacttcgatgtctggggcgcagggaccacggtcaccgtctcctcacgtacggtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctga

SEQ ID NO: 38 - DNA, 2625 bp, Artificial Sequence; identical to SEQ ID NO: 37
  Synthetic Polynucleotide

SEQ ID NO: 39 - DNA, 2640 bp, Artificial Sequence; identical to SEQ ID NO: 33
  Synthetic Polynucleotide

SEQ ID NO: 40 - DNA, 2637 bp, Artificial Sequence; identical to SEQ ID NO: 34
  Synthetic Polynucleotide

SEQ ID NO: 41 - DNA, 2637 bp, Artificial Sequence; identical to SEQ ID NO: 35
  Synthetic Polynucleotide

SEQ ID NO: 42 - DNA, 2625 bp, Artificial Sequence; identical to SEQ ID NO: 36
  Synthetic Polynucleotide

SEQ ID NO: 43 - DNA, 2625 bp, Artificial Sequence; identical to SEQ ID NO: 37
  Synthetic Polynucleotide

SEQ ID NO: 44 - protein, 119 aa, Artificial Sequence
  Synthetic Polypeptide
QVQLQQSGAELMKPGASVKISCKATGYTFSNHWIEWVKERPGHGLEWIGEILLGSGRAHYNEKFKGKATFTADTSSNTAYMQLSSLTSEDSAVYYCARHYRYDGWFAYWGQGTPVTVSA

SEQ ID NO: 45 - protein, 8 aa, Artificial Sequence
  Synthetic Polypeptide
GYTFSNHW

SEQ ID NO: 46 - protein, 8 aa, Artificial Sequence
  Synthetic Polypeptide
ILLGSGRA

SEQ ID NO: 47 - protein, 12 aa, Artificial Sequence
  Synthetic Polypeptide
ARHYRYDGWFAY

SEQ ID NO: 48 - protein, 111 aa, Artificial Sequence
  Synthetic Polypeptide
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 49 - protein, 10 aa, Artificial Sequence
  Synthetic Polypeptide
KSVSTSGYSY

SEQ ID NO: 50 - protein, 3 aa, Artificial Sequence
  Synthetic Polypeptide
LAS

SEQ ID NO: 51 - protein, 9 aa, Artificial Sequence
  Synthetic Polypeptide
QHSRELPYT

SEQ ID NO: 52 - protein, 118 aa, Artificial Sequence
  Synthetic Polypeptide
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCATYYRYDGWFAYWGQGTLVTVSA

SEQ ID NO: 53 - protein, 8 aa, Artificial Sequence
  Synthetic Polypeptide
GFSLTSYG

SEQ ID NO: 54 - protein, 7 aa, Artificial Sequence
  Synthetic Polypeptide
IWSGGST

SEQ ID NO: 55 - protein, 12 aa, Artificial Sequence
  Synthetic Polypeptide
ATYYRYDGWFAY

SEQ ID NO: 56 - protein, 111 aa, Artificial Sequence
  Synthetic Polypeptide
DIVLTQSPASLAVSLGQRATISCRASKSVSTSGYSYMHWYQQKPGQPPKLLIYLASNLESGVPARFSGSGSGTDFTLNIHPVEEEDAATYSCQHSRELPWTFGGGTKLEIK

SEQ ID NO: 57 - protein, 10 aa, Artificial Sequence
  Synthetic Polypeptide
KSVSTSGYSY

SEQ ID NO: 58 - protein, 3 aa, Artificial Sequence
  Synthetic Polypeptide
LAS

SEQ ID NO: 59 - protein, 9 aa, Artificial Sequence
  Synthetic Polypeptide
QHSRELPWT

SEQ ID NO: 60 - protein, 117 aa, Artificial Sequence
  Synthetic Polypeptide
EVQLQQSGAELVKPGASVKLSCTASGFNIKDSYMHWVKQRPEQGLEWIGRIDPANANPKYDPKFQGKATITTDTSSNTAYLQLSSLTSEDTAVYYCARDYGGYFDVWGAGTTVTVSS

SEQ ID NO: 61 - protein, 8 aa, Artificial Sequence
  Synthetic Polypeptide
GFNIKDSY

SEQ ID NO: 62 - protein, 8 aa, Artificial Sequence
  Synthetic Polypeptide
IDPANANP

SEQ ID NO: 63 - protein, 10 aa, Artificial Sequence
  Synthetic Polypeptide
ARDYGGYFDV

SEQ ID NO: 64 - protein, 107 aa, Artificial Sequence
  Synthetic Polypeptide
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 65 - protein, 6 aa, Artificial Sequence
  Synthetic Polypeptide
DHINNW

SEQ ID NO: 66 - protein, 3 aa, Artificial Sequence
  Synthetic Polypeptide
GAT

SEQ ID NO: 67 - protein, 9 aa, Artificial Sequence
  Synthetic Polypeptide
QQYWSTPWT

SEQ ID NO: 68 - protein, 245 aa, Artificial Sequence
  Synthetic Polypeptide
QVQLQQSGAELMKPGASVKISCKATGYTFSNHWIEWVKERPGHGLEWIGEILLGSGRAHYNEKFKGKATFTADTSSNTAYMQLSSLTSEDSAVYYCARHYRYDGWFAYWGQGTPVTVSAGGGGSGGGGSGGGASDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIK

SEQ ID NO: 69 - protein, 245 aa, Artificial Sequence
  Synthetic Polypeptide
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIKGGGGSGGGGSGGGASQVQLQQSGAELMKPGASVKISCKATGYTFSNHWIEWVKERPGHGLEWIGEILLGSGRAHYNEKFKGKATFTADTSSNTAYMQLSSLTSEDSAVYYCARHYRYDGWFAYWGQGTPVTVSA

SEQ ID NO: 70 - protein, 244 aa, Artificial Sequence
  Synthetic Polypeptide
QVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCATYYRYDGWFAYWGQGTLVTVSAGGGGSGGGGSGGGASDIVLTQSPASLAVSLGQRATISCRASKSVSTSGYSYMHWYQQKPGQPPKLLIYLASNLESGVPARFSGSGSGTDFTLNIHPVEEEDAATYSCQHSRELPWTFGGGTKLEIK

SEQ ID NO: 71 - protein, 244 aa, Artificial Sequence
  Synthetic Polypeptide
DIVLTQSPASLAVSLGQRATISCRASKSVSTSGYSYMHWYQQKPGQPPKLLIYLASNLESGVPARFSGSGSGTDFTLNIHPVEEEDAATYSCQHSRELPWTFGGGTKLEIKGGGGSGGGGSGGGASQVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCATYYRYDGWFAYWGQGTLVTVSA

SEQ ID NO: 72 - protein, 239 aa, Artificial Sequence
  Synthetic Polypeptide
EVQLQQSGAELVKPGASVKLSCTASGFNIKDSYMHWVKQRPEQGLEWIGRIDPANANPKYDPKFQGKATITTDTSSNTAYLQLSSLTSEDTAVYYCARDYGGYFDVWGAGTTVTVSSGGGGSGGGGSGGGASDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIK

SEQ ID NO: 73 - protein, 239 aa, Artificial Sequence
  Synthetic Polypeptide
DIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKGGGGSGGGGSGGGASEVQLQQSGAELVKPGASVKLSCTASGFNIKDSYMHWVKQRPEQGLEWIGRIDPANANPKYDPKFQGKATITTDTSSNTAYLQLSSLTSEDTAVYYCARDYGGYFDVWGAGTTVTVSS

SEQ ID NO: 74 - protein, 15 aa, Artificial Sequence
  Synthetic Polypeptide
GGGGSGGGGSGGGAS

SEQ ID NO: 75 - protein, 27 aa, Artificial Sequence
  Synthetic Polypeptide
FWVLVVVGGVLACYSLLVTVAFIIFWV

SEQ ID NO: 76 - protein, 66 aa, Artificial Sequence
  Synthetic Polypeptide
TTTPAPRPPTPAPTIASQPLSLRPEACRPAAGGAVHTRGLDFACDIYIWAPLAGTCGVLLLSLVIT

SEQ ID NO: 77 - protein, 27 aa, Artificial Sequence
  Synthetic Polypeptide
IISFFLALTSTALLFLLFFLTLRFSVV

SEQ ID NO: 78 - protein, 21 aa, Artificial Sequence
  Synthetic Polypeptide
LLAGLVAADAVASLLIVGAVF

SEQ ID NO: 79 - protein, 21 aa, Artificial Sequence
  Synthetic Polypeptide
GVLAGIVMGDLVLTVLIALAV

SEQ ID NO: 80 - protein, 21 aa, Artificial Sequence
  Synthetic Polypeptide
AVMIIFRIGMAVAIFCCFFFP

SEQ ID NO: 81 - protein, 114 aa, Artificial Sequence
  Synthetic Polypeptide
TRKKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 82 - protein, 114 aa, Artificial Sequence
  Synthetic Polypeptide
KRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 83 - protein, 42 aa, Artificial Sequence
  Synthetic Polypeptide
KRGRKKLLYIFKQPFMRPVQTTQEEDGCSCRFPEEEEGGCEL

SEQ ID NO: 84 - protein, 24 aa, Artificial Sequence
  Synthetic Polypeptide
LCARPRRSPAQEDGKVYINMPGRG

SEQ ID NO: 85 - protein, 52 aa, Artificial Sequence
  Synthetic Polypeptide
YFLGRLVPRGRGAAEAATRKQRITETESPYQELQGQRSDVYSDLNTQRPYYK

SEQ ID NO: 86 - protein, 51 aa, Artificial Sequence
  Synthetic Polypeptide
SANERCKSKVVPCRQKQWRTSFDSKKLDLNYNHFESMEWSHRSRRGRIWGM

SEQ ID NO: 87 - protein, 227 aa, Artificial Sequence
  Synthetic Polypeptide
TVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEAL

SEQ ID NO: 88 - protein, 39 aa, Artificial Sequence
  Synthetic Polypeptide
IEVMYPPPYLDNEKSNGTIIHVKGKHLCPSPLFPGPSKP

SEQ ID NO: 89 - protein, 160 aa, Artificial Sequence
  Synthetic Polypeptide
ISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 90 - protein, 20 aa, Artificial Sequence
  Synthetic Polypeptide
QCTNYALLKLAGDVESNPGP

SEQ ID NO: 91 - protein, 18 aa, Artificial Sequence
  Synthetic Polypeptide
EGRGSLLTCGDVEENPGP

SEQ ID NO: 92 - protein, 19 aa, Artificial Sequence
  Synthetic Polypeptide
ATNFSLLKQAGDVEENPGP

SEQ ID NO: 93 - protein, 22 aa, Artificial Sequence
  Synthetic Polypeptide
VKQTLNFDLLKLAGDVESNPGP

SEQ ID NO: 94 - protein, 879 aa, Artificial Sequence
  Synthetic Polypeptide
MGMALPVTALLLPLALLLHAARPQVQLQQSGAELMKPGASVKISCKATGYTFSNHWIEWVKERPGHGLEWIGEILLGSGRAHYNEKFKGKATFTADTSSNTAYMQLSSLTSEDSAVYYCARHYRYDGWFAYWGQGTPVTVSAGGGGSGGGGSGGGASDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIKRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 95 - protein, 879 aa, Artificial Sequence
  Synthetic Polypeptide
MGMALPVTALLLPLALLLHAARPDIVLTQSPASLAVSLGQRATISCRASKSVSTSGYSYMHWYQQKPGQPPKLLIYLASNLESGVPARFSGSGSGTDFTLNIHPVEEEDAATYYCQHSRELPYTFGGGTKLEIKGGGGSGGGGSGGGASQVQLQQSGAELMKPGASVKISCKATGYTFSNHWIEWVKERPGHGLEWIGEILLGSGRAHYNEKFKGKATFTADTSSNTAYMQLSSLTSEDSAVYYCARHYRYDGWFAYWGQGTPVTVSARTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 96 - protein, 878 aa, Artificial Sequence
  Synthetic Polypeptide
MGMALPVTALLLPLALLLHAARPQVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCATYYRYDGWFAYWGQGTLVTVSAGGGGSGGGGSGGGASDIVLTQSPASLAVSLGQRATISCRASKSVSTSGYSYMHWYQQKPGQPPKLLIYLASNLESGVPARFSGSGSGTDFTLNIHPVEEEDAATYSCQHSRELPWTFGGGTKLEIKRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 97 - protein, 878 aa, Artificial Sequence
  Synthetic Polypeptide
MGMALPVTALLLPLALLLHAARPDIVLTQSPASLAVSLGQRATISCRASKSVSTSGYSYMHWYQQKPGQPPKLLIYLASNLESGVPARFSGSGSGTDFTLNIHPVEEEDAATYSCQHSRELPWTFGGGTKLEIKGGGGSGGGGSGGGASQVQLKQSGPGLVQPSQSLSITCTVSGFSLTSYGVHWVRQSPGKGLEWLGVIWSGGSTDYNAAFISRLSISKDNSKSQVFFKMNSLQANDTAIYYCATYYRYDGWFAYWGQGTLVTVSARTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 98 - protein, 874 aa, Artificial Sequence
  Synthetic Polypeptide
MGMALPVTALLLPLALLLHAARPEVQLQQSGAELVKPGASVKLSCTASGFNIKDSYMHWVKQRPEQGLEWIGRIDPANANPKYDPKFQGKATITTDTSSNTAYLQLSSLTSEDTAVYYCARDYGGYFDVWGAGTTVTVSSGGGGSGGGGSGGGASDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKPRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 99 - protein, 874 aa, Artificial Sequence
  Synthetic Polypeptide
MGMALPVTALLLPLALLLHAARPDIQMTQSSSYLSVSLGGRVTITCKASDHINNWLAWYQQKPGNAPRLLISGATSLETGVPSRFSGSGSGKDYTLSITSLQTEDVATYYCQQYWSTPWTFGGGTKLEIKPGGGGSGGGGSGGGASEVQLQQSGAELVKPGASVKLSCTASGFNIKDSYMHWVKQRPEQGLEWIGRIDPANANPKYDPKFQGKATITTDTSSNTAYLQLSSLTSEDTAVYYCARDYGGYFDVWGAGTTVTVSSRTVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 100 - protein, 874 aa, Artificial Sequence; identical to SEQ ID NO: 99
  Synthetic Polypeptide

SEQ ID NO: 101 - protein, 879 aa, Artificial Sequence; identical to SEQ ID NO: 95
  Synthetic Polypeptide

SEQ ID NO: 102 - protein, 878 aa, Artificial Sequence; identical to SEQ ID NO: 96
  Synthetic Polypeptide

SEQ ID NO: 103 - protein, 878 aa, Artificial Sequence; identical to SEQ ID NO: 97
  Synthetic Polypeptide

SEQ ID NO: 104 - protein, 874 aa, Artificial Sequence; identical to SEQ ID NO: 98
  Synthetic Polypeptide

SEQ ID NO: 105 - protein, 874 aa, Artificial Sequence; identical to SEQ ID NO: 99
  Synthetic Polypeptide

(19) United States
(12) Patent Application Publication
(10) Pub. No.: US 2024/0269285 A1
(43) Pub. Date: Aug. 15, 2024

(54) POLYPEPTIDES TARGETING CD70-POSITIVE CANCERS

(71) Applicant: BOARD OF REGENTS, THE UNIVERSITY OF TEXAS SYSTEM, Austin, TX (US)

(72) Inventors: Katy REZVANI, Houston, TX (US); Laura del Carmen BOVER, Houston, TX (US); Rafet BASAR, Houston, TX (US); Sunil ACHARYA, Houston, TX (US); Long VIEN, Houston, TX (US); Nadima UPRETY, Houston, TX (US); Emily ENSLEY, Houston, TX (US)

(73) Assignee: BOARD OF REGENTS, THE UNIVERSITY OF TEXAS SYSTEM, Austin, TX (US)

(21) Appl. No.: 18/567,967

(22) PCT Filed: Jun. 29, 2022
(86) PCT No.: PCT/US2022/035441

§ 371 Date: Dec. 7, 2023

Related U.S. Application Data
(60) Provisional application No. 63/216,753, filed on Jun. 30, 2021.

(51) Int. Cl. A61K 39/00; A61K 38/20; A61K 39/395; C07K 14/54; C07K 16/28

(52) U.S. Cl. CPC A61K 39/464438; A61K 38/2086; A61K 39/39558; A61K 39/4611; A61K 39/4613; A61K 39/4631; A61K 39/4635; C07K 14/5443; C07K 16/2875; C07K 2317/92; C07K 2319/33

(57) ABSTRACT

Embodiments of the present disclosure include methods and compositions related to CD70-targeting polypeptides. In certain aspects, anti-CD70 antibodies are disclosed. In some aspects, disclosed are chimeric receptors engineered to bind to CD70. Also disclosed are immune cell engagers comprising a CD70-binding region and one or more immune cell binding regions. Cells (e.g., NNK cells, T cells) expressing CD70-targeting peptides are described. Also described are therapeutic methods using polypeptides of the disclosure.

### CROSS-REFERENCE TO RELATED APPLICATIONS

This application claims priority to U.S. Provisional Application Ser. No. 63/216,753, filed Jun. 30, 2021, which is incorporated by reference herein in its entirety.

### SEQUENCE LISTING

The instant application contains a Sequence Listing which has been submitted in ASCII format and is hereby incorporated by reference in its entirety. Said ASCII copy, created on Jun. 27, 2022, is named “MDACP1300WO_Sequence_Listing.txt” and is 178 kilobytes in size.

### BACKGROUND

### I. Technical Field

Embodiments of the disclosure include at least the fields of cell biology, molecular biology, immunology, and medicine, including cancer medicine.

### II. Background

The CD70 protein is a member of the tumor necrosis factor (TNF) superfamily. Expression of this protein is restricted in normal tissues. CD70 is not a lineage-specific marker and is only briefly expressed in subsets of highly-activated T-lymphocytes, B-lymphocytes, and dendritic cells. CD70 is reportedly absent in non-lymphoid tissues [1], although the aberrant expression of CD70 is linked to various solid tumors, including ovarian and breast cancer, renal cell carcinoma, and glioblastoma [2]. Targeting of CD70 has been described, resulting in tumor regression and clearance in hematological malignancies [3-8], as this marker is present in Hodgkin's Disease and in several other lymphomas of B- and T-cell origin. Because CD70 has been identified as aberrantly expressed in many solid malignancies, it has also been explored as a viable tumor biomarker for targeted therapy against solid tumors.

There exists a need for methods and compositions for targeting CD70 and CD70-positive tumors for cancer treatment.

### SUMMARY

Embodiments of the disclosure encompass methods and compositions related to polypeptides that target CD70, including antibodies and engineered polypeptides such as chimeric antigen receptors (CARs), immune cell engagers (e.g., bispecific or multispecific engagers), and the like. In certain aspects, disclosed are anti-CD70 antibodies and methods for use in treatment of various conditions, including cancer. In further aspects, disclosed are immune cell engagers comprising a CD70-binding region and an immune cell binding region (e.g., CD3-binding region, CD16-binding region, NCR-binding region, etc.). Also disclosed are engineered polypeptides such as CARs and TCRs comprising a CD70-binding region. In specific embodiments, the polypeptides of the disclosure that target CD70 are comprised on the surface of cells of any kind, including immune cells.

Embodiments of the present disclosure include polynucleotides, polypeptides, vectors, expression constructs, engineered receptors, chimeric antigen receptors, immune cell engagers, antibodies, antibody fragments, pharmaceutical compositions, methods for generating an antibody, methods for generating a CAR, methods for generating an immune cell engager, methods for generating a CAR T cell, methods for generating a CAR NK cell, and methods for treating a subject for cancer. Polypeptides of the disclosure can include at least 1, 2, 3, or more of: an antigen binding region, a CD70-binding region, a variable heavy chain region, a variable light chain region, a transmembrane domain, an intracellular domain, a costimulatory domain, a hinge region, a signal peptide, a polypeptide linker, and an immune cell binding region. Any one of more of the preceeding components may be excluded from polypeptides of the disclosure in certain embodiments.

In some embodiments, the disclosed polypeptides comprise a heavy chain variable region (V_{H}). In some embodiments, a polypeptide of the disclosure comprises a V_{H} comprising one or more CDRs having at least, at most, exactly, or between any two of 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:45, SEQ ID NO:46, SEQ ID NO:47, SEQ ID NO:53, SEQ ID NO:54, SEQ ID NO:55, SEQ ID NO:61, SEQ ID NO:62, or SEQ ID NO:63. In some embodiments, the V_{H} comprises SEQ ID NO:45, SEQ ID NO:46, SEQ ID NO:47, SEQ ID NO:53, SEQ ID NO:54, SEQ ID NO:55, SEQ ID NO:61, SEQ ID NO:62, or SEQ ID NO:63, or any combination thereof. In some embodiments, the V_{H} comprises an amino acid sequence having at least, at most, exactly, or between any two of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:44, SEQ ID NO:52, or SEQ ID NO:60. In some embodiments, the V_{H} comprises SEQ ID NO:44, SEQ ID NO:52, or SEQ ID NO:60.

In some embodiments, the disclosed polypeptides comprise a light chain variable region (V_{L}). In some embodiments, a polypeptide of the disclosure comprises a V_{L} comprising one or more CDRs having at least, at most, exactly, or between any two of 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:49, SEQ ID NO:50, SEQ ID NO:51, SEQ ID NO:57, SEQ ID NO:58, SEQ ID NO:59, SEQ ID NO:65, SEQ ID NO:66, or SEQ ID NO:67. In some embodiments, the V_{L} comprises SEQ ID NO:49, SEQ ID NO:50, SEQ ID NO:51, SEQ ID NO:57, SEQ ID NO:58, SEQ ID NO:59, SEQ ID NO:65, SEQ ID NO:66, or SEQ ID NO:67, or any combination thereof. In some embodiments, the V_{L} comprises an amino acid sequence having at least, at most, exactly, or between any two of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with SEQ ID NO:48, SEQ ID NO:56, or SEQ ID NO:64. In some embodiments, the V_{L} comprises SEQ ID NO:48, SEQ ID NO:56, or SEQ ID NO:64. Disclosed are polypeptides comprising any combination of one or more V_{H} and one or more V_{L}. Any one or more V_{H} and/or V_{L} described herein may be excluded from polypeptides of the disclosure in certain embodiments.

In particular aspects, disclosed are polypeptides (e.g., antibodies, chimeric antigen receptors, immune cell engagers) comprising a sequence having at least, at most, exactly, or between any two of 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99%, 99.1%, 99.2%, 99.3%, 99.4%, 99.5%, 99.6%, 99.7%, 99.8%, or 99.9% sequence identity with any of SEQ ID NOs:44-74. In some embodiments, disclosed are polypeptides comprising any one or more of SEQ ID NOs:44-74.

Also presented herein are vectors comprising a polynucleotide of the disclosure. Vectors contemplated herein include viral vectors (e.g., adenoviral vectors, adeno-associated viral vectors, lentiviral vectors, and retroviral vectors) and non-viral vectors (e.g., plasmids).

Embodiments of the disclosure include immune cells of any kind comprising any polynucleotide and/or polypeptide encompassed herein. In specific embodiments, the immune cell is a NK cell, T cell, gamma delta (γδ) T cell, alpha beta (αβ) T cell, invariant NKT (iNKT) cell, B cell, macrophage, MSC, or dendritic cell. In cases wherein the immune cell is an NK cell, the NK cell may be derived from cord blood (including pooled cord blood units), peripheral blood, induced pluripotent stem cells, bone marrow, and/or from a cell line. In specific aspects, the NK cell line is NK-92 cell line or another NK cell line derived from a tumor or from a healthy NK cell or a progenitor cell. In cases where the immune cell is a T cell, the T cell may be derived from cord blood (including pooled cord blood units), peripheral blood, induced pluripotent stem cells, bone marrow, and/or from a cell line.

In specific embodiments, the immune cell is an NK cell, such as one derived from cord blood, such as from a cord blood mononuclear cell. The NK cell may be a CD56⁺ NK cell, in specific cases. The NK cells may express one or more exogenously provided cytokines, such as IL-15, IL-2, IL-12, IL-18, IL-21, IL-23, IL-7, or a combination thereof. Particular embodiments include populations of immune cells of any kind of the disclosure, and the cells may be present in a suitable medium or a suitable carrier of any kind.

Methods of treating or preventing cancer of any kind are encompassed herein, including by administering cells expressing particular anti-CD70 polypeptides (e.g., antibodies, CARs, immune cell engagers) at a therapeutically effective amount to ameliorate or prevent the cancer, or reduce the risk of the cancer, reduce the severity of the cancer, prevent metastasis or risk thereof, or delay the onset of the cancer.

In some embodiments, disclosed is a method of killing CD70-positive cells in an individual comprising administering to the individual an effective amount of cells harboring any polynucleotide and/or polypeptide of the disclosure (e.g., an anti-CD70 antibody of the disclosure, a CD70 CAR of the disclosure). In specific embodiments, the cells are NK cells, T cells, gamma delta T cells, alpha beta T cells, invariant NKT (iNKT) cells, B cells, macrophages, mesenchymal stromal cells (MSCs), or dendritic cells. NK cells may be derived from cord blood, peripheral blood, induced pluripotent stem cells, hematopoietic stem cells, bone marrow, or from a cell line. NK cells may be derived from cord blood mononuclear cells. In some cases, the CD70-positive cells are cancer cells, including from hematopoietic cancers or solid tumors. The cells may be allogeneic or autologous with respect to the individual, who may or may not be a human. The cells may be administered to the individual by injection, intravenously, intraarterially, intraperitoneally, intratracheally, intratumorally, intramuscularly, endoscopically, intralesionally, intracranially, percutaneously, subcutaneously, regionally, by perfusion, in a tumor microenvironment, or a combination thereof.

In particular embodiments of the methods, the cells may be administered to the individual once or more than once. The duration of time between administrations of the cells to the individual may be 1-24 hours, 1-7 days, 1-4 weeks, 1-12 months, or 1 or more years. The methods may further comprise the step of providing to the individual an effective amount of an additional therapy, such as surgery, radiation, gene therapy, immunotherapy, and/or hormone therapy. The additional therapy may comprise one or more antibodies or antibody-based agents, in some cases. In some aspects to the methods, they may further comprising the step of identifying CD70-positive cells in the individual.

Disclosed herein, in some aspects, is an anti-CD70 antibody comprising: (a) a heavy chain variable region (V_{H}) comprising: (i) a CDR-H1 comprising an amino acid sequence having at least 80% sequence identity to SEQ ID NO:45; (ii) a CDR-H2 comprising an amino acid sequence having at least 80% sequence identity to SEQ ID NO:46; and (iii) a CDR-H3 comprising an amino acid sequence having at least 80% sequence identity to SEQ ID NO:47; and (b) a light chain variable region (V_{L}) comprising: (i) a CDR-L1 comprising an amino acid sequence having at least 80% sequence identity to SEQ ID NO:49; (ii) a CDR-L2 comprising an amino acid sequence having at least 80% sequence identity to SEQ ID NO:50; and (iii) a CDR-L3 comprising an amino acid sequence having at least 80% sequence identity to SEQ ID NO:51. In some embodiments, the CDR-H1 comprises SEQ ID NO:45; the CDR-H2 comprises SEQ ID NO:46; and/or the CDR-H3 comprises SEQ ID NO:47. In some embodiments, the CDR-L1 comprises SEQ ID NO:49; the CDR-L2 comprises SEQ ID NO:50; and/or the CDR-L3 comprises SEQ ID NO:51. In some embodiments, the V_{H} comprises an amino acid sequence having at least 85% identity to SEQ ID NO:44; an amino acid sequence having at least 90% identity to SEQ ID NO:44; or an amino acid sequence having at least 95% identity to SEQ ID NO:44. In specific embodiments, the V_{H} comprises SEQ ID NO:44. In some embodiments, the V_{L} comprises an amino acid sequence having at least 85% identity to SEQ ID NO:48; an amino acid sequence having at least 90% identity to SEQ ID NO:48; or an amino acid sequence having at least 95% identity to SEQ ID NO:48. In some embodiments, the V_{L} comprises SEQ ID NO:48.

Disclosed herein, in some aspects, is an anti-CD70 antibody comprising: (a) a V_{H} comprising: (i) a CDR-H1 comprising an amino acid sequence having at least 80% sequence identity to SEQ ID NO:53; (ii) a CDR-H2 comprising an amino acid sequence having at least 80% sequence identity to SEQ ID NO:54; and (iii) a CDR-H3 comprising an amino acid sequence having at least 80% sequence identity to SEQ ID NO:55; and (b) a V_{L} comprising: (i) a CDR-L1 comprising an amino acid sequence having at least 80% sequence identity to SEQ ID NO:57; (ii) a CDR-L2 comprising an amino acid sequence having at least 80% sequence identity to SEQ ID NO:58; and (iii) a CDR-L3 comprising an amino acid sequence having at least 80% sequence identity to SEQ ID NO:59. In some embodiments, the CDR-H1 comprises SEQ ID NO:53; the CDR-H2 comprises SEQ ID NO:54; and/or the CDR-H3 comprises SEQ ID NO:55. In some embodiments, the CDR-L1 comprises SEQ ID NO:57; the CDR-L2 comprises SEQ ID NO:58; and/or the CDR-L3 comprises SEQ ID NO:59. In some embodiments, the V_{H} comprises an amino acid sequence having at least 85% identity to SEQ ID NO:52; an amino acid sequence having at least 90% identity to SEQ ID NO:52; or an amino acid sequence having at least 95% identity to SEQ ID NO:52. In some embodiments, the V_{H} comprises SEQ ID NO:52. In some embodiments, the V_{L} comprises an amino acid sequence having at least 85% identity to SEQ ID NO:56; an amino acid sequence having at least 90% identity to SEQ ID NO:56; or an amino acid sequence having at least 95% identity to SEQ ID NO:56. In some embodiments, the V_{L} comprises SEQ ID NO:56.

Disclosed herein, in some aspects, is an anti-CD70 antibody comprising: (a) a V_{H} comprising: (i) a CDR-H1 comprising an amino acid sequence having at least 80% sequence identity to SEQ ID NO:61; (ii) a CDR-H2 comprising an amino acid sequence having at least 80% sequence identity to SEQ ID NO:62; and (iii) a CDR-H3 comprising an amino acid sequence having at least 80% sequence identity to SEQ ID NO:63; and (b) a V_{L} comprising: (i) a CDR-L1 comprising an amino acid sequence having at least 80% sequence identity to SEQ ID NO:65; (ii) a CDR-L2 comprising an amino acid sequence having at least 80% sequence identity to SEQ ID NO:66; and (iii) a CDR-L3 comprising an amino acid sequence having at least 80% sequence identity to SEQ ID NO:67. In some embodiments, the CDR-H1 comprises SEQ ID NO:61; the CDR-H2 comprises SEQ ID NO:62; and/or the CDR-H3 comprises SEQ ID NO:63. In some embodiments, the CDR-L1 comprises SEQ ID NO:65; the CDR-L2 comprises SEQ ID NO:66; and/or the CDR-L3 comprises SEQ ID NO:67. In some embodiments, the V_{H} comprises an amino acid sequence having at least 85% identity to SEQ ID NO:60; the V_{H} comprises an amino acid sequence having at least 90% identity to SEQ ID NO:60; or the V_{H} comprises an amino acid sequence having at least 95% identity to SEQ ID NO:60. In some embodiments, the V_{H} comprises SEQ ID NO:60. In some embodiments, the V_{L} comprises an amino acid sequence having at least 85% identity to SEQ ID NO:64; the V_{L} comprises an amino acid sequence having at least 90% identity to SEQ ID NO:64; or the V_{L} comprises an amino acid sequence having at least 95% identity to SEQ ID NO:64. In some embodiments, the V_{L} comprises SEQ ID NO:64.

In some embodiments of the anti-CD70 antibodies described herein, the anti-CD70 antibody is encoded by a polynucleotide. In some embodiments, the polynucleotide encoding any one of the anti-CD70 antibodies described herein is comprised in a vector.

Disclosed herein, in some aspects, is a method for generating any one of the anti-CD70 antibodies described herein, comprising (a) providing a polynucleotide encoding for the antibody to a cell and (b) subjecting the cell to conditions sufficient to express the antibody from the polynucleotide.

Disclosed herein, in some aspects, is a pharmaceutical composition comprising: (a) any one of the anti-CD70 antibodies described herein, a polynucleotide encoding any one of the anti-CD70 antibodies described herein, or a vector comprising the polynucleotide encoding any one of the anti-CD70 antibodies described herein; and (b) a pharmaceutically acceptable excipient. In some embodiments, the pharmaceutical composition further comprises an additional therapeutic. In some embodiments, the additional therapeutic is a chemotherapeutic.

Disclosed herein, in some aspects, is a method for treating a subject for cancer, the method comprising administering to the subject a therapeutically effective amount of a composition comprising any one of the anti-CD70 antibodies described herein, a polynucleotide encoding any one of the anti-CD70 antibodies described herein, or a vector comprising the polynucleotide encoding any one of the anti-CD70 antibodies described herein. In some embodiments, the subject has a CD70⁺ cancer. In some embodiments, the subject has lymphoma, leukemia, multiple myeloma, glioblastoma, mesothelioma, head and neck cancers, osteosarcoma, melanoma, non-small cell lung cancer, renal cell carcinoma, pancreatic cancer, ovarian cancer, germ cell tumors, or breast cancer. In some embodiments, the method further comprises administering to the subject an additional therapy. In some embodiments, additional therapy is radiotherapy, chemotherapy, or immunotherapy.

Disclosed herein, in some aspects, is a polynucleotide encoding a CD70-specific engineered receptor, the receptor comprising: (a) an antigen binding region comprising: (i) a V_{H} comprising: (1) a CDR-H1 comprising an amino acid sequence having at least 80% sequence identity to SEQ ID NO:45; (2) a CDR-H2 comprising an amino acid sequence having at least 80% sequence identity to SEQ ID NO:46; and (3) a CDR-H3 comprising an amino acid sequence having at least 80% sequence identity to SEQ ID NO:47; and (ii) a V_{L} comprising: (1) a CDR-L1 comprising an amino acid sequence having at least 80% sequence identity to SEQ ID NO:49; (2) a CDR-L2 comprising an amino acid sequence having at least 80% sequence identity to SEQ ID NO:50; and (3) a CDR-L3 comprising an amino acid sequence having at least 80% sequence identity to SEQ ID NO:51; (b) a transmembrane domain; and (c) an intracellular domain. In some embodiments, the CDR-H1 comprises SEQ ID NO:45; the CDR-H2 comprises SEQ ID NO:46; and/or the CDR-H3 comprises SEQ ID NO:47. In some embodiments, the CDR-L1 comprises SEQ ID NO:49; the CDR-L2 comprises SEQ ID NO:50; and/or the CDR-L3 comprises SEQ ID NO:51. In some embodiments, the V_{H} comprises an amino acid sequence having at least 85% identity to SEQ ID NO:44; an amino acid sequence having at least 90% identity to SEQ ID NO:44; or an amino acid sequence having at least 95% identity to SEQ ID NO:44. In some embodiments, the V_{H} comprises SEQ ID NO:44. In some embodiments, the V_{L} comprises an amino acid sequence having at least 85% identity to SEQ ID NO:48; an amino acid sequence having at least 90% identity to SEQ ID NO:48; or an amino acid sequence having at least 95% identity to SEQ ID NO:48. In some embodiments, the V_{L} comprises SEQ ID NO:48. In some embodiments, the antigen binding region comprises a linker. In some embodiments, the linker comprises SEQ ID NO:74. In some embodiments, the region of the polynucleotide encoding the V_{H} is upstream of the region of the polynucleotide encoding the V_{L} in a 5′ to 3′ direction, and the antigen binding region comprises SEQ ID NO:68. In some embodiments, the region of the polynucleotide encoding the V_{L} is upstream of the region of the polynucleotide encoding the V_{H} in a 5′ to 3′ direction, and the antigen binding region comprises SEQ ID NO:69.

Disclosed herein, in some aspects, is a polynucleotide encoding a CD70-specific engineered receptor, the receptor comprising: (a) an antigen binding region comprising: (i) a V_{H} comprising: (1) a CDR-H1 comprising an amino acid sequence having at least 80% sequence identity to SEQ ID NO:53; (2) a CDR-H2 comprising an amino acid sequence having at least 80% sequence identity to SEQ ID NO:54; and (3) a CDR-H3 comprising an amino acid sequence having at least 80% sequence identity to SEQ ID NO:55; and (ii) a V_{L} comprising: (1) a CDR-L1 comprising an amino acid sequence having at least 80% sequence identity to SEQ ID NO:57; (2) a CDR-L2 comprising an amino acid sequence having at least 80% sequence identity to SEQ ID NO:58 and (3) a CDR-L3 comprising an amino acid sequence having at least 80% sequence identity to SEQ ID NO:59; (b) a transmembrane domain; and (c) an intracellular domain. In some embodiments, the CDR-H1 comprises SEQ ID NO:53; the CDR-H2 comprises SEQ ID NO:54; and/or the CDR-H3 comprises SEQ ID NO:55. In some embodiments, the CDR-L1 comprises SEQ ID NO:57; the CDR-L2 comprises SEQ ID NO:58; and/or the CDR-L3 comprises SEQ ID NO:59. In some embodiments, the V_{H} comprises an amino acid sequence having at least 85% identity to SEQ ID NO:52; an amino acid sequence having at least 90% identity to SEQ ID NO:52; or an amino acid sequence having at least 95% identity to SEQ ID NO:52. In some embodiments, the V_{H} comprises SEQ ID NO:52. In some embodiments, the V_{L} comprises an amino acid sequence having at least 85% identity to SEQ ID NO:56; an amino acid sequence having at least 90% identity to SEQ ID NO:56; or an amino acid sequence having at least 95% identity to SEQ ID NO:56. In some embodiments, the V_{L} comprises SEQ ID NO:56. In some embodiments, the antigen binding region comprises a linker. In some embodiments, the linker comprises SEQ ID NO:74. In some embodiments, the region of the polynucleotide encoding the V_{H} is upstream of the region of the polynucleotide encoding the V_{L} in a 5′ to 3′ direction, and the antigen binding region comprises SEQ ID NO:70. In some embodiments, the region of the polynucleotide encoding the V_{L} is upstream of the region of the polynucleotide encoding the V_{H} in a 5′ to 3′ direction, and the antigen binding region comprises SEQ ID NO:71.

Disclosed herein, in some aspects, is a polynucleotide encoding a CD70-specific engineered receptor, the receptor comprising: (a) an antigen binding region comprising: (i) a V_{H} comprising: (1) a CDR-H1 comprising an amino acid sequence having at least 80% sequence identity to SEQ ID NO:61; (2) a CDR-H2 comprising an amino acid sequence having at least 80% sequence identity to SEQ ID NO:62 and (3) a CDR-H3 comprising an amino acid sequence having at least 80% sequence identity to SEQ ID NO:63; and (ii) a V_{L} comprising: (1) a CDR-L1 comprising an amino acid sequence having at least 80% sequence identity to SEQ ID NO:65; (2) a CDR-L2 comprising an amino acid sequence having at least 80% sequence identity to SEQ ID NO:66; and (3) a CDR-L3 comprising an amino acid sequence having at least 80% sequence identity to SEQ ID NO:67; (b) a transmembrane domain; and (c) an intracellular domain. In some embodiments, the CDR-H1 comprises SEQ ID NO:61; the CDR-H2 comprises SEQ ID NO:62; and/or the CDR-H3 comprises SEQ ID NO:63. In some embodiments, the CDR-L1 comprises SEQ ID NO:65; the CDR-L2 comprises SEQ ID NO:66; and/or the CDR-L3 comprises SEQ ID NO:67. In some embodiments, the V_{H} comprises an amino acid sequence having at least 85% identity to SEQ ID NO:60; an amino acid sequence having at least 90% identity to SEQ ID NO:60; or an amino acid sequence having at least 95% identity to SEQ ID NO:60. In some embodiments, the V_{H} comprises SEQ ID NO:60. In some embodiments, the V_{L} comprises an amino acid sequence having at least 85% identity to SEQ ID NO:64; an amino acid sequence having at least 90% identity to SEQ ID NO:64; or an amino acid sequence having at least 95% identity to SEQ ID NO:64. In some embodiments, the V_{L} comprises SEQ ID NO:64. In some embodiments, the region of the polynucleotide encoding the V_{H} is upstream of the region of the polynucleotide encoding the V_{L} in a 5′ to 3′ direction, and the antigen binding region comprises SEQ ID NO:72. In some embodiments, the region of the polynucleotide encoding the V_{L} is upstream of the region of the polynucleotide encoding the V_{H} in a 5′ to 3′ direction, and the antigen binding region comprises SEQ ID NO:73. In some embodiments of the polynucleotide encoding a CD70-specific engineered receptor described herein, the antigen binding region comprises a linker. In some embodiments, the linker comprises SEQ ID NO:74.

In some embodiments of the polynucleotide encoding a CD70-specific engineered receptor described herein, the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12. In some embodiments, the transmembrane domain is a CD28 transmembrane domain. In some embodiments, the transmembrane domain comprises SEQ ID NO:75.

In some embodiments of the polynucleotide encoding a CD70-specific engineered receptor described herein, the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80. In some embodiments, the intracellular domain is a CD28 intracellular domain. In some embodiments, the intracellular domain is a CD3 zeta intracellular domain. In some embodiments, the intracellular domain comprises SEQ ID NO:81 or SEQ ID NO:82. In some embodiments, the engineered receptor comprises two or more intracellular domains. In some embodiments, the two or more intracellular domains comprise a CD3 zeta intracellular domain and an additional intracellular domain selected from a CD28, DAP10, DAP12, 4-1BB, NKG2D, and 2B4 intracellular domain. In some embodiments, the two or more intracellular domains comprise a CD3 zeta intracellular domain and a CD28 intracellular domain.

In some embodiments of the polynucleotide encoding a CD70-specific engineered receptor described herein, the polynucleotide further comprises a signal peptide. In some embodiments, the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4. In some embodiments, the signal peptide is a CD8 signal peptide.

In some embodiments of the polynucleotide encoding a CD70-specific engineered receptor described herein, the polynucleotide further comprises a hinge between the antigen binding domain and the transmembrane domain. In some embodiments, the hinge is an IgG hinge, a CD28 hinge, or a CD8a hinge. In some embodiments, the hinge is IgG1 hinge, IgG2 hinge, IgG3 hinge, or IgG4 hinge. In some embodiments, the hinge is an IgG1 hinge.

In some embodiments of the polynucleotide encoding a CD70-specific engineered receptor described herein, the polynucleotide further encodes an additional polypeptide. In some embodiments, the additional polypeptide is a therapeutic protein or a protein that enhances cell activity, expansion, and/or persistence. In some embodiments, the additional polypeptide is a suicide gene, a cytokine, or a human or viral protein that enhances proliferation, expansion and/or metabolic fitness. In some embodiments, the additional polypeptide is a cytokine. In some embodiments, the cytokine is IL-15, IL-2, IL-12, IL-18, IL-21, IL-23, or IL-7. In some embodiments, the cytokine is IL-15.

In some embodiments of the polynucleotide encoding a CD70-specific engineered receptor described herein, the CD70-specific engineered receptor is a chimeric antigen receptor (CAR). In some embodiments of the polynucleotide encoding a CD70-specific engineered receptor described herein, the CD70-specific engineered receptor is a T cell receptor.

In some embodiments of the polynucleotide encoding a CD70-specific engineered receptor described herein, the polynucleotide is comprised in a vector. In some embodiments, the vector is a viral vector. In some embodiments, the viral vector is an adenoviral vector, adeno-associated viral vector, lentiviral vector, or retroviral vector. In some embodiments, the vector is a non-viral vector. In some embodiments, the non-viral vector is a plasmid.

In some embodiments of the polynucleotide encoding a CD70-specific engineered receptor described herein, the polynucleotide or a vector comprising the polynucleotide is comprised in an immune cell. In some embodiments, the immune cell is a natural killer (NK) cell, T cell, gamma delta T cell, alpha beta T cell, invariant NKT (iNKT) cell, B cell, macrophage, mesenchymal stromal cell, or dendritic cell. In some embodiments, the immune cell is an NK cell. In some embodiments, the NK cell is derived from cord blood, peripheral blood, induced pluripotent stem cells, hematopoietic stem cells, bone marrow, or from a cell line. In some embodiments, the NK cell is derived from a cell line, wherein the NK cell line is NK-92. In some embodiments, the NK cell is derived from a cord blood mononuclear cell. In some embodiments, the NK cell is a CD56⁺ NK cell. In some embodiments, the NK cell expresses a recombinant cytokine. In some embodiments, the cytokine is IL-15, IL-2, IL-12, IL-18, IL-21, IL-7, or IL-23. In some embodiments, the cytokine is IL-15. Also disclosed herein is a population of immune cells comprising the immune cells comprising a polynucleotide encoding a CD70-specific engineered receptor described herein or a vector comprising the polynucleotide.

Disclosed herein, in some aspects, is a method of killing CD70-positive cells in an individual, comprising administering to the individual an effective amount of cells harboring a polynucleotide encoding a CD70-specific engineered receptor described herein.

Disclosed herein, in some aspects, is a method for treating a subject for cancer, the method comprising administering to the subject a therapeutically effective amount of an immune cell comprising a polynucleotide encoding a CD70-specific engineered receptor described herein or a vector comprising the polynucleotide, or a population of immune cells comprising the immune cells comprising a polynucleotide encoding a CD70-specific engineered receptor described herein or a vector comprising the polynucleotide. In some embodiments, the subject has a CD70⁺ cancer. In some embodiments, the subject has lymphoma, leukemia, multiple myeloma, glioblastoma, mesothelioma, head and neck cancers, osteosarcoma, melanoma, non-small cell lung cancer, renal cell carcinoma, pancreatic cancer, ovarian cancer, germ cell tumors, or breast cancer. In some embodiments, the method further comprises administering to the subject an additional therapy. In some embodiments, the additional therapy is radiotherapy, chemotherapy, or immunotherapy.

Disclosed herein, in some aspects, is a pharmaceutical composition comprising: (a) an immune cell comprising a polynucleotide encoding a CD70-specific engineered receptor described herein or a vector comprising the polynucleotide encoding a CD70-specific engineered receptor described herein, or a population of immune cells comprising immune cells comprising a polynucleotide encoding a CD70-specific engineered receptor described herein or a vector comprising the polynucleotide encoding a CD70-specific engineered receptor described herein; and (b) a pharmaceutically acceptable excipient. In some embodiments, the pharmaceutical composition further comprises an additional therapeutic. In some embodiments, the additional therapeutic is a chemotherapeutic.

Disclosed herein, in some aspects, is an immune cell engager comprising: (a) a CD70-binding region comprising: (i) a V_{H} comprising: (1) a CDR-H1 comprising an amino acid sequence having at least 80% sequence identity to SEQ ID NO:45; (2) a CDR-H2 comprising an amino acid sequence having at least 80% sequence identity to SEQ ID NO:46; and (3) a CDR-H3 comprising an amino acid sequence having at least 80% sequence identity to SEQ ID NO:47; and (ii) a V_{L} comprising: (1) a CDR-L1 comprising an amino acid sequence having at least 80% sequence identity to SEQ ID NO:49; (2) a CDR-L2 comprising an amino acid sequence having at least 80% sequence identity to SEQ ID NO:50; and (3) a CDR-L3 comprising an amino acid sequence having at least 80% sequence identity to SEQ ID NO:51; and (b) an immune cell binding region. In some embodiments, the CDR-H1 comprises SEQ ID NO:45; the CDR-H2 comprises SEQ ID NO:46; and/or the CDR-H3 comprises SEQ ID NO:47. In some embodiments, the CDR-L1 comprises SEQ ID NO:49; the CDR-L2 comprises SEQ ID NO:50; and/or the CDR-L3 comprises SEQ ID NO:51. In some embodiments, the V_{H} comprises an amino acid sequence having at least 85% identity to SEQ ID NO:44; an amino acid sequence having at least 90% identity to SEQ ID NO:44; or an amino acid sequence having at least 95% identity to SEQ ID NO:44. In some embodiments, the V_{H} comprises SEQ ID NO:44. In some embodiments, the V_{L} comprises an amino acid sequence having at least 85% identity to SEQ ID NO:48; an amino acid sequence having at least 90% identity to SEQ ID NO:48; or an amino acid sequence having at least 95% identity to SEQ ID NO:48. In some embodiments, the V_{L} comprises SEQ ID NO:48. In some embodiments, the CD70-binding region comprises a linker. In some embodiments, the linker comprises SEQ ID NO:74. In some embodiments, the V_{H} of the CD70-binding region is upstream of the V_{L} of the CD70-binding region in a 5′ to 3′ direction, and the CD70-binding region comprises SEQ ID NO:68. In some embodiments, the V_{L} of the CD70-binding region is upstream of the V_{H} of the CD70-binding region in a 5′ to 3′ direction, and the CD70-binding region comprises SEQ ID NO:69.

Disclosed herein, in some aspects, is an immune cell engager comprising: (a) a CD70-binding region comprising: (i) a V_{H} comprising: (1) a CDR-H1 comprising an amino acid sequence having at least 80% sequence identity to SEQ ID NO:53; (2) a CDR-H2 comprising an amino acid sequence having at least 80% sequence identity to SEQ ID NO:54; and (3) a CDR-H3 comprising an amino acid sequence having at least 80% sequence identity to SEQ ID NO:55; and (ii) a V_{L} comprising: (1) a CDR-L1 comprising an amino acid sequence having at least 80% sequence identity to SEQ ID NO:57; (2) a CDR-L2 comprising an amino acid sequence having at least 80% sequence identity to SEQ ID NO:58; and (3) a CDR-L3 comprising an amino acid sequence having at least 80% sequence identity to SEQ ID NO:59; and (b) an immune cell binding region. In some embodiments, the CDR-H1 comprises SEQ ID NO:53; the CDR-H2 comprises SEQ ID NO:54; and/or the CDR-H3 comprises SEQ ID NO:55. In some embodiments, the CDR-L1 comprises SEQ ID NO:57; the CDR-L2 comprises SEQ ID NO:58; and/or the CDR-L3 comprises SEQ ID NO:59. In some embodiments, the V_{H} comprises an amino acid sequence having at least 85% identity to SEQ ID NO:52; an amino acid sequence having at least 90% identity to SEQ ID NO:52; or an amino acid sequence having at least 95% identity to SEQ ID NO:52. In some embodiments, the V_{H} comprises SEQ ID NO:52. In some embodiments, the V_{L} comprises an amino acid sequence having at least 85% identity to SEQ ID NO:56; an amino acid sequence having at least 90% identity to SEQ ID NO:56; or an amino acid sequence having at least 95% identity to SEQ ID NO:56. In some embodiments, the V_{L} comprises SEQ ID NO:56. In some embodiments, the CD70-binding region comprises a linker. In some embodiments, the linker comprises SEQ ID NO:74. In some embodiments, the V_{H} of the CD70-binding region is upstream of the V_{L} of the CD70-binding region in a 5′ to 3′ direction, and the CD70-binding region comprises SEQ ID NO:70. In some embodiments, the V_{L} of the CD70-binding region is upstream of the V_{H} of the CD70-binding region in a 5′ to 3′ direction, and the CD70-binding region comprises SEQ ID NO:71.

Disclosed herein, in some aspects, is an immune cell engager comprising: (a) a CD70-binding region comprising: (i) a V_{H} comprising: (1) a CDR-H1 comprising an amino acid sequence having at least 80% sequence identity to SEQ ID NO:61; (2) a CDR-H2 comprising an amino acid sequence having at least 80% sequence identity to SEQ ID NO:62; and (3) a CDR-H3 comprising an amino acid sequence having at least 80% sequence identity to SEQ ID NO:63; and (ii) a V_{L} comprising: (1) a CDR-L1 comprising an amino acid sequence having at least 80% sequence identity to SEQ ID NO:65 (2) a CDR-L2 comprising an amino acid sequence having at least 80% sequence identity to SEQ ID NO:66; and (3) a CDR-L3 comprising an amino acid sequence having at least 80% sequence identity to SEQ ID NO:67; and (b) an immune cell binding region. In some embodiments, the CDR-H1 comprises SEQ ID NO:61; the CDR-H2 comprises SEQ ID NO:62; and/or the CDR-H3 comprises SEQ ID NO:63. In some embodiments, the CDR-L1 comprises SEQ ID NO:65; the CDR-L2 comprises SEQ ID NO:66; and/or the CDR-L3 comprises SEQ ID NO:67. In some embodiments, the V_{H} comprises an amino acid sequence having at least 85% identity to SEQ ID NO:60; an amino acid sequence having at least 90% identity to SEQ ID NO:60; or an amino acid sequence having at least 95% identity to SEQ ID NO:60. In some embodiments, the V_{H} comprises SEQ ID NO:60. In some embodiments, the V_{L} comprises an amino acid sequence having at least 85% identity to SEQ ID NO:64; an amino acid sequence having at least 90% identity to SEQ ID NO:64; or an amino acid sequence having at least 95% identity to SEQ ID NO:64. In some embodiments, the V_{L} comprises SEQ ID NO:64. In some embodiments, the CD70-binding region comprises a linker. In some embodiments, the linker comprises SEQ ID NO:74. In some embodiments, the V_{H} of the CD70-binding region is upstream of the V_{L} of the CD70-binding region in a 5′ to 3′ direction, and the antigen binding region comprises SEQ ID NO:72. In some embodiments, the V_{L} of the CD70-binding region is upstream of the V_{H} of the CD70-binding region in a 5′ to 3′ direction, and the antigen binding region comprises SEQ ID NO:73.

In some embodiments of the immune cell engagers disclosed herein, the immune cell binding region specifically binds to a protein expressed on a surface of an immune cell. In some embodiments, the immune cell is an NK cell, T cell, gamma delta T cell, alpha beta T cell, iNKT cell, B cell, macrophage, mesenchymal stromal cell, or dendritic cell. In some embodiments, the immune cell is a T cell. In some embodiments, the immune cell binding region specifically binds to CD3, the T cell receptor (TCR), CD28, Ox40, 4-1 BB, CD2, CD5, CD95, CD27, IL-7R, ICOS, IL2Rβ, CD45, CD48, and CD137. In some embodiments, the immune cell binding region specifically binds to CD3. In some embodiments, the immune cell binding region comprises an scFv from an anti-CD3 antibody. In some embodiments, the immune cell is an NK cell. In some embodiments, the immune cell binding region specifically binds to CD16A, NKp46, or NKG2D. In some embodiments, the immune cell binding region specifically binds to CD16A. In some embodiments, the immune cell binding region comprises an scFv from an anti-CD16A antibody.

In some embodiments of the immune cell engagers described herein, the immune cell engager is encoded by a polynucleotide. In some embodiments, a polynucleotide encoding an immune cell engager described herein is comprised in a vector.

Disclosed herein, in some aspects, is a method for generating an immune cell engager described herein, comprising (a) providing a polynucleotide encoding for the immune cell engager to a cell and (b) subjecting the cell to conditions sufficient to express the immune cell engager from the polynucleotide.

Disclosed herein, in some aspects, is a pharmaceutical composition comprising: (a) an immune cell engager described herein, a polynucleotide encoding an immune cell engager described herein, or a vector comprising a polynucleotide encoding an immune cell engager described herein; and (b) a pharmaceutically acceptable excipient. In some embodiments, the pharmaceutical composition further comprises an additional therapeutic. In some embodiments, the additional therapeutic is a chemotherapeutic.

Disclosed herein, in some aspects, is a method for treating a subject for cancer, the method comprising administering to the subject a therapeutically effective amount of a composition comprising an immune cell engager described herein, a polynucleotide encoding an immune cell engager described herein, or a vector comprising a polynucleotide encoding an immune cell engager described herein. In some embodiments, the subject has a CD70⁺ cancer. In some embodiments, the subject has lymphoma, leukemia, multiple myeloma, glioblastoma, mesothelioma, head and neck cancers, osteosarcoma, melanoma, non-small cell lung cancer, renal cell carcinoma, pancreatic cancer, ovarian cancer, germ cell tumors, or breast cancer. In some embodiments, the method further comprises administering to the subject an additional therapy. In some embodiments, the additional therapy is radiotherapy, chemotherapy, or immunotherapy.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Any embodiment discussed with respect to one aspect of the disclosure applies to other aspects of the disclosure as well and vice versa. For example, any step in a method described herein can apply to any other method. Moreover, any method described herein may have an exclusion of any step or combination of steps. Aspects of an embodiment set forth in the Examples are also embodiments that may be implemented in the context of embodiments discussed elsewhere in a different Example or elsewhere in the application, such as in the Summary, Detailed Description, Claims, and Brief Description of the Drawings.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows results from an ELISA cell-based assay for monoclonal anti-human CD70 antibodies.

FIG. 2 provides validation of CD70 antibody clones binding to CD70 antigen on the cell surface.

FIGS. 3A-3B show schematics of vector maps containing a CD70 CAR, which is generated from the scFv sequence of m6-CD70 antibody clone.

FIGS. 4A-4B show schematics of vector maps containing a CD70 CAR, which is generated from the scFv sequence of m7-CD70 antibody clone.

FIGS. 5A-5B show schematics of vector maps containing a CD70 CAR, which is generated from the scFv sequence of m14-CD70 antibody clone.

FIGS. 6A-6B show the transduction efficiency of CBNK cells transduced with various CD70 CAR constructs.

FIG. 7 shows results from a cell proliferation assay of CBNK cells transduced with various CD70 CAR constructs.

FIG. 8 shows CD107a expression in CBNK cells transduced with various CD70 constructs when co-cultured with various cancer cells.

FIG. 9 shows interferon gamma production in CBNK cells transduced with various CD70 constructs when co-cultured with various cancer cells.

FIG. 10 shows tumor necrosis factor alpha production in CBNK cells transduced with various CD70 constructs when co-cultured with various cancer cells.

FIGS. 11A-11B show results from a chromium release assay to assess the cytotoxic activity of CBNK cells transduced with various CD70 CAR constructs against Raji and Karpas cells.

FIGS. 12A-12B show that CD70 CAR CBNK cells reduced the tumor burden in a mouse model of multiple myeloma (MM1.s).

FIGS. 13A-13B show that CD70 CAR CBNK cells reduced tumor burden in a mouse model of acute myeloid leukemia (MOLM-14).

FIGS. 14A-14B show that CD70 CAR T cells generated from the m14-CD70 clone was better at reducing tumor burden in a mouse model of acute myeloid leukemia (MOLM-14), compared to CD70 CAR T cells generated from ARGX-110.

FIGS. 15A-15B show results from a chromium release assay to assess the cytotoxic activity of T cells transduced with CD70 CAR constructs against Raji and Mec-1 cells.

FIG. 16 shows that CD70 CAR T cells improved cytotoxicity against SKOV3 cells when compared to non-transduced T cells.

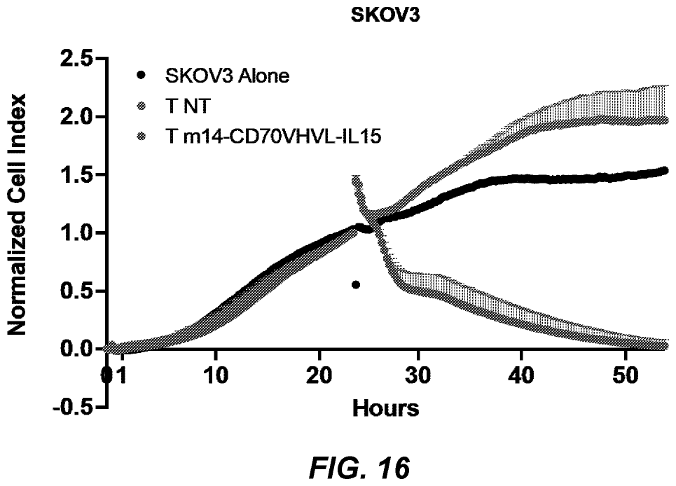

### DETAILED DESCRIPTION

The present disclosure is based, at least in part, on the development of CD70 binding polypeptides, including scFvs, portions thereof, and various polypeptides (e.g., antibodies, CARs, engagers) comprising such scFvs or portions thereof. Accordingly, provided herein, in certain embodiments, are methods and compositions concerning antibodies, antibody fragments, and engineered polypeptides for therapy to target cancers including CD70-positive cancer. Certain aspects of the present disclosure are directed to anti-CD70 antibodies and therapeutic methods of use. Further aspects of the present disclosure are directed to CD70-targeted polypeptides (e.g., chimeric antigen receptors or T cell receptors) and therapeutic methods of use. Also disclosed, in certain embodiments, are immune cell engagers (e.g., bispecific engagers) targeting CD70 and one or more immune cell targets (e.g., CD3, CD16A, NCRs). Additionally, described are methods for cancer treatment comprising use of CD70-targeted polypeptides of the disclosure and cells comprising such polypeptides.

### I. Examples of Definitions

In keeping with long-standing patent law convention, the words “a” and “an” when used in the present specification in concert with the word comprising, including the claims, may mean “one” but is also consistent with the meaning of “one or more,” “at least one,” and “one or more than one.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Any method in the context of a therapeutic, diagnostic, or physiologic purpose or effect may also be described in “use” claim language such as “use of” any compound, composition, or agent discussed herein for achieving or implementing a described therapeutic, diagnostic, or physiologic purpose or effect.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate a deviation of 10% of the value(s) to which it is attached.

Recitation of ranges of values herein is merely intended to serve as a shorthand method of referring individually to each separate value falling within the range. Unless otherwise indicated herein, each individual value is incorporated into the specification as if it was individually recited herein.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

The term “isolated” as used herein refers to molecules or biologicals or cellular materials being substantially free from other materials. In one aspect, the term “isolated” refers to nucleic acid, such as DNA or RNA, or protein or polypeptide, or cell or cellular organelle, or tissue or organ, separated from other DNAs or RNAs, or proteins or polypeptides, or cells or cellular organelles, or tissues or organs, respectively, such as that are present in the natural source. The term “isolated” also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to polypeptides that are isolated from other cellular proteins and is meant to encompass both purified and recombinant polypeptides. The term “isolated” is also used herein to refer to cells or tissues that are isolated from other cells or tissues and is meant to encompass both cultured and engineered cells or tissues.

“Sequence identity” between two amino acid sequences indicates the percentage of amino acids that are identical between the sequences. “Sequence identity” between two nucleic acid sequences indicates the percentage of nucleotides that are identical between the sequences.

The terms “% identical,” “% identity,” or similar terms are intended to refer, in particular, to the percentage of nucleotides or amino acids which are identical in an optimal alignment between the sequences to be compared. Said percentage is purely statistical, and the differences between the two sequences may be but are not necessarily randomly distributed over the entire length of the sequences to be compared. Comparisons of two sequences are usually carried out by comparing the sequences, after optimal alignment, with respect to a segment or “window of comparison,” in order to identify local regions of corresponding sequences. The optimal alignment for a comparison may be carried out manually or with the aid of the local homology algorithm by Smith and Waterman, 1981, Ads App. Math. 2, 482, with the aid of the local homology algorithm by Neddleman and Wunsch, 1970, J. Mol. Biol. 48, 443, with the aid of the similarity search algorithm by Pearson and Lipman, 1988, Proc. Natl Acad. Sci. USA 88, 2444, or with the aid of computer programs using said algorithms (GAP, BESTFIT, FASTA, BLAST P, BLAST N, and TFASTA in Wisconsin Genetics Software Package, Genetics Computer Group). In some aspects, percent identity of two sequences is determined using the BLASTN or BLASTP algorithm, as available on the United States National Center for Biotechnology Information (NCBI) website.

Percentage identity is obtained by determining the number of identical positions at which the sequences to be compared correspond, dividing this number by the number of positions compared (e.g., the number of positions in the reference sequence) and multiplying this result by 100.

In some aspects, the degree of similarity or identity is given for a region that is at least, at most, exactly, or between any two of about 50%, about 60%, about 70%, about 80%, about 90%, or about 100% of the entire length of the reference sequence. For example, if the reference nucleic acid sequence consists of 200 nucleotides, the degree of identity is given for at least, at most, exactly, or between any two of about 100, about 120, about 140, about 160, about 180, or about 200 nucleotides, in some aspects, continuous nucleotides. In some aspects, the degree of similarity or identity is given for the entire length of the reference sequence.

As used herein, “prevent,” and similar words such as “prevented,” “preventing,” etc., indicate an approach for preventing, inhibiting, or reducing the likelihood of the occurrence or recurrence of a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms, and/or burden of a disease or condition prior to onset or recurrence of the disease or condition.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

The terms “inhibiting,” “decreasing,” or “reducing” or any variation of these terms includes any measurable decrease (e.g., a 5%, 10%, 15%, 20%, 25%, 30%, 35%, 40%, 45%, 50%, 55%, 60%, 65%, 70%, 75%, 80%, 85%, 90%, 95%, or 99% decrease) or complete inhibition to achieve a desired result. The terms “improve,” “promote,” or “increase” or any variation of these terms includes any measurable increase (e.g., a 5%, 10%, 15%, 20%, 25%, 30%, 35%, 40%, 45%, 50%, 55%, 60%, 65%, 70%, 75%, 80%, 85%, 90%, 95%, or 99% increase) to achieve a desired result or production of a protein or molecule.

As will be understood from context, “risk” of a disease, disorder, and/or condition refers to a likelihood that a particular individual will develop the disease, disorder, and/or condition. In some embodiments, risk is expressed as a percentage. In some embodiments, risk is, is at least, or is at most from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, risk may reflect one or more genetic attributes, e.g., which may predispose an individual toward development (or not) of a particular disease, disorder and/or condition. In some embodiments, risk may reflect one or more epigenetic events or attributes and/or one or more lifestyle or environmental events or attributes. Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

The term “sample,” as used herein, generally refers to a biological sample. The sample may be taken from tissue or cells from an individual. In some examples, the sample may comprise, or be derived from, a tissue biopsy, blood (e.g., whole blood), blood plasma, extracellular fluid, dried blood spots, cultured cells, discarded tissue. The sample may have been isolated from the source prior to collection. Non-limiting examples include blood, cerebral spinal fluid, pleural fluid, amniotic fluid, lymph fluid, saliva, urine, stool, tears, sweat, or mucosal excretions, and other bodily fluids isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may be treated with RNases. The sample may contain tissues or cells that are intact, fragmented, or partially degraded.

The term “subject,” as used herein, generally refers to an individual having a biological sample that is undergoing processing or analysis and, in specific cases, has or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as benign or malignant neoplasias, or cancer. The subject may being undergoing or having undergone treatment. The subject may be asymptomatic. The subject may be healthy individuals but that are desirous of prevention of cancer. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies.

### II. Antibodies

Aspects of the disclosure relate to anti-CD70 antibodies and fragments thereof. The term “antibody” refers to an intact immunoglobulin of any isotype, or a fragment thereof that can compete with the intact antibody for specific binding to the target antigen, and includes chimeric, humanized, fully human, and bispecific antibodies. As used herein, the terms “antibody” or “immunoglobulin” are used interchangeably and refer to any of several classes of structurally related proteins that function as part of the immune response of an animal, including IgG, IgD, IgE, IgA, IgM, and related proteins, as well as polypeptides comprising antibody CDR domains that retain antigen-binding activity.

The term “antigen” refers to a molecule or a portion of a molecule capable of being bound by a selective binding agent, such as an antibody. An antigen may possess one or more epitopes that are capable of interacting with different antibodies.

The term “epitope” includes any region or portion of a molecule capable eliciting an immune response by binding to an immunoglobulin or to a T-cell receptor. Epitope determinants may include chemically active surface groups such as amino acids, sugar side chains, phosphoryl or sulfonyl groups, and may have specific three-dimensional structural characteristics and/or specific charge characteristics. Generally, antibodies specific for a particular target antigen will preferentially recognize an epitope on the target antigen within a complex mixture.

The epitope regions of a given polypeptide can be identified using many different epitope mapping techniques are well known in the art, including: x-ray crystallography, nuclear magnetic resonance spectroscopy, site-directed mutagenesis mapping, protein display arrays, see, e.g., Epitope Mapping Protocols, (Johan Rockberg and Johan Nilvebrant, Ed., 2018) Humana Press, New York, N.Y. Such techniques are known in the art and described in, e.g., U.S. Pat. No. 4,708,871; Geysen et al. Proc. Natl. Acad. Sci. USA 81:3998-4002 (1984); Geysen et al. Proc. Natl. Acad. Sci. USA 82:178-182 (1985); Geysen et al. Molec. Immunol. 23:709-715 (1986 See, e.g., Epitope Mapping Protocols, supra. Additionally, antigenic regions of proteins can also be predicted and identified using standard antigenicity and hydropathy plots.

An intact antibody is generally composed of two full-length heavy chains and two full-length light chains, but in some instances may include fewer chains, such as antibodies naturally occurring in camelids that may comprise only heavy chains. Antibodies as disclosed herein may be derived solely from a single source or may be “chimeric,” that is, different portions of the antibody may be derived from two different antibodies. For example, the variable or CDR regions may be derived from a rat or murine source, while the constant region is derived from a different animal source, such as a human. The antibodies or binding fragments may be produced in hybridomas, by recombinant DNA techniques, or by enzymatic or chemical cleavage of intact antibodies. Unless otherwise indicated, the term “antibody” includes derivatives, variants, fragments, and muteins thereof, examples of which are described below (Sela-Culang et al. Front Immunol. 2013; 4: 302; 2013)

The term “light chain” includes a full-length light chain and fragments thereof having sufficient variable region sequence to confer binding specificity. A full-length light chain may have a molecular weight of around 25,000 Daltons and includes a variable region domain (abbreviated herein as V_{L}), and a constant region domain (abbreviated herein as CL). There are two classifications of light chains, identified as kappa (x) and lambda (k). The term “V_{L} fragment” means a fragment of the light chain of a monoclonal antibody that includes all or part of the light chain variable region, including CDRs. A V_{L} fragment can further include light chain constant region sequences. The variable region domain of the light chain is at the amino-terminus of the polypeptide.

The term “heavy chain” includes a full-length heavy chain and fragments thereof having sufficient variable region sequence to confer binding specificity. A full-length heavy chain may have a molecular weight of around 50,000 Daltons and includes a variable region domain (abbreviated herein as V_{H}), and three constant region domains (abbreviated herein as C_{H}1, C_{H}2, and C_{H}3). The term “V_{H} fragment” means a fragment of the heavy chain of a monoclonal antibody that includes all or part of the heavy chain variable region, including CDRs. A V_{H} fragment can further include heavy chain constant region sequences. The number of heavy chain constant region domains will depend on the isotype. The V_{H} domain is at the amino-terminus of the polypeptide, and the C_{H} domains are at the carboxy-terminus, with the C_{H}3 being closest to the —COH end. The isotype of an antibody can be IgM, IgD, IgG, IgA, or IgE and is defined by the heavy chains present of which there are five classifications: mu (μ), delta (δ), gamma (γ), alpha (α), or epsilon (ε) chains, respectively. IgG has several subtypes, including, but not limited to, IgG1, IgG2, IgG3, and IgG4. IgM subtypes include IgM1 and IgM2. IgA subtypes include IgA1 and IgA2.

Antibodies can be whole immunoglobulins of any isotype or classification, chimeric antibodies, or hybrid antibodies with specificity to two or more antigens. They may also be fragments (e.g., F(ab′)2, Fab′, Fab, Fv, and the like), including hybrid fragments. An immunoglobulin also includes natural, synthetic, or genetically engineered proteins that act like an antibody by binding to specific antigens to form a complex. The term antibody includes genetically engineered or otherwise modified forms of immunoglobulins, such as those described elsewhere herein.

The term “monomer” means an antibody containing only one Ig unit. Monomers are the basic functional units of antibodies. The term “dimer” means an antibody containing two Ig units attached to one another via constant domains of the antibody heavy chains (the Fc, or fragment crystallizable, region). The complex may be stabilized by a joining (J) chain protein. The term “multimer” means an antibody containing more than two Ig units attached to one another via constant domains of the antibody heavy chains (the Fc region). The complex may be stabilized by a joining (J) chain protein.

The term “bivalent antibody” means an antibody that comprises two antigen-binding sites. The two binding sites may have the same antigen specificities or they may be bispecific, meaning the two antigen-binding sites have different antigen specificities.

Bispecific antibodies are a class of antibodies that have two paratopes with different binding sites for two or more distinct epitopes. In some embodiments, bispecific antibodies can be biparatopic, wherein a bispecific antibody may specifically recognize a different epitope from the same antigen. In some embodiments, bispecific antibodies can be constructed from a pair of different single domain antibodies termed “nanobodies”. Single domain antibodies are sourced and modified from cartilaginous fish and camelids. Nanobodies can be joined together by a linker using techniques typical to a person skilled in the art; such methods for selection and joining of nanobodies are described in PCT Publication No. WO2015044386A1, No. WO2010037838A2, and Bever et al., Anal Chem. 86:7875-7882 (2014), each of which are specifically incorporated herein by reference in their entirety.

Bispecific antibodies can be constructed as: a whole IgG, Fab′2, Fab′PEG, a diabody, or alternatively as scFv. Diabodies and scFvs can be constructed without an Fc region, using only variable domains, potentially reducing the effects of anti-idiotypic reaction. Bispecific antibodies may be produced by a variety of methods including, but not limited to, fusion of hybridomas or linking of Fab′ fragments. See, e.g., Songsivilai and Lachmann, Clin. Exp. Immunol. 79:315-321 (1990); Kostelny et al., J. Immunol. 148:1547-1553 (1992), each of which are specifically incorporated by reference in their entirety.

In certain aspects, the antigen-binding domain may be multispecific or heterospecific by multimerizing with V_{H} and V_{L} region pairs that bind a different antigen. For example, the antibody may bind to, or interact with, (a) a cell surface antigen, (b) an Fc receptor on the surface of an effector cell, or (c) at least one other component. Accordingly, aspects may include, but are not limited to, bispecific, trispecific, tetraspecific, and other multispecific antibodies or antigen-binding fragments thereof that are directed to epitopes and to other targets, such as Fc receptors on effector cells.

In some embodiments, multispecific antibodies can be used and directly linked via a short flexible polypeptide chain, using routine methods known in the art. One such example is diabodies that are bivalent, bispecific antibodies in which the V_{H} and V_{L} domains are expressed on a single polypeptide chain, and utilize a linker that is too short to allow for pairing between domains on the same chain, thereby forcing the domains to pair with complementary domains of another chain creating two antigen binding sites. The linker functionality is applicable for embodiments of triabodies, tetrabodies, and higher order antibody multimers. (see, e.g., Hollinger et al., Proc Natl. Acad. Sci. USA 90:6444-6448 (1993); Polijak et al., Structure 2:1121-1123 (1994); Todorovska et al., J. Immunol. Methods 248:47-66 (2001)).

Bispecific diabodies, as opposed to bispecific whole antibodies, may also be advantageous because they can be readily constructed and expressed in E. coli. Diabodies (and other polypeptides such as antibody fragments) of appropriate binding specificities can be readily selected using phage display (WO94/13804) from libraries. If one arm of the diabody is kept constant, for instance, with a specificity directed against a protein, then a library can be made where the other arm is varied and an antibody of appropriate specificity selected. Bispecific whole antibodies may be made by alternative engineering methods as described in Ridgeway et al., (Protein Eng., 9:616-621, 1996) and Krah et al., (N Biotechnol. 39:167-173, 2017), each of which is hereby incorporated by reference in their entirety.

Heteroconjugate antibodies are composed of two covalently linked monoclonal antibodies with different specificities. See, e.g., U.S. Pat. No. 6,010,902, incorporated herein by reference in its entirety.

The part of the Fv fragment of an antibody molecule that binds with high specificity to the epitope of the antigen is referred to herein as the “paratope.” The paratope consists of the amino acid residues that make contact with the epitope of an antigen to facilitate antigen recognition. Each of the two Fv fragments of an antibody is composed of the two variable domains, V_{H} and V_{L}, in dimerized configuration. The primary structure of each of the variable domains includes three hypervariable loops separated by, and flanked by, Framework Regions (FR). The hypervariable loops are the regions of highest primary sequences variability among the antibody molecules from any mammal. The term hypervariable loop is sometimes used interchangeably with the term “Complementarity Determining Region (CDR).” The length of the hypervariable loops (or CDRs) varies between antibody molecules. The framework regions of all antibody molecules from a given mammal have high primary sequence similarity/consensus. The consensus of framework regions can be used by one skilled in the art to identify both the framework regions and the hypervariable loops (or CDRs) which are interspersed among the framework regions. The hypervariable loops are given identifying names which distinguish their position within the polypeptide, and on which domain they occur. CDRs in the V_{L} domain are identified as L1 (also CDR-L1), L2 (also CDR-L2), and L3 (also CDR-L3), with L1 occurring at the most distal end and L3 occurring closest to the CL domain. The CDRs may also be given the names CDR-1, CDR-2, and CDR-3. The L3 (CDR-3) is generally the region of highest variability among all antibody molecules produced by a given organism. The CDRs are regions of the polypeptide chain arranged linearly in the primary structure, and separated from each other by Framework Regions. The amino terminal (N-terminal) end of the V_{L} chain is named FR1. The region identified as FR2 occurs between L1 and L2 hypervariable loops. FR3 occurs between L2 and L3 hypervariable loops, and the FR4 region is closest to the CL domain. This structure and nomenclature is repeated for the V_{H} chain, which includes three CDRs identified as H1 (CDR-H1), H2 (CDR-H2), and H3 (CDR-H3). The majority of amino acid residues in the variable domains, or Fv fragments (V_{H} and V_{L}), are part of the framework regions (approximately 85%). The three dimensional, or tertiary, structure of an antibody molecule is such that the framework regions are more internal to the molecule and provide the majority of the structure, with the CDRs on the extrenal surface of the molecule.

Several methods have been developed and can be used by one skilled in the art to identify the exact amino acids that constitute each of these regions. This can be done using any of a number of multiple sequence alignment methods and algorithms, which identify the conserved amino acid residues that make up the framework regions, therefore identifying the CDRs that may vary in length but are located between framework regions. Three commonly used methods have been developed for identification of the CDRs of antibodies: Kabat (as described in T. T. Wu and E. A. Kabat, “AN ANALYSIS OF THE SEQUENCES OF THE VARIABLE REGIONS OF BENCE JONES PROTEINS AND MYELOMA LIGHT CHAINS AND THEIR IMPLICATIONS FOR ANTIBODY COMPLEMENTARITY,” J Exp Med, vol. 132, no. 2, pp. 211-250, August 1970); Chothia (as described in C. Chothia et al., “Conformations of immunoglobulin hypervariable regions,” Nature, vol. 342, no. 6252, pp. 877-883, December 1989); and IMGT (as described in M.-P. Lefranc et al., “IMGT unique numbering for immunoglobulin and T cell receptor variable domains and Ig superfamily V-like domains,” Developmental & Comparative Immunology, vol. 27, no. 1, pp. 55-77, January 2003). These methods each include unique numbering systems for the identification of the amino acid residues that constitute the variable regions. In most antibody molecules, the amino acid residues that actually contact the epitope of the antigen occur in the CDRs, although in some cases, residues within the framework regions contribute to antigen binding.

One skilled in the art can use any of several methods to determine the paratope of an antibody. These methods include: 1) Computational predictions of the tertiary structure of the antibody/epitope binding interactions based on the chemical nature of the amino acid sequence of the antibody variable region and composition of the epitope; 2) Hydrogen-deuterium exchange and mass spectroscopy; 3) Polypeptide fragmentation and peptide mapping approaches in which one generates multiple overlapping peptide fragments from the full length of the polypeptide and evaluates the binding affinity of these peptides for the epitope; 4) Antibody Phage Display Library analysis in which the antibody Fab fragment encoding genes of the mammal are expressed by bacteriophage in such a way as to be incorporated into the coat of the phage. This population of Fab expressing phage are then allowed to interact with the antigen which has been immobilized or may be expressed in by a different exogenous expression system. Non-binding Fab fragments are washed away, thereby leaving only the specific binding Fab fragments attached to the antigen. The binding Fab fragments can be readily isolated and the genes which encode them determined. This approach can also be used for smaller regions of the Fab fragment including Fv fragments or specific V_{H} and V_{L} domains as appropriate.

In certain aspects, affinity matured antibodies are enhanced with one or more modifications in one or more CDRs thereof that result in an improvement in the affinity of the antibody for a target antigen as compared to a parent antibody that does not possess those alteration(s). Certain affinity matured antibodies will have nanomolar or picomolar affinities for the target antigen. Affinity matured antibodies are produced by procedures known in the art, e.g., Marks et al., Bio/Technology 10:779 (1992) describes affinity maturation by V_{H} and V_{L} domain shuffling, random mutagenesis of CDR and/or framework residues employed in phage display is described by Rajpal et al., PNAS. 24: 8466-8471 (2005) and Thie et al., Methods Mol Biol. 525:309-22 (2009) in conjugation with computation methods as demonstrated in Tiller et al., Front. Immunol. 8:986 (2017).

Chimeric immunoglobulins describe the products of fused genes derived from different species; “humanized” chimeras generally have the framework region (FR) from human immunoglobulins and one or more CDRs are from a non-human source.

In certain aspects, portions of the heavy and/or light chain are identical or homologous to corresponding sequences from another particular species or belonging to a particular antibody class or subclass, while the remainder of the chain(s) is identical or homologous to corresponding sequences in antibodies derived from another species or belonging to another antibody class or subclass, as well as fragments of such antibodies, so long as they exhibit the desired biological activity. U.S. Pat. No. 4,816,567; and Morrison et al., Proc. Natl. Acad. Sci. USA 81:6851 (1984). For methods relating to chimeric antibodies, see, e.g., U.S. Pat. No. 4,816,567; and Morrison et al., Proc. Natl. Acad. Sci. USA 81:6851-6855 (1985), each of which are specifically incorporated herein by reference in their entirety. CDR grafting is described, for example, in U.S. Pat. Nos. 6,180,370, 5,693,762, 5,693,761, 5,585,089, and 5,530,101, which are all hereby incorporated by reference for all purposes.

In some embodiments, minimizing the antibody polypeptide sequence from the non-human species optimizes a chimeric antibody function and reduces immunogenicity. Specific amino acid residues from non-antigen recognizing regions of the non-human antibody may be modified to be homologous to corresponding residues in a human antibody or isotype. One example is the “CDR-grafted” antibody, in which an antibody comprises one or more CDRs from a particular species or belonging to a specific antibody class or subclass, while the remainder of the antibody chain(s) is identical or homologous to a corresponding sequence in antibodies derived from another species or belonging to another antibody class or subclass. For use in humans, the V region composed of CDR1, CDR2, and partial CDR3 for both the light and heavy chain variance region from a non-human immunoglobulin, are grafted with a human antibody framework region, replacing the naturally occurring antigen receptors of the human antibody with the non-human CDRs. In some instances, corresponding non-human residues replace framework region residues of the human immunoglobulin. Furthermore, humanized antibodies may comprise residues that are not found in the recipient antibody or in the donor antibody to further refine performance. The humanized antibody may also comprise at least a portion of an immunoglobulin constant region (Fc), typically that of a human immunoglobulin. See, e.g., Jones et al., Nature 321:522 (1986); Riechmann et al., Nature 332:323 (1988); Presta, Curr. Op. Struct. Biol. 2:593 (1992); Vaswani and Hamilton, Ann. Allergy, Asthma and Immunol. 1:105 (1998); Harris, Biochem. Soc. Transactions 23; 1035 (1995); Hurle and Gross, Curr. Op. Biotech. 5:428 (1994); Verhoeyen et al., Science 239:1534-36 (1988).

Intrabodies are intracellularly localized immunoglobulins that bind to intracellular antigens as opposed to secreted antibodies, which bind antigens in the extracellular space.

Polyclonal antibody preparations typically include different antibodies against different determinants (epitopes). In order to produce polyclonal antibodies, a host, such as a rabbit or goat, is immunized with the antigen or antigen fragment, generally with an adjuvant and, if necessary, coupled to a carrier. Antibodies to the antigen are subsequently collected from the sera of the host. The polyclonal antibody can be affinity purified against the antigen rendering it monospecific.

Monoclonal antibodies or “mAb” refer to an antibody obtained from a population of homogeneous antibodies from an exclusive parental cell, e.g., the population is identical except for naturally occurring mutations that may be present in minor amounts. Each monoclonal antibody is directed against a single antigenic determinant.

### A. Functional Antibody Fragments and Antigen-Binding Fragments

### 1. Antigen-Binding Fragments

Certain aspects of the disclosure relate to antibody fragments, such as antibody fragments that bind to CD70. The term functional antibody fragment includes antigen-binding fragments of an antibody that retain the ability to specifically bind to an antigen. These fragments are constituted of various arrangements of the variable region heavy chain (V_{H}; also “heavy chain variable region”) and/or light chain (V_{L}; also “light chain variable region”); and in some embodiments, include constant region heavy chain 1 (CHI) and light chain (CL). In some embodiments, they lack the Fc region constituted of heavy chain 2 (C_{H}2) and 3 (C_{H}3) domains. Embodiments of antigen binding fragments and the modifications thereof may include: (i) the Fab fragment type constituted with the V_{L}, V_{H}, CL, and C_{H}1 domains; (ii) the Fd fragment type constituted with the V_{H} and C_{H}1 domains; (iii) the Fv fragment type constituted with the V_{H} and V_{L} domains; (iv) the single domain fragment type, dAb, (Ward, 1989; McCafferty et al., 1990; Holt et al., 2003) constituted with a single V_{H} or V_{L} domain; (v) isolated complementarity determining region (CDR) regions. Such terms are described, for example, in Harlow and Lane, Antibodies: A Laboratory Manual, Cold Spring Harbor Laboratory, N Y (1989); Molec. Biology and Biotechnology: A Comprehensive Desk Reference (Myers, R. A. (ed.), New York: VCH Publisher, Inc.); Huston et al., Cell Biophysics, 22:189-224 (1993); Pluckthun and Skerra, Meth. Enzymol., 178:497-515 (1989) and in Day, E. D., Advanced Immunochemistry, 2d ed., Wiley-Liss, Inc. New York, N.Y. (1990); Antibodies, 4:259-277 (2015). The citations in this paragraph are all incorporated by reference.

Antigen-binding fragments also include fragments of an antibody that retain exactly, at least, or at most 1, 2, or 3 complementarity determining regions (CDRs) from a light chain variable region. Fusions of CDR-containing sequences to an Fc region (or a C_{H}2 or C_{H}3 region thereof) are included within the scope of this definition including, for example, scFv fused, directly or indirectly, to an Fc region are included herein.

The term Fab fragment means a monovalent antigen-binding fragment of an antibody containing the V_{L}, V_{H}, CL and C_{H}1 domains. The term Fab′ fragment means a monovalent antigen-binding fragment of a monoclonal antibody that is larger than a Fab fragment. For example, a Fab′ fragment includes the V_{L}, V_{H}, CL and C_{H}1 domains and all or part of the hinge region. The term F(ab′)2 fragment means a bivalent antigen-binding fragment of a monoclonal antibody comprising two Fab′ fragments linked by a disulfide bridge at the hinge region. An F(ab′)2 fragment includes, for example, all or part of the two V_{H} and V_{L} domains, and can further include all or part of the two CL and C_{H}1 domains.

The term Fd fragment means a fragment of the heavy chain of a monoclonal antibody, which includes all or part of the V_{H}, including the CDRs. An Fd fragment can further include C_{H}1 region sequences.

The term Fv fragment means a monovalent antigen-binding fragment of a monoclonal antibody, including all or part of the V_{L} and V_{H}, and absent of the CL and C_{H}1 domains. The V_{L} and V_{H} include, for example, the CDRs. Single-chain antibodies (sFv or scFv) are Fv molecules in which the V_{L} and V_{H} regions have been connected by a flexible linker to form a single polypeptide chain, which forms an antigen-binding fragment. Single chain antibodies are discussed in detail in International Patent Application Publication No. WO 88/01649 and U.S. Pat. Nos. 4,946,778 and 5,260,203, the disclosures of which are herein incorporated by reference. The term (scFv)2 means bivalent or bispecific sFv polypeptide chains that include oligomerization domains at their C-termini, separated from the sFv by a hinge region (Pack et al. 1992). The oligomerization domain comprises self-associating a-helices, e.g., leucine zippers, which can be further stabilized by additional disulfide bonds. (scFv)2 fragments are also known as “miniantibodies” or “minibodies.”

A single domain antibody is an antigen-binding fragment containing only a V_{H} or the V_{L} domain. In some instances, two or more V_{H} regions are covalently joined with a peptide linker to create a bivalent domain antibody. The two V_{H} regions of a bivalent domain antibody may target the same or different antigens.

### 2. Fragment Crystallizable Region, Fc

An Fc region contains two heavy chain fragments comprising the C_{H}2 and C_{H}3 domains of an antibody. The two heavy chain fragments are held together by two or more disulfide bonds and by hydrophobic interactions of the C_{H}3 domains. The term “Fc polypeptide” as used herein includes native and mutein forms of polypeptides derived from the Fc region of an antibody. Truncated forms of such polypeptides containing the hinge region that promotes dimerization are included.

### B. Polypeptides with Antibody CDRs & Scaffolding Domains that Display the CDRs

Antigen-binding peptide scaffolds, such as complementarity-determining regions (CDRs), are used to generate protein-binding molecules in accordance with the embodiments. Generally, a person skilled in the art can determine the type of protein scaffold on which to graft at least one of the CDRs. It is known that scaffolds, optimally, must meet a number of criteria such as: good phylogenetic conservation; known three-dimensional structure; small size; few or no post-transcriptional modifications; and/or be easy to produce, express, and purify. Skerra, J Mol Recognit, 13:167-87 (2000).

The protein scaffolds can be sourced from, but not limited to: fibronectin type III FN3 domain (known as “monobodies”), fibronectin type III domain 10, lipocalin, anticalin, Z-domain of protein A of Staphylococcus aureus, thioredoxin A or proteins with a repeated motif such as the “ankyrin repeat”, the “armadillo repeat”, the “leucine-rich repeat” and the “tetratricopeptide repeat”. Such proteins are described in US Patent Publication Nos. 2010/0285564, 2006/0058510, 2006/0088908, 2005/0106660, and PCT Publication No. WO2006/056464, each of which are specifically incorporated herein by reference in their entirety. Scaffolds derived from toxins from scorpions, insects, plants, mollusks, etc., and the protein inhibiters of neuronal NO synthase (PIN) may also be used.

### C. Antibody Binding

The term “selective binding agent” refers to a molecule that binds to an antigen. Non-limiting examples include antibodies, antigen-binding fragments, scFv, Fab, Fab′, F(ab′)2, single chain antibodies, peptides, peptide fragments and proteins.

The term “binding” refers to a direct association between two molecules, due to, for example, covalent, electrostatic, hydrophobic, and ionic and/or hydrogen-bond interactions, including interactions such as salt bridges and water bridges. “Immunologically reactive” means that the selective binding agent or antibody of interest will bind with antigens present in a biological sample. The term “immune complex” refers the combination formed when an antibody or selective binding agent binds to an epitope on an antigen.

### 1. Affinity/Avidity

The term “affinity” refers the strength with which an antibody or selective binding agent binds an epitope. In antibody binding reactions, this is expressed as the affinity constant (Ka or ka sometimes referred to as the association constant) for any given antibody or selective binding agent. Affinity is measured as a comparison of the binding strength of the antibody to its antigen relative to the binding strength of the antibody to an unrelated amino acid sequence. Affinity can be expressed as, for example, 20-fold greater binding ability of the antibody to its antigen then to an unrelated amino acid sequence. As used herein, the term “avidity” refers to the resistance of a complex of two or more agents to dissociation after dilution. The terms “immunoreactive” and “preferentially binds” are used interchangeably herein with respect to antibodies and selective binding agents.

There are several experimental methods that can be used by one skilled in the art to evaluate the binding affinity of any given antibody or selective binding agent for its antigen. This is generally done by measuring the equilibrium dissociation constant (KD or Kd), using the equation KD=koff/kon=[A][B]/[AB]. The term koff is the rate of dissociation between the antibody and antigen per unit time, and is related to the concentration of antibody and antigen present in solution in the unbound form at equilibrium. The term kon is the rate of antibody and antigen association per unit time, and is related to the concentration of the bound antigen-antibody complex at equilibrium. The units used for measuring the KD are mol/L (molarity, or M), or concentration. The Ka of an antibody is the opposite of the KD, and is determined by the equation Ka=1/KD. Examples of some experimental methods that can be used to determine the KD value are: enzyme-linked immunosorbent assays (ELISA), isothermal titration calorimetry (ITC), fluorescence anisotropy, surface plasmon resonance (SPR), and affinity capillary electrophoresis (ACE). The affinity constant (Ka) of an antibody is the opposite of the KD, and is determined by the equation Ka=1/KD.

Antibodies deemed useful in certain embodiments may have an affinity constant (Ka) of about, at least about, or at most about 10⁶, 10⁷, 10⁸, 10⁹, or 10¹⁰ M or any range derivable therein. Similarly, in some embodiments, antibodies may have a dissociation constant of about, at least about or at most about 10⁻⁶, 10⁻⁷, 10⁻⁸, 10⁻⁹, 10⁻¹⁰ M, or any range derivable therein. These values are reported for antibodies discussed herein and the same assay may be used to evaluate the binding properties of such antibodies. An antibody of the invention is said to “specifically bind” its target antigen when the dissociation constant (KD) is ≤10⁻⁸ M. The antibody specifically binds antigen with “high affinity” when the KD is ≤5×10⁻⁹ M, and with “very high affinity” when the KD is ≤5×10⁻¹⁰ M.

### 2. Epitope Specificity

The epitope of an antigen is the specific region of the antigen for which an antibody has binding affinity. In the case of protein or polypeptide antigens, the epitope is the specific residues (or specified amino acids or protein segment) that the antibody binds with high affinity. An antibody does not necessarily contact every residue within the protein. Nor does every single amino acid substitution or deletion within a protein necessarily affect binding affinity. For purposes of this specification and the accompanying claims, the terms “epitope” and “antigenic determinant” are used interchangeably to refer to the site on an antigen to which B and/or T cells respond or recognize. Polypeptide epitopes can be formed from both contiguous amino acids and noncontiguous amino acids juxtaposed by tertiary folding of a polypeptide. An epitope typically includes at least 3, and typically 5-10 amino acids in a unique spatial conformation.

Epitope specificity of an antibody can be determined in a variety of ways. One approach, for example, involves testing a collection of overlapping peptides of about 15 amino acids spanning the full sequence of the protein and differing in increments of a small number of amino acids (e.g., 3 to 30 amino acids). The peptides are immobilized in separate wells of a microtiter dish. Immobilization can be accomplished, for example, by biotinylating one terminus of the peptides. This process may affect the antibody affinity for the epitope, therefore different samples of the same peptide can be biotinylated at the N and C terminus and immobilized in separate wells for the purposes of comparison. This is useful for identifying end-specific antibodies. Optionally, additional peptides can be included terminating at a particular amino acid of interest. This approach is useful for identifying end-specific antibodies to internal fragments. An antibody or antigen-binding fragment is screened for binding to each of the various peptides. The epitope is defined as a segment of amino acids that is common to all peptides to which the antibody shows high affinity binding.

### 3. Modification of Antibody Antigen-Binding Domains

It is understood that the antibodies of the present disclosure may be modified, such that they are substantially identical to the antibody polypeptide sequences, or fragments thereof, and still bind the epitopes of the present disclosure. Polypeptide sequences are “substantially identical” when optimally aligned using such programs as Clustal Omega, IGBLAST, GAP or BESTFIT using default gap weights, they share at least 80% sequence identity, at least 90% sequence identity, at least 95% sequence identity, at least 96% sequence identity, at least 97% sequence identity, at least 98% sequence identity, or at least 99% sequence identity or any range therein.

As discussed herein, minor variations in the amino acid sequences of antibodies or antigen-binding regions thereof are contemplated as being encompassed by the present disclosure, providing that the variations in the amino acid sequence maintain at least 75%, more preferably at least 80%, at least 90%, at least 95%, at least 96%, at least 97%, at least 98% and most preferably at least 99% sequence identity. In particular, conservative amino acid replacements are contemplated.

Conservative replacements are those that take place within a family of amino acids that are related in their side chains. Genetically encoded amino acids are generally divided into families based on the chemical nature of the side chain; e.g., acidic (aspartate, glutamate), basic (lysine, arginine, histidine), nonpolar (alanine, valine, leucine, isoleucine, proline, phenylalanine, methionine, tryptophan), and uncharged polar (glycine, asparagine, glutamine, cysteine, serine, threonine, tyrosine). For example, it is reasonable to expect that an isolated replacement of a leucine moiety with an isoleucine or valine moiety, or a similar replacement of an amino acid with a structurally related amino acid in the same family, will not have a major effect on the binding or properties of the resulting molecule, especially if the replacement does not involve an amino acid within a framework site. Whether an amino acid change results in a functional peptide can readily be determined by assaying the specific activity of the polypeptide derivative. Standard ELISA, Surface Plasmon Resonance (SPR), Bio-layer interferometry (BLI)), or other antibody binding assays can be performed by one skilled in the art to make a quantitative comparison of antigen binging affinity between the unmodified antibody and any polypeptide derivatives with conservative substitutions generated through any of several methods available to one skilled in the art.

Fragments or analogs of antibodies or immunoglobulin molecules can be readily prepared by those skilled in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Standard methods to identify protein sequences that fold into a known three-dimensional structure are available to those skilled in the art; Dill and McCallum., Science 338:1042-1046 (2012). Several algorithms for predicting protein structures and the gene sequences that encode these have been developed, and many of these algorithms can be found at the National Center for Biotechnology Information (on the World Wide Web at ncbi.nlm.nih.gov/guide/proteins/) and at the Bioinformatics Resource Portal (on the World Wide Web at expasy.org/proteomics). Thus, the foregoing examples demonstrate that those of skill in the art can recognize sequence motifs and structural conformations that may be used to define structural and functional domains in accordance with the invention.

Framework modifications can be made to antibodies to decrease immunogenicity, for example, by “backmutating” one or more framework residues to a corresponding germline sequence.

It is also contemplated that the antigen-binding domain may be multi-specific or multivalent by multimerizing the antigen-binding domain with V_{H} and V_{L} region pairs that bind either the same antigen (multi-valent) or a different antigen (multi-specific).

### D. Chemical Modification of Antibodies

In some aspects, also contemplated are glycosylation variants of antibodies, wherein the number and/or type of glycosylation site(s) has been altered compared to the amino acid sequences of the parent polypeptide. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861). In certain embodiments, antibody protein variants comprise a greater or a lesser number of N-linked glycosylation sites than the native antibody. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate or alter this sequence will prevent addition of an N-linked carbohydrate chain present in the native polypeptide. For example, the glycosylation can be reduced by the deletion of an Asn or by substituting the Asn with a different amino acid. In other embodiments, one or more new N-linked glycosylation sites are created. Antibodies typically have an N-linked glycosylation site in the Fc region.

Additional antibody variants include cysteine variants, wherein one or more cysteine residues in the parent or native amino acid sequence are deleted from or substituted with another amino acid (e.g., serine). Cysteine variants are useful, inter alia, when antibodies must be refolded into a biologically active conformation. Cysteine variants may have fewer cysteine residues than the native antibody and typically have an even number to minimize interactions resulting from unpaired cysteines.

In some aspects, the polypeptides can be pegylated to increase biological half-life by reacting the polypeptide with polyethylene glycol (PEG) or a reactive ester or aldehyde derivative of PEG, under conditions in which one or more PEG groups become attached to the polypeptide. Polypeptide pegylation may be carried out by an acylation reaction or an alkylation reaction with a reactive PEG molecule (or an analogous reactive water-soluble polymer). Methods for pegylating proteins are known in the art and can be applied to the polypeptides of the present disclosure to obtain PEGylated derivatives of antibodies. See, e.g., EP 0 154 316 and EP 0 401 384. As used herein, the term “polyethylene glycol” is intended to encompass any of the forms of PEG that have been used to derivatize other proteins.

### 1. Conjugation

Derivatives of the antibodies and antigen binding fragments that are described herein are also provided. The derivatized antibody or fragment thereof may comprise any molecule or substance that imparts a desired property to the antibody or fragment. The derivatized antibody can comprise, for example, a detectable (or labeling) moiety (e.g., a radioactive, colorimetric, antigenic, or enzymatic molecule, or a detectable bead), a molecule that binds to another molecule (e.g., biotin or streptavidin), a therapeutic or diagnostic moiety (e.g., a radioactive, cytotoxic, or pharmaceutically active moiety), or a molecule that increases the suitability of the antibody for a particular use (e.g., administration to a subject, such as a human subject, or other in vivo or in vitro uses).

Optionally, an antibody or an immunological portion of an antibody can be chemically conjugated to, or expressed as, a fusion protein with other proteins. In some aspects, polypeptides may be chemically modified by conjugating or fusing the polypeptide to serum protein, such as human serum albumin, to increase half-life of the resulting molecule. See, e.g., EP 0322094 and EP 0 486 525. In some aspects, the polypeptides may be conjugated to a diagnostic agent and used diagnostically, for example, to monitor the development or progression of a disease and determine the efficacy of a given treatment regimen. In some aspects, the polypeptides may also be conjugated to a therapeutic agent to provide a therapy in combination with the therapeutic effect of the polypeptide. Additional suitable conjugated molecules include ribonuclease (RNase), DNase I, an antisense nucleic acid, an inhibitory RNA molecule such as a siRNA molecule, an immunostimulatory nucleic acid, aptamers, ribozymes, triplex forming molecules, and external guide sequences. The functional nucleic acid molecules may act as effectors, inhibitors, modulators, and stimulators of a specific activity possessed by a target molecule, or the functional nucleic acid molecules may possess a de novo activity independent of any other molecules.

In some aspects, disclosed are antibodies and antibody-like molecules that are linked to at least one agent to form an antibody conjugate or payload. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules include toxins, therapeutic enzymes, antibiotics, radiolabeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles, or ligands.

a. Conjugate Types

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to be detected, and/or further quantified if desired. Examples of detectable labels include, but not limited to, radioactive isotopes, fluorescers, semiconductor nanocrystals, chemiluminescers, chromophores, enzymes, enzyme substrates, enzyme cofactors, enzyme inhibitors, dyes, metal ions, metal sols, ligands (e.g., biotin, streptavidin or haptens) and the like. Particular examples of labels are, but not limited to, horseradish peroxidase (HRβ), fluorescein, FITC, rhodamine, dansyl, umbelliferone, dimethyl acridinium ester (DMAE), Texas red, luminol, NADPH and α- or β-galactosidase. Antibody conjugates include those intended primarily for use in vitro, where the antibody is linked to a secondary binding ligand and/or to an enzyme to generate a colored product upon contact with a chromogenic substrate. Examples of suitable enzymes include, but are not limited to, urease, alkaline phosphatase, (horseradish) hydrogen peroxidase, or glucose oxidase. Preferred secondary binding ligands are biotin and/or avidin and streptavidin compounds. The uses of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference. Molecules containing azido groups may also be used to form covalent bonds to proteins through reactive nitrene intermediates that are generated by low intensity ultraviolet light (Potter & Haley, 1983).

In some aspects, contemplated are immunoconjugates comprising an antibody or antigen-binding fragment thereof conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). In this way, the agent of interest can be targeted directly to cells bearing cell surface antigen. The antibody and agent may be associated through non-covalent interactions such as through electrostatic forces, or by covalent bonds. Various linkers, known in the art, can be employed in order to form the immunoconjugate. Additionally, the immunoconjugate can be provided in the form of a fusion protein. In one aspect, an antibody may be conjugated to various therapeutic substances in order to target the cell surface antigen. Examples of conjugated agents include, but are not limited to, metal chelate complexes, drugs, toxins and other effector molecules, such as cytokines, lymphokines, chemokines, immunomodulators, radiosensitizers, asparaginase, carboranes, and radioactive halogens.

In antibody drug conjugates (ADC), an antibody (Ab) is conjugated to one or more drug moieties (D) through a linker (L). The ADC may be prepared by several routes, employing organic chemistry reactions, conditions, and reagents known to those skilled in the art, including: (1) reaction of a nucleophilic group of an antibody with a bivalent linker reagent, to form Ab-L, via a covalent bond, followed by reaction with a drug moiety D; and (2) reaction of a nucleophilic group of a drug moiety with a bivalent linker reagent, to form D-L, via a covalent bond, followed by reaction with the nucleophilic group of an antibody. Antibody drug conjugates may also be produced by modification of the antibody to introduce electrophilic moieties, which can react with nucleophilic substituents on the linker reagent or drug. Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate. In yet another aspect, the antibody may be conjugated to a “receptor” (such as streptavidin) for utilization in tumor or cancer cell pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

Examples of an antibody-drug conjugates known to a person skilled in the art are pro-drugs useful for the local delivery of cytotoxic or cytostatic agents, i.e. drugs to kill or inhibit tumor cells in the treatment of cancer (Syrigos and Epenetos, Anticancer Res. 19:605-614 (1999); Niculescu-Duvaz and Springer, Adv. Drg. Del. Rev. 26:151-172 (1997); U.S. Pat. No. 4,975,278). In contrast, systematic administration of these unconjugated drug agents may result in unacceptable levels of toxicity to normal cells as well as the target tumor cells (Baldwin et al., Lancet 1:603-5 (1986); Thorpe, (1985) “Antibody Carriers of Cytotoxic Agents in Cancer Therapy: A Review,” In: Monoclonal Antibodies '84: Biological and Clinical Applications, A. Pincera et al., (eds.) pp. 475-506). Both polyclonal antibodies and monoclonal antibodies have been reported as useful in these strategies (Rowland et al., Cancer Immunol. Immunother. 21:183-87 (1986)).

In certain aspects, ADC include covalent or aggregative conjugates of antibodies, or antigen-binding fragments thereof, with other proteins or polypeptides, such as by expression of recombinant fusion proteins comprising heterologous polypeptides fused to the N-terminus or C-terminus of an antibody polypeptide. For example, the conjugated peptide may be a heterologous signal (or leader) polypeptide, e.g., the yeast alpha-factor leader, or a peptide such as an epitope tag (e.g., V5-His). Antibody-containing fusion proteins may comprise peptides added to facilitate purification or identification of the antibody (e.g., poly-His). An antibody polypeptide also can be linked to the FLAG® (Sigma-Aldrich, St. Louis, Mo.) peptide as described in Hopp et al., Bio/Technology 6:1204 (1988), and U.S. Pat. No. 5,011,912. Oligomers that contain one or more antibody polypeptides may be employed as antagonists. Oligomers may be in the form of covalently linked or non-covalently linked dimers, trimers, or higher oligomers. Oligomers comprising two or more antibody polypeptides are contemplated for use. Other oligomers include heterodimers, homotrimers, heterotrimers, homotetramers, heterotetramers, etc. In certain aspects, oligomers comprise multiple antibody polypeptides joined via covalent or non-covalent interactions between peptide moieties fused to the antibody polypeptides. Such peptides may be peptide linkers (spacers), or peptides that have the property of promoting oligomerization. Leucine zippers and certain polypeptides derived from antibodies are among the peptides that can promote oligomerization of antibody polypeptides attached thereto, as described in more detail below.

b. Conjugation Methodology

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates may also be made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis(p-azidobenzoyl)hexanediamine), bis-diazonium derivatives (such as bos(p-diazoniumbenzoyl)-ethylenediamine), diisocyanates (such as toluene 2,6-diisocyanate), and bis-active fluorine compounds (such as 1,5-difluoro-2,4-dinitrobenzene). In some aspects, derivatization of immunoglobulins by selectively introducing sulfhydryl groups in the Fc region of an immunoglobulin, using reaction conditions that do not alter the antibody combining site, are contemplated. Antibody conjugates produced according to this methodology are disclosed to exhibit improved longevity, specificity, and sensitivity (U.S. Pat. No. 5,196,066, incorporated herein by reference). Site-specific attachment of effector or reporter molecules, wherein the reporter or effector molecule is conjugated to a carbohydrate residue in the Fc region has also been disclosed in the literature (O'Shannessy et al., 1987).

### III. Antibody Production

### A. Antibody Production

Methods for preparing and characterizing antibodies for use in diagnostic and detection assays, for purification, and for use as therapeutics are well known in the art as disclosed in, for example, U.S. Pat. Nos. 4,011,308; 4,722,890; 4,016,043; 3,876,504; 3,770,380; and 4,372,745 (see, e.g., Antibodies: A Laboratory Manual, Cold Spring Harbor Laboratory, 1988; incorporated herein by reference). These antibodies may be polyclonal or monoclonal antibody preparations, monospecific antisera, human antibodies, hybrid or chimeric antibodies, such as humanized antibodies, altered antibodies, F(ab′)2 fragments, Fab fragments, Fv fragments, single-domain antibodies, dimeric or trimeric antibody fragment constructs, minibodies, or functional fragments thereof which bind to the antigen in question. In certain aspects, polypeptides, peptides, and proteins and immunogenic fragments thereof for use in various embodiments can also be synthesized in solution or on a solid support in accordance with conventional techniques. See, for example, Stewart and Young, (1984); Tarn et al, (1983); Merrifield, (1986); and Barany and Merrifield (1979), each incorporated herein by reference.

Briefly, a polyclonal antibody is prepared by immunizing an animal with an antigen or a portion thereof and collecting antisera from that immunized animal. The antigen may be altered compared to an antigen sequence found in nature. In some embodiments, a variant or altered antigenic peptide or polypeptide is employed to generate antibodies. Inocula are typically prepared by dispersing the antigenic composition in a physiologically tolerable diluent to form an aqueous composition. Antisera is subsequently collected by methods known in the arts, and the serum may be used as-is for various applications or else the desired antibody fraction may be purified by well-known methods, such as affinity chromatography (Harlow and Lane, Antibodies: A Laboratory Manual 1988).

Methods of making monoclonal antibodies are also well known in the art (Kohler and Milstein, 1975; Harlow and Lane, 1988, U.S. Pat. No. 4,196,265, herein incorporated by reference in its entirety for all purposes). Typically, this technique involves immunizing a suitable animal with a selected immunogenic composition, e.g., a purified or partially purified protein, polypeptide, peptide or domain. Resulting antibody-producing B-cells from the immunized animal, dissociated splenocytes, and/or dissociated lymph nodes are then induced to fuse with cells from an immortalized cell line to form hybridomas. Myeloma cell lines suited for use in hybridoma-producing fusion procedures preferably are non-antibody-producing and have high fusion efficiency and enzyme deficiencies that render then incapable of growing in certain selective media that support the growth of only the desired fused cells (hybridomas). Typically, the fusion partner includes a property that allows selection of the resulting hybridomas using specific media. For example, fusion partners can be hypoxanthine/aminopterin/thymidine (HAT)-sensitive. Methods for generating hybrids of antibody-producing spleen or lymph node cells and myeloma cells usually comprise mixing somatic cells with myeloma cells in the presence of an agent or agents (chemical or electrical) that promote the fusion of cell membranes. Next, selection of hybridomas can be performed by culturing the cells by single-clone dilution in microtiter plates, followed by testing the individual clonal supernatants (after about two to three weeks) for the desired reactivity. Fusion procedures for making hybridomas, immunization protocols, and techniques for isolation of immunized splenocytes for fusion are known in the art.

Other techniques for producing monoclonal antibodies include the viral or oncogenic transformation of B-lymphocytes, a molecular cloning approach may be used to generate a nucleic acid or polypeptide, the selected lymphocyte antibody method (SLAM) (see, e.g., Babcook et al., Proc. Natl. Acad. Sci. USA 93:7843-7848 (1996), the preparation of combinatorial immunoglobulin phagemid libraries from RNA isolated from the spleen of the immunized animal and selection of phagemids expressing appropriate antibodies, or producing a cell expressing an antibody from a genomic sequence of the cell comprising a modified immunoglobulin locus using Cre-mediated site-specific recombination (see, e.g., U.S. Pat. No. 6,091,001) or performing single B cell cloning to isolate nucleic acids specific of V_{H} and V_{L} chains from immunoglobulins, from either immunized animals or human individuals bearing tumors or known to be affected by certain pathologies or infectious diseases that trigger a humoral immune response and have plasma B cells circulating, or from PBMCs frozen from similar individuals (see Von Boehmer L, Liu C, Ackerman S, Gitlin A D, Wang Q, Gazumyan A, Nussenzweig M C. (2016) Sequencing and cloning of antigen-specific antibodies from mouse memory B cells. Nature Protocols 11:1908-1923, incorporated by reference herein in its entirety).

Monoclonal antibodies may be further purified using filtration, centrifugation, and various chromatographic methods such as HPLC or affinity chromatography. Monoclonal antibodies may be further screened or optimized for properties relating to specificity, avidity, half-life, immunogenicity, binding association, binding disassociation, or overall functional properties relative to being a treatment for infection. Thus, monoclonal antibodies may have alterations in the amino acid sequence of CDRs, including insertions, deletions, or substitutions with a conserved or non-conserved amino acid.

The immunogenicity of a particular immunogen composition can be enhanced by the use of non-specific stimulators of the immune response, known as adjuvants. Adjuvants that may be used in accordance with embodiments include, but are not limited to, IL-1, IL-2, IL-4, IL-7, IL-12, 7-interferon, GMCSF, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, and/or aluminum hydroxide adjuvant. In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as 0-interferon, IL-2, or IL-12, or genes encoding proteins involved in immune helper functions, such as B-7. A phage-display system can be used to expand antibody molecule populations in vitro. Saiki, et al., Nature 324:163 (1986); Scharf et al., Science 233:1076 (1986); U.S. Pat. Nos. 4,683,195 and 4,683,202; Yang et al., J Mol Biol. 254:392 (1995); Barbas, III et al., Methods: Comp. Meth Enzymol. (1995) 8:94; Barbas, III et al., Proc Natl Acad Sci USA 88:7978 (1991).

### B. Fully Human Antibody Production

Methods are available for making fully human antibodies. Using fully human antibodies can minimize the immunogenic and allergic responses that may be caused by administering non-human monoclonal antibodies to humans as therapeutic agents. In one embodiment, human antibodies may be produced in a non-human transgenic animal, e.g., a transgenic mouse capable of producing multiple isotypes of human antibodies to protein (e.g., IgG, IgA, and/or IgE) by undergoing V-D-J recombination and isotype switching. Accordingly, this aspect applies to antibodies, antibody fragments, and pharmaceutical compositions thereof, but also non-human transgenic animals, B-cells, host cells, and hybridomas that produce monoclonal antibodies. Applications of human antibodies include, but are not limited to, detect a cell expressing an anticipated protein, either in vivo or in vitro, pharmaceutical preparations containing the antibodies of the present disclosure, and methods of treating disorders by administering the antibodies.

Fully human antibodies can be produced by immunizing transgenic animals (e.g., mice) that are capable of producing a repertoire of human antibodies in the absence of endogenous immunoglobulin production. Antigens for this purpose typically have six or more contiguous amino acids, and optionally are conjugated to a carrier, such as a hapten. See, for example, Jakobovits et al., Proc. Natl. Acad. Sci. USA 90:2551-2555 (1993); Jakobovits et al., Nature 362:255-258 (1993); Bruggermann et al., Year in Immunol. 7:33 (1993). In one example, transgenic animals are produced by incapacitating the endogenous mouse immunoglobulin loci encoding the mouse heavy and light immunoglobulin chains therein, and inserting into the mouse genome large fragments of human genome DNA containing loci that encode human heavy and light chain proteins. Partially modified animals, which have less than the full complement of human immunoglobulin loci, are then crossbred to obtain an animal having all of the desired immune system modifications. When administered an immunogen, these transgenic animals produce antibodies that are immunospecific for the immunogen but have human rather than murine amino acid sequences, including the variable regions. For further details of such methods, see, for example, International Patent Application Publication Nos. WO 96/33735 and WO 94/02602, which are hereby incorporated by reference in their entirety. Additional methods relating to transgenic mice for making human antibodies are described in U.S. Pat. Nos. 5,545,807; 6,713,610; 6,673,986; 6,162,963; 6,300,129; 6,255,458; 5,877,397; 5,874,299 and 5,545,806; in International Patent Application Publication Nos. WO 91/10741 and WO 90/04036; and in European Patent Nos. EP 546073B1 and EP 546073A1, all of which are hereby incorporated by reference in their entirety for all purposes.

The transgenic mice described above, referred to herein as “HuMAb” mice, contain a human immunoglobulin gene minilocus that encodes unrearranged human heavy (and y) and x light chain immunoglobulin sequences, together with targeted mutations that inactivate the endogenous and x chain loci (Lonberg et al., Nature 368:856-859 (1994)). Accordingly, the mice exhibit reduced expression of mouse IgM or κ chains and in response to immunization, the introduced human heavy and light chain transgenes undergo class switching and somatic mutation to generate high affinity human IgG κ monoclonal antibodies (Lonberg et al., supra; Lonberg and Huszar, Intern. Ref. Immunol. 13:65-93 (1995); Harding and Lonberg, Ann. N.Y. Acad. Sci. 764:536-546 (1995)). The preparation of HuMAb mice is described in detail in Taylor et al., Nucl. Acids Res. 20:6287-6295 (1992); Chen et al., Int. Immunol. 5:647-656 (1993); Tuaillon et al., J. Immunol. 152:2912-2920 (1994); Lonberg et al., supra; Lonberg, Handbook of Exp. Pharmacol. 113:49-101 (1994); Taylor et al., Int. Immunol. 6:579-591 (1994); Lonberg and Huszar, Intern. Ref. Immunol. 13:65-93 (1995); Harding and Lonberg, Ann. N.Y. Acad. Sci. 764:536-546 (1995); Fishwild et al., Nat. Biotechnol. 14:845-851 (1996); the foregoing references are herein incorporated by reference in their entirety for all purposes. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807; as well as International Patent Application Publication Nos. WO 93/1227; WO 92/22646; and WO 92/03918, the disclosures of all of which are hereby incorporated by reference in their entirety for all purposes. Technologies utilized for producing human antibodies in these transgenic mice are disclosed also in WO 98/24893, and Mendez et al., Nat. Genetics 15:146-156 (1997), which are herein incorporated by reference. For example, the HCo7 and HCo12 transgenic mice strains can be used to generate human antibodies.

Using hybridoma technology, antigen-specific humanized monoclonal antibodies with the desired specificity can be produced and selected from the transgenic mice such as those described above. Such antibodies may be cloned and expressed using a suitable vector and host cell, or the antibodies can be harvested from cultured hybridoma cells. Fully human antibodies can also be derived from phage-display libraries (as disclosed in Hoogenboom et al., J. Mol. Biol. 227:381 (1991); and Marks et al., J. Mol. Biol. 222:581 (1991)). One such technique is described in International Patent Application Publication No. WO 99/10494 (herein incorporated by reference), which describes the isolation of high affinity and functional agonistic antibodies for MPL- and msk-receptors using such an approach. If the B cell donor is human, humanized or fully human antibodies can also be obtained by performing single B cell cloning to isolate nucleic acids specific of V_{H} and V_{L} chains from immunoglobulins, from either immunized animals or human individuals bearing tumors or known to be affected by certain pathologies or infectious diseases that trigger a humoral immune response and have plasma B cells circulating, or from PBMCs frozen from similar individuals (see Von Boehmer L, Liu C, Ackerman S, Gitlin A D, Wang Q, Gazumyan A, Nussenzweig M C. (2016) Sequencing and cloning of antigen-specific antibodies from mouse memory B cells. Nature Protocols 11:1908-1923, incorporated by reference herein in its entirety).

### C. Antibody Fragments Production

Antibody fragments that retain the ability to recognize the antigen of interest will also find use herein. A number of antibody fragments are known in the art that comprise antigen-binding sites capable of exhibiting immunological binding properties of an intact antibody molecule and can be subsequently modified by methods known in the arts. Functional fragments, including only the variable regions of the heavy and light chains, can also be produced using standard techniques such as recombinant production or preferential proteolytic cleavage of immunoglobulin molecules. These fragments are known as Fv. See, e.g., Inbar et al., Proc. Nat. Acad. Sci. USA 69:2659-2662 (1972); Hochman et al., Biochem. 15:2706-2710 (1976); and Ehrlich et al., Biochem. 19:4091-4096 (1980).

Single-chain variable fragments (scFvs) may be prepared by fusing DNA encoding a peptide linker between DNAs encoding the two variable domain polypeptides (V_{L} and V_{H}). scFvs can form antigen-binding monomers, or they can form multimers (e.g., dimers, trimers, or tetramers), depending on the length of a flexible linker between the two variable domains (Kortt et al., Prot. Eng. 10:423 (1997); Kort et al., Biomol. Eng. 18:95-108 (2001)). By combining different V_{L}- and V_{H}-comprising polypeptides, one can form multimeric scFvs that bind to different epitopes (Kriangkum et al., Biomol. Eng. 18:31-40 (2001)). Antigen-binding fragments are typically produced by recombinant DNA methods known to those skilled in the art. Although the two domains of the Fv fragment, V_{L} and V_{H}, are coded for by separate genes, they can be joined using recombinant methods by a synthetic linker that enables them to be made as a single chain polypeptide (known as single chain Fv (sFv or scFv); see e.g., Bird et al., Science 242:423-426 (1988); and Huston et al., Proc. Natl. Acad. Sci. USA 85:5879-5883 (1988). Design criteria include determining the appropriate length to span the distance between the C-terminus of one chain and the N-terminus of the other, wherein the linker is generally formed from small hydrophilic amino acid residues that do not tend to coil or form secondary structures. Suitable linkers generally comprise polypeptide chains of alternating sets of glycine and serine residues, and may include glutamic acid and lysine residues inserted to enhance solubility. Antigen-binding fragments are screened for utility in the same manner as intact antibodies. Such fragments include those obtained by amino-terminal and/or carboxy-terminal deletions, where the remaining amino acid sequence is substantially identical to the corresponding positions in the naturally occurring sequence deduced, for example, from a full-length cDNA sequence.

Antibodies may also be generated using peptide analogs of the epitopic determinants disclosed herein, which may consist of non-peptide compounds having properties analogous to those of the template peptide. These types of non-peptide compound are termed “peptide mimetics” or “peptidomimetics”. Fauchere, J. Adv. Drug Res. 15:29 (1986); Veber and Freidinger TINS p. 392 (1985); and Evans et al., J. Med. Chem. 30:1229 (1987). Liu et al. (2003) also describe “antibody like binding peptidomimetics” (ABiPs), which are peptides that act as pared-down antibodies and have certain advantages of longer serum half-life as well as less cumbersome synthesis methods. These analogs can be peptides, non-peptides or combinations of peptide and non-peptide regions. Fauchere, Adv. Drug Res. 15:29 (1986); Veber and Freidiner, TINS p. 392 (1985); and Evans et al., J. Med. Chem. 30:1229 (1987), which are incorporated herein by reference in their entirety for any purpose. Peptide mimetics that are structurally similar to therapeutically useful peptides may be used to produce a similar therapeutic or prophylactic effect. Such compounds are often developed with the aid of computerized molecular modeling. Generally, peptidomimetics of the disclosure are proteins that are structurally similar to an antibody displaying a desired biological activity, such as the ability to bind a protein, but have one or more peptide linkages optionally replaced by a linkage selected from: —CH₂NH—, —CH₂S, —CH₂—CH₂—, —CH═CH— (cis and trans), —COCH₂—, —CH(OH)CH₂—, and —CH₂SO— by methods well known in the art. Systematic substitution of one or more amino acids of a consensus sequence with a D-amino acid of the same type (e.g., D-lysine in place of L-lysine) may be used in certain embodiments of the disclosure to generate more stable proteins. In addition, constrained peptides comprising a consensus sequence or a substantially identical consensus sequence variation may be generated by methods known in the art (Rizo and Gierasch, Ann. Rev. Biochem. 61:387 (1992), incorporated herein by reference), for example, by adding internal cysteine residues capable of forming intramolecular disulfide bridges which cyclize the peptide.

Once generated, a phage display library can be used to improve the immunological binding affinity of the Fab molecules using known techniques. See, e.g., Figini et al., J. Mol. Biol. 239:68 (1994). The coding sequences for the heavy and light chain portions of the Fab molecules selected from the phage display library can be isolated or synthesized and cloned into any suitable vector or replicon for expression. Any suitable expression system can be used.

### IV. Polypeptides

As used herein, a “protein,” “peptide,” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, at least, at most, exactly, or between any two of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The polypeptides, proteins, or polynucleotides encoding such polypeptides or proteins of the disclosure may include at least, at most, exactly, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 (or any derivable range therein) or more variant amino acids or nucleotide substitutions or be at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with at least, at most, exactly, or between any two of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more contiguous amino acids or nucleotides, or any range derivable therein, of SEQ ID NOs:1-105.

In some embodiments, the protein or polypeptide may comprise amino acids 1 to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124,125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) of SEQ ID NOs:44-105.

In some embodiments, the protein or polypeptide may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123,124, 125, 126, 127,128, 129, 130, 131, 132, 133, 134, 135, 136, 137, 138, 139, 140, 141, 142, 143, 144, 145, 146,147, 148, 149, 150, 151, 152, 153,154, 155, 156, 157, 158, 159, 160, 161,162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000, (or any derivable range therein) contiguous amino acids of SEQ ID NOs:44-105.

In some embodiments, the polypeptide or protein may comprise at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122,123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids of SEQ ID NOs:44-105 that are at least, at most, exactly, or between any two of 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% (or any derivable range therein) similar, identical, or homologous with one of SEQ ID NOs:44-105.

In some aspects there is a polypeptide starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124,125, 126, 127, 128,129, 130, 131, 132, 133, 134, 135, 136, 137, 138, 139, 140, 141, 142, 143, 144, 145, 146, 147,148, 149, 150,151, 152, 153, 154,155, 156, 157, 158, 159, 160, 161, 162,163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 of any of SEQ ID NOs:44-105 and comprising at least, at most, or exactly 2, 3, 4, 5, 6, 7, 8,9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123,124, 125, 126, 127,128, 129, 130,131, 132, 133, 134,135, 136, 137, 138, 139, 140, 141, 142,143, 144, 145, 146,147, 148, 149,150, 151, 152, 153,154, 155, 156, 157, 158, 159, 160, 161,162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, or 1000 (or any derivable range therein) contiguous amino acids or nucleotides of any of SEQ ID NOs:1-105.

The nucleotide as well as the protein, polypeptide, and peptide sequences for various genes have been previously disclosed, and may be found in the recognized computerized databases. Two commonly used databases are the National Center for Biotechnology Information's GENBANK® and GENPEPT® databases (on the World Wide Web at ncbi.nlm.nih.gov/) and The Universal Protein Resource (UNIPROT®; on the World Wide Web at uniprot.org). The coding regions for these genes may be amplified and/or expressed using the techniques disclosed herein or as would be known to those of ordinary skill in the art.

It is contemplated that in compositions of the disclosure, there is between about 0.001 mg and about 10 mg of total polypeptide, peptide, and/or protein per ml. The concentration of protein in a composition can be at least, at most, exactly, or between any two of about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

### A. Variant Polypeptides

The following is a discussion of changing the amino acid subunits of a protein to create an equivalent, or even improved, variant polypeptide or peptide. For example, certain amino acids may be substituted for other amino acids in a protein or polypeptide sequence with or without appreciable loss of interactive binding capacity with structures such as, for example, antigen-binding regions of antibodies or binding sites on substrate molecules. Since it is the interactive capacity and nature of a protein that defines that protein's functional activity, certain amino acid substitutions can be made in a protein sequence and in its corresponding DNA coding sequence, and nevertheless produce a protein with similar or desirable properties. It is thus contemplated by the inventors that various changes may be made in the DNA sequences of genes which encode proteins without appreciable loss of their biological utility or activity.

The term “functionally equivalent codon” is used herein to refer to codons that encode the same amino acid, such as the six different codons for arginine. Also considered are “neutral substitutions” or “neutral mutations” which refers to a change in the codon or codons that encode biologically equivalent amino acids.

Amino acid sequence variants of the disclosure can be substitutional, insertional, or deletion variants. A variation in a polypeptide of the disclosure may affect at least, at most, exactly, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more non-contiguous or contiguous amino acids of the protein or polypeptide, as compared to wild-type. A variant can comprise an amino acid sequence that is at least, at most, exactly, or between any two of 50%, 60%, 70%, 80%, 90%, 91%, 92%, 93%, 94%, 95%, 96%, 97%, 98%, or 99% identical to any sequence provided or referenced herein. A variant can include at least, at most, exactly, or between any two of 2, 3, 4, 5, 6, 7, 8, 9, 10, 11, 12, 13, 14, 15, 16, 17, 18, 19, 20, or more substitute amino acids.

It also will be understood that amino acid and nucleic acid sequences may include additional residues, such as additional N- or C-terminal amino acids, or 5′ or 3′ sequences, respectively, and yet still be essentially identical as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

Deletion variants typically lack one or more residues of the native or wild type protein. Individual residues can be deleted or a number of contiguous amino acids can be deleted. A stop codon may be introduced (by substitution or insertion) into an encoding nucleic acid sequence to generate a truncated protein.

Insertional mutants typically involve the addition of amino acid residues at a non-terminal point in the polypeptide. This may include the insertion of one or more amino acid residues. Terminal additions may also be generated and can include fusion proteins which are multimers or concatemers of one or more peptides or polypeptides described or referenced herein.

Substitutional variants typically contain the exchange of one amino acid for another at one or more sites within the protein or polypeptide, and may be designed to modulate one or more properties of the polypeptide, with or without the loss of other functions or properties. Substitutions may be conservative, that is, one amino acid is replaced with one of similar chemical properties. “Conservative amino acid substitutions” may involve exchange of a member of one amino acid class with another member of the same class. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Conservative amino acid substitutions may encompass non-naturally occurring amino acid residues, which are typically incorporated by chemical peptide synthesis rather than by synthesis in biological systems. These include peptidomimetics or other reversed or inverted forms of amino acid moieties.

Alternatively, substitutions may be “non-conservative”, such that a function or activity of the polypeptide is affected. Non-conservative changes typically involve substituting an amino acid residue with one that is chemically dissimilar, such as a polar or charged amino acid for a nonpolar or uncharged amino acid, and vice versa. Non-conservative substitutions may involve the exchange of a member of one of the amino acid classes for a member from another class.

### B. Considerations for Substitutions

One skilled in the art can determine suitable variants of polypeptides as set forth herein using well-known techniques. One skilled in the art may identify suitable areas of the molecule that may be changed without destroying activity by targeting regions not believed to be important for activity. The skilled artisan will also be able to identify amino acid residues and portions of the molecules that are conserved among similar proteins or polypeptides. In further embodiments, areas that may be important for biological activity or for structure may be subject to conservative amino acid substitutions without significantly altering the biological activity or without adversely affecting the protein or polypeptide structure.

In making such changes, the hydropathy index of amino acids may be considered. The hydropathy profile of a protein is calculated by assigning each amino acid a numerical value (“hydropathy index”) and then repetitively averaging these values along the peptide chain. Each amino acid has been assigned a value based on its hydrophobicity and charge characteristics. They are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (1.6); histidine (−3.2); glutamate (−3.5); glutamine (−3.5); aspartate (−3.5); asparagine (−3.5); lysine (−3.9); and arginine (−4.5). The importance of the hydropathy amino acid index in conferring interactive biologic function on a protein is generally understood in the art (Kyte et al., J. Mol. Biol. 157:105-131 (1982)). It is accepted that the relative hydropathic character of the amino acid contributes to the secondary structure of the resultant protein or polypeptide, which in turn defines the interaction of the protein or polypeptide with other molecules, for example, enzymes, substrates, receptors, DNA, antibodies, antigens, and others. It is also known that certain amino acids may be substituted for other amino acids having a similar hydropathy index or score, and still retain a similar biological activity. In making changes based upon the hydropathy index, in certain embodiments, the substitution of amino acids whose hydropathy indices are within ±2 is included. In some aspects of the present disclosure, those that are within ±1 are included, and in other aspects of the present disclosure, those within ±0.5 are included.

It also is understood in the art that the substitution of like amino acids can be effectively made based on hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. In certain embodiments, the greatest local average hydrophilicity of a protein, as governed by the hydrophilicity of its adjacent amino acids, correlates with its immunogenicity and antigen binding, that is, as a biological property of the protein. The following hydrophilicity values have been assigned to these amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); and tryptophan (−3.4). In making changes based upon similar hydrophilicity values, in certain embodiments, the substitution of amino acids whose hydrophilicity values are within +2 are included, in other embodiments, those which are within +1 are included, and in still other embodiments, those within 0.5 are included. In some instances, one may also identify epitopes from primary amino acid sequences based on hydrophilicity. These regions are also referred to as “epitopic core regions.” It is understood that an amino acid can be substituted for another having a similar hydrophilicity value and still produce a biologically equivalent and immunologically equivalent protein.

Additionally, one skilled in the art can review structure-function studies identifying residues in similar polypeptides or proteins that are important for activity or structure. In view of such a comparison, one can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar proteins or polypeptides. In view of such information, one skilled in the art may predict the alignment of amino acid residues of an antibody with respect to its three-dimensional structure. One skilled in the art may choose not to make changes to amino acid residues predicted to be on the surface of the protein, since such residues may be involved in important interactions with other molecules. Moreover, one skilled in the art may generate test variants containing a single amino acid substitution at each desired amino acid residue. These variants can then be screened using standard assays for binding and/or activity, thus yielding information gathered from such routine experiments, which may allow one skilled in the art to determine the amino acid positions where further substitutions should be avoided either alone or in combination with other mutations. Various tools available to determine secondary structure can be found on the world wide web at expasy.org/proteomics/protein_structure.

In some embodiments of the disclosure, amino acid substitutions are made that: (1) reduce susceptibility to proteolysis, (2) reduce susceptibility to oxidation, (3) alter binding affinity for forming protein complexes, (4) alter ligand or antigen binding affinities, and/or (5) confer or modify other physicochemical or functional properties on such polypeptides. For example, single or multiple amino acid substitutions (in certain embodiments, conservative amino acid substitutions) may be made in the naturally occurring sequence. Substitutions can be made in that portion of the antibody that lies outside the domain(s) forming intermolecular contacts. In such embodiments, conservative amino acid substitutions can be used that do not substantially change the structural characteristics of the protein or polypeptide (e.g., one or more replacement amino acids that do not disrupt the secondary structure that characterizes the native antibody).

### C. Sequences

The amino acid sequence of certain polypeptides, including antibodies, chimeric antigen receptors, chimeric polypeptides, immune cell engagers, and portions, regions, and domains thereof, are provided in Table 1.

TABLE 1

| | SEQ | |
|---|---|---|
| Name | ID NO: | Sequence |
| m6-CD70V_{H} | 44 | QVQLQQSGAELMKPGASVKISCKATGYTFSNHWIEWVKE |
| | | RPGHGLEWIGEILLGSGRAHYNEKFKGKATFTADTSSNTA |
| | | YMQLSSLTSEDSAVYYCARHYRYDGWFAYWGQGTPVTV |
| | | SA |
| m6-CD70V_{H} | 45 | GYTFSNHW |
| CDRH1 | | |
| m6-CD70V_{H} | 46 | ILLGSGRA |
| CDRH2 | | |
| m6-CD70V_{H} | 47 | ARHYRYDGWFAY |
| CDRH3 | | |
| m6-CD70V_{L} | 48 | DIVLTQSPASLAVSLGQRATISCRASKSVSTSGYSYMHWY |
| | | QQKPGQPPKLLIYLASNLESGVPARFSGSGSGTDFTLNIHP |
| | | VEEEDAATYYCQHSRELPYTFGGGTKLEIK |
| m6-CD70V_{L} | 49 | KSVSTSGYSY |
| CDRL1 | | |
| m6-CD70V_{L} | 50 | LAS |
| CDRL2 | | |
| m6-CD70V_{L} | 51 | QHSRELPYT |
| CDRL3 | | |
| m7-CD70V_{H} | 52 | QVQLKQSGPGLVQPSQSLSITCTVSGFSLTSYGVHWVRQS |
| | | PGKGLEWLGVIWSGGSTDYNAAFISRLSISKDNSKSQVFFK |
| | | MNSLQANDTAIYYCATYYRYDGWFAYWGQGTLVTVSA |
| m7-CD70V_{H} | 53 | GFSLTSYG |
| CDRH1 | | |
| m7-CD70V_{H} | 54 | IWSGGST |
| CDRH2 | | |
| m7-CD70V_{H} | 55 | ATYYRYDGWFAY |
| CDRH3 | | |
| m7-CD70V_{L} | 56 | DIVLTQSPASLAVSLGQRATISCRASKSVSTSGYSYMHWY |
| | | QQKPGQPPKLLIYLASNLESGVPARFSGSGSGTDFTLNIHP |
| | | VEEEDAATYSCQHSRELPWTFGGGTKLEIK |
| m7-CD70V_{L} | 57 | KSVSTSGYSY |
| CDRL1 | | |
| m7-CD70V_{L} | 58 | LAS |
| CDRL2 | | |
| m7-CD70V_{L} | 59 | QHSRELPWT |
| CDRL3 | | |
| m14-CD70V_{H} | 60 | EVQLQQSGAELVKPGASVKLSCTASGFNIKDSYMHWVKQ |
| | | RPEQGLEWIGRIDPANANPKYDPKFQGKATITTDTSSNTA |
| | | YLQLSSLTSEDTAVYYCARDYGGYFDVWGAGTTVTVSS |
| m14-CD70V_{H} | 61 | GFNIKDSY |
| CDRH1 | | |
| m14-CD70V_{H} | 62 | IDPANANP |
| CDRH2 | | |
| m14-CD70V_{H} | 63 | ARDYGGYFDV |
| CDRH3 | | |
| m14-CD70V_{L} | 64 | DIQMTQSSSYLSVSLGGRVTITCKASDHINNWLAWYQQKP |
| | | GNAPRLLISGATSLETGVPSRFSGSGSGKDYTLSITSLQTED |
| | | VATYYCQQYWSTPWTFGGGTKLEIK |
| m14-CD70V_{L} | 65 | DHINNW |
| CDRL1 | | |
| m14-CD70V_{L} | 66 | GAT |
| CDRL2 | | |
| m14-CD70V_{L} | 67 | QQYWSTPWT |
| CDRL3 | | |
| m6-CD70 (V_{H}- | 68 | QVQLQQSGAELMKPGASVKISCKATGYTFSNHWIEWVKE |
| linker-V_{L}) | | RPGHGLEWIGEILLGSGRAHYNEKFKGKATFTADTSSNTA |
| | | YMQLSSLTSEDSAVYYCARHYRYDGWFAYWGQGTPVTV |
| | | SAGGGGSGGGGSGGGASDIVLTQSPASLAVSLGQRATISC |
| | | RASKSVSTSGYSYMHWYQQKPGQPPKLLIYLASNLESGVP |
| | | ARFSGSGSGTDFTLNIHPVEEEDAATYYCQHSRELPYTFGG |
| | | GTKLEIK |
| m6-CD70 (V_{L}- | 69 | DIVLTQSPASLAVSLGQRATISCRASKSVSTSGYSYMHWY |
| linker-V_{H}) | | QQKPGQPPKLLIYLASNLESGVPARFSGSGSGTDFTLNIHP |
| | | VEEEDAATYYCQHSRELPYTFGGGTKLEIKGGGGSGGGGS |
| | | GGGASQVQLQQSGAELMKPGASVKISCKATGYTFSNHWI |
| | | EWVKERPGHGLEWIGEILLGSGRAHYNEKFKGKATFTAD |
| | | TSSNTAYMQLSSLTSEDSAVYYCARHYRYDGWFAYWGQ |
| | | GTPVTVSA |
| m7-CD70 (V_{H}- | 70 | QVQLKQSGPGLVQPSQSLSITCTVSGFSLTSYGVHWVRQS |
| linker-V_{L}) | | PGKGLEWLGVIWSGGSTDYNAAFISRLSISKDNSKSQVFFK |
| | | MNSLQANDTAIYYCATYYRYDGWFAYWGQGTLVTVSAG |
| | | GGGSGGGGSGGGASDIVLTQSPASLAVSLGQRATISCRAS |
| | | KSVSTSGYSYMHWYQQKPGQPPKLLIYLASNLESGVPARF |
| | | SGSGSGTDFTLNIHPVEEEDAATYSCQHSRELPWTFGGGT |
| | | KLEIK |
| m7-CD70 (V_{L}- | 71 | DIVLTQSPASLAVSLGQRATISCRASKSVSTSGYSYMHWY |
| linker-V_{H}) | | QQKPGQPPKLLIYLASNLESGVPARFSGSGSGTDFTLNIHP |
| | | VEEEDAATYSCQHSRELPWTFGGGTKLEIKGGGGSGGGG |
| | | SGGGASQVQLKQSGPGLVQPSQSLSITCTVSGFSLTSYGVH |
| | | WVRQSPGKGLEWLGVIWSGGSTDYNAAFISRLSISKDNSK |
| | | SQVFFKMNSLQANDTAIYYCATYYRYDGWFAYWGQGTL |
| | | VTVSA |
| m14-CD70 | 72 | EVQLQQSGAELVKPGASVKLSCTASGFNIKDSYMHWVKQ |
| (V_{H}-linker-V_{L}) | | RPEQGLEWIGRIDPANANPKYDPKFQGKATITTDTSSNTA |
| | | YLQLSSLTSEDTAVYYCARDYGGYFDVWGAGTTVTVSSG |
| | | GGGSGGGGSGGGASDIQMTQSSSYLSVSLGGRVTITCKAS |
| | | DHINNWLAWYQQKPGNAPRLLISGATSLETGVPSRFSGSG |
| | | SGKDYTLSITSLQTEDVATYYCQQYWSTPWTFGGGTKLEI |
| | | K |
| m14-CD70 (V_{L}- | 73 | DIQMTQSSSYLSVSLGGRVTITCKASDHINNWLAWYQQKP |
| linker-V_{H}) | | GNAPRLLISGATSLETGVPSRFSGSGSGKDYTLSITSLQTED |
| | | VATYYCQQYWSTPWTFGGGTKLEIKGGGGSGGGGSGGG |
| | | ASEVQLQQSGAELVKPGASVKLSCTASGFNIKDSYMHWV |
| | | KQRPEQGLEWIGRIDPANANPKYDPKFQGKATITTDTSSN |
| | | TAYLQLSSLTSEDTAVYYCARDYGGYFDVWGAGTTVTVS |
| | | S |
| Linker | 74 | GGGGSGGGGSGGGAS |
| CD28 | 75 | FWVLVVVGGVLACYSLLVTVAFIIFWV |
| transmembrane | | |
| domain | | |
| CD8 | 76 | TTTPAPRPPTPAPTIASQPLSLRPEACRPAAGGAVHTRGLD |
| transmembrane | | FACDIYIWAPLAGTCGVLLLSLVIT |
| domain | | |
| 4-1BB | 77 | IISFFLALTSTALLFLLFFLTLRFSVV |
| transmembrane | | |
| domain | | |
| DAP10 | 78 | LLAGLVAADAVASLLIVGAVF |
| transmembrane | | |
| domain | | |
| DAP12 | 79 | GVLAGIVMGDLVLTVLIALAV |
| transmembrane | | |
| domain | | |
| NKG2D | 80 | AVMIIFRIGMAVAIFCCFFFP |
| transmembrane | | |
| domain | | |
| CD3ζ | 81 | TRKKFSRSADAPAYQQGQNQLYNELNLGRREEYDVLDKR |
| intracellular | | RGRDPEMGGKPRRKNPQEGLYNELQKDKMAEAYSEIGM |
| domain-1 | | KGERRRGKGHDGLYQGLSTATKDTYDALHMQALPPRG |
| CD3ζ | 82 | KRVKFSRSADAPAYQQGQNQLYNELNLGRREEYDVLDKR |
| intracellular | | RGRDPEMGGKPRRKNPQEGLYNELQKDKMAEAYSEIGM |
| domain-2 | | KGERRRGKGHDGLYQGLSTATKDTYDALHMQALPPRG |
| 4-1BB | 83 | KRGRKKLLYIFKQPFMRPVQTTQEEDGCSCRFPEEEEGGC |
| intracellular | | EL |
| domain | | |
| DAP10 | 84 | LCARPRRSPAQEDGKVYINMPGRG |
| intracellular | | |
| domain | | |
| DAP12 | 85 | YFLGRLVPRGRGAAEAATRKQRITETESPYQELQGQRSDV |
| intracellular | | YSDLNTQRPYYK |
| domain | | |
| NKG2D | 86 | SANERCKSKVVPCRQKQWRTSFDSKKLDLNYNHFESME |
| intracellular | | WSHRSRRGRIWGM |
| domain | | |
| IgG Hinge | 87 | TVTVSSQDPAEPKSPDKTHTCPPCPAPELLGGPSVFLFPPKP |
| | | KDTLMISRTPEVTCVVVDVSHEDPEVKFNWYVDGVEVHN |
| | | AKTKPREEQYNSTYRVVSVLTVLHQDWLNGKEYKCKVS |
| | | NKALPAPIEKTISKAKGQPREPQVYTLPPSRDELTKNQVSL |
| | | TCLVKGFYPSDIAVEWESNGQPENNYKTTPPVLDSDGSFF |
| | | LYSKLTVDKSRWQQGNVFSCSVMHEAL |
| CD28 Hinge | 88 | IEVMYPPPYLDNEKSNGTIIHVKGKHLCPSPLFPGPSKP |
| IL-15 | 89 | ISKPHLRSISIQCYLCLLLNSHFLTEAGIHVFILGCFSAGLPK |
| | | TEANWVNVISDLKKIEDLIQSMHIDATLYTESDVHPSCKVT |
| | | AMKCFLLELQVISLESGDASIHDTVENLIILANNSLSSNGN |
| | | VTESGCKECEELEEKNIKEFLQSFVHIVQMFINTS |
| E2A | 90 | QCTNYALLKLAGDVESNPGP |
| T2A | 91 | EGRGSLLTCGDVEENPGP |
| P2A | 92 | ATNFSLLKQAGDVEENPGP |
| F2A | 93 | VKQTLNFDLLKLAGDVESNPGP |

### D. CD70

CD70, also known as CD70 antigen, CD27 ligand, and Tumor necrosis factor ligand superfamily member 7, is encoded by the CD70 gene (also known as TNFSF7). A CD70 mRNA sequence is provided by RefSeq accession number NM_001252. A CD70 protein sequence is provided by RefSeq accession number NP_001243.

### V. Nucleic Acids

In certain embodiments, nucleic acid sequences can exist in a variety of instances such as: isolated segments and recombinant vectors of incorporated sequences or recombinant polynucleotides encoding one or both chains of an antibody, or a fragment, derivative, mutein, or variant thereof, polynucleotides encoding a chimeric polypeptide, polynucleotides encoding a chimeric antigen receptor, polynucleotides encoding an immune cell engager, polynucleotides sufficient for use as hybridization probes, PCR primers or sequencing primers for identifying, analyzing, mutating or amplifying a polynucleotide encoding a polypeptide, anti-sense nucleic acids for inhibiting expression of a polynucleotide, and complementary sequences of the foregoing described herein. Nucleic acids that encode the epitope to which certain of the antibodies provided herein are also provided. Nucleic acids encoding fusion proteins that include these peptides are also provided. The nucleic acids can be single-stranded or double-stranded and can comprise RNA and/or DNA nucleotides and artificial variants thereof (e.g., peptide nucleic acids).

The term “polynucleotide” refers to a nucleic acid molecule that either is recombinant or has been isolated from total genomic nucleic acid. Included within the term “polynucleotide” are oligonucleotides (nucleic acids 100 residues or less in length), recombinant vectors, including, for example, plasmids, cosmids, phage, viruses, and the like. Polynucleotides include, in certain aspects, regulatory sequences, isolated substantially away from their naturally occurring genes or protein encoding sequences. Polynucleotides may be single-stranded (coding or antisense) or double-stranded, and may be RNA, DNA (genomic, cDNA or synthetic), analogs thereof, or a combination thereof. Additional coding or non-coding sequences may, but need not, be present within a polynucleotide.

In this respect, the term “gene,” “polynucleotide,” or “nucleic acid” is used to refer to a nucleic acid that encodes a protein, polypeptide, or peptide (including any sequences required for proper transcription, post-translational modification, or localization). As will be understood by those in the art, this term encompasses genomic sequences, expression cassettes, cDNA sequences, and smaller engineered nucleic acid segments that express, or may be adapted to express, proteins, polypeptides, domains, peptides, fusion proteins, and mutants. A nucleic acid encoding all or part of a polypeptide may contain a contiguous nucleic acid sequence encoding all or a portion of such a polypeptide. It also is contemplated that a particular polypeptide may be encoded by nucleic acids containing variations having slightly different nucleic acid sequences but, nonetheless, encode the same or substantially similar protein.

In certain embodiments, there are polynucleotide variants having substantial identity to the sequences disclosed herein; those comprising at least, at most, exactly, or between any two of 70%, 75%, 80%, 85%, 90%, 95%, 96%, 97%, 98%, or 99% or higher sequence identity, including all values and ranges there between, compared to a polynucleotide sequence provided herein using the methods described herein (e.g., BLAST analysis using standard parameters). In certain aspects, the isolated polynucleotide will comprise a nucleotide sequence encoding a polypeptide that has at least 90%, preferably 95% and above, identity to an amino acid sequence described herein, over the entire length of the sequence; or a nucleotide sequence complementary to said isolated polynucleotide.

The nucleic acid segments, regardless of the length of the coding sequence itself, may be combined with other nucleic acid sequences, such as promoters, polyadenylation signals, additional restriction enzyme sites, multiple cloning sites, other coding segments, and the like, such that their overall length may vary considerably. The nucleic acids can be any length. They can be, for example, at least, at most, exactly, or between any two of 5, 10, 15, 20, 25, 30, 35, 40, 45, 50, 75, 100, 125, 175, 200, 250, 300, 350, 400, 450, 500, 750, 1000, 1500, 3000, 5000 or more nucleotides in length, and/or can comprise one or more additional sequences, for example, regulatory sequences, and/or be a part of a larger nucleic acid, for example, a vector. It is therefore contemplated that a nucleic acid fragment of almost any length may be employed, with the total length preferably being limited by the ease of preparation and use in the intended recombinant nucleic acid protocol. In some cases, a nucleic acid sequence may encode a polypeptide sequence with additional heterologous coding sequences, for example to allow for purification of the polypeptide, transport, secretion, post-translational modification, or for therapeutic benefits such as targeting or efficacy. As discussed above, a tag or other heterologous polypeptide may be added to the modified polypeptide-encoding sequence, wherein “heterologous” refers to a polypeptide that is not the same as the modified polypeptide.

### A. Mutation

Changes can be introduced by mutation into a nucleic acid, thereby leading to changes in the amino acid sequence of a polypeptide (e.g., an antibody or antibody derivative, a chimeric polypeptide, etc.) that it encodes. Mutations can be introduced using any technique known in the art. In one embodiment, one or more particular amino acid residues are changed using, for example, a site-directed mutagenesis protocol. In another embodiment, one or more randomly selected residues are changed using, for example, a random mutagenesis protocol. However it is made, a mutant polypeptide can be expressed and screened for a desired property.

Mutations can be introduced into a nucleic acid without significantly altering the biological activity of a polypeptide that it encodes. For example, one can make nucleotide substitutions leading to amino acid substitutions at non-essential amino acid residues. Alternatively, one or more mutations can be introduced into a nucleic acid that selectively changes the biological activity of a polypeptide that it encodes. See, eg., Romain Studer et al., Biochem. J. 449:581-594 (2013). For example, the mutation can quantitatively or qualitatively change the biological activity. Examples of quantitative changes include increasing, reducing or eliminating the activity. Examples of qualitative changes include altering the antigen specificity of an antibody.

### B. Probes

In another aspect, nucleic acid molecules are suitable for use as primers or hybridization probes for the detection of nucleic acid sequences. A nucleic acid molecule can comprise only a portion of a nucleic acid sequence encoding a full-length polypeptide, for example, a fragment that can be used as a probe or primer or a fragment encoding an active portion of a given polypeptide.

In another embodiment, the nucleic acid molecules may be used as probes or PCR primers for specific sequences. For instance, a nucleic acid molecule probe may be used in diagnostic methods or a nucleic acid molecule PCR primer may be used to amplify regions of DNA that could be used, inter alia, to isolate nucleic acid sequences for use in producing variable domains of antibodies. See, eg., Gaily Kivi et al., BMC Biotechnol. 16:2 (2016). In some embodiments, the nucleic acid molecules are oligonucleotides. In some embodiments, the oligonucleotides are from highly variable regions of the heavy and light chains of the antibody of interest. In some embodiments, the oligonucleotides encode all or part of one or more CDRs.

Probes based on the desired sequence of a nucleic acid can be used to detect the nucleic acid or similar nucleic acids, for example, transcripts encoding a polypeptide of interest. The probe can comprise a label group, e.g., a radioisotope, a fluorescent compound, an enzyme, or an enzyme co-factor. Such probes can be used to identify a cell that expresses the polypeptide.

### C. Sequences

The nucleic acid sequences encoding certain polypeptides, including antibodies, chimeric antigen receptors, chimeric polypeptides, immune cell engagers, and portions, regions, and domains thereof, are provided in Table 2.

TABLE 2

| | SEQ | |
|---|---|---|
| Name | ID NO: | Sequence |
| m6-CD70V_{H} | 1 | CAGGTTCAGCTGCAGCAGTCTGGAGCTGAGCTGATGAAGC |
| | | CTGGGGCCTCAGTGAAGATATCCTGCAAGGCAACTGGCTA |
| | | CACATTCAGTAACCACTGGATAGAGTGGGTTAAGGAAAG |
| | | GCCTGGACATGGCCTGGAGTGGATTGGAGAGATTTTACTT |
| | | GGAAGTGGTAGAGCTCATTATAATGAGAAGTTCAAGGGC |
| | | AAGGCCACATTCACTGCAGATACATCCTCCAACACAGCCT |
| | | ACATGCAACTCAGCAGCCTGACATCTGAGGACTCTGCCGT |
| | | CTATTACTGTGCAAGACACTATAGGTACGACGGGTGGTTT |
| | | GCTTACTGGGGCCAAGGGACTCCGGTCACTGTCTCTGCA |
| m6-CD70V_{H} | 2 | GGCTACACATTCAGTAACCACTGG |
| CDRH1 | | |
| m6-CD70V_{H} | 3 | ATTTTACTTGGAAGTGGTAGAGCT |
| CDRH2 | | |
| m6-CD70V_{H} | 4 | GCAAGACACTATAGGTACGACGGGTGGTTTGCTTAC |
| CDRH3 | | |
| m6-CD70V_{L} | 5 | GACATTGTGCTGACACAGTCTCCTGCTTCCTTAGCTGTATC |
| | | TCTGGGGCAGAGGGCCACCATCTCATGCAGGGCCAGCAA |
| | | AAGTGTCAGTACATCTGGCTATAGTTATATGCACTGGTAC |
| | | CAACAGAAACCAGGACAGCCACCCAAACTCCTCATCTATC |
| | | TTGCATCCAACCTAGAATCTGGGGTCCCTGCCAGGTTCAG |
| | | TGGCAGTGGGTCTGGGACAGACTTCACCCTCAACATCCAT |
| | | CCTGTGGAGGAGGAGGATGCTGCAACCTATTACTGTCAGC |
| | | ACAGTAGGGAGCTTCCGTACACGTTCGGAGGGGGGACCA |
| | | AGCTGGAAATAAAA |
| m6-CD70V_{L} | 6 | AAAAGTGTCAGTACATCTGGCTATAGTTAT |
| CDRL1 | | |
| m6-CD70V_{L} | 7 | CTTGCATCC |
| CDRL2 | | |
| m6-CD70V_{L} | 8 | CAGCACAGTAGGGAGCTTCCGTACACG |
| CDRL3 | | |
| m7-CD70V_{H} | 9 | CAGGTGCAGCTGAAGCAGTCAGGACCTGGCCTAGTGCAG |
| | | CCCTCACAGAGCCTGTCCATCACCTGCACAGTCTCTGGTTT |
| | | CTCATTAACTAGCTATGGTGTACACTGGGTTCGCCAGTCTC |
| | | CAGGAAAGGGTCTGGAGTGGCTGGGAGTGATATGGAGTG |
| | | GTGGAAGCACAGACTATAATGCAGCTTTCATATCCAGACT |
| | | GAGCATCAGCAAGGACAATTCCAAGAGCCAAGTTTTCTTT |
| | | AAAATGAACAGTCTGCAAGCTAATGACACAGCCATATATT |
| | | ACTGTGCCACGTACTATAGGTACGACGGGTGGTTTGCTTA |
| | | CTGGGGCCAAGGGACTCTGGTCACTGTCTCTGCA |
| m7-CD70V_{H} | 10 | GGTTTCTCATTAACTAGCTATGGT |
| CDRH1 | | |
| m7-CD70V_{H} | 11 | ATATGGAGTGGTGGAAGCACA |
| CDRH2 | | |
| m7-CD70V_{H} | 12 | GCCACGTACTATAGGTACGACGGGTGGTTTGCTTAC |
| CDRH3 | | |
| m7-CD70V_{L} | 13 | GACATTGTGCTGACACAGTCTCCTGCTTCCTTAGCTGTATC |
| | | TCTGGGGCAGAGGGCCACCATCTCATGCAGGGCCAGCAA |
| | | AAGTGTCAGTACATCTGGCTATAGTTATATGCACTGGTAT |
| | | CAACAGAAACCAGGACAGCCACCCAAACTCCTCATCTATC |
| | | TTGCATCCAACCTAGAATCTGGGGTCCCTGCCAGGTTCAG |
| | | TGGCAGTGGGTCTGGGACAGACTTCACCCTCAACATCCAT |
| | | CCTGTGGAGGAGGAGGATGCTGCAACCTATTCCTGTCAGC |
| | | ACAGTAGGGAGCTTCCGTGGACGTTCGGTGGAGGCACCA |
| | | AGCTGGAAATCAAA |
| m7-CD70V_{L} | 14 | AAAAGTGTCAGTACATCTGGCTATAGTTAT |
| CDRL1 | | |
| m7-CD70V_{L} | 15 | CTTGCATCC |
| CDRL2 | | |
| m7-CD70V_{L} | 16 | CAGCACAGTAGGGAGCTTCCGTGGACG |
| CDRL3 | | |
| m14-CD70V_{H} | 17 | GAGGTTCAGCTGCAGCAGTCTGGGGCAGAGCTTGTGAAGC |
| | | CAGGGGCCTCAGTCAAGTTGTCCTGCACAGCTTCTGGCTT |
| | | CAACATTAAAGACAGCTATATGCACTGGGTGAAGCAGAG |
| | | GCCTGAACAGGGCCTGGAGTGGATTGGAAGGATTGATCCT |
| | | GCGAATGCTAATCCTAAATATGACCCGAAGTTCCAGGGCA |
| | | AGGCCACTATAACAACAGACACATCCTCCAACACAGCCTA |
| | | CCTGCAGCTCAGCAGCCTGACATCTGAGGACACTGCCGTC |
| | | TATTACTGTGCTAGAGACTACGGAGGGTACTTCGATGTCT |
| | | GGGGCGCAGGGACCACGGTCACCGTCTCCTCA |
| m14-CD70V_{H} | 18 | GGCTTCAACATTAAAGACAGCTAT |
| CDRH1 | | |
| m14-CD70V_{H} | 19 | ATTGATCCTGCGAATGCTAATCCTAAA |
| CDRH2 | | |
| m14-CD70V_{H} | 20 | GCTAGAGACTACGGAGGGTACTTCGATGTC |
| CDRH3 | | |
| m14-CD70V_{L} | 21 | GACATCCAGATGACACAATCTTCATCCTACTTGTCTGTATC |
| | | TCTAGGAGGCAGAGTCACCATTACTTGCAAGGCAAGTGAC |
| | | CACATTAATAATTGGTTAGCCTGGTATCAGCAGAAACCAG |
| | | GAAATGCTCCTAGGCTCTTAATATCTGGTGCAACCAGTTT |
| | | GGAAACTGGGGTTCCTTCAAGATTCAGTGGCAGTGGATCT |
| | | GGAAAGGATTACACTCTCAGCATTACCAGTCTTCAGACTG |
| | | AAGATGTTGCCACTTATTACTGTCAACAGTATTGGAGTAC |
| | | TCCGTGGACGTTCGGTGGAGGCACCAAGCTGGAAATCAA |
| | | AC |
| m14-CD70V_{L} | 22 | GACCACATTAATAATTGG |
| CDRL1 | | |
| m14-CD70V_{L} | 23 | GGTGCAACC |
| CDRL2 | | |
| m14-CD70V_{L} | 24 | CAACAGTATTGGAGTACTCCGTGGACG |
| CDRL3 | | |
| m6-CD70 (V_{H}- | 25 | CAGGTTCAGCTGCAGCAGTCTGGAGCTGAGCTGATGAAGC |
| linker-V_{L}) | | CTGGGGCCTCAGTGAAGATATCCTGCAAGGCAACTGGCTA |
| | | CACATTCAGTAACCACTGGATAGAGTGGGTTAAGGAAAG |
| | | GCCTGGACATGGCCTGGAGTGGATTGGAGAGATTTTACTT |
| | | GGAAGTGGTAGAGCTCATTATAATGAGAAGTTCAAGGGC |
| | | AAGGCCACATTCACTGCAGATACATCCTCCAACACAGCCT |
| | | ACATGCAACTCAGCAGCCTGACATCTGAGGACTCTGCCGT |
| | | CTATTACTGTGCAAGACACTATAGGTACGACGGGTGGTTT |
| | | GCTTACTGGGGCCAAGGGACTCCGGTCACTGTCTCTGCAG |
| | | GTGGAGGCGGTTCAGGCGGAGGTGGCTCTGGCGGTGGCG |
| | | CTAGCGACATTGTGCTGACACAGTCTCCTGCTTCCTTAGCT |
| | | GTATCTCTGGGGCAGAGGGCCACCATCTCATGCAGGGCCA |
| | | GCAAAAGTGTCAGTACATCTGGCTATAGTTATATGCACTG |
| | | GTACCAACAGAAACCAGGACAGCCACCCAAACTCCTCATC |
| | | TATCTTGCATCCAACCTAGAATCTGGGGTCCCTGCCAGGT |
| | | TCAGTGGCAGTGGGTCTGGGACAGACTTCACCCTCAACAT |
| | | CCATCCTGTGGAGGAGGAGGATGCTGCAACCTATTACTGT |
| | | CAGCACAGTAGGGAGCTTCCGTACACGTTCGGAGGGGGG |
| | | ACCAAGCTGGAAATAAAA |
| m6-CD70 (V_{L}- | 26 | GACATTGTGCTGACACAGTCTCCTGCTTCCTTAGCTGTATC |
| linker-V_{H}) | | TCTGGGGCAGAGGGCCACCATCTCATGCAGGGCCAGCAA |
| | | AAGTGTCAGTACATCTGGCTATAGTTATATGCACTGGTAC |
| | | CAACAGAAACCAGGACAGCCACCCAAACTCCTCATCTATC |
| | | TTGCATCCAACCTAGAATCTGGGGTCCCTGCCAGGTTCAG |
| | | TGGCAGTGGGTCTGGGACAGACTTCACCCTCAACATCCAT |
| | | CCTGTGGAGGAGGAGGATGCTGCAACCTATTACTGTCAGC |
| | | ACAGTAGGGAGCTTCCGTACACGTTCGGAGGGGGGACCA |
| | | AGCTGGAAATAAAAGGTGGAGGCGGTTCAGGCGGAGGTG |
| | | GCTCTGGCGGTGGCGCTAGCCAGGTTCAGCTGCAGCAGTC |
| | | TGGAGCTGAGCTGATGAAGCCTGGGGCCTCAGTGAAGAT |
| | | ATCCTGCAAGGCAACTGGCTACACATTCAGTAACCACTGG |
| | | ATAGAGTGGGTTAAGGAAAGGCCTGGACATGGCCTGGAG |
| | | TGGATTGGAGAGATTTTACTTGGAAGTGGTAGAGCTCATT |
| | | ATAATGAGAAGTTCAAGGGCAAGGCCACATTCACTGCAG |
| | | ATACATCCTCCAACACAGCCTACATGCAACTCAGCAGCCT |
| | | GACATCTGAGGACTCTGCCGTCTATTACTGTGCAAGACAC |
| | | TATAGGTACGACGGGTGGTTTGCTTACTGGGGCCAAGGGA |
| | | CTCCGGTCACTGTCTCTGCA |
| m7-CD70 (V_{H}- | 27 | CAGGTGCAGCTGAAGCAGTCAGGACCTGGCCTAGTGCAG |
| linker-V_{L}) | | CCCTCACAGAGCCTGTCCATCACCTGCACAGTCTCTGGTTT |
| | | CTCATTAACTAGCTATGGTGTACACTGGGTTCGCCAGTCTC |
| | | CAGGAAAGGGTCTGGAGTGGCTGGGAGTGATATGGAGTG |
| | | GTGGAAGCACAGACTATAATGCAGCTTTCATATCCAGACT |
| | | GAGCATCAGCAAGGACAATTCCAAGAGCCAAGTTTTCTTT |
| | | AAAATGAACAGTCTGCAAGCTAATGACACAGCCATATATT |
| | | ACTGTGCCACGTACTATAGGTACGACGGGTGGTTTGCTTA |
| | | CTGGGGCCAAGGGACTCTGGTCACTGTCTCTGCAGGTGGA |
| | | GGCGGTTCAGGCGGAGGTGGCTCTGGCGGTGGCGCTAGC |
| | | GACATTGTGCTGACACAGTCTCCTGCTTCCTTAGCTGTATC |
| | | TCTGGGGCAGAGGGCCACCATCTCATGCAGGGCCAGCAA |
| | | AAGTGTCAGTACATCTGGCTATAGTTATATGCACTGGTAT |
| | | CAACAGAAACCAGGACAGCCACCCAAACTCCTCATCTATC |
| | | TTGCATCCAACCTAGAATCTGGGGTCCCTGCCAGGTTCAG |
| | | TGGCAGTGGGTCTGGGACAGACTTCACCCTCAACATCCAT |
| | | CCTGTGGAGGAGGAGGATGCTGCAACCTATTCCTGTCAGC |
| | | ACAGTAGGGAGCTTCCGTGGACGTTCGGTGGAGGCACCA |
| | | AGCTGGAAATCAAA |
| m7-CD70 (V_{L}- | 28 | GACATTGTGCTGACACAGTCTCCTGCTTCCTTAGCTGTATC |
| linker-V_{H}) | | TCTGGGGCAGAGGGCCACCATCTCATGCAGGGCCAGCAA |
| | | AAGTGTCAGTACATCTGGCTATAGTTATATGCACTGGTAT |
| | | CAACAGAAACCAGGACAGCCACCCAAACTCCTCATCTATC |
| | | TTGCATCCAACCTAGAATCTGGGGTCCCTGCCAGGTTCAG |
| | | TGGCAGTGGGTCTGGGACAGACTTCACCCTCAACATCCAT |
| | | CCTGTGGAGGAGGAGGATGCTGCAACCTATTCCTGTCAGC |
| | | ACAGTAGGGAGCTTCCGTGGACGTTCGGTGGAGGCACCA |
| | | AGCTGGAAATCAAAGGTGGAGGCGGTTCAGGCGGAGGTG |
| | | GCTCTGGCGGTGGCGCTAGCCAGGTGCAGCTGAAGCAGTC |
| | | AGGACCTGGCCTAGTGCAGCCCTCACAGAGCCTGTCCATC |
| | | ACCTGCACAGTCTCTGGTTTCTCATTAACTAGCTATGGTGT |
| | | ACACTGGGTTCGCCAGTCTCCAGGAAAGGGTCTGGAGTGG |
| | | CTGGGAGTGATATGGAGTGGTGGAAGCACAGACTATAAT |
| | | GCAGCTTTCATATCCAGACTGAGCATCAGCAAGGACAATT |
| | | CCAAGAGCCAAGTTTTCTTTAAAATGAACAGTCTGCAAGC |
| | | TAATGACACAGCCATATATTACTGTGCCACGTACTATAGG |
| | | TACGACGGGTGGTTTGCTTACTGGGGCCAAGGGACTCTGG |
| | | TCACTGTCTCTGCA |
| m14-CD70 | 29 | GAGGTTCAGCTGCAGCAGTCTGGGGCAGAGCTTGTGAAGC |
| (V_{H}-linker-V_{L}) | | CAGGGGCCTCAGTCAAGTTGTCCTGCACAGCTTCTGGCTT |
| | | CAACATTAAAGACAGCTATATGCACTGGGTGAAGCAGAG |
| | | GCCTGAACAGGGCCTGGAGTGGATTGGAAGGATTGATCCT |
| | | GCGAATGCTAATCCTAAATATGACCCGAAGTTCCAGGGCA |
| | | AGGCCACTATAACAACAGACACATCCTCCAACACAGCCTA |
| | | CCTGCAGCTCAGCAGCCTGACATCTGAGGACACTGCCGTC |
| | | TATTACTGTGCTAGAGACTACGGAGGGTACTTCGATGTCT |
| | | GGGGCGCAGGGACCACGGTCACCGTCTCCTCAGGTGGAG |
| | | GCGGTTCAGGCGGAGGTGGCTCTGGCGGTGGCGCTAGCG |
| | | ACATCCAGATGACACAATCTTCATCCTACTTGTCTGTATCT |
| | | CTAGGAGGCAGAGTCACCATTACTTGCAAGGCAAGTGACC |
| | | ACATTAATAATTGGTTAGCCTGGTATCAGCAGAAACCAGG |
| | | AAATGCTCCTAGGCTCTTAATATCTGGTGCAACCAGTTTG |
| | | GAAACTGGGGTTCCTTCAAGATTCAGTGGCAGTGGATCTG |
| | | GAAAGGATTACACTCTCAGCATTACCAGTCTTCAGACTGA |
| | | AGATGTTGCCACTTATTACTGTCAACAGTATTGGAGTACT |
| | | CCGTGGACGTTCGGTGGAGGCACCAAGCTGGAAATCAAA |
| | | C |
| m14-CD70 (V_{L}- | 30 | GACATCCAGATGACACAATCTTCATCCTACTTGTCTGTATC |
| linker-V_{H}) | | TCTAGGAGGCAGAGTCACCATTACTTGCAAGGCAAGTGAC |
| | | CACATTAATAATTGGTTAGCCTGGTATCAGCAGAAACCAG |
| | | GAAATGCTCCTAGGCTCTTAATATCTGGTGCAACCAGTTT |
| | | GGAAACTGGGGTTCCTTCAAGATTCAGTGGCAGTGGATCT |
| | | GGAAAGGATTACACTCTCAGCATTACCAGTCTTCAGACTG |
| | | AAGATGTTGCCACTTATTACTGTCAACAGTATTGGAGTAC |
| | | TCCGTGGACGTTCGGTGGAGGCACCAAGCTGGAAATCAA |
| | | ACGGTGGAGGCGGTTCAGGCGGAGGTGGCTCTGGCGGTG |
| | | GCGCTAGCGAGGTTCAGCTGCAGCAGTCTGGGGCAGAGCT |
| | | TGTGAAGCCAGGGGCCTCAGTCAAGTTGTCCTGCACAGCT |
| | | TCTGGCTTCAACATTAAAGACAGCTATATGCACTGGGTGA |
| | | AGCAGAGGCCTGAACAGGGCCTGGAGTGGATTGGAAGGA |
| | | TTGATCCTGCGAATGCTAATCCTAAATATGACCCGAAGTT |
| | | CCAGGGCAAGGCCACTATAACAACAGACACATCCTCCAA |
| | | CACAGCCTACCTGCAGCTCAGCAGCCTGACATCTGAGGAC |
| | | ACTGCCGTCTATTACTGTGCTAGAGACTACGGAGGGTACT |
| | | TCGATGTCTGGGGCGCAGGGACCACGGTCACCGTCTCCTC |
| | | A |
| Linker | 31 | GGTGGAGGCGGTTCAGGCGGAGGTGGCTCTGGCGGTGGC |
| | | GCTAGC |

### VII. Obtaining Encoded Polypeptide Embodiments

In some aspects, there are nucleic acid molecules encoding antibody polypeptides (e.g., heavy or light chain, variable domain only, or full-length), chimeric polypeptides, chimeric antigen receptors, or other polypeptides described herein. These may be generated by methods known in the art, e.g., isolated from B cells of mice that have been immunized and isolated, phage display, expressed in any suitable recombinant expression system and allowed to assemble to form antibody molecules.

### A. Expression

The nucleic acid molecules may be used to express large quantities of recombinant antibodies or to produce chimeric antibodies, single chain antibodies, immunoadhesins, diabodies, mutated antibodies, and other antibody derivatives. If the nucleic acid molecules are derived from a non-human, non-transgenic animal, the nucleic acid molecules may be used for antibody humanization.

### 1. Vectors

In some aspects, contemplated are expression vectors comprising a nucleic acid molecule encoding a polypeptide of the desired sequence or a portion thereof (e.g., a fragment containing one or more CDRs or one or more variable region domains). Expression vectors comprising the nucleic acid molecules may encode the heavy chain, light chain, or the antigen-binding portion thereof. In some aspects, expression vectors comprising nucleic acid molecules may encode fusion proteins, modified antibodies, antibody fragments, and probes thereof. In addition to control sequences that govern transcription and translation, vectors and expression vectors may contain nucleic acid sequences that serve other functions as well.

In some embodiments, to express antibodies, or antigen-binding fragments thereof, DNAs encoding partial or full-length light and heavy chains are inserted into expression vectors such that the gene area is operatively linked to transcriptional and translational control sequences. In some aspects, a vector that encodes a functionally complete human C_{H} or C_{L} immunoglobulin sequence with appropriate restriction sites engineered so that any V_{H} or V_{L} sequence can be easily inserted and expressed. In some embodiments, expression vectors used in any of the host cells contain sequences for plasmid or virus maintenance and for cloning and expression of exogenous nucleotide sequences. Such sequences, collectively referred to as “flanking sequences” may include one or more of the following operatively linked nucleotide sequences: a promoter, one or more enhancer sequences, an origin of replication, a transcriptional termination sequence, a complete intron sequence containing a donor and acceptor splice site, a sequence encoding a leader sequence for polypeptide secretion, a ribosome binding site, a polyadenylation sequence, a polylinker region for inserting the nucleic acid encoding the polypeptide to be expressed, and a selectable marker element. Such sequences and methods of using the same are well known in the art.

### 2. Expression Systems

Numerous expression systems exist that comprise at least a part or all of the expression vectors discussed above. Prokaryote- and/or eukaryote-based systems can be employed for use with an embodiment to produce nucleic acid sequences, or their cognate polypeptides, proteins and peptides. Commercially and widely available systems include in but are not limited to bacterial, mammalian, yeast, and insect cell systems. Different host cells have characteristic and specific mechanisms for the post-translational processing and modification of proteins. Appropriate cell lines or host systems can be chosen to ensure the correct modification and processing of the foreign protein expressed. Those skilled in the art are able to express a vector to produce a nucleic acid sequence or its cognate polypeptide, protein, or peptide using an appropriate expression system.

### 3. Methods of Gene Transfer

Suitable methods for nucleic acid delivery to effect expression of compositions are anticipated to include virtually any method by which a nucleic acid (e.g., DNA, including viral and nonviral vectors) can be introduced into a cell, a tissue or an organism, as described herein or as would be known to one of ordinary skill in the art. Such methods include, but are not limited to, direct delivery of DNA such as by injection (U.S. Pat. No. 5,994,624,5,981,274, 5,945,100, 5,780,448, 5,736,524, 5,702,932, 5,656,610, 5,589,466 and 5,580,859, each incorporated herein by reference), including microinjection (Harland and Weintraub, 1985; U.S. Pat. No. 5,789,215, incorporated herein by reference); by electroporation (U.S. Pat. No. 5,384,253, incorporated herein by reference); by calcium phosphate precipitation (Graham and Van Der Eb, 1973; Chen and Okayama, 1987; Rippe et al., 1990); by using DEAE dextran followed by polyethylene glycol (Gopal, 1985); by direct sonic loading (Fechheimer et al., 1987); by liposome mediated transfection (Nicolau and Sene, 1982; Fraley et al., 1979; Nicolau et al., 1987; Wong et al., 1980; Kaneda et al., 1989; Kato et al., 1991); by microprojectile bombardment (PCT Application Nos. WO 94/09699 and 95/06128; U.S. Pat. Nos. 5,610,042; 5,322,783, 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium mediated transformation (U.S. Pat. Nos. 5,591,616 and 5,563,055, each incorporated herein by reference); or by PEG mediated transformation of protoplasts (Omirulleh et al., 1993; U.S. Pat. Nos. 4,684,611 and 4,952,500, each incorporated herein by reference); by desiccation/inhibition mediated DNA uptake (Potrykus et al., 1985). Other methods include viral transduction, such as gene transfer by lentiviral or retroviral transduction.

### 4. Host Cells

In another aspect, contemplated are the use of host cells into which a recombinant expression vector has been introduced. Antibodies can be expressed in a variety of cell types. An expression construct encoding an antibody can be transfected into cells according to a variety of methods known in the art. Vector DNA can be introduced into prokaryotic or eukaryotic cells via conventional transformation or transfection techniques. Some vectors may employ control sequences that allow it to be replicated and/or expressed in both prokaryotic and eukaryotic cells. In certain aspects, the antibody expression construct can be placed under control of a promoter that is linked to T-cell activation, such as one that is controlled by NFAT-1 or NF-κB, both of which are transcription factors that can be activated upon T-cell activation. Control of antibody expression allows T cells, such as tumor-targeting T cells, to sense their surroundings and perform real-time modulation of cytokine signaling, both in the T cells themselves and in surrounding endogenous immune cells. One of skill in the art would understand the conditions under which to incubate host cells to maintain them and to permit replication of a vector. Also understood and known are techniques and conditions that would allow large-scale production of vectors, as well as production of the nucleic acids encoded by vectors and their cognate polypeptides, proteins, or peptides.

For stable transfection of mammalian cells, it is known, depending upon the expression vector and transfection technique used, only a small fraction of cells may integrate the foreign DNA into their genome. In order to identify and select these integrants, a selectable marker (e.g., for resistance to antibiotics) is generally introduced into the host cells along with the gene of interest. Cells stably transfected with the introduced nucleic acid can be identified by drug selection (e.g., cells that have incorporated the selectable marker gene will survive, while the other cells die), among other methods known in the arts.

### B. Isolation

A nucleic acid molecule encoding either or both of the entire heavy and light chains of an antibody or the variable regions thereof may be obtained from any source that produces antibodies. Methods of isolating mRNA encoding an antibody are well known in the art. See e.g., Sambrook et al., supra. The sequences of human heavy and light chain constant region genes are also known in the art. See, e.g., Kabat et al., 1991, supra. Nucleic acid molecules encoding full-length heavy and/or light chains may then be expressed in a cell into which they have been introduced and an antibody isolated.

### VIII. Genetically Engineered Receptors

Immune cells of the present disclosure can be genetically engineered to express one or more antigen-binding receptors that target CD70, such as engineered CARs or, alternatively, engineered TCRs. For example, the immune cells may be immune cells that are modified to express a CAR and/or TCR having antigenic specificity for CD70. Other CARs and/or TCRs may be expressed by the same cells as the CD70 antigen receptor-expressing cells, and they may be directed to different antigens. In some aspects, the immune cells are engineered to express the CD70-specific CAR or CD70-specific TCR by knock-in of the CAR or TCR using, for example, CRISPR/Cas technology.

Suitable methods of modification of cells are known in the art. See, for instance, Sambrook and Ausubel, supra. For example, the cells may be transduced to express a CAR or TCR having antigenic specificity for a cancer antigen using transduction techniques described in Heemskerk et al., 2008 and Johnson et al., 2009.

In some embodiments, the cells comprise one or more nucleic acids introduced via genetic engineering that encode one or more antigen-targeting receptors (at least one of which is directed against CD70), and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g., chimeric).

Exemplary antigen receptors, including CARs and recombinant TCRs, as well as methods for engineering and introducing the receptors into cells, include those described, for example, in international patent application publication numbers WO200014257, WO2013126726, WO2012/129514, WO2014031687, WO2013/166321, WO2013/071154, WO2013/123061 U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., 2013; Davila et al., 2013; Turtle et al., 2012; Wu et al., 2012. In some aspects, the genetically engineered antigen receptors include a CAR as described in U.S. Pat. No. 7,446,190, and those described in International Patent Application Publication No.: WO/2014055668 A1.

### A. Chimeric Antigen Receptors

In particular embodiments, a CD70-specific CAR is utilized that comprises at least: a) one or more intracellular signaling domains, b) a transmembrane domain, and c) an extracellular domain comprising at least one antigen binding region that specifically binds CD70. In some embodiments the antigen binding region is an antibody or functional fragment thereof. In other cases the antigen binding region of the CAR is not an antibody or functional fragment thereof (such as a ligand for CD70 such as CD27). In some embodiments, the antigen binding region of the CAR does not comprise an extracellular domain, or antigen binding portion thereof, from CD27. In some embodiments, the CD70-specific CAR binds only CD70, whereas in other cases the CAR as a single polypeptide is bispecific by comprising two or more antigen binding domains, one of which that binds CD70 and the other of which binds another, non-identical antigen.

In some embodiments, the engineered antigen receptors include CARs, including activating or stimulatory CARs, or costimulatory CARs (see WO2014/055668). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

It is contemplated that the chimeric construct can be introduced into immune cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric CAR construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

Certain embodiments of the present disclosure concern the use of nucleic acids, including nucleic acids encoding a CD70-specific CAR polypeptide, including in some cases a CAR that has been humanized to reduce immunogenicity (hCAR), comprising at least one intracellular signaling domain, a transmembrane domain, and an extracellular domain comprising one or more signaling motifs. In certain embodiments, the CD70-specific CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CD70 CAR nucleic acids may be used to enhance cellular immunotherapy for human patients. In a specific embodiment, the disclosure includes a full-length CD70-specific CAR cDNA or coding region. The antigen binding regions or domain can comprise a fragment of the V_{H} and V_{L} chains of a single-chain variable fragment (scFv) derived from a particular human monoclonal antibody. The fragment can also be any number of different antigen binding domains of a human antigen-specific antibody. In a more specific embodiment, the fragment is a CD70-specific scFv encoded by a sequence that is optimized for human codon usage for expression in human cells.

The arrangement could be multimeric, such as a diabody or multimers. The multimers may be formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the C_{H}2 or C_{H}3 domain from human immunoglobulin. One could also use the hinge, C_{H}2 and C_{H}3 region of a human immunoglobulin that has been modified to improve dimerization. One could also use just the hinge portion of an immunoglobulin. One could also use portions of CD8alpha or CD28.

In some embodiments, CD70-specific CAR is constructed with specificity for CD70, such as CD70 being expressed on a diseased cell type. Thus, the CAR typically includes in its extracellular portion one or more CD70-binding molecules, such as one or more antigen-binding fragments, domains, antibody variable domains, and/or antibody molecules of any kind.

In some embodiments, the CD70-specific CAR includes an antigen-binding portion or portions of an antibody molecule, such as a single-chain antibody fragment (scFv) derived from the variable heavy (V_{H}) and variable light (V_{L}) chains of a monoclonal antibody (mAb). In specific embodiments, the antibody or functional fragment thereof is or is derived from 41D12, 2H5. The antibody may also be one that is generated de novo against CD70, and the scFv sequence may be obtained, or derived, from such de novo antibodies.

In certain embodiments, the anti-CD70 CAR comprises an extracellular domain that is or comprises a ligand for CD70. In specific embodiments, the anti-CD70 CAR comprises an extracellular domain from CD27, or fragments or mimetics thereof. In certain embodiments, the anti-CD70 CAR does not comprise an extracellular domain from CD27.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof, as it is found that introns stabilize the mRNA. Also, it may be further advantageous to use endogenous or exogenous non-coding regions to stabilize the mRNA.

In some aspects, the antigen-specific binding, or recognition, component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex. The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, DAP12, DAP10, NKG2D, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, a KIR such as KIR2DL4, GITR/CD357, and so forth. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In some embodiments, the CD70 CAR nucleic acid comprises a sequence encoding other costimulatory receptors, such as a transmembrane domain and one or more intracellular signaling domains. In addition to a primary T cell activation signal, such as may be initiated by CD3ζ and/or FcεRIγ, an additional stimulatory signal for immune effector cell proliferation and effector function following engagement of the chimeric receptor with the target antigen may be utilized. For example, part or all of a human costimulatory receptor for enhanced activation of cells may be utilized that could help improve in vivo persistence and improve the therapeutic success of the adoptive immunotherapy. Examples include costimulatory domains from molecules such as DAP12, DAP10, NKG2D, CD2, CD28, CD27, 4-1BB, (CD137), OX40, ICOS, (CD278), CD30, HVEM, CD40, LFA-1 (CD11a/CD18), ICAM-1, and/or a portion of a CD70 cytoplasmic domain capable of inducing an activating signal, although in specific alternative embodiments any one of these listed may be excluded from use in the CAR.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ξ, or other combinations), (iii) CARs with variable lengths of extracellular domains connecting the CD70-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

### B. Examples of Specific CAR Embodiments

In particular embodiments, specific CD70 targeting CAR molecules are encompassed herein. In some cases, the CD70 binding domain of the CAR is a scFv, and any scFv that binds to CD70 may be utilized herein. In cases wherein an anti-CD70 scFv is utilized in the extracellular domain of the CAR, the variable heavy chain and the variable light chain for the scFv may be in any order in N-terminal to C-terminal direction. For example, the variable heavy chain may be on the N-terminal side of the variable light chain, or vice versa. The variable heavy chain and the variable light chain may be separated by a linker. The scFv and/or ligand that binds CD70 in the CAR may or may not be codon optimized. In particular embodiments, a vector encodes a CD70-specific CAR and also encodes one or more other molecules. For example, a vector may encode a CD70-specific CAR and also may encode another protein of interest, such as another engineered antigen receptor, a suicide gene, and/or a particular cytokine.

On the same molecule, the CD70-specific CAR may comprise one or more antigen-specific extracellular domains, a specific hinge, a specific transmembrane domain, one or more specific costimulatory domains, and one or more specific activation signals. When more than one antigen-specific extracellular domain is utilized, such as for targeting two different antigens (one of which is CD70), there may be a linker between the two antigen-specific extracellular domains.

In particular embodiments of specific CAR molecules, a CAR may utilize DAP10, DAP12, 4-1BB, NKG2D, or other costimulatory domains (which may be referred to herein as an intracytoplasmic domain). In some cases, CD3ζ is utilized without any costimulatory domains. In particular embodiments of specific CAR molecules, a CAR may utilize any suitable transmembrane domain, such as from DAP12, DAP10, 4-1BB, 2B4, OX40, CD27, NKG2D, CD8, or CD28.

In particular embodiments, there is an expression construct comprising a sequence that encodes a particular CD70-specific engineered receptor. In particular embodiments, any CD70 CAR may comprise one of SEQ ID NOs:68-73.

Examples of specific sequence embodiments are provided below.

### 1. Antigen-Specific Extracellular Domains

Examples of specific sequence embodiments are provided below.

Example CD70-binding region amino acid sequences are as follows:

(SEQ ID NO: 68)
QVQLQQSGAELMKPGASVKISCKATGYTFSNHWIEWVKERPGHGLEWIG
EILLGSGRAHYNEKFKGKATFTADTSSNTAYMQLSSLTSEDSAVYYCAR
HYRYDGWFAYWGQGTPVTVSAGGGGSGGGGSGGGASDIVLTQSPASLAV
SLGQRATISCRASKSVSTSGYSYMHWYQQKPGQPPKLLIYLASNLESGV
PARFSGSGSGTDFTLNIHPVEEEDAATYYCQHSRELPYTFGGGTKLEIK

Any polypeptide encompassed by the present disclosure may comprise SEQ ID NO:68 or a sequence that is at least, at most, exactly, or between any two of 70, 75, 80, 85, 90, 95, 96, 97, 98, 99, or more % identical to SEQ ID NO:68.

(SEQ ID NO: 69)
DIVLTQSPASLAVSLGQRATISCRASKSVSTSGYSYMHWYQQKPGQPPK
LLIYLASNLESGVPARFSGSGSGTDFTLNIHPVEEEDAATYYCQHSREL
PYTFGGGTKLEIKGGGGSGGGGSGGGASQVQLQQSGAELMKPGASVKIS
CKATGYTFSNHWIEWVKERPGHGLEWIGEILLGSGRAHYNEKFKGKATF
TADTSSNTAYMQLSSLTSEDSAVYYCARHYRYDGWFAYWGQGTPVTVSA

Any polypeptide encompassed by the present disclosure may comprise SEQ ID NO:69 or a sequence that is at least, at most, exactly, or between any two of 70, 75, 80, 85, 90, 95, 96, 97, 98, 99, or more % identical to SEQ ID NO:69.

(SEQ ID NO: 70)
QVQLKQSGPGLVQPSQSLSITCTVSGFSLTSYGVHWVRQSPGKGL
EWLGVIWSGGSTDYNAAFISRLSISKDNSKSQVFFKMNSLQANDT
AIYYCATYYRYDGWFAYWGQGTLVTVSAGGGGSGGGGSGGGASDI
VLTQSPASLAVSLGQRATISCRASKSVSTSGYSYMHWYQQKPGQP
PKLLIYLASNLESGVPARFSGSGSGTDFTLNIHPVEEEDAATYSC
QHSRELPWTFGGGTKLEIK

Any polypeptide encompassed by the present disclosure may comprise SEQ ID NO:70 or a sequence that is at least, at most, exactly, or between any two of 70, 75, 80, 85, 90, 95, 96, 97, 98, 99, or more % identical to SEQ ID NO:70.

(SEQ ID NO: 71)
DIVLTQSPASLAVSLGQRATISCRASKSVSTSGYSYMHWYQQKPG
QPPKLLIYLASNLESGVPARFSGSGSGTDFTLNIHPVEEEDAATY
SCQHSRELPWTFGGGTKLEIKGGGGSGGGGSGGGASQVQLKQSGP
GLVQPSQSLSITCTVSGFSLTSYGVHWVRQSPGKGLEWLGVIWSG
GSTDYNAAFISRLSISKDNSKSQVFFKMNSLQANDTAIYYCATYY
RYDGWFAYWGQGTLVTVSA

Any polypeptide encompassed by the present disclosure may comprise SEQ ID NO:71 or a sequence that is at least, at most, exactly, or between any two of 70, 75, 80, 85, 90, 95, 96, 97, 98, 99, or more % identical to SEQ ID NO:71.

(SEQ ID NO: 72)
EVQLQQSGAELVKPGASVKLSCTASGFNIKDSYMHWVKQRPEQGL
EWIGRIDPANANPKYDPKFQGKATITTDTSSNTAYLQLSSLTSED
TAVYYCARDYGGYFDVWGAGTTVTVSSGGGGSGGGGSGGGASDIQ
MTQSSSYLSVSLGGRVTITCKASDHINNWLAWYQQKPGNAPRLLI
SGATSLETGVPSRFSGSGSGKDYTLSITSLQTEDVATYYCQQYWS
TPWTFGGGTKLEIK

Any polypeptide encompassed by the present disclosure may comprise SEQ ID NO:72 or a sequence that is at least, at most, exactly, or between any two of 70, 75, 80, 85, 90, 95, 96, 97, 98, 99, or more % identical to SEQ ID NO:72.

(SEQ ID NO: 73)
DIQMTQSSSYLSVSLGGRVTITCKASDHINNWLAWYQQKPGNAPR
LLISGATSLETGVPSRFSGSGSGKDYTLSITSLQTEDVATYYCQQ
YWSTPWTFGGGTKLEIKGGGGSGGGGSGGGASEVQLQQSGAELVK
PGASVKLSCTASGFNIKDSYMHWVKQRPEQGLEWIGRIDPANANP
KYDPKFQGKATITTDTSSNTAYLQLSSLTSEDTAVYYCARDYGGY
FDVWGAGTTVTVSS

Any polypeptide encompassed by the present disclosure may comprise SEQ ID NO:73 or a sequence that is at least, at most, exactly, or between any two of 70, 75, 80, 85, 90, 95, 96, 97, 98, 99, or more % identical to SEQ ID NO:73.

In specific examples, a CD70-binding region that is utilized in a CAR molecule of the disclosure comprises, consists of, or consists essentially of amino acids 1-50, 1-51, 1-52, 1-53, 1-54, 1-55, 1-56, 1-57, 1-58, 1-59, 1-60, 1-61, 1-62, 1-63, 1-64, 1-65, 1-66, 1-67, 1-68, 1-69, 1-70, 1-71, 1-72, 1-73, 1-74, 1-75, 1-76, 1-77, 1-78, 1-79, 1-80, 1-81, 1-82, 1-83, 1-84, 1-85, 1-86, 1-87, 1-88, 1-89, 1-90, 1-91, 1-92, 1-93, 1-94, 1-95, 1-96, 1-97, 1-98, 1-99, 1-100, 1-101, 1-102, 1-103, 1-104, 1-105, 1-106, 1-107, 1-108, 1-109, 1-110, 1-111, 1-112, 1-113, 1-114, 1-115, 1-116, 1-117, 1-118, 1-119, 1-120, 1-121, 1-122, 1-123, 1-124, 1-125, 1-126, 1-127, 1-128, 1-129, 1-130, 1-131, 1-132, 1-133, 1-134, 1-135, 1-136, 1-137, 1-138, 1-139, 1-140, 1-141, 1-142, 1-143, 1-144, 1-145, 1-146, 1-147, 1-148, 1-149, 1-150, 1-151, 1-152, 1-153, 1-154, 1-155, 1-156, 1-157, 1-158, 1-159, 1-160, 1-161, 1-162, 1-163, 1-164, 1-165, 1-166, 1-167, 1-168, 1-169, 1-170, 1-171, 1-172, 1-173, 1-174, 1-175, 1-176, 1-177, 1-178, 1-179, 1-180, 1-181, 1-182, 1-183, 1-184, 1-185, 1-186, 1-187, 1-188, 1-189, 1-190, 1-191, 1-192, 1-193, 1-194, 1-195, 1-196, 1-197, 1-198, 1-199, 1-200, 1-201, 1-202, 1-203, 1-204, 1-205, 1-206, 1-207, 1-208, 1-209, 1-210, 1-211, 1-212, 1-213, 1-214, 1-215, 1-216, 1-217, 1-218, 1-219, 1-220, or all of SEQ ID NOs:68-73; in specific embodiments, such amino acids in these ranges are contiguous. In some embodiments, a region of SEQ ID NOs:68-73 is utilized that has truncation at the N-terminus, such as at least, at most, exactly, or between any two of 1, 2, 3, 4, 5, 6, 7, 8, 9, 10, 11, 12, 13, 14, 15, 16, 17, 18, 19, 20, or more amino acids from the N-terminus. In certain cases, there is truncation at that N-terminus of at least, at most, exactly, or between any two of 1, 2, 3, 4, 5, 6, 7, 8, 9, 10, 11, 12, 13, 14, 15, 16, 17, 18, 19, 20, or more amino acids and there is truncation at the C-terminus.

A CD70-binding region of the disclosure may comprise (a) a V_{H} selected from SEQ ID NO:44, SEQ ID NO:52, or SEQ ID NO:60; and (b) a V_{L} selected from SEQ ID NO:48, SEQ ID NO:56, or SEQ ID NO:64. Any of the preceeding V_{H} sequences may be combined with any of the preceeding V_{L} sequences in a CD70-binding region of a polypeptide (e.g., CAR) of the disclosure. In some embodiments, a CD70-binding region comprises a V_{H} comprising SEQ ID NO:44 and a V_{L} comprising SEQ ID NO:48. In some embodiments, a CD70-binding region comprises a V_{H} comprising SEQ ID NO:52 and a V_{L} comprising SEQ ID NO:56. In some embodiments, a CD70-binding region comprises a V_{H} comprising SEQ ID NO:60 and a V_{L} comprising SEQ ID NO:64. A CD70 binding region may comprise a V_{L} and V_{H} separated by a polypeptide linker. A linker may comprise, for example, SEQ ID NO:74.

### 2. Transmembrane Domains

Any suitable transmembrane domain may be utilized in a CD70-specific CAR of the disclosure. Examples include at least transmembrane domains from DAP10, DAP12, CD28, NKG2D, CD3 epsilon, CD4, CD5, CD8, CD9, CD16, CD22, CD28, CD33, CD37, CD45, CD64, CD80, CD86, CD134, CD137, or CD154, from a T-cell receptor a or b chain, from a CD3 zeta chain, from ICOS, functional derivatives thereof, and combinations thereof. In specific cases, a transmembrane domain from DAP10, DAP12, CD28, CD8, or NKG2D is utilized. In some embodiments, a transmembrane domain from CD70. Examples of particular transmembrane domain sequences may be used, as follows:

CD28 transmembrane domain amino acid sequence:

(SEQ ID NO: 75)
FWVLVVVGGVLACYSLLVTVAFIIFWV

CD8 transmembrane domain amino acid sequence:

(SEQ ID NO: 76)
TTTPAPRPPTPAPTIASQPLSLRPEACRPAAGGAVHTRGLDFACD
IYIWAPLAGTCGVLLLSLVIT

4-1BB transmembrane domain amino acid sequence:

(SEQ ID NO: 77)
IISFFLALTSTALLFLLFFLTLRFSVV

DAP10 transmembrane domain amino acid sequence:

(SEQ ID NO: 78)
LLAGLVAADAVASLLIVGAVF

DAP12 transmembrane domain amino acid sequence:

(SEQ ID NO: 79)
GVLAGIVMGDLVLTVLIALAV

NKG2D transmembrane domain amino acid sequence:

(SEQ ID NO: 80)
AVMIIFRIGMAVAIFCCFFFP

Any polypeptide encompassed by the present disclosure may comprise one of SEQ ID NOs:75-80 or a sequence that is at least, at most, exactly, or between any two of 70, 75, 80, 85, 90, 95, 96, 97, 98, 99, or more % identical to one of SEQ ID NOs:75-80.

### 3. Intracellular Domains

One or more intracellular domains (which may also be referred to herein as signal activation domains or costimulatory domains, in appropriate cases) may or may not be utilized in specific anti-CD70 CARs of the disclosure. Specific examples include intracellular domains from CD3 zeta, 4-1BB, NKG2D, OX-40, CD27, DAP10, DAP12, B7-1/CD80, CD28, 2B4, 4-1BBL, B7-2/CD86, CTLA-4, B7-H1/PD-L1, ICOS, B7-H2, PD-1, B7-H3, PD-L2, B7-H4, PDCD6, BTLA, or a combination thereof.

Examples of particular intracellular domains which may be used in a CAR of the disclosure are as follows:

An example CD3ζ intracellular domain amino acid sequence:

(SEQ ID NO: 81)
TRKKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRG

An example CD3ζ intracellular domain amino acid sequence:

(SEQ ID NO: 82)
KRVKFSRSADAPAYQQGQNQLYNELNLGRREEYDVLDKRRGRDPE
MGGKPRRKNPQEGLYNELQKDKMAEAYSEIGMKGERRRGKGHDGL
YQGLSTATKDTYDALHMQALPPRG

4-1BB intracellular domain amino acid sequence:

(SEQ ID NO: 83)
KRGRKKLLYIFKQPFMRPVQTTQEEDGCSCRFPEEEEGGCEL

DAP10 intracellular domain amino acid sequence:

(SEQ ID NO: 84)
LCARPRRSPAQEDGKVYINMPGRG

DAP12 intracellular domain amino acid sequence:

(SEQ ID NO: 85)
YFLGRLVPRGRGAAEAATRKQRITETESPYQELQGQRSDVYSDLN
TQRPYYK

NKG2D intracellular domain amino acid sequence:

(SEQ ID NO: 86)
SANERCKSKVVPCRQKQWRTSFDSKKLDLNYNHFESMEWSHRSRRGRIWG
M

Any polypeptide encompassed by the present disclosure may comprise SEQ ID NOs:81-86 or a sequence that is at least, at most, exactly, or between any two of 70, 75, 80, 85, 90, 95, 96, 97, 98, 99, or more % identical to one of SEQ ID NOs:81-86.

### 4. Hinge

In some embodiments of the CARs, there is a hinge region between the one or more extracellular antigen binding domains and the transmembrane domain. In specific embodiments, the hinge is of a particular length, such as 10-20, 10-15, 11-20, 11-15, 12-20, 12-15, or 15-20 amino acids in length, for example. The hinge may be any suitable hinge and includes a hinge from IgG, CD8, or CD28, in some cases. In specific embodiments, the hinge is a small flexible polypeptide that connects C_{H}2-C_{H}3 and C_{H}1 domains of IgG Fc. For example, one may utilize C_{H}2-C_{H}3 hinge (part or all) from various IgG subclasses (IgG1-4, either modified or not). However, in some cases the entire C_{H}2-C_{H}3 hinge is not utilized but instead a portion of the hinge is used (such as C_{H}3 by itself or part of C_{H}3 by itself). In particular embodiments, the C_{H}2-C_{H}3 hinge derived from IgG1 is utilized, and in some cases the entire C_{H}2-C_{H}3 hinge is used (all 229 amino acids), only the C_{H}3 hinge (119 amino acids) is used, or a short hinge (12 amino acids) is used.

In specific cases, one can modify the identity or length of the spacer and/or hinge to optimize efficiency of the CAR. See, for example, Hudecek et al. (2014) and Jonnalagadda et al. (2015) In specific embodiments, the CD70 CAR utilizes IgG4 hinge+C_{H}3 or utilizes CD8a stalk, for example.

Thus, in specific embodiments the IgG hinge region that is utilized is typically IgG1 or IgG4, and in some cases the CAR comprises the C_{H}2-C_{H}3 domain of IgG Fc. The use of the IgG Fc domain can provide flexibility to the CAR, has low immunogenicity, facilitates detection of CAR expression using anti-Fc reagents, and allows removal of one or more C_{H}2 or C_{H}3 modules to accommodate different spacer lengths. However, in one embodiment mutations in certain spacers to avoid FcTR binding may improve CAR+ T cell engraftment and antitumor efficacy to avoid binding of soluble and cell surface Fc gamma receptors, for example, yet maintain the activity to mediate antigen-specific lysis. For example, one can employ IgG4-Fc spacers that have either been modified in the C_{H}2 region. For example, the C_{H}2 region may be mutated, including point mutations and/or deletions. Specific modifications have been demonstrated at two sites (L235E; N297Q) within the C_{H}2 region and/or incorporate a C_{H}2 deletion (Jonnalagadda et al, 2015). In specific embodiments, one may employ the IgG4 hinge-C_{H}2-C_{H}3 domain (229 aa in length) or only the hinge domain (12 aa in length) (Hudececk et al., 2015).

In specific embodiments, the hinge is from IgG, CD28, CD-8 alpha, 4-1BB, 0X40, CD3-zeta, T cell receptor a or b chain, a CD3 zeta chain, CD28, CD3e, CD45, CD4, CD5, CD8, CD9, CD16, CD22, CD33, CD37, CD64, CD80, CD86, CD134, CD137, ICOS, or CD154.

Examples of specific sequences of hinges that may be utilized include at least the following:

### IgG Hinge Amino Acid Sequence:

(SEQ ID NO: 87)
TVTVSSQDPAEPKSPDKTHTCPPCPAPELLGGPSVFLFPPKPKDTLMISR
TPEVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFF
LYSKLTVDKSRWQQGNVFSCSVMHEAL

### CD28 Hinge Amino Acid Sequence:

| (SEQ ID NO: 88) | |
|---|---|
| | IEVMYPPPYLDNEKSNGTIIHVKGKHLCPSPLFPGPSKP |

Any polypeptide encompassed by the present disclosure may comprise SEQ ID NO:87 or 88 or a sequence that is at least, at most, exactly, or between any two of 70, 75, 80, 85, 90, 95, 96, 97, 98, 99, or more % identical to SEQ ID NO:87 or 88.

### 5. Other Proteins

In some embodiments, one or more other proteins are utilized with an anti-CD70 CAR of the disclosure. The one or more other proteins may be utilized for any reason, including to facilitate efficacy of the CAR itself and/or of any kind of cells expressing the CAR. In some cases, the other protein facilitates treatment of an individual receiving cells expressing the CAR as therapy, whether or not the other protein(s) directly or indirectly impact activity of the CAR or the cells. In some cases, the other protein is one or more antibodies or one or more bispecific or multispecific immune cell engagers. In some cases, the other protein is a suicide gene, one or more cytokines, or both. In specific embodiments, the one or more other proteins are produced from one or more vectors and ultimately are produced as separate polypeptides. In specific embodiments, the one or more other proteins are produced from the same vector and ultimately are produced as separate polypeptides. For example, the anti-CD70 CAR and the other protein(s) may be separated by a 2A sequence or by an IRES.

In specific embodiments, a cytokine such as IL-15 is utilized in conjunction with the anti-CD70 CAR.

One example of an IL-15 sequence is as follows:

### IL-15 Amino Acid Sequence:

(SEQ ID NO: 89)
ISKPHLRSISIQCYLCLLLNSHFLTEAGIHVFILGCFSAGLPKTEANWVN
VISDLKKIEDLIQSMHIDATLYTESDVHPSCKVTAMKCFLLELQVISLES
GDASIHDTVENLIILANNSLSSNGNVTESGCKECEELEEKNIKEFLQSFV
HIVQMFINTS

Any polypeptide encompassed by the present disclosure may comprise SEQ ID NO:89 or a sequence that is at least, at most, exactly, or between any two of 70, 75, 80, 85, 90, 95, 96, 97, 98, 99, or more % identical to SEQ ID NO:89.

In cases where the CAR and another protein in the same vector are intended to be produced into two different polypeptides, a specific 2A sequence may be utilized.

An E2A amino acid sequence may be utilized as follows:

| (SEQ ID NO: 90) | |
|---|---|
| | QCTNYALLKLAGDVESNPGP |

Other 2A examples may be utilized and are as follows:

| | T2A: |
|---|---|
| (SEQ ID NO: 91) | |
| | EGRGSLLTCGDVEENPGP |
| | P2A: |
| (SEQ ID NO: 92) | |
| | ATNFSLLKQAGDVEENPGP |
| | F2A: |
| (SEQ ID NO: 93) | |
| | VKQTLNFDLLKLAGDVESNPGP |

Any polypeptide encompassed by the present disclosure may comprise SEQ ID NOs:90-93 or a sequence that is at least, at most, exactly, or between any two of 70, 75, 80, 85, 90, 95, 96, 97, 98, 99, or more % identical to SEQ ID NOs:90-93.

The disclosure also encompasses specific CAR molecules, including for expression in any type of immune effector cells (e.g., T cells, NK cells, NKT cells, etc.).

In some embodiments, an anti-CD70 CAR comprising a CD70-binding domain, an IgG1 hinge, a CD28 intracellular domain, and a CD3ζ intracellular domain is utilized. In a vector, the CAR may be expressed with IL-15, such as may be separated from the CAR by a 2A sequence.

In specific examples, such a CAR and IL-15 construct may have the any one of the following nucleotide or amino acid sequences, or a sequence that is at least, at most, exactly, or between any two of 70, 75, 80, 85, 90, 95, 96, 97, 98, 99, or more % identical to the following nucleotide or amino acid sequences:

m6-1CAR70VHVLCD28Z15 Having an IgG Hinge

(SEQ ID NO: 32)
ATGGGGATGGCCCTGCCTGTGACAGCTCTGCTCCTCCCTCTGGCCCTGCT
GCTCCATGCCGCCAGACCCCAGGTTCAGCTGCAGCAGTCTGGAGCTGAGC
TGATGAAGCCTGGGGCCTCAGTGAAGATATCCTGCAAGGCAACTGGCTAC
ACATTCAGTAACCACTGGATAGAGTGGGTTAAGGAAAGGCCTGGACATGG
CCTGGAGTGGATTGGAGAGATTTTACTTGGAAGTGGTAGAGCTCATTATA
ATGAGAAGTTCAAGGGCAAGGCCACATTCACTGCAGATACATCCTCCAAC
ACAGCCTACATGCAACTCAGCAGCCTGACATCTGAGGACTCTGCCGTCTA
TTACTGTGCAAGACACTATAGGTACGACGGGTGGTTTGCTTACTGGGGCC
AAGGGACTCCGGTCACTGTCTCTGCAGGTGGAGGCGGTTCAGGCGGAGGT
GGCTCTGGCGGTGGCGCTAGCGACATTGTGCTGACACAGTCTCCTGCTTC
CTTAGCTGTATCTCTGGGGCAGAGGGCCACCATCTCATGCAGGGCCAGCA
AAAGTGTCAGTACATCTGGCTATAGTTATATGCACTGGTACCAACAGAAA
CCAGGACAGCCACCCAAACTCCTCATCTATCTTGCATCCAACCTAGAATC
TGGGGTCCCTGCCAGGTTCAGTGGCAGTGGGTCTGGGACAGACTTCACCC
TCAACATCCATCCTGTGGAGGAGGAGGATGCTGCAACCTATTACTGTCAG
CACAGTAGGGAGCTTCCGTACACGTTCGGAGGGGGGACCAAGCTGGAAAT
AAAACGTACGGTCACTGTCTCTTCACAGGATCCCGCCGAGCCCAAATCTC
CTGACAAAACTCACACATGCCCACCGTGCCCAGCACCTGAACTCCTGGGG
GGACCGTCAGTCTTCCTCTTCCCCCCAAAACCCAAGGACACCCTCATGAT
CTCCCGGACCCCTGAGGTCACATGCGTGGTGGTGGACGTGAGCCACGAAG
ACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGT
CAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCA
AAGGCTTCTATCCCAGCGACATCGCCGTGGAGTGGGAGAGCAATGGGCAA
CCGGAGAACAACTACAAGACCACGCCTCCCGTGCTGGACTCCGACGGCTC
CTTCTTCCTCTACAGCAAGCTCACCGTGGACAAGAGCAGGTGGCAGCAGG
GGAACGTCTTCTCATGCTCCGTGATGCATGAGGCTCTGCACAACCACTAC
ACGCAGAAGAGCCTCTCCCTGTCTCCGGGTAAAAAAGATCCCAAATTTTG
GGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTG
CACAGTGACTACATGAACATGACTCCCCGCCGCCCCGGGCCCACCCGCAA
GCATTACCAGCCCTATGCCCCACCACGCGACTTCGCAGCCTATCGCTCAC
GCGTGAAGTTCAGCAGGAGCGCAGACGCCCCCGCGTACCAGCAGGGCCAG
AACCAGCTCTATAACGAGCTCAATCTAGGACGAAGAGAGGAGTACGATGT
TTTGGACAAAAGACGTGGCCGGGACCCTGAGATGGGGGGAAAGCCGAGAA
GGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAA
GGGGCACGATGGCCTTTACCAGGGTCTCAGTACAGCCACCAAGGACACCT
ACGACGCCCTTCACATGCAGGCCCTGCCCCCTCGCGGACCGCAGTGTACT
AATTATGCTCTCTTGAAATTGGCTGGAGATGTTGAGAGCAATCCCGGGCC
CATGCGCATTAGCAAGCCCCACCTGCGGAGCATCAGCATCCAGTGCTACC
TGTGCCTGCTGCTGAACAGCCACTTCCTGACCGAGGCCGGCATCCACGTG
TTCATCCTGGGCTGCTTCAGCGCCGGACTGCCCAAGACCGAGGCCAACTG
GGTGAACGTGATCAGCGACCTGAAGAAGATCGAGGACCTGATCCAGAGCA
TGCACATCGACGCCACCCTGTACACCGAGAGCGACGTGCACCCCAGCTGC
AAGGTGACCGCCATGAAGTGCTTTCTGCTGGAACTGCAGGTGATCAGCCT
GGAAAGCGGCGACGCCAGCATCCACGACACCGTGGAGAACCTGATCATCC
TGGCCAACAACAGCCTGAGCAGCAACGGCAACGTGACCGAGAGCGGCTGC
AAAGAGTGCGAGGAACTGGAAGAGAAGAACATCAAAGAGTTTCTGCAGAG
CTTCGTGCACATCGTGCAGATGTTCATCAACACCAGCTGA

A corresponding amino acid sequence for m6-1CAR70VHVLCD28Z15 having an IgG hinge is as follows:

(SEQ ID NO: 94)
MGMALPVTALLLPLALLLHAARPQVQLQQSGAELMKPGASVKISCKATGY
TFSNHWIEWVKERPGHGLEWIGEILLGSGRAHYNEKFKGKATFTADTSSN
TAYMQLSSLTSEDSAVYYCARHYRYDGWFAYWGQGTPVTVSAGGGGSGGG
GSGGGASDIVLTQSPASLAVSLGQRATISCRASKSVSTSGYSYMHWYQQK
PGQPPKLLIYLASNLESGVPARFSGSGSGTDFTLNIHPVEEEDAATYYCQ
HSRELPYTFGGGTKLEIKRTVTVSSQDPAEPKSPDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGKKDPKFWVLVVVGGVLACYSLLVTVAFIIFWVRSKRSRLL
HSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPRGPQCT
NYALLKLAGDVESNPGPMRISKPHLRSISIQCYLCLLLNSHFLTEAGIHV
FILGCFSAGLPKTEANWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS

m6-1CAR70VLVHCD28Z15 Having an IgG Hinge

(SEQ ID NO: 33)
ATGGGGATGGCCCTGCCTGTGACAGCTCTGCTCCTCCCTCTGGCCCTGCT
GCTCCATGCCGCCAGACCCGACATTGTGCTGACACAGTCTCCTGCTTCCT
TAGCTGTATCTCTGGGGCAGAGGGCCACCATCTCATGCAGGGCCAGCAAA
AGTGTCAGTACATCTGGCTATAGTTATATGCACTGGTACCAACAGAAACC
AGGACAGCCACCCAAACTCCTCATCTATCTTGCATCCAACCTAGAATCTG
GGGTCCCTGCCAGGTTCAGTGGCAGTGGGTCTGGGACAGACTTCACCCTC
AACATCCATCCTGTGGAGGAGGAGGATGCTGCAACCTATTACTGTCAGCA
CAGTAGGGAGCTTCCGTACACGTTCGGAGGGGGGACCAAGCTGGAAATAA
AAGGTGGAGGCGGTTCAGGCGGAGGTGGCTCTGGCGGTGGCGCTAGCCAG
GTTCAGCTGCAGCAGTCTGGAGCTGAGCTGATGAAGCCTGGGGCCTCAGT
GAAGATATCCTGCAAGGCAACTGGCTACACATTCAGTAACCACTGGATAG
AGTGGGTTAAGGAAAGGCCTGGACATGGCCTGGAGTGGATTGGAGAGATT
TTACTTGGAAGTGGTAGAGCTCATTATAATGAGAAGTTCAAGGGCAAGGC
CACATTCACTGCAGATACATCCTCCAACACAGCCTACATGCAACTCAGCA
GCCTGACATCTGAGGACTCTGCCGTCTATTACTGTGCAAGACACTATAGG
TACGACGGGTGGTTTGCTTACTGGGGCCAAGGGACTCCGGTCACTGTCTC
TGCACGTACGGTCACTGTCTCTTCACAGGATCCCGCCGAGCCCAAATCTC
CTGACAAAACTCACACATGCCCACCGTGCCCAGCACCTGAACTCCTGGGG
GGACCGTCAGTCTTCCTCTTCCCCCCAAAACCCAAGGACACCCTCATGAT
CTCCCGGACCCCTGAGGTCACATGCGTGGTGGTGGACGTGAGCCACGAAG
ACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGT
CAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCA
AAGGCTTCTATCCCAGCGACATCGCCGTGGAGTGGGAGAGCAATGGGCAA
CCGGAGAACAACTACAAGACCACGCCTCCCGTGCTGGACTCCGACGGCTC
CTTCTTCCTCTACAGCAAGCTCACCGTGGACAAGAGCAGGTGGCAGCAGG
GGAACGTCTTCTCATGCTCCGTGATGCATGAGGCTCTGCACAACCACTAC
ACGCAGAAGAGCCTCTCCCTGTCTCCGGGTAAAAAAGATCCCAAATTTTG
GGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTG
CACAGTGACTACATGAACATGACTCCCCGCCGCCCCGGGCCCACCCGCAA
GCATTACCAGCCCTATGCCCCACCACGCGACTTCGCAGCCTATCGCTCAC
GCGTGAAGTTCAGCAGGAGCGCAGACGCCCCCGCGTACCAGCAGGGCCAG
AACCAGCTCTATAACGAGCTCAATCTAGGACGAAGAGAGGAGTACGATGT
TTTGGACAAAAGACGTGGCCGGGACCCTGAGATGGGGGGAAAGCCGAGAA
GGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAA
GGGGCACGATGGCCTTTACCAGGGTCTCAGTACAGCCACCAAGGACACCT
ACGACGCCCTTCACATGCAGGCCCTGCCCCCTCGCGGACCGCAGTGTACT
AATTATGCTCTCTTGAAATTGGCTGGAGATGTTGAGAGCAATCCCGGGCC
CATGCGCATTAGCAAGCCCCACCTGCGGAGCATCAGCATCCAGTGCTACC
TGTGCCTGCTGCTGAACAGCCACTTCCTGACCGAGGCCGGCATCCACGTG
TTCATCCTGGGCTGCTTCAGCGCCGGACTGCCCAAGACCGAGGCCAACTG
GGTGAACGTGATCAGCGACCTGAAGAAGATCGAGGACCTGATCCAGAGCA
TGCACATCGACGCCACCCTGTACACCGAGAGCGACGTGCACCCCAGCTGC
AAGGTGACCGCCATGAAGTGCTTTCTGCTGGAACTGCAGGTGATCAGCCT
GGAAAGCGGCGACGCCAGCATCCACGACACCGTGGAGAACCTGATCATCC
TGGCCAACAACAGCCTGAGCAGCAACGGCAACGTGACCGAGAGCGGCTGC
AAAGAGTGCGAGGAACTGGAAGAGAAGAACATCAAAGAGTTTCTGCAGAG
CTTCGTGCACATCGTGCAGATGTTCATCAACACCAGCTGA.

A corresponding amino acid sequence for m6-1CAR70VLVHCD28Z15 having an IgG hinge is as follows:

(SEQ ID NO: 95)
MGMALPVTALLLPLALLLHAARPDIVLTQSPASLAVSLGQRATISCRASK
SVSTSGYSYMHWYQQKPGQPPKLLIYLASNLESGVPARFSGSGSGTDFTL
NIHPVEEEDAATYYCQHSRELPYTFGGGTKLEIKGGGGSGGGGSGGGASQ
VQLQQSGAELMKPGASVKISCKATGYTFSNHWIEWVKERPGHGLEWIGEI
LLGSGRAHYNEKFKGKATFTADTSSNTAYMQLSSLTSEDSAVYYCARHYR
YDGWFAYWGQGTPVTVSARTVTVSSQDPAEPKSPDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGKKDPKFWVLVVVGGVLACYSLLVTVAFIIFWVRSKRSRLL
HSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPRGPQCT
NYALLKLAGDVESNPGPMRISKPHLRSISIQCYLCLLLNSHFLTEAGIHV
FILGCFSAGLPKTEANWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS.

m6-1CAR70VHVLCD28Z15 Having an CD28 Hinge

(SEQ ID NO: 38)
ATGGGGATGGCCCTGCCTGTGACAGCTCTGCTCCTCCCTCTGGCCCTGCT
GCTCCATGCCGCCAGACCCGACATCCAGATGACACAATCTTCATCCTACT
TGTCTGTATCTCTAGGAGGCAGAGTCACCATTACTTGCAAGGCAAGTGAC
CACATTAATAATTGGTTAGCCTGGTATCAGCAGAAACCAGGAAATGCTCC
TAGGCTCTTAATATCTGGTGCAACCAGTTTGGAAACTGGGGTTCCTTCAA
GATTCAGTGGCAGTGGATCTGGAAAGGATTACACTCTCAGCATTACCAGT
CTTCAGACTGAAGATGTTGCCACTTATTACTGTCAACAGTATTGGAGTAC
TCCGTGGACGTTCGGTGGAGGCACCAAGCTGGAAATCAAACccGGTGGAG
GCGGTTCAGGCGGAGGTGGCTCTGGCGGTGGCGCTAGCGAGGTTCAGCTG
CAGCAGTCTGGGGCAGAGCTTGTGAAGCCAGGGGCCTCAGTCAAGTTGTC
CTGCACAGCTTCTGGCTTCAACATTAAAGACAGCTATATGCACTGGGTGA
AGCAGAGGCCTGAACAGGGCCTGGAGTGGATTGGAAGGATTGATCCTGCG
AATGCTAATCCTAAATATGACCCGAAGTTCCAGGGCAAGGCCACTATAAC
AACAGACACATCCTCCAACACAGCCTACCTGCAGCTCAGCAGCCTGACAT
CTGAGGACACTGCCGTCTATTACTGTGCTAGAGACTACGGAGGGTACTTC
GATGTCTGGGGCGCAGGGACCACGGTCACCGTCTCCTCACGTACGGTCAC
TGTCTCTTCACAGGATCCCGCCGAGCCCAAATCTCCTGACAAAACTCACA
CATGCCCACCGTGCCCAGCACCTGAACTCCTGGGGGGACCGTCAGTCTTC
CTCTTCCCCCCAAAACCCAAGGACACCCTCATGATCTCCCGGACCCCTGA
GGTCACATGCGTGGTGGTGGACGTGAGCCACGAAGACCCTGAGGTCAAGT
TCAACTGGTACGTGGACGGCGTGGAGGTGCATAATGCCAAGACAAAGCCG
CGGGAGGAGCAGTACAACAGCACGTACCGTGTGGTCAGCGTCCTCACCGT
CCTGCACCAGGACTGGCTGAATGGCAAGGAGTACAAGTGCAAGGTCTCCA
ACAAAGCCCTCCCAGCCCCCATCGAGAAAACCATCTCCAAAGCCAAAGGG
CAGCCCCGAGAACCACAGGTGTACACCCTGCCCCCATCCCGGGATGAGCT
GACCAAGAACCAGGTCAGCCTGACCTGCCTGGTCAAAGGCTTCTATCCCA
GCGACATCGCCGTGGAGTGGGAGAGCAATGGGCAACCGGAGAACAACTAC
AAGACCACGCCTCCCGTGCTGGACTCCGACGGCTCCTTCTTCCTCTACAG
CAAGCTCACCGTGGACAAGAGCAGGTGGCAGCAGGGGAACGTCTTCTCAT
GCTCCGTGATGCATGAGGCTCTGCACAACCACTACACGCAGAAGAGCCTC
TCCCTGTCTCCGGGTAAAAAAGATCCCAAATTTTGGGTGCTGGTGGTGGT
TGGTGGAGTCCTGGCTTGCTATAGCTTGCTAGTAACAGTGGCCTTTATTA
TTTTCTGGGTGAGGAGTAAGAGGAGCAGGCTCCTGCACAGTGACTACATG
AACATGACTCCCCGCCGCCCCGGGCCCACCCGCAAGCATTACCAGCCCTA
TGCCCCACCACGCGACTTCGCAGCCTATCGCTCACGCGTGAAGTTCAGCA
GGAGCGCAGACGCCCCCGCGTACCAGCAGGGCCAGAACCAGCTCTATAAC
GAGCTCAATCTAGGACGAAGAGAGGAGTACGATGTTTTGGACAAAAGACG
TGGCCGGGACCCTGAGATGGGGGGAAAGCCGAGAAGGAAGAACCCTCAGG
AAGGCCTGTACAATGAACTGCAGAAAGATAAGATGGCGGAGGCCTACAGT
GAGATTGGGATGAAAGGCGAGCGCCGGAGGGGCAAGGGGCACGATGGCCT
TTACCAGGGTCTCAGTACAGCCACCAAGGACACCTACGACGCCCTTCACA
TGCAGGCCCTGCCCCCTCGCGGACCGCAGTGTACTAATTATGCTCTCTTG
AAATTGGCTGGAGATGTTGAGAGCAATCCCGGGCCCATGCGCATTAGCAA
GCCCCACCTGCGGAGCATCAGCATCCAGTGCTACCTGTGCCTGCTGCTGA
ACAGCCACTTCCTGACCGAGGCCGGCATCCACGTGTTCATCCTGGGCTGC
TTCAGCGCCGGACTGCCCAAGACCGAGGCCAACTGGGTGAACGTGATCAG
CGACCTGAAGAAGATCGAGGACCTGATCCAGAGCATGCACATCGACGCCA
CCCTGTACACCGAGAGCGACGTGCACCCCAGCTGCAAGGTGACCGCCATG
AAGTGCTTTCTGCTGGAACTGCAGGTGATCAGCCTGGAAAGCGGCGACGC
CAGCATCCACGACACCGTGGAGAACCTGATCATCCTGGCCAACAACAGCC
TGAGCAGCAACGGCAACGTGACCGAGAGCGGCTGCAAAGAGTGCGAGGAA
CTGGAAGAGAAGAACATCAAAGAGTTTCTGCAGAGCTTCGTGCACATCGT
GCAGATGTTCATCAACACCAGCTGA

A corresponding amino acid sequence for m6-1CAR70VHVLCD28Z15 having an CD28 hinge is as follows:

(SEQ ID NO: 100)
MGMALPVTALLLPLALLLHAARPDIQMTQSSSYLSVSLGGRVTITCKASD
HINNWLAWYQQKPGNAPRLLISGATSLETGVPSRFSGSGSGKDYTLSITS
LQTEDVATYYCQQYWSTPWTFGGGTKLEIKPGGGGSGGGGSGGGASEVQL
QQSGAELVKPGASVKLSCTASGFNIKDSYMHWVKQRPEQGLEWIGRIDPA
NANPKYDPKFQGKATITTDTSSNTAYLQLSSLTSEDTAVYYCARDYGGYF
DVWGAGTTVTVSSRTVTVSSQDPAEPKSPDKTHTCPPCPAPELLGGPSVF
LFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNY
KTTPPVLDSDGSFFLYSKLTVDKSRWQQGNVFSCSVMHEALHNHYTQKSL
SLSPGKKDPKFWVLVVVGGVLACYSLLVTVAFIIFWVRSKRSRLLHSDYM
NMTPRRPGPTRKHYQPYAPPRDFAAYRSRVKFSRSADAPAYQQGQNQLYN
ELNLGRREEYDVLDKRRGRDPEMGGKPRRKNPQEGLYNELQKDKMAEAYS
EIGMKGERRRGKGHDGLYQGLSTATKDTYDALHMQALPPRGPQCTNYALL
KLAGDVESNPGPMRISKPHLRSISIQCYLCLLLNSHFLTEAGIHVFILGC
FSAGLPKTEANWVNVISDLKKIEDLIQSMHIDATLYTESDVHPSCKVTAM
KCFLLELQVISLESGDASIHDTVENLIILANNSLSSNGNVTESGCKECEE
LEEKNIKEFLQSFVHIVQMFINTS

m6-1CAR70VLVHCD28Z15 Having an CD28 Hinge

(SEQ ID NO: 39)
ATGGGGATGGCCCTGCCTGTGACAGCTCTGCTCCTCCCTCTGGCCCTGCT
GCTCCATGCCGCCAGACCCGACATTGTGCTGACACAGTCTCCTGCTTCCT
TAGCTGTATCTCTGGGGCAGAGGGCCACCATCTCATGCAGGGCCAGCAAA
AGTGTCAGTACATCTGGCTATAGTTATATGCACTGGTACCAACAGAAACC
AGGACAGCCACCCAAACTCCTCATCTATCTTGCATCCAACCTAGAATCTG
GGGTCCCTGCCAGGTTCAGTGGCAGTGGGTCTGGGACAGACTTCACCCTC
AACATCCATCCTGTGGAGGAGGAGGATGCTGCAACCTATTACTGTCAGCA
CAGTAGGGAGCTTCCGTACACGTTCGGAGGGGGGACCAAGCTGGAAATAA
AAGGTGGAGGCGGTTCAGGCGGAGGTGGCTCTGGCGGTGGCGCTAGCCAG
GTTCAGCTGCAGCAGTCTGGAGCTGAGCTGATGAAGCCTGGGGCCTCAGT
GAAGATATCCTGCAAGGCAACTGGCTACACATTCAGTAACCACTGGATAG
AGTGGGTTAAGGAAAGGCCTGGACATGGCCTGGAGTGGATTGGAGAGATT
TTACTTGGAAGTGGTAGAGCTCATTATAATGAGAAGTTCAAGGGCAAGGC
CACATTCACTGCAGATACATCCTCCAACACAGCCTACATGCAACTCAGCA
GCCTGACATCTGAGGACTCTGCCGTCTATTACTGTGCAAGACACTATAGG
TACGACGGGTGGTTTGCTTACTGGGGCCAAGGGACTCCGGTCACTGTCTC
TGCACGTACGGTCACTGTCTCTTCACAGGATCCCGCCGAGCCCAAATCTC
CTGACAAAACTCACACATGCCCACCGTGCCCAGCACCTGAACTCCTGGGG
GGACCGTCAGTCTTCCTCTTCCCCCCAAAACCCAAGGACACCCTCATGAT
CTCCCGGACCCCTGAGGTCACATGCGTGGTGGTGGACGTGAGCCACGAAG
ACCCTGAGGTCAAGTTCAACTGGTACGTGGACGGCGTGGAGGTGCATAAT
GCCAAGACAAAGCCGCGGGAGGAGCAGTACAACAGCACGTACCGTGTGGT
CAGCGTCCTCACCGTCCTGCACCAGGACTGGCTGAATGGCAAGGAGTACA
AGTGCAAGGTCTCCAACAAAGCCCTCCCAGCCCCCATCGAGAAAACCATC
TCCAAAGCCAAAGGGCAGCCCCGAGAACCACAGGTGTACACCCTGCCCCC
ATCCCGGGATGAGCTGACCAAGAACCAGGTCAGCCTGACCTGCCTGGTCA
AAGGCTTCTATCCCAGCGACATCGCCGTGGAGTGGGAGAGCAATGGGCAA
CCGGAGAACAACTACAAGACCACGCCTCCCGTGCTGGACTCCGACGGCTC
CTTCTTCCTCTACAGCAAGCTCACCGTGGACAAGAGCAGGTGGCAGCAGG
GGAACGTCTTCTCATGCTCCGTGATGCATGAGGCTCTGCACAACCACTAC
ACGCAGAAGAGCCTCTCCCTGTCTCCGGGTAAAAAAGATCCCAAATTTTG
GGTGCTGGTGGTGGTTGGTGGAGTCCTGGCTTGCTATAGCTTGCTAGTAA
CAGTGGCCTTTATTATTTTCTGGGTGAGGAGTAAGAGGAGCAGGCTCCTG
CACAGTGACTACATGAACATGACTCCCCGCCGCCCCGGGCCCACCCGCAA
GCATTACCAGCCCTATGCCCCACCACGCGACTTCGCAGCCTATCGCTCAC
GCGTGAAGTTCAGCAGGAGCGCAGACGCCCCCGCGTACCAGCAGGGCCAG
AACCAGCTCTATAACGAGCTCAATCTAGGACGAAGAGAGGAGTACGATGT
TTTGGACAAAAGACGTGGCCGGGACCCTGAGATGGGGGGAAAGCCGAGAA
GGAAGAACCCTCAGGAAGGCCTGTACAATGAACTGCAGAAAGATAAGATG
GCGGAGGCCTACAGTGAGATTGGGATGAAAGGCGAGCGCCGGAGGGGCAA
GGGGCACGATGGCCTTTACCAGGGTCTCAGTACAGCCACCAAGGACACCT
ACGACGCCCTTCACATGCAGGCCCTGCCCCCTCGCGGACCGCAGTGTACT
AATTATGCTCTCTTGAAATTGGCTGGAGATGTTGAGAGCAATCCCGGGCC
CATGCGCATTAGCAAGCCCCACCTGCGGAGCATCAGCATCCAGTGCTACC
TGTGCCTGCTGCTGAACAGCCACTTCCTGACCGAGGCCGGCATCCACGTG
TTCATCCTGGGCTGCTTCAGCGCCGGACTGCCCAAGACCGAGGCCAACTG
GGTGAACGTGATCAGCGACCTGAAGAAGATCGAGGACCTGATCCAGAGCA
TGCACATCGACGCCACCCTGTACACCGAGAGCGACGTGCACCCCAGCTGC
AAGGTGACCGCCATGAAGTGCTTTCTGCTGGAACTGCAGGTGATCAGCCT
GGAAAGCGGCGACGCCAGCATCCACGACACCGTGGAGAACCTGATCATCC
TGGCCAACAACAGCCTGAGCAGCAACGGCAACGTGACCGAGAGCGGCTGC
AAAGAGTGCGAGGAACTGGAAGAGAAGAACATCAAAGAGTTTCTGCAGAG
CTTCGTGCACATCGTGCAGATGTTCATCAACACCAGCTGA.

A corresponding amino acid sequence for m6-1CAR70VLVHCD28Z15 having an CD28 hinge is as follows:

(SEQ ID NO: 101)
MGMALPVTALLLPLALLLHAARPDIVLTQSPASLAVSLGQRATISCRASK
SVSTSGYSYMHWYQQKPGQPPKLLIYLASNLESGVPARFSGSGSGTDFTL
NIHPVEEEDAATYYCQHSRELPYTFGGGTKLEIKGGGGSGGGGSGGGASQ
VQLQQSGAELMKPGASVKISCKATGYTFSNHWIEWVKERPGHGLEWIGEI
LLGSGRAHYNEKFKGKATFTADTSSNTAYMQLSSLTSEDSAVYYCARHYR
YDGWFAYWGQGTPVTVSARTVTVSSQDPAEPKSPDKTHTCPPCPAPELLG
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGKKDPKFWVLVVVGGVLACYSLLVTVAFIIFWVRSKRSRLL
HSDYMNMTPRRPGPTRKHYQPYAPPRDFAAYRSRVKFSRSADAPAYQQGQ
NQLYNELNLGRREEYDVLDKRRGRDPEMGGKPRRKNPQEGLYNELQKDKM
AEAYSEIGMKGERRRGKGHDGLYQGLSTATKDTYDALHMQALPPRGPQCT
NYALLKLAGDVESNPGPMRISKPHLRSISIQCYLCLLLNSHFLTEAGIHV
FILGCFSAGLPKTEANWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS.

m7-1CAR70VHVLCD28Z15 Having a IgG Hinge

(SEQ ID NO: 34)
ATGGGGATGGCCCTGCCTGTGACAGCTCTGCTCCTCCCTCTGGCCCTGCT
GCTCCATGCCGCCAGACCCCAGGTGCAGCTGAAGCAGTCAGGACCTGGCC
TAGTGCAGCCCTCACAGAGCCTGTCCATCACCTGCACAGTCTCTGGTTTC
TCATTAACTAGCTATGGTGTACACTGGGTTCGCCAGTCTCCAGGAAAGGG
TCTGGAGTGGCTGGGAGTGATATGGAGTGGTGGAAGCACAGACTATAATG
CAGCTTTCATATCCAGACTGAGCATCAGCAAGGACAATTCCAAGAGCCAA
GTTTTCTTTAAAATGAACAGTCTGCAAGCTAATGACACAGCCATATATTA
CTGTGCCACGTACTATAGGTACGACGGGTGGTTTGCTTACTGGGGCCAAG
GGACTCTGGTCACTGTCTCTGCAGGTGGAGGCGGTTCAGGCGGAGGTGGC
TCTGGCGGTGGCGCTAGCGACATTGTGCTGACACAGTCTCCTGCTTCCTT
AGCTGTATCTCTGGGGCAGAGGGCCACCATCTCATGCAGGGCCAGCAAAA
GTGTCAGTACATCTGGCTATAGTTATATGCACTGGTATCAACAGAAACCA
GGACAGCCACCCAAACTCCTCATCTATCTTGCATCCAACCTAGAATCTGG
GGTCCCTGCCAGGTTCAGTGGCAGTGGGTCTGGGACAGACTTCACCCTCA
ACATCCATCCTGTGGAGGAGGAGGATGCTGCAACCTATTCCTGTCAGCAC
AGTAGGGAGCTTCCGTGGACGTTCGGTGGAGGCACCAAGCTGGAAATCAA
ACGTACGGTCACTGTCTCTTCACAGGATCCCGCCGAGCCCAAATCTCCTG
ACAAAACTCACACATGCCCACCGTGCCCAGCACCTGAACTCCTGGGGGGA
CCGTCAGTCTTCCTCTTCCCCCCAAAACCCAAGGACACCCTCATGATCTC
CCGGACCCCTGAGGTCACATGCGTGGTGGTGGACGTGAGCCACGAAGACC
CTGAGGTCAAGTTCAACTGGTACGTGGACGGCGTGGAGGTGCATAATGCC
AAGACAAAGCCGCGGGAGGAGCAGTACAACAGCACGTACCGTGTGGTCAG
CGTCCTCACCGTCCTGCACCAGGACTGGCTGAATGGCAAGGAGTACAAGT
GCAAGGTCTCCAACAAAGCCCTCCCAGCCCCCATCGAGAAAACCATCTCC
AAAGCCAAAGGGCAGCCCCGAGAACCACAGGTGTACACCCTGCCCCCATC
CCGGGATGAGCTGACCAAGAACCAGGTCAGCCTGACCTGCCTGGTCAAAG
GCTTCTATCCCAGCGACATCGCCGTGGAGTGGGAGAGCAATGGGCAACCG
GAGAACAACTACAAGACCACGCCTCCCGTGCTGGACTCCGACGGCTCCTT
CTTCCTCTACAGCAAGCTCACCGTGGACAAGAGCAGGTGGCAGCAGGGGA
ACGTCTTCTCATGCTCCGTGATGCATGAGGCTCTGCACAACCACTACACG
CAGAAGAGCCTCTCCCTGTCTCCGGGTAAAAAAGATCCCAAATTTTGGGT
GCTGGTGGTGGTTGGTGGAGTCCTGGCTTGCTATAGCTTGCTAGTAACAG
TGGCCTTTATTATTTTCTGGGTGAGGAGTAAGAGGAGCAGGCTCCTGCAC
AGTGACTACATGAACATGACTCCCCGCCGCCCCGGGCCCACCCGCAAGCA
TTACCAGCCCTATGCCCCACCACGCGACTTCGCAGCCTATCGCTCACGCG
TGAAGTTCAGCAGGAGCGCAGACGCCCCCGCGTACCAGCAGGGCCAGAAC
CAGCTCTATAACGAGCTCAATCTAGGACGAAGAGAGGAGTACGATGTTTT
GGACAAAAGACGTGGCCGGGACCCTGAGATGGGGGGAAAGCCGAGAAGGA
AGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGG
GCACGATGGCCTTTACCAGGGTCTCAGTACAGCCACCAAGGACACCTACG
ACGCCCTTCACATGCAGGCCCTGCCCCCTCGCGGACCGCAGTGTACTAAT
TATGCTCTCTTGAAATTGGCTGGAGATGTTGAGAGCAATCCCGGGCCCAT
GCGCATTAGCAAGCCCCACCTGCGGAGCATCAGCATCCAGTGCTACCTGT
GCCTGCTGCTGAACAGCCACTTCCTGACCGAGGCCGGCATCCACGTGTTC
ATCCTGGGCTGCTTCAGCGCCGGACTGCCCAAGACCGAGGCCAACTGGGT
GAACGTGATCAGCGACCTGAAGAAGATCGAGGACCTGATCCAGAGCATGC
ACATCGACGCCACCCTGTACACCGAGAGCGACGTGCACCCCAGCTGCAAG
GTGACCGCCATGAAGTGCTTTCTGCTGGAACTGCAGGTGATCAGCCTGGA
AAGCGGCGACGCCAGCATCCACGACACCGTGGAGAACCTGATCATCCTGG
CCAACAACAGCCTGAGCAGCAACGGCAACGTGACCGAGAGCGGCTGCAAA
GAGTGCGAGGAACTGGAAGAGAAGAACATCAAAGAGTTTCTGCAGAGCTT
CGTGCACATCGTGCAGATGTTCATCAACACCAGCTGA.

A corresponding amino acid sequence for m7-1CAR70VHVLCD28Z15 having a IgG hinge is as follows:

(SEQ ID NO: 96)
MGMALPVTALLLPLALLLHAARPQVQLKQSGPGLVQPSQSLSITC
TVSGFSLTSYGVHWVRQSPGKGLEWLGVIWSGGSTDYNAAFISRL
SISKDNSKSQVFFKMNSLQANDTAIYYCATYYRYDGWFAYWGQGT
LVTVSAGGGGSGGGGSGGGASDIVLTQSPASLAVSLGQRATISCR
ASKSVSTSGYSYMHWYQQKPGQPPKLLIYLASNLESGVPARFSGS
GSGTDFTLNIHPVEEEDAATYSCQHSRELPWTFGGGTKLEIKRTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWVLVVVGGVLACYSLLVTVAFIIFW
VRSKRSRLLHSDYMNMTPRRPGPTRKHYQPYAPPRDFAAYRSRVK
FSRSADAPAYQQGQNQLYNELNLGRREEYDVLDKRRGRDPEMGGK
PRRKNPQEGLYNELQKDKMAEAYSEIGMKGERRRGKGHDGLYQGL
STATKDTYDALHMQALPPRGPQCTNYALLKLAGDVESNPGPMRIS
KPHLRSISIQCYLCLLLNSHFLTEAGIHVFILGCFSAGLPKTEAN
WVNVISDLKKIEDLIQSMHIDATLYTESDVHPSCKVTAMKCFLLE
LQVISLESGDASIHDTVENLIILANNSLSSNGNVTESGCKECEEL
EEKNIKEFLQSFVHIVQMFINTS.

m7-1CAR70VLVHCD28Z15 Having a IgG Hinge

(SEQ ID NO: 35)
ATGGGGATGGCCCTGCCTGTGACAGCTCTGCTCCTCCCTCTGGCC
CTGCTGCTCCATGCCGCCAGACCCGACATTGTGCTGACACAGTCT
CCTGCTTCCTTAGCTGTATCTCTGGGGCAGAGGGCCACCATCTCA
TGCAGGGCCAGCAAAAGTGTCAGTACATCTGGCTATAGTTATATG
CACTGGTATCAACAGAAACCAGGACAGCCACCCAAACTCCTCATC
TATCTTGCATCCAACCTAGAATCTGGGGTCCCTGCCAGGTTCAGT
GGCAGTGGGTCTGGGACAGACTTCACCCTCAACATCCATCCTGTG
GAGGAGGAGGATGCTGCAACCTATTCCTGTCAGCACAGTAGGGAG
CTTCCGTGGACGTTCGGTGGAGGCACCAAGCTGGAAATCAAAGGT
GGAGGCGGTTCAGGCGGAGGTGGCTCTGGCGGTGGCGCTAGCCAG
GTGCAGCTGAAGCAGTCAGGACCTGGCCTAGTGCAGCCCTCACAG
AGCCTGTCCATCACCTGCACAGTCTCTGGTTTCTCATTAACTAGC
TATGGTGTACACTGGGTTCGCCAGTCTCCAGGAAAGGGTCTGGAG
TGGCTGGGAGTGATATGGAGTGGTGGAAGCACAGACTATAATGCA
GCTTTCATATCCAGACTGAGCATCAGCAAGGACAATTCCAAGAGC
CAAGTTTTCTTTAAAATGAACAGTCTGCAAGCTAATGACACAGCC
ATATATTACTGTGCCACGTACTATAGGTACGACGGGTGGTTTGCT
TACTGGGGCCAAGGGACTCTGGTCACTGTCTCTGCACGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGGTGCTGGTGGTGGTTGGTGGAGTCCTG
GCTTGCTATAGCTTGCTAGTAACAGTGGCCTTTATTATTTTCTGG
GTGAGGAGTAAGAGGAGCAGGCTCCTGCACAGTGACTACATGAAC
ATGACTCCCCGCCGCCCCGGGCCCACCCGCAAGCATTACCAGCCC
TATGCCCCACCACGCGACTTCGCAGCCTATCGCTCACGCGTGAAG
TTCAGCAGGAGCGCAGACGCCCCCGCGTACCAGCAGGGCCAGAAC
CAGCTCTATAACGAGCTCAATCTAGGACGAAGAGAGGAGTACGAT
GTTTTGGACAAAAGACGTGGCCGGGACCCTGAGATGGGGGGAAAG
CCGAGAAGGAAGAACCCTCAGGAAGGCCTGTACAATGAACTGCAG
AAAGATAAGATGGCGGAGGCCTACAGTGAGATTGGGATGAAAGGC
GAGCGCCGGAGGGGCAAGGGGCACGATGGCCTTTACCAGGGTCTC
AGTACAGCCACCAAGGACACCTACGACGCCCTTCACATGCAGGCC
CTGCCCCCTCGCGGACCGCAGTGTACTAATTATGCTCTCTTGAAA
TTGGCTGGAGATGTTGAGAGCAATCCCGGGCCCATGCGCATTAGC
AAGCCCCACCTGCGGAGCATCAGCATCCAGTGCTACCTGTGCCTG
CTGCTGAACAGCCACTTCCTGACCGAGGCCGGCATCCACGTGTTC
ATCCTGGGCTGCTTCAGCGCCGGACTGCCCAAGACCGAGGCCAAC
TGGGTGAACGTGATCAGCGACCTGAAGAAGATCGAGGACCTGATC
CAGAGCATGCACATCGACGCCACCCTGTACACCGAGAGCGACGTG
CACCCCAGCTGCAAGGTGACCGCCATGAAGTGCTTTCTGCTGGAA
CTGCAGGTGATCAGCCTGGAAAGCGGCGACGCCAGCATCCACGAC
ACCGTGGAGAACCTGATCATCCTGGCCAACAACAGCCTGAGCAGC
AACGGCAACGTGACCGAGAGCGGCTGCAAAGAGTGCGAGGAACTG
GAAGAGAAGAACATCAAAGAGTTTCTGCAGAGCTTCGTGCACATC
GTGCAGATGTTCATCAACACCAGCTGA.

A corresponding amino acid sequence for m7-1CAR70VLVHCD28Z15 having a IgG hinge is as follows:

(SEQ ID NO: 97)
MGMALPVTALLLPLALLLHAARPDIVLTQSPASLAVSLGQRATIS
CRASKSVSTSGYSYMHWYQQKPGQPPKLLIYLASNLESGVPARFS
GSGSGTDFTLNIHPVEEEDAATYSCQHSRELPWTFGGGTKLEIKG
GGGSGGGGSGGGASQVQLKQSGPGLVQPSQSLSITCTVSGFSLTS
YGVHWVRQSPGKGLEWLGVIWSGGSTDYNAAFISRLSISKDNSKS
QVFFKMNSLQANDTAIYYCATYYRYDGWFAYWGQGTLVTVSARTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWVLVVVGGVLACYSLLVTVAFIIFW
VRSKRSRLLHSDYMNMTPRRPGPTRKHYQPYAPPRDFAAYRSRVK
FSRSADAPAYQQGQNQLYNELNLGRREEYDVLDKRRGRDPEMGGK
PRRKNPQEGLYNELQKDKMAEAYSEIGMKGERRRGKGHDGLYQGL
STATKDTYDALHMQALPPRGPQCTNYALLKLAGDVESNPGPMRIS
KPHLRSISIQCYLCLLLNSHFLTEAGIHVFILGCFSAGLPKTEAN
WVNVISDLKKIEDLIQSMHIDATLYTESDVHPSCKVTAMKCFLLE
LQVISLESGDASIHDTVENLIILANNSLSSNGNVTESGCKECEEL
EEKNIKEFLQSFVHIVQMFINTS.

m7-1CAR70VHVLCD28Z15 Having a CD28 Hinge

(SEQ ID NO: 40)
ATGGGGATGGCCCTGCCTGTGACAGCTCTGCTCCTCCCTCTGGCC
CTGCTGCTCCATGCCGCCAGACCCCAGGTGCAGCTGAAGCAGTCA
GGACCTGGCCTAGTGCAGCCCTCACAGAGCCTGTCCATCACCTGC
ACAGTCTCTGGTTTCTCATTAACTAGCTATGGTGTACACTGGGTT
CGCCAGTCTCCAGGAAAGGGTCTGGAGTGGCTGGGAGTGATATGG
AGTGGTGGAAGCACAGACTATAATGCAGCTTTCATATCCAGACTG
AGCATCAGCAAGGACAATTCCAAGAGCCAAGTTTTCTTTAAAATG
AACAGTCTGCAAGCTAATGACACAGCCATATATTACTGTGCCACG
TACTATAGGTACGACGGGTGGTTTGCTTACTGGGGCCAAGGGACT
CTGGTCACTGTCTCTGCAGGTGGAGGCGGTTCAGGCGGAGGTGGC
TCTGGCGGTGGCGCTAGCGACATTGTGCTGACACAGTCTCCTGCT
TCCTTAGCTGTATCTCTGGGGCAGAGGGCCACCATCTCATGCAGG
GCCAGCAAAAGTGTCAGTACATCTGGCTATAGTTATATGCACTGG
TATCAACAGAAACCAGGACAGCCACCCAAACTCCTCATCTATCTT
GCATCCAACCTAGAATCTGGGGTCCCTGCCAGGTTCAGTGGCAGT
GGGTCTGGGACAGACTTCACCCTCAACATCCATCCTGTGGAGGAG
GAGGATGCTGCAACCTATTCCTGTCAGCACAGTAGGGAGCTTCCG
TGGACGTTCGGTGGAGGCACCAAGCTGGAAATCAAACGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGGTGCTGGTGGTGGTTGGTGGAGTCCTG
GCTTGCTATAGCTTGCTAGTAACAGTGGCCTTTATTATTTTCTGG
GTGAGGAGTAAGAGGAGCAGGCTCCTGCACAGTGACTACATGAAC
ATGACTCCCCGCCGCCCCGGGCCCACCCGCAAGCATTACCAGCCC
TATGCCCCACCACGCGACTTCGCAGCCTATCGCTCACGCGTGAAG
TTCAGCAGGAGCGCAGACGCCCCCGCGTACCAGCAGGGCCAGAAC
CAGCTCTATAACGAGCTCAATCTAGGACGAAGAGAGGAGTACGAT
GTTTTGGACAAAAGACGTGGCCGGGACCCTGAGATGGGGGGAAAG
CCGAGAAGGAAGAACCCTCAGGAAGGCCTGTACAATGAACTGCAG
AAAGATAAGATGGCGGAGGCCTACAGTGAGATTGGGATGAAAGGC
GAGCGCCGGAGGGGCAAGGGGCACGATGGCCTTTACCAGGGTCTC
AGTACAGCCACCAAGGACACCTACGACGCCCTTCACATGCAGGCC
CTGCCCCCTCGCGGACCGCAGTGTACTAATTATGCTCTCTTGAAA
TTGGCTGGAGATGTTGAGAGCAATCCCGGGCCCATGCGCATTAGC
AAGCCCCACCTGCGGAGCATCAGCATCCAGTGCTACCTGTGCCTG
CTGCTGAACAGCCACTTCCTGACCGAGGCCGGCATCCACGTGTTC
ATCCTGGGCTGCTTCAGCGCCGGACTGCCCAAGACCGAGGCCAAC
TGGGTGAACGTGATCAGCGACCTGAAGAAGATCGAGGACCTGATC
CAGAGCATGCACATCGACGCCACCCTGTACACCGAGAGCGACGTG
CACCCCAGCTGCAAGGTGACCGCCATGAAGTGCTTTCTGCTGGAA
CTGCAGGTGATCAGCCTGGAAAGCGGCGACGCCAGCATCCACGAC
ACCGTGGAGAACCTGATCATCCTGGCCAACAACAGCCTGAGCAGC
AACGGCAACGTGACCGAGAGCGGCTGCAAAGAGTGCGAGGAACTG
GAAGAGAAGAACATCAAAGAGTTTCTGCAGAGCTTCGTGCACATC
GTGCAGATGTTCATCAACACCAGCTGA.

A corresponding amino acid sequence for m7-1CAR70VHVLCD28Z15 having a CD28 hinge is as follows:

(SEQ ID NO: 102)
MGMALPVTALLLPLALLLHAARPQVQLKQSGPGLVQPSQSLSITC
TVSGFSLTSYGVHWVRQSPGKGLEWLGVIWSGGSTDYNAAFISRL
SISKDNSKSQVFFKMNSLQANDTAIYYCATYYRYDGWFAYWGQGT
LVTVSAGGGGSGGGGSGGGASDIVLTQSPASLAVSLGQRATISCR
ASKSVSTSGYSYMHWYQQKPGQPPKLLIYLASNLESGVPARFSGS
GSGTDFTLNIHPVEEEDAATYSCQHSRELPWTFGGGTKLEIKRTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWVLVVVGGVLACYSLLVTVAFIIFW
VRSKRSRLLHSDYMNMTPRRPGPTRKHYQPYAPPRDFAAYRSRVK
FSRSADAPAYQQGQNQLYNELNLGRREEYDVLDKRRGRDPEMGGK
PRRKNPQEGLYNELQKDKMAEAYSEIGMKGERRRGKGHDGLYQGL
STATKDTYDALHMQALPPRGPQCTNYALLKLAGDVESNPGPMRIS
KPHLRSISIQCYLCLLLNSHFLTEAGIHVFILGCFSAGLPKTEAN
WVNVISDLKKIEDLIQSMHIDATLYTESDVHPSCKVTAMKCFLLE
LQVISLESGDASIHDTVENLIILANNSLSSNGNVTESGCKECEEL
EEKNIKEFLQSFVHIVQMFINTS.

m7-1CAR70VLVHCD28Z15 Having a CD28 Hinge

(SEQ ID NO: 41)
ATGGGGATGGCCCTGCCTGTGACAGCTCTGCTCCTCCCTCTGGCC
CTGCTGCTCCATGCCGCCAGACCCGACATTGTGCTGACACAGTCT
CCTGCTTCCTTAGCTGTATCTCTGGGGCAGAGGGCCACCATCTCA
TGCAGGGCCAGCAAAAGTGTCAGTACATCTGGCTATAGTTATATG
CACTGGTATCAACAGAAACCAGGACAGCCACCCAAACTCCTCATC
TATCTTGCATCCAACCTAGAATCTGGGGTCCCTGCCAGGTTCAGT
GGCAGTGGGTCTGGGACAGACTTCACCCTCAACATCCATCCTGTG
GAGGAGGAGGATGCTGCAACCTATTCCTGTCAGCACAGTAGGGAG
CTTCCGTGGACGTTCGGTGGAGGCACCAAGCTGGAAATCAAAGGT
GGAGGCGGTTCAGGCGGAGGTGGCTCTGGCGGTGGCGCTAGCCAG
GTGCAGCTGAAGCAGTCAGGACCTGGCCTAGTGCAGCCCTCACAG
AGCCTGTCCATCACCTGCACAGTCTCTGGTTTCTCATTAACTAGC
TATGGTGTACACTGGGTTCGCCAGTCTCCAGGAAAGGGTCTGGAG
TGGCTGGGAGTGATATGGAGTGGTGGAAGCACAGACTATAATGCA
GCTTTCATATCCAGACTGAGCATCAGCAAGGACAATTCCAAGAGC
CAAGTTTTCTTTAAAATGAACAGTCTGCAAGCTAATGACACAGCC
ATATATTACTGTGCCACGTACTATAGGTACGACGGGTGGTTTGCT
TACTGGGGCCAAGGGACTCTGGTCACTGTCTCTGCACGTACGGTC
ACTGTCTCTTCACAGGATCCCGCCGAGCCCAAATCTCCTGACAAA
ACTCACACATGCCCACCGTGCCCAGCACCTGAACTCCTGGGGGGA
CCGTCAGTCTTCCTCTTCCCCCCAAAACCCAAGGACACCCTCATG
ATCTCCCGGACCCCTGAGGTCACATGCGTGGTGGTGGACGTGAGC
CACGAAGACCCTGAGGTCAAGTTCAACTGGTACGTGGACGGCGTG
GAGGTGCATAATGCCAAGACAAAGCCGCGGGAGGAGCAGTACAAC
AGCACGTACCGTGTGGTCAGCGTCCTCACCGTCCTGCACCAGGAC
TGGCTGAATGGCAAGGAGTACAAGTGCAAGGTCTCCAACAAAGCC
CTCCCAGCCCCCATCGAGAAAACCATCTCCAAAGCCAAAGGGCAG
CCCCGAGAACCACAGGTGTACACCCTGCCCCCATCCCGGGATGAG
CTGACCAAGAACCAGGTCAGCCTGACCTGCCTGGTCAAAGGCTTC
TATCCCAGCGACATCGCCGTGGAGTGGGAGAGCAATGGGCAACCG
GAGAACAACTACAAGACCACGCCTCCCGTGCTGGACTCCGACGGC
TCCTTCTTCCTCTACAGCAAGCTCACCGTGGACAAGAGCAGGTGG
CAGCAGGGGAACGTCTTCTCATGCTCCGTGATGCATGAGGCTCTG
CACAACCACTACACGCAGAAGAGCCTCTCCCTGTCTCCGGGTAAA
AAAGATCCCAAATTTTGGGTGCTGGTGGTGGTTGGTGGAGTCCTG
GCTTGCTATAGCTTGCTAGTAACAGTGGCCTTTATTATTTTCTGG
GTGAGGAGTAAGAGGAGCAGGCTCCTGCACAGTGACTACATGAAC
ATGACTCCCCGCCGCCCCGGGCCCACCCGCAAGCATTACCAGCCC
TATGCCCCACCACGCGACTTCGCAGCCTATCGCTCACGCGTGAAG
TTCAGCAGGAGCGCAGACGCCCCCGCGTACCAGCAGGGCCAGAAC
CAGCTCTATAACGAGCTCAATCTAGGACGAAGAGAGGAGTACGAT
GTTTTGGACAAAAGACGTGGCCGGGACCCTGAGATGGGGGGAAAG
CCGAGAAGGAAGAACCCTCAGGAAGGCCTGTACAATGAACTGCAG
AAAGATAAGATGGCGGAGGCCTACAGTGAGATTGGGATGAAAGGC
GAGCGCCGGAGGGGCAAGGGGCACGATGGCCTTTACCAGGGTCTC
AGTACAGCCACCAAGGACACCTACGACGCCCTTCACATGCAGGCC
CTGCCCCCTCGCGGACCGCAGTGTACTAATTATGCTCTCTTGAAA
TTGGCTGGAGATGTTGAGAGCAATCCCGGGCCCATGCGCATTAGC
AAGCCCCACCTGCGGAGCATCAGCATCCAGTGCTACCTGTGCCTG
CTGCTGAACAGCCACTTCCTGACCGAGGCCGGCATCCACGTGTTC
ATCCTGGGCTGCTTCAGCGCCGGACTGCCCAAGACCGAGGCCAAC
TGGGTGAACGTGATCAGCGACCTGAAGAAGATCGAGGACCTGATC
CAGAGCATGCACATCGACGCCACCCTGTACACCGAGAGCGACGTG
CACCCCAGCTGCAAGGTGACCGCCATGAAGTGCTTTCTGCTGGAA
CTGCAGGTGATCAGCCTGGAAAGCGGCGACGCCAGCATCCACGAC
ACCGTGGAGAACCTGATCATCCTGGCCAACAACAGCCTGAGCAGC
AACGGCAACGTGACCGAGAGCGGCTGCAAAGAGTGCGAGGAACTG
GAAGAGAAGAACATCAAAGAGTTTCTGCAGAGCTTCGTGCACATC
GTGCAGATGTTCATCAACACCAGCTGA.

A corresponding amino acid sequence for m7-1CAR70VLVHCD28Z15 having a CD28 hinge is as follows:

(SEQ ID NO: 103)
MGMALPVTALLLPLALLLHAARPDIVLTQSPASLAVSLGQRATIS
CRASKSVSTSGYSYMHWYQQKPGQPPKLLIYLASNLESGVPARFS
GSGSGTDFTLNIHPVEEEDAATYSCQHSRELPWTFGGGTKLEIKG
GGGSGGGGSGGGASQVQLKQSGPGLVQPSQSLSITCTVSGFSLTS
YGVHWVRQSPGKGLEWLGVIWSGGSTDYNAAFISRLSISKDNSKS
QVFFKMNSLQANDTAIYYCATYYRYDGWFAYWGQGTLVTVSARTV
TVSSQDPAEPKSPDKTHTCPPCPAPELLGGPSVFLFPPKPKDTLM
ISRTPEVTCVVVDVSHEDPEVKFNWYVDGVEVHNAKTKPREEQYN
STYRVVSVLTVLHQDWLNGKEYKCKVSNKALPAPIEKTISKAKGQ
PREPQVYTLPPSRDELTKNQVSLTCLVKGFYPSDIAVEWESNGQP
ENNYKTTPPVLDSDGSFFLYSKLTVDKSRWQQGNVFSCSVMHEAL
HNHYTQKSLSLSPGKKDPKFWVLVVVGGVLACYSLLVTVAFIIFW
VRSKRSRLLHSDYMNMTPRRPGPTRKHYQPYAPPRDFAAYRSRVK
FSRSADAPAYQQGQNQLYNELNLGRREEYDVLDKRRGRDPEMGGK
PRRKNPQEGLYNELQKDKMAEAYSEIGMKGERRRGKGHDGLYQGL
STATKDTYDALHMQALPPRGPQCTNYALLKLAGDVESNPGPMRIS
KPHLRSISIQCYLCLLLNSHFLTEAGIHVFILGCFSAGLPKTEAN
WVNVISDLKKIEDLIQSMHIDATLYTESDVHPSCKVTAMKCFLLE
LQVISLESGDASIHDTVENLIILANNSLSSNGNVTESGCKECEEL
EEKNIKEFLQSFVHIVQMFINTS.

m14-1CAR701VHVLCD28Z15 Having an IgG Hinge

(SEQ ID NO: 36)
ATGGGGATGGCCCTGCCTGTGACAGCTCTGCTCCTCCCTCTGGCC
CTGCTGCTCCATGCCGCCAGACCCGAGGTTCAGCTGCAGCAGTCT
GGGGCAGAGCTTGTGAAGCCAGGGGCCTCAGTCAAGTTGTCCTGC
ACAGCTTCTGGCTTCAACATTAAAGACAGCTATATGCACTGGGTG
AAGCAGAGGCCTGAACAGGGCCTGGAGTGGATTGGAAGGATTGAT
CCTGCGAATGCTAATCCTAAATATGACCCGAAGTTCCAGGGCAAG
GCCACTATAACAACAGACACATCCTCCAACACAGCCTACCTGCAG
CTCAGCAGCCTGACATCTGAGGACACTGCCGTCTATTACTGTGCT
AGAGACTACGGAGGGTACTTCGATGTCTGGGGCGCAGGGACCACG
GTCACCGTCTCCTCAGGTGGAGGCGGTTCAGGCGGAGGTGGCTCT
GGCGGTGGCGCTAGCGACATCCAGATGACACAATCTTCATCCTAC
TTGTCTGTATCTCTAGGAGGCAGAGTCACCATTACTTGCAAGGCA
AGTGACCACATTAATAATTGGTTAGCCTGGTATCAGCAGAAACCA
GGAAATGCTCCTAGGCTCTTAATATCTGGTGCAACCAGTTTGGAA
ACTGGGGTTCCTTCAAGATTCAGTGGCAGTGGATCTGGAAAGGAT
TACACTCTCAGCATTACCAGTCTTCAGACTGAAGATGTTGCCACT
TATTACTGTCAACAGTATTGGAGTACTCCGTGGACGTTCGGTGGA
GGCACCAAGCTGGAAATCAAACccCGTACGGTCACTGTCTCTTCA
CAGGATCCCGCCGAGCCCAAATCTCCTGACAAAACTCACACATGC
CCACCGTGCCCAGCACCTGAACTCCTGGGGGGACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCAAGGTCTCCAACAAAGCCCTCCCAGCCCCC
ATCGAGAAAACCATCTCCAAAGCCAAAGGGCAGCCCCGAGAACCA
CAGGTGTACACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAACCGGAGAACAACTAC
AAGACCACGCCTCCCGTGCTGGACTCCGACGGCTCCTTCTTCCTC
TACAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTAAAAAAGATCCCAAA
TTTTGGGTGCTGGTGGTGGTTGGTGGAGTCCTGGCTTGCTATAGC
TTGCTAGTAACAGTGGCCTTTATTATTTTCTGGGTGAGGAGTAAG
AGGAGCAGGCTCCTGCACAGTGACTACATGAACATGACTCCCCGC
CGCCCCGGGCCCACCCGCAAGCATTACCAGCCCTATGCCCCACCA
CGCGACTTCGCAGCCTATCGCTCACGCGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAA
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
GGACCGCAGTGTACTAATTATGCTCTCTTGAAATTGGCTGGAGAT
GTTGAGAGCAATCCCGGGCCCATGCGCATTAGCAAGCCCCACCTG
CGGAGCATCAGCATCCAGTGCTACCTGTGCCTGCTGCTGAACAGC
CACTTCCTGACCGAGGCCGGCATCCACGTGTTCATCCTGGGCTGC
TTCAGCGCCGGACTGCCCAAGACCGAGGCCAACTGGGTGAACGTG
ATCAGCGACCTGAAGAAGATCGAGGACCTGATCCAGAGCATGCAC
ATCGACGCCACCCTGTACACCGAGAGCGACGTGCACCCCAGCTGC
AAGGTGACCGCCATGAAGTGCTTTCTGCTGGAACTGCAGGTGATC
AGCCTGGAAAGCGGCGACGCCAGCATCCACGACACCGTGGAGAAC
CTGATCATCCTGGCCAACAACAGCCTGAGCAGCAACGGCAACGTG
ACCGAGAGCGGCTGCAAAGAGTGCGAGGAACTGGAAGAGAAGAAC
ATCAAAGAGTTTCTGCAGAGCTTCGTGCACATCGTGCAGATGTTC
ATCAACACCAGCTGA.

A corresponding amino acid sequence for m14-1CAR701VHVLCD28Z15 having an IgG hinge is as follows:

(SEQ ID NO: 98)
MGMALPVTALLLPLALLLHAARPEVQLQQSGAELVKPGASVKLSC
TASGFNIKDSYMHWVKQRPEQGLEWIGRIDPANANPKYDPKFQGK
ATITTDTSSNTAYLQLSSLTSEDTAVYYCARDYGGYFDVWGAGTT
VTVSSGGGGSGGGGSGGGASDIQMTQSSSYLSVSLGGRVTITCKA
SDHINNWLAWYQQKPGNAPRLLISGATSLETGVPSRFSGSGSGKD
YTLSITSLQTEDVATYYCQQYWSTPWTFGGGTKLEIKPRTVTVSS
QDPAEPKSPDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPGKKDPKFWVLVVVGGVLACYSLLVTVAFIIFWVRSK
RSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTAT
KDTYDALHMQALPPRGPQCTNYALLKLAGDVESNPGPMRISKPHL
RSISIQCYLCLLLNSHFLTEAGIHVFILGCFSAGLPKTEANWVNV
ISDLKKIEDLIQSMHIDATLYTESDVHPSCKVTAMKCFLLELQVI
SLESGDASIHDTVENLIILANNSLSSNGNVTESGCKECEELEEKN
IKEFLQSFVHIVQMFINTS.

m14-1CAR701VLVHCD28Z15 Having an IgG Hinge

(SEQ ID NO: 37)
ATGGGGATGGCCCTGCCTGTGACAGCTCTGCTCCTCCCTCTGGCC
CTGCTGCTCCATGCCGCCAGACCCGACATCCAGATGACACAATCT
TCATCCTACTTGTCTGTATCTCTAGGAGGCAGAGTCACCATTACT
TGCAAGGCAAGTGACCACATTAATAATTGGTTAGCCTGGTATCAG
CAGAAACCAGGAAATGCTCCTAGGCTCTTAATATCTGGTGCAACC
AGTTTGGAAACTGGGGTTCCTTCAAGATTCAGTGGCAGTGGATCT
GGAAAGGATTACACTCTCAGCATTACCAGTCTTCAGACTGAAGAT
GTTGCCACTTATTACTGTCAACAGTATTGGAGTACTCCGTGGACG
TTCGGTGGAGGCACCAAGCTGGAAATCAAACccGGTGGAGGCGGT
TCAGGCGGAGGTGGCTCTGGCGGTGGCGCTAGCGAGGTTCAGCTG
CAGCAGTCTGGGGCAGAGCTTGTGAAGCCAGGGGCCTCAGTCAAG
TTGTCCTGCACAGCTTCTGGCTTCAACATTAAAGACAGCTATATG
CACTGGGTGAAGCAGAGGCCTGAACAGGGCCTGGAGTGGATTGGA
AGGATTGATCCTGCGAATGCTAATCCTAAATATGACCCGAAGTTC
CAGGGCAAGGCCACTATAACAACAGACACATCCTCCAACACAGCC
TACCTGCAGCTCAGCAGCCTGACATCTGAGGACACTGCCGTCTAT
TACTGTGCTAGAGACTACGGAGGGTACTTCGATGTCTGGGGCGCA
GGGACCACGGTCACCGTCTCCTCACGTACGGTCACTGTCTCTTCA
CAGGATCCCGCCGAGCCCAAATCTCCTGACAAAACTCACACATGC
CCACCGTGCCCAGCACCTGAACTCCTGGGGGGACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCAAGGTCTCCAACAAAGCCCTCCCAGCCCCC
ATCGAGAAAACCATCTCCAAAGCCAAAGGGCAGCCCCGAGAACCA
CAGGTGTACACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAACCGGAGAACAACTAC
AAGACCACGCCTCCCGTGCTGGACTCCGACGGCTCCTTCTTCCTC
TACAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTAAAAAAGATCCCAAA
TTTTGGGTGCTGGTGGTGGTTGGTGGAGTCCTGGCTTGCTATAGC
TTGCTAGTAACAGTGGCCTTTATTATTTTCTGGGTGAGGAGTAAG
AGGAGCAGGCTCCTGCACAGTGACTACATGAACATGACTCCCCGC
CGCCCCGGGCCCACCCGCAAGCATTACCAGCCCTATGCCCCACCA
CGCGACTTCGCAGCCTATCGCTCACGCGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAA
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
GGACCGCAGTGTACTAATTATGCTCTCTTGAAATTGGCTGGAGAT
GTTGAGAGCAATCCCGGGCCCATGCGCATTAGCAAGCCCCACCTG
CGGAGCATCAGCATCCAGTGCTACCTGTGCCTGCTGCTGAACAGC
CACTTCCTGACCGAGGCCGGCATCCACGTGTTCATCCTGGGCTGC
TTCAGCGCCGGACTGCCCAAGACCGAGGCCAACTGGGTGAACGTG
ATCAGCGACCTGAAGAAGATCGAGGACCTGATCCAGAGCATGCAC
ATCGACGCCACCCTGTACACCGAGAGCGACGTGCACCCCAGCTGC
AAGGTGACCGCCATGAAGTGCTTTCTGCTGGAACTGCAGGTGATC
AGCCTGGAAAGCGGCGACGCCAGCATCCACGACACCGTGGAGAAC
CTGATCATCCTGGCCAACAACAGCCTGAGCAGCAACGGCAACGTG
ACCGAGAGCGGCTGCAAAGAGTGCGAGGAACTGGAAGAGAAGAAC
ATCAAAGAGTTTCTGCAGAGCTTCGTGCACATCGTGCAGATGTTC
ATCAACACCAGCTGA.

A corresponding amino acid sequence for m14-1CAR701VLVHCD28Z15 having an IgG hinge is as follows:

(SEQ ID NO: 99)
MGMALPVTALLLPLALLLHAARPDIQMTQSSSYLSVSLGGRVTIT
CKASDHINNWLAWYQQKPGNAPRLLISGATSLETGVPSRFSGSGS
GKDYTLSITSLQTEDVATYYCQQYWSTPWTFGGGTKLEIKPGGGG
SGGGGSGGGASEVQLQQSGAELVKPGASVKLSCTASGFNIKDSYM
HWVKQRPEQGLEWIGRIDPANANPKYDPKFQGKATITTDTSSNTA
YLQLSSLTSEDTAVYYCARDYGGYFDVWGAGTTVTVSSRTVTVSS
QDPAEPKSPDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPGKKDPKFWVLVVVGGVLACYSLLVTVAFIIFWVRSK
RSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTAT
KDTYDALHMQALPPRGPQCTNYALLKLAGDVESNPGPMRISKPHL
RSISIQCYLCLLLNSHFLTEAGIHVFILGCFSAGLPKTEANWVNV
ISDLKKIEDLIQSMHIDATLYTESDVHPSCKVTAMKCFLLELQVI
SLESGDASIHDTVENLIILANNSLSSNGNVTESGCKECEELEEKN
IKEFLQSFVHIVQMFINTS.

m14-1CAR701VHVLCD28Z15 Having an CD28 Hinge

(SEQ ID NO: 42)
ATGGGGATGGCCCTGCCTGTGACAGCTCTGCTCCTCCCTCTGGCC
CTGCTGCTCCATGCCGCCAGACCCGAGGTTCAGCTGCAGCAGTCT
GGGGCAGAGCTTGTGAAGCCAGGGGCCTCAGTCAAGTTGTCCTGC
ACAGCTTCTGGCTTCAACATTAAAGACAGCTATATGCACTGGGTG
AAGCAGAGGCCTGAACAGGGCCTGGAGTGGATTGGAAGGATTGAT
CCTGCGAATGCTAATCCTAAATATGACCCGAAGTTCCAGGGCAAG
GCCACTATAACAACAGACACATCCTCCAACACAGCCTACCTGCAG
CTCAGCAGCCTGACATCTGAGGACACTGCCGTCTATTACTGTGCT
AGAGACTACGGAGGGTACTTCGATGTCTGGGGCGCAGGGACCACG
GTCACCGTCTCCTCAGGTGGAGGCGGTTCAGGCGGAGGTGGCTCT
GGCGGTGGCGCTAGCGACATCCAGATGACACAATCTTCATCCTAC
TTGTCTGTATCTCTAGGAGGCAGAGTCACCATTACTTGCAAGGCA
AGTGACCACATTAATAATTGGTTAGCCTGGTATCAGCAGAAACCA
GGAAATGCTCCTAGGCTCTTAATATCTGGTGCAACCAGTTTGGAA
ACTGGGGTTCCTTCAAGATTCAGTGGCAGTGGATCTGGAAAGGAT
TACACTCTCAGCATTACCAGTCTTCAGACTGAAGATGTTGCCACT
TATTACTGTCAACAGTATTGGAGTACTCCGTGGACGTTCGGTGGA
GGCACCAAGCTGGAAATCAAACCCCGTACGGTCACTGTCTCTTCA
CAGGATCCCGCCGAGCCCAAATCTCCTGACAAAACTCACACATGC
CCACCGTGCCCAGCACCTGAACTCCTGGGGGGACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCAAGGTCTCCAACAAAGCCCTCCCAGCCCCC
ATCGAGAAAACCATCTCCAAAGCCAAAGGGCAGCCCCGAGAACCA
CAGGTGTACACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAACCGGAGAACAACTAC
AAGACCACGCCTCCCGTGCTGGACTCCGACGGCTCCTTCTTCCTC
TACAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTAAAAAAGATCCCAAA
TTTTGGGTGCTGGTGGTGGTTGGTGGAGTCCTGGCTTGCTATAGC
TTGCTAGTAACAGTGGCCTTTATTATTTTCTGGGTGAGGAGTAAG
AGGAGCAGGCTCCTGCACAGTGACTACATGAACATGACTCCCCGC
CGCCCCGGGCCCACCCGCAAGCATTACCAGCCCTATGCCCCACCA
CGCGACTTCGCAGCCTATCGCTCACGCGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAA
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
GGACCGCAGTGTACTAATTATGCTCTCTTGAAATTGGCTGGAGAT
GTTGAGAGCAATCCCGGGCCCATGCGCATTAGCAAGCCCCACCTG
CGGAGCATCAGCATCCAGTGCTACCTGTGCCTGCTGCTGAACAGC
CACTTCCTGACCGAGGCCGGCATCCACGTGTTCATCCTGGGCTGC
TTCAGCGCCGGACTGCCCAAGACCGAGGCCAACTGGGTGAACGTG
ATCAGCGACCTGAAGAAGATCGAGGACCTGATCCAGAGCATGCAC
ATCGACGCCACCCTGTACACCGAGAGCGACGTGCACCCCAGCTGC
AAGGTGACCGCCATGAAGTGCTTTCTGCTGGAACTGCAGGTGATC
AGCCTGGAAAGCGGCGACGCCAGCATCCACGACACCGTGGAGAAC
CTGATCATCCTGGCCAACAACAGCCTGAGCAGCAACGGCAACGTG
ACCGAGAGCGGCTGCAAAGAGTGCGAGGAACTGGAAGAGAAGAAC
ATCAAAGAGTTTCTGCAGAGCTTCGTGCACATCGTGCAGATGTTC
ATCAACACCAGCTGA.

A corresponding amino acid sequence for m14-1CAR701VHVLCD28Z15 having an CD28 hinge is as follows:

(SEQ ID NO: 104)
MGMALPVTALLLPLALLLHAARPEVQLQQSGAELVKPGASVKLSC
TASGFNIKDSYMHWVKQRPEQGLEWIGRIDPANANPKYDPKFQGK
ATITTDTSSNTAYLQLSSLTSEDTAVYYCARDYGGYFDVWGAGTT
VTVSSGGGGSGGGGSGGGASDIQMTQSSSYLSVSLGGRVTITCKA
SDHINNWLAWYQQKPGNAPRLLISGATSLETGVPSRFSGSGSGKD
YTLSITSLQTEDVATYYCQQYWSTPWTFGGGTKLEIKPRTVTVSS
QDPAEPKSPDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPGKKDPKFWVLVVVGGVLACYSLLVTVAFIIFWVRSK
RSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTAT
KDTYDALHMQALPPRGPQCTNYALLKLAGDVESNPGPMRISKPHL
RSISIQCYLCLLLNSHFLTEAGIHVFILGCFSAGLPKTEANWVNV
ISDLKKIEDLIQSMHIDATLYTESDVHPSCKVTAMKCFLLELQVI
SLESGDASIHDTVENLIILANNSLSSNGNVTESGCKECEELEEKN
IKEFLQSFVHIVQMFINTS.

m14-1CAR701VLVHCD28Z15 Having an CD28 Hinge

(SEQ ID NO: 43)
ATGGGGATGGCCCTGCCTGTGACAGCTCTGCTCCTCCCTCTGGCC
CTGCTGCTCCATGCCGCCAGACCCGACATCCAGATGACACAATCT
TCATCCTACTTGTCTGTATCTCTAGGAGGCAGAGTCACCATTACT
TGCAAGGCAAGTGACCACATTAATAATTGGTTAGCCTGGTATCAG
CAGAAACCAGGAAATGCTCCTAGGCTCTTAATATCTGGTGCAACC
AGTTTGGAAACTGGGGTTCCTTCAAGATTCAGTGGCAGTGGATCT
GGAAAGGATTACACTCTCAGCATTACCAGTCTTCAGACTGAAGAT
GTTGCCACTTATTACTGTCAACAGTATTGGAGTACTCCGTGGACG
TTCGGTGGAGGCACCAAGCTGGAAATCAAACCCGGTGGAGGCGGT
TCAGGCGGAGGTGGCTCTGGCGGTGGCGCTAGCGAGGTTCAGCTG
CAGCAGTCTGGGGCAGAGCTTGTGAAGCCAGGGGCCTCAGTCAAG
TTGTCCTGCACAGCTTCTGGCTTCAACATTAAAGACAGCTATATG
CACTGGGTGAAGCAGAGGCCTGAACAGGGCCTGGAGTGGATTGGA
AGGATTGATCCTGCGAATGCTAATCCTAAATATGACCCGAAGTTC
CAGGGCAAGGCCACTATAACAACAGACACATCCTCCAACACAGCC
TACCTGCAGCTCAGCAGCCTGACATCTGAGGACACTGCCGTCTAT
TACTGTGCTAGAGACTACGGAGGGTACTTCGATGTCTGGGGCGCA
GGGACCACGGTCACCGTCTCCTCACGTACGGTCACTGTCTCTTCA
CAGGATCCCGCCGAGCCCAAATCTCCTGACAAAACTCACACATGC
CCACCGTGCCCAGCACCTGAACTCCTGGGGGGACCGTCAGTCTTC
CTCTTCCCCCCAAAACCCAAGGACACCCTCATGATCTCCCGGACC
CCTGAGGTCACATGCGTGGTGGTGGACGTGAGCCACGAAGACCCT
GAGGTCAAGTTCAACTGGTACGTGGACGGCGTGGAGGTGCATAAT
GCCAAGACAAAGCCGCGGGAGGAGCAGTACAACAGCACGTACCGT
GTGGTCAGCGTCCTCACCGTCCTGCACCAGGACTGGCTGAATGGC
AAGGAGTACAAGTGCAAGGTCTCCAACAAAGCCCTCCCAGCCCCC
ATCGAGAAAACCATCTCCAAAGCCAAAGGGCAGCCCCGAGAACCA
CAGGTGTACACCCTGCCCCCATCCCGGGATGAGCTGACCAAGAAC
CAGGTCAGCCTGACCTGCCTGGTCAAAGGCTTCTATCCCAGCGAC
ATCGCCGTGGAGTGGGAGAGCAATGGGCAACCGGAGAACAACTAC
AAGACCACGCCTCCCGTGCTGGACTCCGACGGCTCCTTCTTCCTC
TACAGCAAGCTCACCGTGGACAAGAGCAGGTGGCAGCAGGGGAAC
GTCTTCTCATGCTCCGTGATGCATGAGGCTCTGCACAACCACTAC
ACGCAGAAGAGCCTCTCCCTGTCTCCGGGTAAAAAAGATCCCAAA
TTTTGGGTGCTGGTGGTGGTTGGTGGAGTCCTGGCTTGCTATAGC
TTGCTAGTAACAGTGGCCTTTATTATTTTCTGGGTGAGGAGTAAG
AGGAGCAGGCTCCTGCACAGTGACTACATGAACATGACTCCCCGC
CGCCCCGGGCCCACCCGCAAGCATTACCAGCCCTATGCCCCACCA
CGCGACTTCGCAGCCTATCGCTCACGCGTGAAGTTCAGCAGGAGC
GCAGACGCCCCCGCGTACCAGCAGGGCCAGAACCAGCTCTATAAC
GAGCTCAATCTAGGACGAAGAGAGGAGTACGATGTTTTGGACAAA
AGACGTGGCCGGGACCCTGAGATGGGGGGAAAGCCGAGAAGGAAG
AACCCTCAGGAAGGCCTGTACAATGAACTGCAGAAAGATAAGATG
GCGGAGGCCTACAGTGAGATTGGGATGAAAGGCGAGCGCCGGAGG
GGCAAGGGGCACGATGGCCTTTACCAGGGTCTCAGTACAGCCACC
AAGGACACCTACGACGCCCTTCACATGCAGGCCCTGCCCCCTCGC
GGACCGCAGTGTACTAATTATGCTCTCTTGAAATTGGCTGGAGAT
GTTGAGAGCAATCCCGGGCCCATGCGCATTAGCAAGCCCCACCTG
CGGAGCATCAGCATCCAGTGCTACCTGTGCCTGCTGCTGAACAGC
CACTTCCTGACCGAGGCCGGCATCCACGTGTTCATCCTGGGCTGC
TTCAGCGCCGGACTGCCCAAGACCGAGGCCAACTGGGTGAACGTG
ATCAGCGACCTGAAGAAGATCGAGGACCTGATCCAGAGCATGCAC
ATCGACGCCACCCTGTACACCGAGAGCGACGTGCACCCCAGCTGC
AAGGTGACCGCCATGAAGTGCTTTCTGCTGGAACTGCAGGTGATC
AGCCTGGAAAGCGGCGACGCCAGCATCCACGACACCGTGGAGAAC
CTGATCATCCTGGCCAACAACAGCCTGAGCAGCAACGGCAACGTG
ACCGAGAGCGGCTGCAAAGAGTGCGAGGAACTGGAAGAGAAGAAC
ATCAAAGAGTTTCTGCAGAGCTTCGTGCACATCGTGCAGATGTTC
ATCAACACCAGCTGA.

A corresponding amino acid sequence for m14-1CAR701VLVHCD28Z15 having an CD28 hinge is as follows:

(SEQ ID NO: 105)
MGMALPVTALLLPLALLLHAARPDIQMTQSSSYLSVSLGGRVTIT
CKASDHINNWLAWYQQKPGNAPRLLISGATSLETGVPSRFSGSGS
GKDYTLSITSLQTEDVATYYCQQYWSTPWTFGGGTKLEIKPGGGG
SGGGGSGGGASEVQLQQSGAELVKPGASVKLSCTASGFNIKDSYM
HWVKQRPEQGLEWIGRIDPANANPKYDPKFQGKATITTDTSSNTA
YLQLSSLTSEDTAVYYCARDYGGYFDVWGAGTTVTVSSRTVTVSS
QDPAEPKSPDKTHTCPPCPAPELLGGPSVFLFPPKPKDTLMISRT
PEVTCVVVDVSHEDPEVKFNWYVDGVEVHNAKTKPREEQYNSTYR
VVSVLTVLHQDWLNGKEYKCKVSNKALPAPIEKTISKAKGQPREP
QVYTLPPSRDELTKNQVSLTCLVKGFYPSDIAVEWESNGQPENNY
KTTPPVLDSDGSFFLYSKLTVDKSRWQQGNVFSCSVMHEALHNHY
TQKSLSLSPGKKDPKFWVLVVVGGVLACYSLLVTVAFIIFWVRSK
RSRLLHSDYMNMTPRRPGPTRKHYQPYAPPRDFAAYRSRVKFSRS
ADAPAYQQGQNQLYNELNLGRREEYDVLDKRRGRDPEMGGKPRRK
NPQEGLYNELQKDKMAEAYSEIGMKGERRRGKGHDGLYQGLSTAT
KDTYDALHMQALPPRGPQCTNYALLKLAGDVESNPGPMRISKPHL
RSISIQCYLCLLLNSHFLTEAGIHVFILGCFSAGLPKTEANWVNV
ISDLKKIEDLIQSMHIDATLYTESDVHPSCKVTAMKCFLLELQVI
SLESGDASIHDTVENLIILANNSLSSNGNVTESGCKECEELEEKN
IKEFLQSFVHIVQMFINTS.

### C. T Cell Receptor (TCR)

In some embodiments, a CD70-targeting genetically engineered antigen receptor includes recombinant TCRs and/or TCRs cloned from naturally occurring T cells, or one or more portions thereof. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form.

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the ap form or 76 form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable α chain and variable p chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the j-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., α-chain constant domain or Ca, typically amino acids 117 to 259 based on Kabat, 1-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the a and p chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ζ chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains α and β (or optionally γ and δ) or it may be a single chain TCR construct. In some embodiments, the TCR is a heterodimer containing two separate chains (α and ρ chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

### IX. Cytokines

One or more cytokines may be utilized with one or more CD70-targeting genetically engineered receptors, such as CD70-specific CARs. In some cases, one or more cytokines are present on the same vector molecule as the engineered receptor, although in other cases they are on separate vector molecules. In particular embodiments, one or more cytokines are co-expressed from the same vector as the engineered receptor. One or more cytokines may be produced as a separate polypeptide from the CD70-specific receptor. As one example, Interleukin-15 (IL-15), is utilized. IL-15 may be employed because, for example, it is tissue restricted and only under pathologic conditions is it observed at any level in the serum, or systemically. IL-15 possesses several attributes that are desirable for adoptive therapy. IL-15 is a homeostatic cytokine that induces development and cell proliferation of natural killer cells, promotes the eradication of established tumors via alleviating functional suppression of tumor-resident cells, and inhibits activation-induced cell death. In addition to IL-15, other cytokines are envisioned. These include, but are not limited to, cytokines, chemokines, and other molecules that contribute to the activation and proliferation of cells used for human application. As one example, the one or more cytokines are IL-15, IL-12, IL-2, IL-18, IL-21, IL-23, IL-7, or combination thereof. NK cells expressing IL-15 may be utilized and are capable of continued supportive cytokine signaling, which is useful for their survival post-infusion.

In specific embodiments, NK cells express one or more exogenously provided cytokines. The cytokine may be exogenously provided to the NK cells because it is expressed from an expression vector within the cell and/or because it is provided in a culture medium of the cells. In an alternative case, an endogenous cytokine in the cell is upregulated upon manipulation of regulation of expression of the endogenous cytokine, such as genetic recombination at the promoter site(s) of the cytokine. In cases wherein the cytokine is provided on an expression construct to the cell, the cytokine may be encoded from the same vector as a suicide gene. The cytokine may be expressed as a separate polypeptide molecule from a suicide gene and as a separate polypeptide from an engineered receptor of the cell. In some embodiments, the present disclosure concerns co-utilization of CAR and/or TCR vectors with IL-15, particularly in NK cells.

### X. Suicide Genes

In particular embodiments, a suicide gene is utilized in conjunction with cell therapy of any kind to control its use and allow for termination of the cell therapy at a desired event and/or time. The suicide gene is employed in transduced cells for the purpose of eliciting death for the transduced cells when needed. The CD70-targeting cells of the present disclosure that have been modified to harbor a vector encompassed by the disclosure may comprise one or more suicide genes. In some embodiments, the term “suicide gene” as used herein is defined as a gene which, upon administration of a prodrug or other agent, effects transition of a gene product to a compound which kills its host cell. In other embodiments, a suicide gene encodes a gene product that is, when desired, targeted by an agent (such as an antibody) that targets the suicide gene product.

Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside. The E. coli purine nucleoside phosphorylase, a so-called suicide gene that converts the prodrug 6-methylpurine deoxyriboside to toxic purine 6-methylpurine, may be used. Other examples of suicide genes used with prodrug therapy are the E. coli cytosine deaminase gene and the HSV thymidine kinase gene.

Exemplary suicide genes also include CD20, CD52, EGFRv3, or inducible caspase 9. In one embodiment, a truncated version of EGFR variant III (EGFRv3) may be used as a suicide antigen that can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

In particular embodiments, vectors that encode the CD70-targeting CAR, or any vector in a NK cell encompassed herein, include one or more suicide genes. The suicide gene may or may not be on the same vector as a CD70-targeting CAR. In cases wherein the suicide gene is present on the same vector as the CD70-targeting CAR, the suicide gene and the CAR may be separated by an IRES or 2A element, for example.

### XI. Immune Cell Engagers

Aspects of the present disclosure are directed to immune cell engagers (also “immune cell engager polypeptides” or “engagers”). An “immune cell engager,” as used herein, describes any polypeptide having at least one antigen binding region and at least one immune cell binding region. An immune cell engager may, when provided to a subject, bind to both an antigen-expressing cell (e.g., a cancer cell expressing a cancer antigen) and an immune cell (e.g., a T cell, an NK cell, etc). In some embodiments, an antigen binding region of an immune cell engager is a CD70-binding region. In some embodiments, an immune cell engager of the present disclosure comprises a CD70-binding region described herein, including, for example, a CD70-binding region comprising any one or more of SEQ ID NOs:44-105.

As used herein, an “immune cell binding region” describes a polypeptide region that specifically binds to a component of an immune cell, including, for example, a protein expressed by an immune cell. In some embodiments, an immune cell binding region of the disclosure is a polypeptide region that specifically binds to a cell surface protein expressed by an immune cell. Immune cells which may be targeted by an immune cell binding region include, but are not limited to, T cells, natural killer (NK) cells, gamma delta (γδ) T cells, alpha beta (αβ) T cells, natural killer T (NKT) cells, B cells, mesenchymal stromal cells, granulocytes, monocytes, macrophages, and dendritic cells. Examples of cell surface proteins which may be targeted by immune cell binding regions of the disclosure include but are not limited to CD2, CD3 and CD3 subunits such as CD3E, CD5, CD28, 4-1BB, OX40, CD2, CD5, CD95, CD27, IL-7R, ICOS, IL2Rβ, CD45, CD48, and CD137 for T cells; CD16A, CD25, CD38, CD44, CD56, CD69, CD94, CS1, DNAM, 2B4, CD2, a KIR, CD335 (NKp46), CD336 (NKp44), CD337 (NKp30), NKp80, NKG2A, NKG2C, NKG2D, DNAM, and NCRs for NK cells; CD18, CD64 and CD89 for granulocytes; CD18, CD32, CD47, CD64, CD89 and mannose receptor for monocytes and macrophages; CD64 and mannose receptor for dendritic cells; as well as CD35. In some embodiments, an immune cell binding region specifically binds to a cell surface protein expressed by a T cell. In some embodiments, an immune cell binding region specifically binds to CD3. The immune cell binding region may specifically bind to the CD3γ chain, CD3δ chain, CD3E chain, or CD3ζ chain of CD3. In some embodiments, an immune cell binding region comprises an scFv from an anti-CD3 antibody. In some embodiments, an immune cell binding region specifically binds to a cell surface protein expressed by an NK cell. In some embodiments, an immune cell binding region specifically binds to CD16A. In some embodiments, an immune cell binding region comprises an scFv from an anti-CD16A antibody.

Nonlimiting examples of immune cell engagers contemplated herein include multispecific antibodies (e.g., bispecific antibodies, trispecific antibodies, tetravalent antibodies, etc.), bispecific T cell engagers (also called “BiTEs”), bispecific killer engagers (also called “BiKEs”), trispecific killer engagers (also called “TriKEs), and the like. Certain immune cell engagers are described in, for example, U.S. Patent Application Publications 2017/0218075, 2019/0040155, and 2020/0405833 (each incorporated by reference in its entirety) and in Goebeler, M E., Bargou, R. C. Nat Rev Clin Oncol 17, 418-434 (2020); Felices, Martin et al. Methods in molecular biology (Clifton, N.J.) vol. 1441 (2016): 333-46; and Tian, Z., et al. J Hematol Oncol 14, 75 (2021) (each incorporated by reference in its entirety).

### XI. Antigens

Aspects of the disclosure are directed to polypeptides (e.g., antibodies, CARs, engagers, etc.) that target one or more particular antigens. Among the antigens targeted by the antibodies and/or engineered polypeptides of the disclosure are those expressed in the context of a disease, condition, or cell type to be targeted. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may be targeted in the present method. The antigen may be associated with certain cancer cells but not associated with non-cancerous cells, in some cases. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include CD19, EBNA, CD123, HER2, CA-125, TRAIL/DR4, CD20, CD22, CD70, CD38, CD123, CLL1, carcinoembryonic antigen, alphafetoprotein, CD56, AKT, Her3, epithelial tumor antigen, CD319 (CS1), ROR1, folate binding protein, HIV-1 envelope glycoprotein gp120, HIV-1 envelope glycoprotein gp41, CD5, CD23, CD30, HERV-K, IL-11Ralpha, kappa chain, lambda chain, CSPG4, CD33, CD47, CLL-1, U5snRNP200, CD200, BAFF-R, BCMA, CD99, p53, mutated p53, Ras, mutated ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, melanoma-associated antigen, BAGE, DAM-6, -10, GAGE-1, -2, -8, GAGE-3, -4, -5, -6, -7B, NA88-A, MC1R, mda-7, gp75, Gp100, PSA, PSM, Tyrosinase, tyrosinase-related protein, TRβ-1, TRβ-2, ART-4, CAMEL, CEA, Cyp-B, hTERT, hTRT, iCE, MUC1, MUC2, Phosphoinositide 3-kinases (PI3Ks), TRK receptors, PRAME, P15, RUl, RU2, SART-1, SART-3, Wilms' tumor antigen (WT1), AFP, -catenin/m, Caspase-8/m, CDK-4/m, ELF2M, GnT-V, G250, HAGE, HSP70-2M, HST-2, KIAA0205, MUM-1, MUM-2, MUM-3, Myosin/m, RAGE, SART-2, TRβ-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), VEGFR2, cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF—B), Notch receptors (e.g., Notchl-4), NY ESO 1, c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SAGE, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGIB, SUNC1, and LRRN1. Examples of sequences for antigens are known in the art, for example, in the GenBank® database: CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, liver, brain, bone, stomach, spleen, testicular, cervical, anal, gall bladder, thyroid, or melanoma cancers, as examples. Exemplary tumor-associated antigens or tumor cell-derived antigens include MAGE 1, 3, and MAGE 4 (or other MAGE antigens such as those disclosed in International Patent Publication No. WO 99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self-peptide hormone, such as whole length gonadotrophin hormone releasing hormone (GnRH), a short 10 amino acid long peptide, useful in the treatment of many cancers.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alpha-fetoprotein.

### XIII. Vectors

CD70-targeting polypeptides (e.g., CARs, chimeric polypeptides, immune cell engagers, etc.) may be delivered to the recipient immune cells by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the immune cell is transduced with a vector encoding the CD70-targeting polypeptide and also requires transduction of another gene or genes into the cell, such as a suicide gene and/or cytokine and/or an optional therapeutic gene product, the CD70-targeting polypeptide, suicide gene, cytokine, and optional therapeutic gene may or may not be comprised on or with the same vector. In some cases, the CD70-targeting polypeptide, suicide gene, cytokine, and optional therapeutic gene are expressed from the same vector molecule, such as the same viral vector molecule. In such cases, the expression of the CD70-targeting polypeptide, suicide gene, cytokine, and optional therapeutic gene may or may not be regulated by the same regulatory element(s). When the CD70-targeting polypeptide, suicide gene, cytokine, and optional therapeutic gene are on the same vector, they may or may not be expressed as separate polypeptides. In cases wherein they are expressed as separate polypeptides, they may be separated on the vector by a 2A element or IRES element (or both kinds may be used on the same vector once or more than once), for example.

### A. General Embodiments

One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure.

### 1. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells may be comprised of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters, for example. In cases wherein the vector is utilized for the generation of cancer therapy, a promoter may be effective under conditions of hypoxia.

### 2. Promoter/Enhancers

The expression constructs provided herein comprise a promoter to drive expression of the antigen receptor and other cistron gene products. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, for example, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at GenBank®, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

### 3. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

As detailed elsewhere herein, certain 2A sequence elements could be used to create linked- or co-expression of genes in the constructs provided in the present disclosure. For example, cleavage sequences could be used to co-express genes by linking open reading frames to form a single cistron. An exemplary cleavage sequence is the equine rhinitis A virus (E2A) or the F2A (Foot-and-mouth disease virus 2A) or a “2A-like” sequence (e.g., Thosea asigna virus 2A; T2A) or porcine teschovirus-1 (P2A). In specific embodiments, in a single vector the multiple 2A sequences are non-identical, although in alternative embodiments the same vector utilizes two or more of the same 2A sequences. Examples of 2A sequences are provided in US 2011/0065779 which is incorporated by reference herein in its entirety.

### 4. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

### 5. Selection and Screenable Markers

In some embodiments, NK cells comprising a CD70-targeting receptor construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

### B. Multicistronic Vectors

In particular embodiments, the CD70-targeting receptor, optional suicide gene, optional cytokine, and/or optional therapeutic gene are expressed from a multicistronic vector (the term “cistron” as used herein refers to a nucleic acid sequence from which a gene product may be produced). In specific embodiments, the multicistronic vector encodes the CD70-targeting receptor, the suicide gene, and at least one cytokine, and/or engineered receptor, such as a T-cell receptor and/or an additional non-CD70-targeting CAR. In some cases, the multicistronic vector encodes at least one CD70-targeting CAR, at least one TNF-alpha mutant, and at least one cytokine. The cytokine may be of a particular type of cytokine, such as human or mouse or any species. In specific cases, the cytokine is IL15, IL12, IL2, IL18, and/or IL21.

In certain embodiments, the present disclosure provides a flexible, modular system (the term “modular” as used herein refers to a cistron or component of a cistron that allows for interchangeability thereof, such as by removal and replacement of an entire cistron or of a component of a cistron, respectively, for example by using standard recombination techniques) utilizing a polycistronic vector having the ability to express multiple cistrons at substantially identical levels. The system may be used for cell engineering allowing for combinatorial expression (including overexpression) of multiple genes. In specific embodiments, one or more of the genes expressed by the vector includes one, two, or more antigen receptors. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise: (1) one or more reporters, for example fluorescent or enzymatic reporters, such as for cellular assays and animal imaging; (2) one or more cytokines or other signaling molecules; and/or (3) a suicide gene.

In specific cases, the vector may comprise at least 4 cistrons separated by cleavage sites of any kind, such as 2A cleavage sites. The vector may or may not be Moloney Murine Leukemia Virus (MoMLV or MMLV)-based including the 3′ and 5′ LTR with the psi packaging sequence in a pUC19 backbone. The vector may comprise 4 or more cistrons with three or more 2A cleavage sites and multiple ORFs for gene swapping. The system allows for combinatorial overexpression of multiple genes (7 or more) that are flanked by restriction site(s) for rapid integration through subcloning, and the system also includes at least three 2A self-cleavage sites, in some embodiments. Thus, the system allows for expression of multiple CARs, TCRs, signaling molecules, cytokines, cytokine receptors, and/or homing receptors. This system may also be applied to other viral and non-viral vectors, including but not limited lentivirus, adenovirus AAV, as well as non-viral plasmids.

The modular nature of the system also enables efficient subcloning of a gene into each of the 4 cistrons in the polycistronic expression vector and the swapping of genes, such as for rapid testing. Restriction sites strategically located in the polycistronic expression vector allow for swapping of genes with efficiency.

Embodiments of the disclosure encompass systems that utilize a polycistronic vector wherein at least part of the vector is modular, for example by allowing removal and replacement of one or more cistrons (or component(s) of one or more cistrons), such as by utilizing one or more restriction enzyme sites whose identity and location are specifically selected to facilitate the modular use of the vector. The vector also has embodiments wherein multiple of the cistrons are translated into a single polypeptide and processed into separate polypeptides, thereby imparting an advantage for the vector to express separate gene products in substantially equimolar concentrations.

The vector of the disclosure is configured for modularity to be able to change one or more cistrons of the vector and/or to change one or more components of one or more particular cistrons. The vector may be designed to utilize unique restriction enzyme sites flanking the ends of one or more cistrons and/or flanking the ends of one or more components of a particular cistron.

Embodiments of the disclosure include polycistronic vectors comprising at least two, at least three, or at least four cistrons each flanked by one or more restriction enzyme sites, wherein at least one cistron encodes for at least one antigen receptor. In some cases, two, three, four, or more of the cistrons are translated into a single polypeptide and cleaved into separate polypeptides, whereas in other cases multiple of the cistrons are translated into a single polypeptide and cleaved into separate polypeptides. Adjacent cistrons on the vector may be separated by a self cleavage site, such as a 2A self cleavage site. In some cases each of the cistrons express separate polypeptides from the vector. On particular cases, adjacent cistrons on the vector are separated by an IRES element.

In certain embodiments, the present disclosure provides a system for cell engineering allowing for combinatorial expression, including overexpression, of multiple cistrons that may include one, two, or more antigen receptors, for example. In particular embodiments, the use of a polycistronic vector as described herein allows for the vector to produce equimolar levels of multiple gene products from the same mRNA. The multiple genes may comprise, but are not limited to, CARs, TCRs, cytokines, chemokines, homing receptors, CRISPR/Cas9-mediated gene mutations, decoy receptors, cytokine receptors, chimeric cytokine receptors, and so forth. The vector may further comprise one or more fluorescent or enzymatic reporters, such as for cellular assays and animal imaging. The vector may also comprise a suicide gene product for termination of cells harboring the vector when they are no longer needed or become deleterious to a host to which they have been provided.

In specific embodiments, the vector is a viral vector (retroviral vector, lentiviral vector, adenoviral vector, or adeno-associated viral vector, for example) or a non-viral vector. The vector may comprise a Moloney Murine Leukemia Virus (MMLV) 5′ LTR, 3′ LTR, and/or psi packaging element. In specific cases, the psi packaging is incorporated between the 5′ LTR and the antigen receptor coding sequence. The vector may or may not comprise pUC19 sequence. In some aspects of the vector, at least one cistron encodes for a cytokine (IL-15, IL-7, IL-21, IL-23, IL-18, IL-12, or IL-2, for example), chemokine, cytokine receptor, and/or homing receptor.

When 2A cleavages sites are utilized in the vector, the 2A cleavage site may comprise a P2A, T2A, E2A and/or F2A site.

A restriction enzyme site may be of any kind and may include any number of bases in its recognition site, such as between 4 and 8 bases; the number of bases in the recognition site may be at least 4, 5, 6, 7, 8, or more. The site when cut may produce a blunt cut or sticky ends. The restriction enzyme may be of Type I, Type II, Type III, or Type IV, for example. Restriction enzyme sites may be obtained from available databases, such as Integrated relational Enzyme database (IntEnz) or BRENDA (The Comprehensive Enzyme Information System).

Exemplary vectors may be circular and by convention, where position 1 (12 o'clock position at the top of the circle, with the rest of the sequence in clock-wise direction) is set at the start of 5′ LTR.

In embodiments wherein self-cleaving 2A peptides are utilized, the 2A peptides may be 18-22 amino-acid (aa)-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-12A), and T2A (Thosea asigna virus 2A) were also identified. The mechanism of 2A-mediated “self-cleavage” was discovered to be ribosome skipping the formation of a glycyl-prolyl peptide bond at the C-terminus of the 2A.

In specific cases, the vector may be a γ-retroviral transfer vector. The retroviral transfer vector may comprises a backbone based on a plasmid, such as the pUC19 plasmid (large fragment (2.63 kb) in between HindIII and EcoRI restriction enzyme sites). The backbone may carry viral components from Moloney Murine Leukemia Virus (MoMLV) including 5′ LTR, psi packaging sequence, and 3′ LTR. LTRs are long terminal repeats found on either side of a retroviral provirus, and in the case of a transfer vector, brackets the genetic cargo of interest, such as CD70-targeting CARs and associated components. The psi packaging sequence, which is a target site for packaging by nucleocapsid, is also incorporated in cis, sandwiched between the 5′ LTR and the CAR coding sequence. Thus, the basic structure of an example of a transfer vector can be configured as such: pUC19 sequence-5′ LTR-psi packaging sequence-genetic cargo of interest-3′ LTR-pUC19 sequence. This system may also be applied to other viral and non-viral vectors, including but not limited lentivirus, adenovirus AAV, as well as non-viral plasmids.

### XIV. Cells

The present disclosure encompasses cells, including immune cells and stem cells of any kind, that harbor at least one vector that encodes a CD70-targeting polypeptide (e.g., a CD70 antibody, CD70 CAR, and/or CD70 engager) and that also may encode at least one cytokine and/or at least one suicide gene. In some cases, different vectors encode the CD70-targeting polypeptide vs. encodes the suicide gene and/or cytokine. The immune cells, including NK cells, may be derived from cord blood (including pooled cord blood from multiple sources), peripheral blood, induced pluripotent stem cells (iPSCs), hematopoietic stem cells (HSCs), bone marrow, or a mixture thereof. The NK cells may be derived from a cell line such as, but not limited to, NK-92 cells, for example. The NK cell may be a cord blood mononuclear cell, such as a CD56⁺ NK cell.

The present disclosure encompasses immune or other cells of any kind, including conventional T cells, gamma-delta T cells, NKT and invariant NK T cells, regulatory T cells, macrophages, B cells, dendritic cells, mesenchymal stromal cells (MSCs), or a mixture thereof.

In some cases, the cells have been expanded in the presence of an effective amount of universal antigen presenting cells (UAPCs), including in any suitable ratio. The cells may be cultured with the UAPCs at a ratio of 10:1 to 1:10; 9:1 to 1:9; 8:1 to 1:8; 7:1 to 1:7; 6:1 to 1:6; 5:1 to 1:5; 4:1 to 1:4; 3:1 to 1:3; 2:1 to 1:2; or 1:1, including at a ratio of 1:2, for example. In some cases, the NK cells were expanded in the presence of IL-2, such as at a concentration of 10-500, 10-400, 10-300, 10-200, 10-100, 10-50, 100-500, 100-400, 100-300, 100-200, 200-500, 200-400, 200-300, 300-500, 300-400, or 400-500 U/mL.

Following genetic modification with the vector(s), the NK cells may be immediately infused or may be stored. In certain aspects, following genetic modification, the cells may be propagated for days, weeks, or months ex vivo as a bulk population within about 1, 2, 3, 4, 5 days or more following gene transfer into cells. In a further aspect, the transfectants are cloned and a clone demonstrating presence of a single integrated or episomally maintained expression cassette or plasmid, and expression of the CD70-targeting polypeptide (e.g., a CD70 antibody, CD70 CAR, and/or CD70 engager) is expanded ex vivo. The clone selected for expansion demonstrates the capacity to specifically recognize and lyse CD70-expressing target cells. The recombinant immune cells may be expanded by stimulation with IL-2, or other cytokines that bind the common gamma-chain (e.g., IL-7, IL-12, IL-15, IL-21, IL-23, and others). The recombinant immune cells may be expanded by stimulation with artificial antigen presenting cells. In a further aspect, the genetically modified cells may be cryopreserved.

Embodiments of the disclosure encompass cells that express one or more CD70-targeting CARs and one or more suicide genes as encompassed herein. The NK cell comprises a recombinant nucleic acid that encodes one or more CD70-targeting CARs and one or more engineered nonsecretable, membrane bound TNF-alpha mutant polypeptides, in specific embodiments. In specific embodiments, in addition to expressing one or more CD70-targeting CARs and TNF-alpha mutant polypeptides, the cell also comprises a nucleic acid that encodes one or more therapeutic gene products.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the CD70-targeting polypeptide (e.g., a CD70 antibody, CD70 CAR, and/or CD70 engager), optional suicide gene, optional cytokine(s), and optional therapeutic gene product(s) (using standard techniques for transduction and expansion for adoptive cell therapy, for example), they may be provided back to the individual from which they were originally sourced. In some cases, the cells are stored for later use for the individual or another individual.

The immune cells may be comprised in a population of cells, and that population may have a majority that are transduced with one or more CD70-targeting polypeptides and/or one or more suicide genes and/or one or more cytokines. A cell population may comprise at least, at most, exactly, or between any two of 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of immune cells that are transduced with one or more CD70-targeting polypeptides and/or one or more suicide genes and/or one or more cytokines. The one or more CD70-targeting polypeptides and/or one or more suicide genes and/or one or more cytokines may be separate polypeptides.

The immune cells may be produced with the one or more CD70-targeting polypeptides and/or one or more suicide genes and/or one or more cytokines for the intent of being modular with respect to a specific purpose. For example, cells may be generated, including for commercial distribution, expressing a CD70-targeting CARs and/or one or more suicide genes and/or one or more cytokines (or distributed with a nucleic acid that encodes the mutant for subsequent transduction), and a user may modify them to express one or more other genes of interest (including therapeutic genes) dependent upon their intended purpose(s). For instance, an individual interested in treating CD70-positive cells, including CD70-positive cancer, may obtain or generate suicide gene-expressing cells (or heterologous cytokine-expressing cells) and modify them to express a receptor comprising a CD70-targeting polypeptide, or vice versa.

In particular embodiments, NK cells are utilized, and the genome of the transduced NK cells expressing the one or more CD70-targeting polypeptides and/or one or more suicide genes and/or one or more cytokines may be modified. The genome may be modified in any manner, but in specific embodiments the genome is modified by CRISPR gene editing, for example. The genome of the cells may be modified to enhance effectiveness of the cells for any purpose.

### XV. Gene Editing of Cells

In particular embodiments, cells comprising at least a CD70-targeting polypeptide are gene edited to modify expression of one or more endogenous genes in the cell. In specific cases, CD70-specific CAR cells are modified to have reduced levels of expression of one or more endogenous genes, including inhibition of expression of one or more endogenous genes (that may be referred to as knocked out). Such cells may or may not be expanded.

In particular cases, one or more endogenous genes of the CD70-specific CAR cells are modified, such as disrupted in expression where the expression is reduced in part or in full. In specific cases, one or more genes are knocked down or knocked out using processes of the disclosure. In specific cases, multiple genes are knocked down or knocked out, and this may or may not occur in the same step in their production. The genes that are edited in the CD70-specific CAR cells may be of any kind, but in specific embodiments the genes are genes whose gene products inhibit activity and/or proliferation of the CD70-specific CAR cells, including CD70-specific CAR NK cells, such as those derived from cord blood, as one example. In specific cases the genes that are edited in the CD70-specific CAR cells allow the CD70-specific CAR cells to work more effectively in a tumor microenvironment. In specific cases, the genes are one or more of NKG2A, SIGLEC-7, LAG3, TIM3, CISH, FOXO1, TGFBR2, TIGIT, CD96, ADORA2, NR3C1, PD1, PDL-1, PDL-2, CD47, SIRPA, SHIP1, ADAM17, RPS6, 4EBP1, CD25, CD40, IL21R, ICAM1, CD95, CD80, CD86, IL10R, CD5, and CD7. In specific embodiments, the TGFBR2 gene is knocked out or knocked down in the CD70-specific CAR cells.

In some embodiments, the gene editing is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins; in some embodiments, CpF1 is utilized instead of Cas9. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. at least, at most, exactly, or between any two of about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least, at most, exactly, or between any two of 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1 (Cas12a) homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). In some cases, Cpf1 (Cas12a) may be used as an endonuclease instead of Cas9. The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is at least, at most, exactly, or between any two of about 50%, 60%, 75%, 80%, 85%, 90%, 95%, 97%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (ILLUMINA®, San Diego, Calif.), SOAP (available at soap. genomics. org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRβ), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### XVI. Therapeutic Methods

The compositions of the disclosure may be used for in vivo, in vitro, or ex vivo administration. The route of administration of the composition may be, for example, intracutaneous, subcutaneous, intravenous, local, topical, and intraperitoneal administrations.

### A. Cancer Therapy

In some embodiments, the disclosed methods comprise administering a cancer therapy to a patient. In some embodiments, the cancer therapy comprises a local cancer therapy. In some embodiments, the cancer therapy excludes a systemic cancer therapy. In some embodiments, the cancer therapy excludes a local therapy. In some embodiments, the cancer therapy comprises a local cancer therapy without the administration of a system cancer therapy. In some embodiments, the cancer therapy comprises an immunotherapy, which may be an immune checkpoint therapy. Any of these cancer therapies may also be excluded. Combinations of these therapies may also be administered. For example, in some cases, immune cells expressing an anti-CD70 CAR of the disclosure can be administered with one or more antibodies or one or more bispecific or multispecific immune cell engagers.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus. In some embodiments, the cancer is recurrent cancer. In some embodiments, the cancer is Stage I cancer. In some embodiments, the cancer is Stage II cancer. In some embodiments, the cancer is Stage III cancer. In some embodiments, the cancer is Stage IV cancer.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

### 1. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumour-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immumotherapies are known in the art, and some are described below.

a. Checkpoint Inhibitors and Combination Treatment

Embodiments of the disclosure may include administration of immune checkpoint inhibitors, which are further described below.

### (1) PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the V_{H} region of nivolumab, pembrolizumab, or pidilizumab, and the CDR1, CDR2 and CDR3 domains of the V_{L} region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### (2) CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or V_{L} domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the V_{H} region of tremelimumab or ipilimumab, and the CDR1, CDR2 and CDR3 domains of the V_{L} region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

### (3) LAG3

Another immune checkpoint that can be targeted in the methods provided herein is the lymphocyte-activation gene 3 (LAG3), also known as CD223 and lymphocyte activating 3. The complete mRNA sequence of human LAG3 has the Genbank accession number NM_002286. LAG3 is a member of the immunoglobulin superfamily that is found on the surface of activated T cells, natural killer cells, B cells, and plasmacytoid dendritic cells. LAG3's main ligand is MHC class II, and it negatively regulates cellular proliferation, activation, and homeostasis of T cells, in a similar fashion to CTLA-4 and PD-1, and has been reported to play a role in Treg suppressive function. LAG3 also helps maintain CD8⁺ T cells in a tolerogenic state and, working with PD-1, helps maintain CD8 exhaustion during chronic viral infection. LAG3 is also known to be involved in the maturation and activation of dendritic cells. Inhibitors of the disclosure may block one or more functions of LAG3 activity.

In some embodiments, the immune checkpoint inhibitor is an anti-LAG3 antibody (e.g., a human antibody, a humanized antibody, or a chimeric antibody), an antigen binding fragment thereof, an immunoadhesin, a fusion protein, or oligopeptide.

Anti-human-LAG3 antibodies (or V_{H} and/or V_{L} domains derived therefrom) suitable for use in the present methods can be generated using methods well known in the art. Alternatively, art recognized anti-LAG3 antibodies can be used. For example, the anti-LAG3 antibodies can include: GSK2837781, IMP321, FS-118, Sym022, TSR-033, MGD013, BI754111, AVA-017, or GSK2831781. The anti-LAG3 antibodies disclosed in: U.S. Pat. No. 9,505,839 (BMS-986016, also known as relatlimab); U.S. Pat. No. 10,711,060 (IMP-701, also known as LAG525); U.S. Pat. No. 9,244,059 (IMP731, also known as H5L7BW); U.S. Pat. No. 10,344,089 (25F7, also known as LAG3.1); WO 2016/028672 (MK-4280, also known as 28G-10); WO 2017/019894 (BAP050); Burova E., et al., J. ImmunoTherapy Cancer, 2016; 4(Supp. 1):P195 (REGN3767); Yu, X., et al., mAbs, 2019; 11:6 (LBL-007) can be used in the methods disclosed herein. These and other anti-LAG-3 antibodies useful in the claimed invention can be found in, for example: WO 2016/028672, WO 2017/106129, WO 2017062888, WO 2009/044273, WO 2018/069500, WO 2016/126858, WO 2014/179664, WO 2016/200782, WO 2015/200119, WO 2017/019846, WO 2017/198741, WO 2017/220555, WO 2017/220569, WO 2018/071500, WO 2017/015560; WO 2017/025498, WO 2017/087589, WO 2017/087901, WO 2018/083087, WO 2017/149143, WO 2017/219995, US 2017/0260271, WO 2017/086367, WO 2017/086419, WO 2018/034227, and WO 2014/140180. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-LAG3 antibody. Accordingly, in one embodiment, the inhibitor comprises the CDR1, CDR2, and CDR3 domains of the V_{H} region of an anti-LAG3 antibody, and the CDR1, CDR2 and CDR3 domains of the V_{L} region of an anti-LAG3 antibody. In another embodiment, the antibody has at least about 70, 75, 80, 85, 90, 95, 97, or 99% (or any derivable range therein) variable region amino acid sequence identity with the above-mentioned antibodies.

### (4) TIM-3

Another immune checkpoint that can be targeted in the methods provided herein is the T-cell immunoglobulin and mucin-domain containing-3 (TIM-3), also known as hepatitis A virus cellular receptor 2 (HAVCR2) and CD3δ6. The complete mRNA sequence of human TIM-3 has the Genbank accession number NM_032782. TIM-3 is found on the surface IFNγ-producing CD4+ Th1 and CD8⁺ Tc1 cells. The extracellular region of TIM-3 consists of a membrane distal single variable immunoglobulin domain (IgV) and a glycosylated mucin domain of variable length located closer to the membrane. TIM-3 is an immune checkpoint and, together with other inhibitory receptors including PD-1 and LAG3, it mediates the T-cell exhaustion. TIM-3 has also been shown as a CD4+ Th1-specific cell surface protein that regulates macrophage activation. Inhibitors of the disclosure may block one or more functions of TIM-3 activity.

In some embodiments, the immune checkpoint inhibitor is an anti-TIM-3 antibody (e.g., a human antibody, a humanized antibody, or a chimeric antibody), an antigen binding fragment thereof, an immunoadhesin, a fusion protein, or oligopeptide.

Anti-human-TIM-3 antibodies (or V_{H} and/or V_{L} domains derived therefrom) suitable for use in the present methods can be generated using methods well known in the art. Alternatively, art recognized anti-TIM-3 antibodies can be used. For example, anti-TIM-3 antibodies including: MBG453, TSR-022 (also known as Cobolimab), and LY3321367 can be used in the methods disclosed herein. These and other anti-TIM-3 antibodies useful in the claimed invention can be found in, for example: U.S. Pat. Nos. 9,605,070, 8,841,418, US2015/0218274, and US 2016/0200815. The teachings of each of the aforementioned publications are hereby incorporated by reference. Antibodies that compete with any of these art-recognized antibodies for binding to LAG3 also can be used.

In some embodiments, the inhibitor comprises the heavy and light chain CDRs or VRs of an anti-TIM-3 antibody. Accordingly, in one embodiment, the inhibitor comprises the CDR1, CDR2, and CDR3 domains of the V_{H} region of an anti-TIM-3 antibody, and the CDR1, CDR2 and CDR3 domains of the V_{L} region of an anti-TIM-3 antibody. In another embodiment, the antibody has at least about 70, 75, 80, 85, 90, 95, 97, or 99% (or any derivable range therein) variable region amino acid sequence identity with the above-mentioned antibodies.

b. Activation of Co-Stimulatory Molecules

In some embodiments, the immunotherapy comprises an activator of a co-stimulatory molecule. In some embodiments, the activator comprises an agonist of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Activators include agonistic antibodies, polypeptides, compounds, and nucleic acids.

c. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as antibody targets.

d. CAR-T Cell and CAR-NK Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell or NK cell. The receptors are called chimeric because they are fused of parts from different sources. CAR-T cell therapy refers to a treatment that uses such transformed T cells for cancer therapy. CAR-NK cell therapy refers to a treatment that uses such transformed NK cells for cancer therapy.

e. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

### 2. Chemotherapies

In some embodiments, the cancer therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m2 to about 20 mg/m2 for 5 days every three weeks for a total of three courses being contemplated in certain embodiments. In some embodiments, the amount of cisplatin delivered to the cell and/or subject in conjunction with the construct comprising an Egr-1 promoter operably linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m2 to about 75 mg/m2 at about 21-day intervals or about 25 mg/m2 to about 30 mg/m2 on each of 2 or 3 successive days repeated at about 3 week to about 4 week intervals or about 20 mg/m2 once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN2), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

### 3. Radiotherapy

In some embodiments, the cancer therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, exactly, or between any two of 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, exactly, or between any two of 1, 2, 3, 4, 5, 6, 7, 8, 9, or 10 does (or any derivable range therein). When more than one dose is administered, the does may be at least, at most, exactly, or between any two of about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, exactly, or between any two of 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, exactly, or between any two of 1, 2, 3, 4, 5, 6, 7, 8, 9, 10, 12, 14, 15, 20, 25, 30, 35, 40, 45, or 50 Gy (or any derivable range therein. In some embodiments, at least, at most, exactly, or between any two of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40,41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, exactly, or between any two of 1, 2, 3, 4, 5, 6, 7, 8, 9, 10, 11, or 12 (or any derivable range therein) fractionated doses are administered per day. In some embodiments, at least, at most, exactly, or between any two of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

### 4. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, at least, at most, exactly, or between any two of every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

### B. Administration of Therapeutic Compositions

The therapeutic agents of the disclosure may be administered by the same route of administration or by different routes of administration. In some embodiments, the cancer therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of at least, at most, exactly, or between any two of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: at least, at most, exactly, or between any two of about 1,2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more administrations. The administrations can be at at least, at most, exactly, or between any two of 1, 2, 3, 4, 5, 6, 7, 8, to 5, 6, 7, 8, 9, 10, 11, or 12 week intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

Proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### 1. Combination Therapy

The therapy provided herein may comprise administration of a combination of therapeutic agents, such as a first cancer therapy and a second cancer therapy. The therapies may be administered in any suitable manner known in the art. For example, the first and second cancer treatment may be administered sequentially (at different times) or concurrently (at the same time). In some embodiments, the first and second cancer treatments are administered in a separate composition. In some embodiments, the first and second cancer treatments are in the same composition.

In some embodiments, the first cancer therapy and the second cancer therapy are administered substantially simultaneously. In some embodiments, the first cancer therapy and the second cancer therapy are administered sequentially. In some embodiments, the first cancer therapy, the second cancer therapy, and a third therapy are administered sequentially. In some embodiments, the first cancer therapy is administered before administering the second cancer therapy. In some embodiments, the first cancer therapy is administered after administering the second cancer therapy.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed.

### XVI. Formulations and Culture of the Cells

In particular embodiments, the cells of the disclosure may be specifically formulated and/or they may be cultured in a particular medium. The cells may be formulated in such a manner as to be suitable for delivery to a recipient without deleterious effects.

The medium in certain aspects can be prepared using a medium used for culturing animal cells as their basal medium, such as any of AIM V, X-VIVO-15, NeuroBasal, EGM2, TeSR, BME, BGJb, CMRL 1066, Glasgow MEM, Improved MEM Zinc Option, IMDM, Medium 199, Eagle MEM, αMEM, DMEM, Ham, RPMI-1640, and Fischer's media, as well as any combinations thereof, but the medium may not be particularly limited thereto as far as it can be used for culturing animal cells. Particularly, the medium may be xeno-free or chemically defined.

The medium can be a serum-containing or serum-free medium, or xeno-free medium. From the aspect of preventing contamination with heterogeneous animal-derived components, serum can be derived from the same animal as that of the stem cell(s). The serum-free medium refers to medium with no unprocessed or unpurified serum and accordingly, can include medium with purified blood-derived components or animal tissue-derived components (such as growth factors).

The medium may contain or may not contain any alternatives to serum. The alternatives to serum can include materials which appropriately contain albumin (such as lipid-rich albumin, bovine albumin, albumin substitutes such as recombinant albumin or a humanized albumin, plant starch, dextrans and protein hydrolysates), transferrin (or other iron transporters), fatty acids, insulin, collagen precursors, trace elements, 2-mercaptoethanol, 3′-thiolgiycerol, or equivalents thereto. The alternatives to serum can be prepared by the method disclosed in International Publication No. 98/30679, for example (incorporated herein in its entirety). Alternatively, any commercially available materials can be used for more convenience. The commercially available materials include knockout Serum Replacement (KSR), Chemically-defined Lipid concentrated (GIBCO™), and GLUTAMAX™ (GIBCO™)

In certain embodiments, the medium may comprise at least, at most, exactly, or between any two of one, two, three, four, five, six, seven, eight, nine, ten, 11, 12, 13, 14, 15, 16, 17, 18, 19, 20 or more of the following: Vitamins such as biotin; DL Alpha Tocopherol Acetate; DL Alpha-Tocopherol; Vitamin A (acetate); proteins such as BSA (bovine serum albumin) or human albumin, fatty acid free Fraction V; Catalase; Human Recombinant Insulin; Human Transferrin; Superoxide Dismutase; Other Components such as Corticosterone; D-Galactose; Ethanolamine HCl; Glutathione (reduced); L-Carnitine HCl; Linoleic Acid; Linolenic Acid; Progesterone; Putrescine 2HCl; Sodium Selenite; and/or T3 (triodo-I-thyronine). In specific embodiments, one or more of these may be explicitly excluded.

In some embodiments, the medium further comprises vitamins. In some embodiments, the medium comprises at least, at most, exactly, or between any two of 1, 2, 3, 4, 5, 6, 7, 8, 9, 10, 11, 12, or 13 of the following (and any range derivable therein): biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, vitamin B12, or the medium includes combinations thereof or salts thereof. In some embodiments, the medium comprises or consists essentially of biotin, DL alpha tocopherol acetate, DL alpha-tocopherol, vitamin A, choline chloride, calcium pantothenate, pantothenic acid, folic acid nicotinamide, pyridoxine, riboflavin, thiamine, inositol, and vitamin B12. In some embodiments, the vitamins include or consist essentially of biotin, DL alpha tocopherol acetate, DL alpha-tocopherol, vitamin A, or combinations or salts thereof. In some embodiments, the medium further comprises proteins. In some embodiments, the proteins comprise albumin or bovine serum albumin, a fraction of BSA, catalase, insulin, transferrin, superoxide dismutase, or combinations thereof. In some embodiments, the medium further comprises one or more of the following: corticosterone, D-Galactose, ethanolamine, glutathione, L-carnitine, linoleic acid, linolenic acid, progesterone, putrescine, sodium selenite, or triodo-I-thyronine, or combinations thereof. In some embodiments, the medium comprises one or more of the following: a B-27® supplement, xeno-free B-27® supplement, GS21TM supplement, or combinations thereof. In some embodiments, the medium comprises or further comprises amino acids, monosaccharides, inorganic ions. In some embodiments, the amino acids comprise arginine, cystine, isoleucine, leucine, lysine, methionine, glutamine, phenylalanine, threonine, tryptophan, histidine, tyrosine, or valine, or combinations thereof. In some embodiments, the inorganic ions comprise sodium, potassium, calcium, magnesium, nitrogen, or phosphorus, or combinations or salts thereof. In some embodiments, the medium further comprises one or more of the following: molybdenum, vanadium, iron, zinc, selenium, copper, or manganese, or combinations thereof. In certain embodiments, the medium comprises or consists essentially of one or more vitamins discussed herein and/or one or more proteins discussed herein, and/or one or more of the following: corticosterone, D-Galactose, ethanolamine, glutathione, L-carnitine, linoleic acid, linolenic acid, progesterone, putrescine, sodium selenite, or triodo-I-thyronine, a B-27® supplement, xeno-free B-27® supplement, GS21TM supplement, an amino acid (such as arginine, cystine, isoleucine, leucine, lysine, methionine, glutamine, phenylalanine, threonine, tryptophan, histidine, tyrosine, or valine), monosaccharide, inorganic ion (such as sodium, potassium, calcium, magnesium, nitrogen, and/or phosphorus) or salts thereof, and/or molybdenum, vanadium, iron, zinc, selenium, copper, or manganese. In specific embodiments, one or more of these may be explicitly excluded.

The medium can also contain one or more externally added fatty acids or lipids, amino acids (such as non-essential amino acids), vitamin(s), growth factors, cytokines, antioxidant substances, 2-mercaptoethanol, pyruvic acid, buffering agents, and/or inorganic salts.. In specific embodiments, one or more of these may be explicitly excluded.

One or more of the medium components may be added at a concentration of at least, at most, exactly, or between any two of 0.1, 0.5, 1, 2, 3, 4, 5, 10, 15, 20, 25, 30, 35, 40, 45, 50, 55, 60, 65, 70, 75, 80, 85, 90, 95, 100, 150, 180, 200, 250 ng/L, ng/ml, μg/ml, mg/ml, or any range derivable therein.

In specific embodiments, the cells of the disclosure are specifically formulated. They may or may not be formulated as a cell suspension. In specific cases they are formulated in a single dose form. They may be formulated for systemic or local administration. In some cases the cells are formulated for storage prior to use, and the cell formulation may comprise one or more cryopreservation agents, such as DMSO (for example, in 5% DMSO). The cell formulation may comprise albumin, including human albumin, with a specific formulation comprising 2.5% human albumin. The cells may be formulated specifically for intravenous administration; for example, they are formulated for intravenous administration over less than one hour. In particular embodiments the cells are in a formulated cell suspension that is stable at room temperature for 1, 2, 3, or 4 hours or more from time of thawing.

In particular embodiments, the cells of the disclosure comprise an exogenous TCR, which may be of a defined antigen specificity. In some embodiments, the TCR can be selected based on absent or reduced alloreactivity to the intended recipient. In the example where the exogenous TCR is non-alloreactive, during T cell differentiation the exogenous TCR suppresses rearrangement and/or expression of endogenous TCR loci through a developmental process called allelic exclusion, resulting in T cells that express only the non-alloreactive exogenous TCR and are thus non-alloreactive. In some embodiments, the choice of exogenous TCR may not necessarily be defined based on lack of alloreactivity. In some embodiments, the endogenous TCR genes have been modified by genome editing so that they do not express a protein. Methods of gene editing such as methods using the CRISPR/Cas9 system are known in the art and described herein.

In some embodiments, the cells of the disclosure further comprise one or more chimeric antigen receptors (CARs). Examples of tumor cell antigens to which a CAR may be directed include at least 5T4, 8H9, αvβ6 integrin, BCMA, B7-H3, B7-H6, CAIX, CA9, CD19, CD20, CD22, CD30, CD33, CD38, CD44, CD44v6, CD44v7/8, CD70, CD123, CD138, CD171, CEA, CSPG4, EGFR, EGFR family including ErbB2 (HER2), EGFRvIII, EGP2, EGP40, ERBB3, ERBB4, ErbB3/4, EPCAM, EphA2, EpCAM, folate receptor-a, FAP, FBP, fetal AchR, FRQ, GD2, G250/CAIX, GD3, Glypican-3 (GPC3), Her2, IL-13R□2, Lambda, Lewis-Y, Kappa, KDR, MAGE, MCSP, Mesothelin, Mucd, Mucl6, NCAM, NKG2D Ligands, NY-ESO-1, PRAME, PSC1, PSCA, PSMA, ROR1, SP17, Survivin, TAG72, TEMs, carcinoembryonic antigen, HMW-MAA, AFP, CA-125, ETA, Tyrosinase, MAGE, laminin receptor, HPV E6, E7, BING-4, Calcium-activated chloride channel 2, Cyclin-B1, 9D7, EphA3, Telomerase, SAP-1, BAGE family, CAGE family, GAGE family, MAGE family, SAGE family, XAGE family, NY-ESO-1/LAGE-1, PAME, SSX-2, Melan-A/MART-1, GP100/pmel17, TRβ-1/-2, P. polypeptide, MC1R, Prostate-specific antigen, β-catenin, BRCA1/2, CML66, Fibronectin, MART-2, TGF-βRII, or VEGF receptors (e.g., VEGFR2), for example. The CAR may be a first, second, third, or more generation CAR. The CAR may be bispecific for any two nonidentical antigens, or it may be specific for more than two nonidentical antigens.

### A. Cells

Certain embodiments relate to cells comprising polypeptides or nucleic acids of the disclosure. In some embodiments the cell is an immune cell. In some embodiments, the cell is a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell (e.g., peripheral blood stem cell) or progenitor cell obtained from an individual.

### XVIII. General Pharmaceutical Compositions

In some embodiments, pharmaceutical compositions are administered to a subject. Different aspects may involve administering an effective amount of a composition to a subject. In some embodiments, an antibody or antigen binding fragment capable of binding to CD70 may be administered to the subject to protect against or treat a condition (e.g., cancer). Alternatively, an expression vector encoding one or more such antibodies or polypeptides or peptides may be given to a subject as a preventative treatment. Additionally, such compositions can be administered in combination with an additional therapeutic agent (e.g., a chemotherapeutic, an immunotherapeutic, a biotherapeutic, etc.). Such compositions will generally be dissolved or dispersed in a pharmaceutically acceptable carrier or aqueous medium.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

The active compounds can be formulated for parenteral administration, e.g., formulated for injection via the intravenous, intramuscular, subcutaneous, or intraperitoneal routes. Typically, such compositions can be prepared as either liquid solutions or suspensions; solid forms suitable for use to prepare solutions or suspensions upon the addition of a liquid prior to injection can also be prepared; and, the preparations can also be emulsified.

The pharmaceutical forms suitable for injectable use include sterile aqueous solutions or dispersions; formulations including, for example, aqueous propylene glycol; and sterile powders for the extemporaneous preparation of sterile injectable solutions or dispersions. In all cases the form must be sterile and must be fluid to the extent that it may be easily injected. It also should be stable under the conditions of manufacture and storage and must be preserved against the contaminating action of microorganisms, such as bacteria and fungi.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of microorganisms can be brought about by various anti-bacterial and anti-fungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various other ingredients enumerated above, as required, followed by filtered sterilization or an equivalent procedure. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques, which yield a powder of the active ingredient, plus any additional desired ingredient from a previously sterile-filtered solution thereof.

Administration of the compositions will typically be via any common route. This includes, but is not limited to oral, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

Upon formulation, solutions will be administered in a manner compatible with the dosage formulation and in such amount as is therapeutically or prophylactically effective. The formulations are easily administered in a variety of dosage forms, such as the type of injectable solutions described above.

### XIX. Kits

Any of the compositions described herein may be comprised in a kit. In a non-limiting example, cells, reagents to produce cells, vectors, and reagents to produce vectors and/or components thereof may be comprised in a kit. In certain embodiments, NK cells may be comprised in a kit, and they may or may not yet express a CD70-targeting receptor, an optional cytokine, or an optional suicide gene. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more a CD70 antibodies, CD70 CARs, and/or CD70 engagers, suicide gene products, and/or cytokines may be included in the kit. Proteins, such as cytokines or antibodies, including monoclonal antibodies, may be included in the kit. Nucleotides that encode components of engineered CD70 antibodies, CD70 CARs, and/or CD70 engagers may be included in the kit, including reagents to generate same.

In particular aspects, the kit comprises an NK cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Monoclonal Anti-Human CD70 Antibodies Bind Human CD70

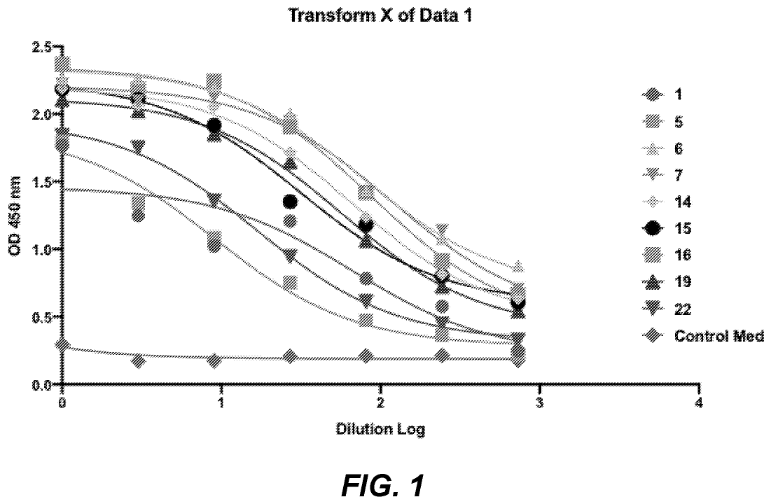

Monoclonal anti-human CD70 antibodies including ml, m5, m6, m7, m14, m15, m16, m19, and m22 clones (m6, m7, and m14 clones described in Table 3 below) were titrated in an ELISA cell-based assay (FIG. 1). A 96 well plate coated with murine fibroblast (L cells) transduced to express the full length human CD70 molecule was incubated with serial dilutions of the purified antibodies and control media. The ELISA was developed with a goat-antimouse IgG-HRP conjugated secondary antibody, and absorbance was measured at 450 nM to determine the half-maximal effective concentration (EC₅₀).

TABLE 3

| | | Nucleotide | Amino Acid |
|---|---|---|---|
| Construct name | Format | SEQ ID NO: | SEQ ID NO: |
| m6 | m6 V_{H} | 1 | 44 |
| | m6 V_{L} | 5 | 48 |
| | m6 V_{H}-linker-V_{L} | 25 | 68 |
| | m6 V_{L}-linker-V_{H} | 26 | 69 |
| m7 | m7 V_{H} | 9 | 52 |
| | m7 V_{L} | 13 | 56 |
| | m7 V_{H}-linker-V_{L} | 27 | 70 |
| | m7 V_{L}-linker-V_{H} | 28 | 71 |
| m14 | m14 V_{H} | 17 | 60 |
| | m14 V_{L} | 21 | 64 |
| | m14 V_{H}-linker-V_{L} | 29 | 72 |
| | m14 V_{L}-linker-V_{H} | 30 | 73 |

### Example 2—Anti-Human CD70 Antibodies Show Stable Expression in NK Cells

Flow cytometry assay was conducted to detect the binding of various ml, m5, m6, m7, m14, m15, m16, m19, and m22 clones CD70 antibody clones to CD70 antigen expressed in various cells.

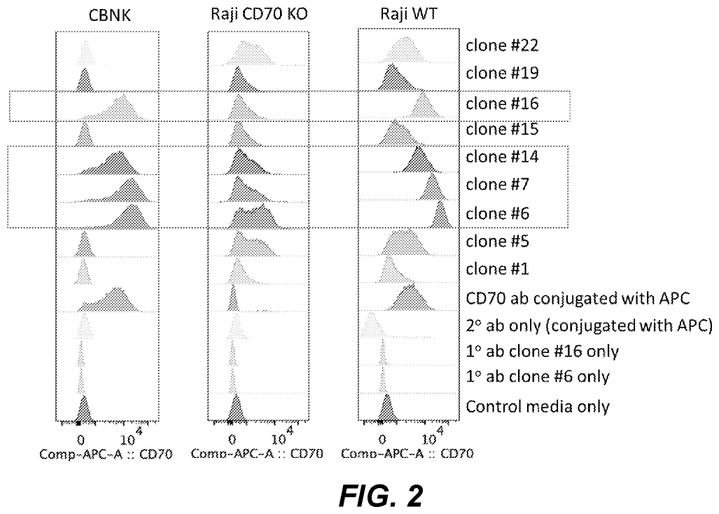

Cord blood derived natural killer (CBNK) cells and Raji wildtype (WT) cells are positive for CD70 antigen expression and Raji CD70 knockout (KO) cells are negative for CD7 expression. Clones m6, m7, m14, and c16 showed enhanced binding to CD7 d antigens in cell surface, and clones m7, m14, and m16 showed specific binding (FIG. 2).

### Example 3—Anti-Human CD7 Antibodies Show Stable Expression in NK Cells

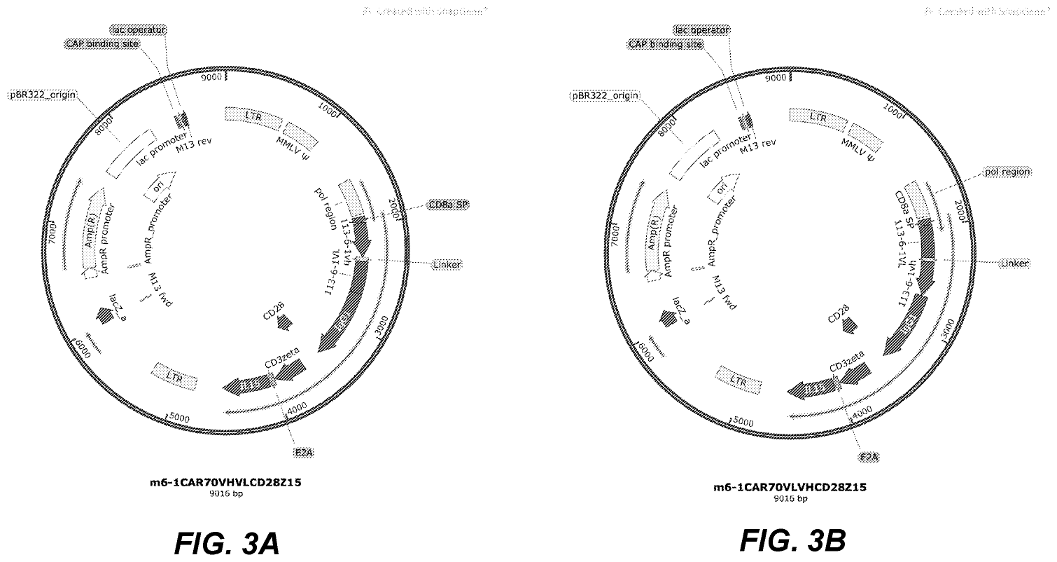

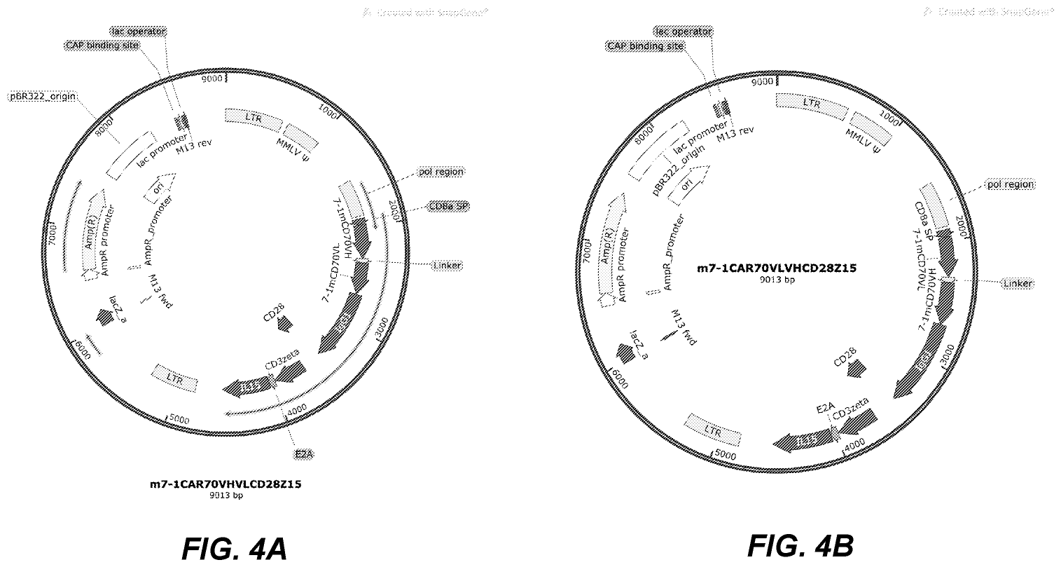

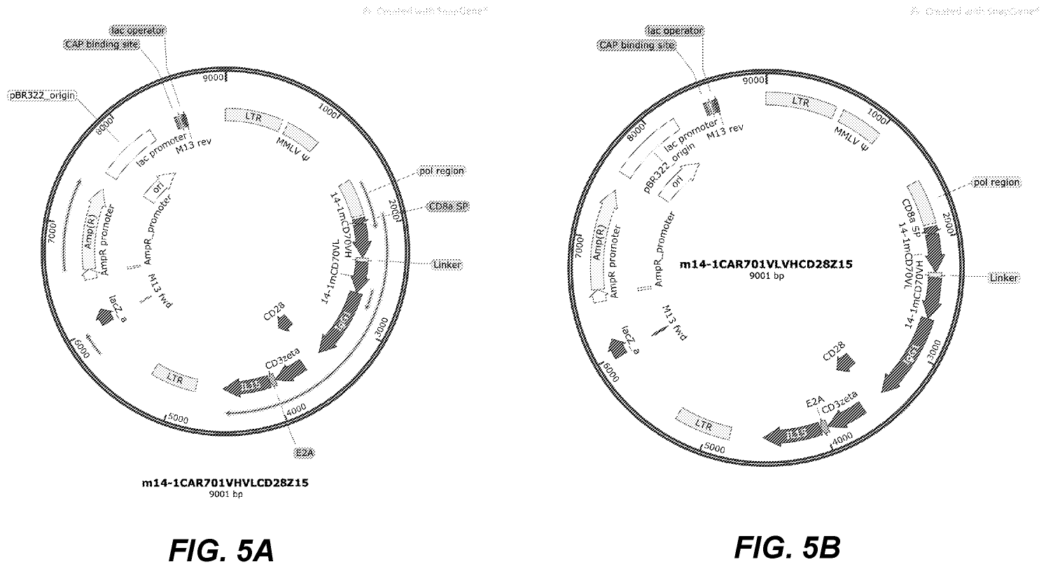

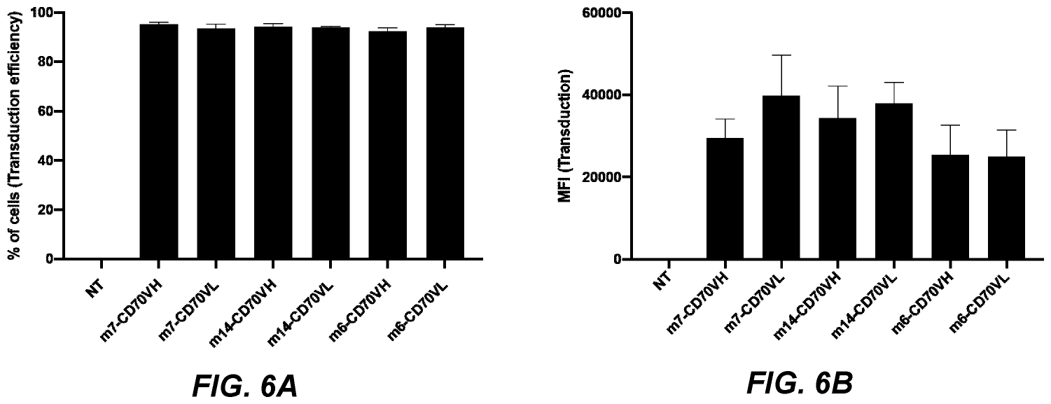

Cord-blood derived NK cells were transduced with various CD7 CAR constructs (illustrated schematically in FIGS. 3-5 and described below in Table 4) and transfection efficiency was measured by flow cytometry. The transduction efficiency based on percent positive cells (FIG. 6A) and mean fluorescent efficiency (MFI) (FIG. 6B) are shown.

TABLE 4

| | | Nucleotide | Amino Acid |
|---|---|---|---|
| Construct name | Format | SEQ ID NO: | SEQ ID NO: |
| m6-1CD70V_{H}VLCD28Z15 | V_{H}-V_{L} with IgG1 | 32 | 94 |
| | hinge - CD28Z.IL15 | | |
| m6-1CD70V_{L}VHCD28Z15 | V_{L}-V_{H} with IgG1 | 33 | 95 |
| | hinge - CD28Z.IL15 | | |
| m7-1CD70V_{H}VLCD28Z15 | V_{H}-V_{L} with IgG1 | 34 | 96 |
| | hinge - CD28Z.IL15 | | |
| m7-1CD70V_{L}VHCD28Z15 | V_{L}-V_{H} with IgG1 | 35 | 97 |
| | hinge - CD28Z.IL15 | | |
| m14- | V_{H}-V_{L} with IgG1 | 36 | 98 |
| 1CD701VHVLCD28Z15 | hinge - CD28Z.IL15 | | |
| m14- | V_{L}-V_{H} with IgG1 | 37 | 99 |
| 1CD701VLVHCD28Z15 | hinge - CD28Z.IL15 | | |
| m6-1CD70V_{H}VLCD28Z15 | V_{H}-V_{L} with CD28 | 38 | 100 |
| | hinge - CD28Z.IL15 | | |
| m6-1CD70V_{L}VHCD28Z15 | V_{L}-V_{H} with CD28 | 39 | 101 |
| | hinge - CD28Z.IL15 | | |
| m7-1CD70V_{H}VLCD28Z15 | V_{H}-V_{L} with CD28 | 40 | 102 |
| | hinge - CD28Z.IL15 | | |
| m7-1CD70V_{L}VHCD28Z15 | V_{L}-V_{H} with CD28 | 41 | 103 |
| | hinge - CD28Z.IL15 | | |
| m14- | V_{H}-V_{L} with CD28 | 42 | 104 |
| 1CD701VHVLCD28Z15 | hinge - CD28Z.IL15 | | |
| m14- | V_{L}-V_{H} with CD28 | 43 | 105 |
| 1CD701VLVHCD28Z15 | hinge - CD28Z.IL15 | | |

### Example 4—Functional Performance of huCD70CAR NK Cells Against Raji Lymphoma Cells

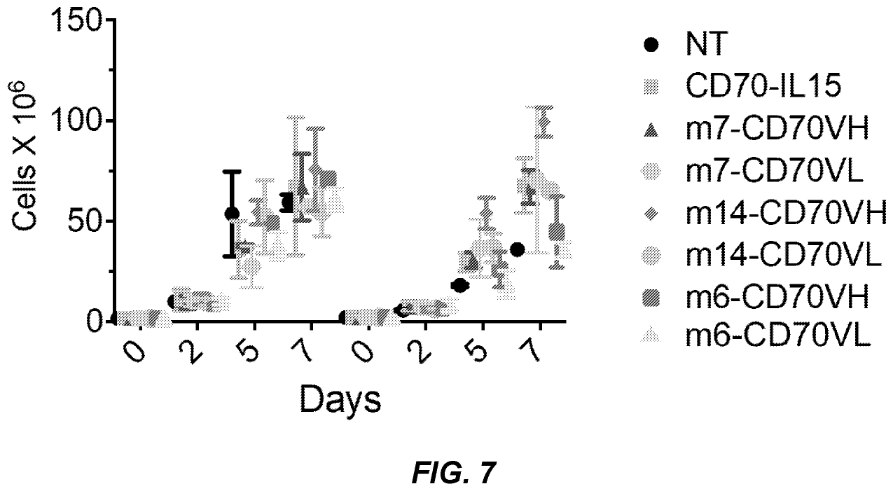

Results from cell proliferation assays of CBNK(cells transduced with various CD70 CAR constructs shown above in Table 4 are shown in FIG. 7. Two million NIK cells were expanded with irradiated uAPC cells in complete Serum-free Stem Cell Growth Medium (SCGM) supplemented with IL,-2. Two cycles of expanded data are shown.

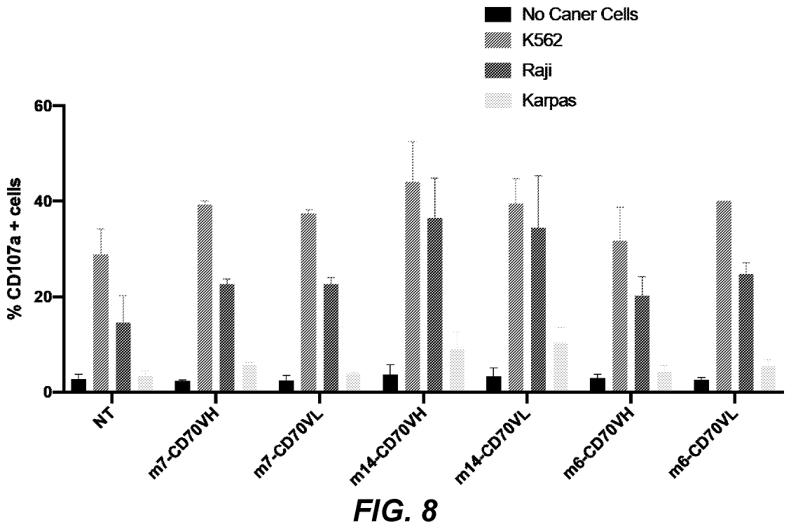

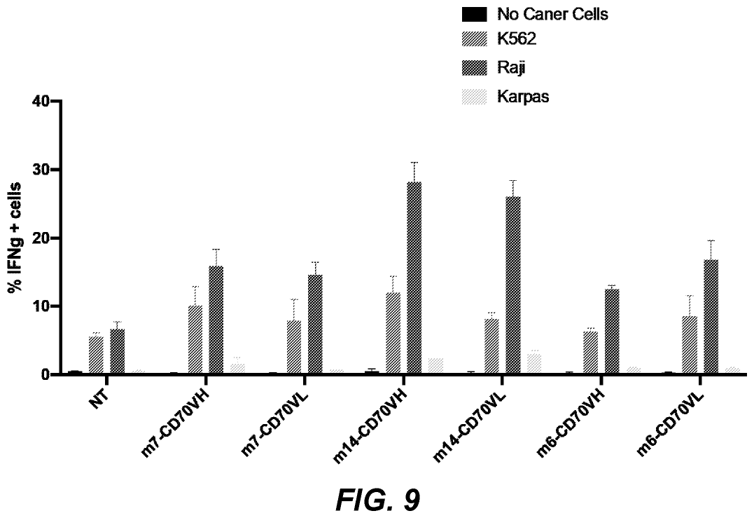

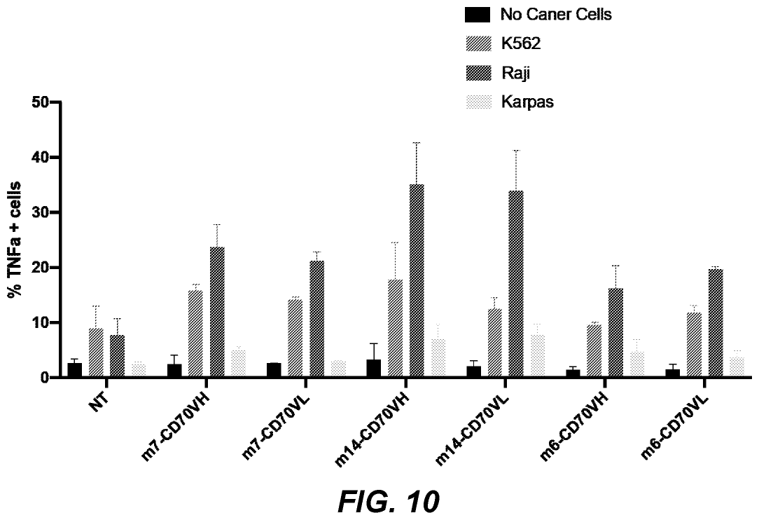

CD107a, Interferon gamma, and tumor necrosis factor alpha production by CBNK cells transduced with various CD70 constructs shown above in Table 4 when co-cultured with various cancer cells was also measured. As shown in FIG. 8, compared to non-transduced (NT) cells, CBNK cells transduced with CD70 CAR showed increased expression of degranulation marker CD107a when co-cultured with Raji and Karpas cells, which have high CD70 expression on their cell surface, suggesting enhanced cytotoxicity against these cells. K562 cells were used as positive control, as these cells are sensitive to CBNK cell killing, and no cancer cells were add to get the baseline expression of CD107a. As shown in FIG. 9, compared to non-transduced (NT) cells, CBNK cells transduced with CD70 CAR showed increased production of interferon gamma (IFNg) when co-cultured with Raji and Karpas cells, which have high CD70 expression on their cell surface. K562 cells were used as positive control, as these cells are sensitive to CBNK cell killing, and no cancer cells were add to get the baseline secretion of IFNg. As shown in FIG. 10, compared to non-transduced (NT) cells, CBNK cells transduced with CD70 CAR showed increased production of tumor necrosis factor alpha (TNFa) when co-cultured with Raji and Karpas cells, which have high CD70 expression on their cell surface. K562 cells are used as positive control, as these cells are sensitive to CBNK cell killing, and no cancer cells were add to get the baseline secretion of TNFa.

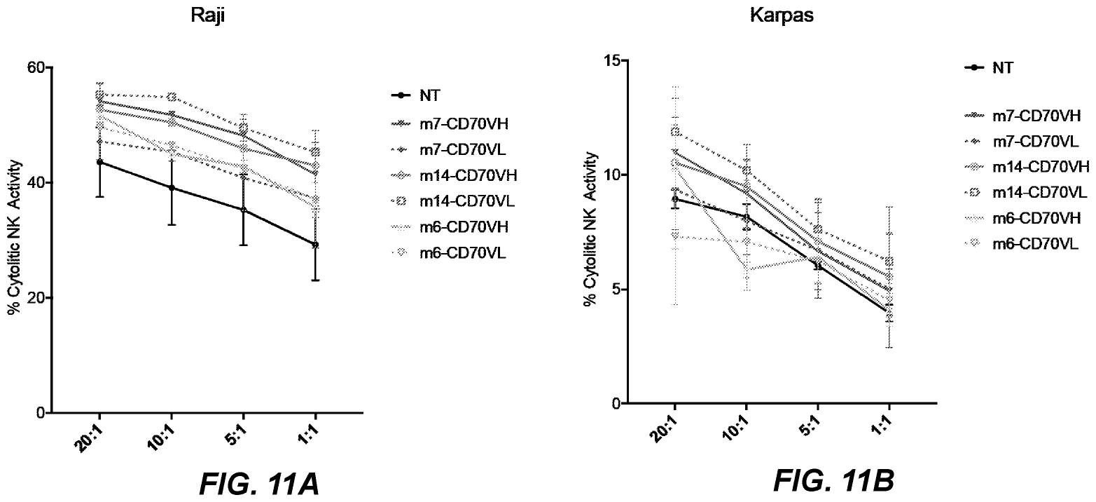

Cytotoxic function CBNK cells transduced with the various CD70 constructs shown above in Table 4 were evaluated against Raji and Karpas cells using chromium release assays. Compared to non-transduced (NT) cells, CBNK cells transduced with various CD70 CARs showed increased cytotoxicity of Raji (FIG. 11A) and Karpas (FIG. 11B) cells (which have high CD70 expression on their cell surface), as shown by chromium release assay, suggesting that CBNK CD70 CAR cells have greater killing activity against cancer cells with high CD70 expression. Various effector to target ratio were used in this assay, as shown in X-axis of both graphs.

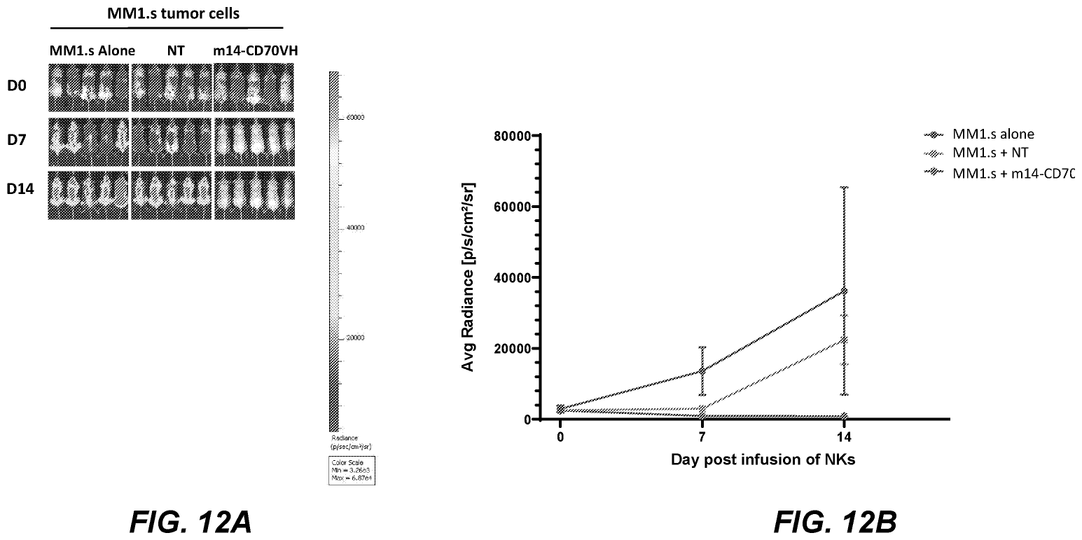

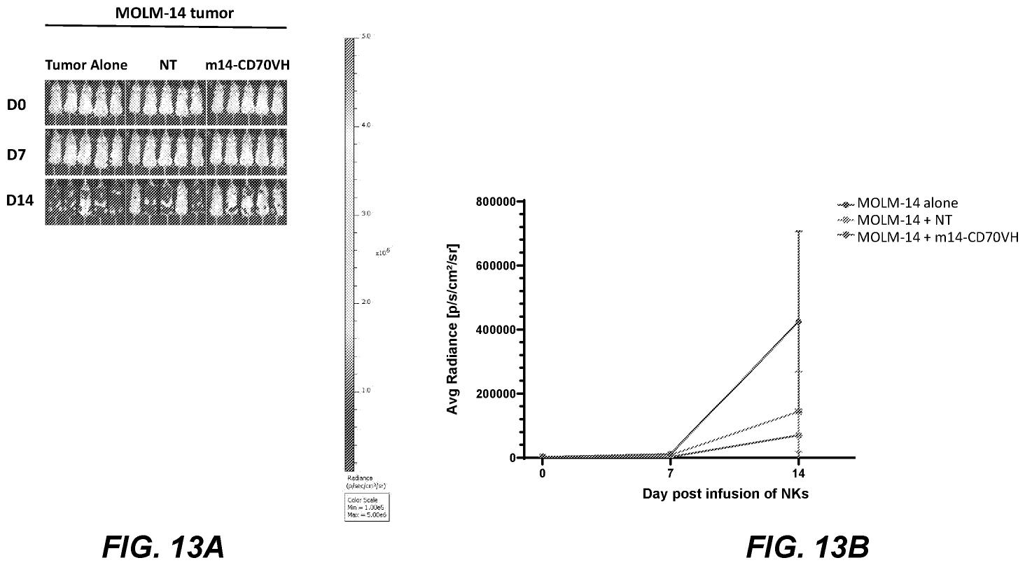

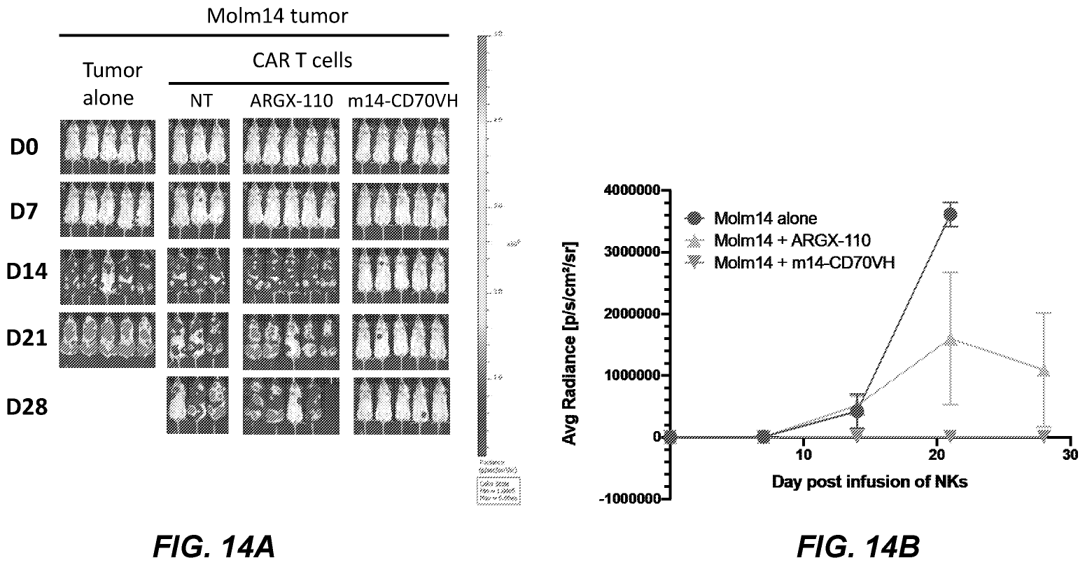

CD70 CAR CBNK cells transduced with the various CD70 constructs shown above in Table 4 were also shown to reduce the tumor burden in a mouse model of multiple myeloma (MM1.s; FIG. 12) and acute myeloid leukemia (MOLM-14; FIG. 13). MM1.s cells transduced with firefly luciferase (FFLuc) were injected into mice and were monitored with bioluminescence imaging among the various groups. Mice in treatment group were injected with 1M of respective CBNK cells, one day post tumor injection. Bioluminescence images (FIG. 12A) of mice in each groups, and quantification (FIG. 12B) of luciferase signal shows that CD70 CAR CBNK cells generated from m14-CD70 clone was better at reducing MM1.s tumor burden when compared to non transduced (NT) CBNK cells. MOLM-14 cells transduced with firefly luciferase (FFLuc) were injected into mice and were monitored with bioluminescence imaging among the various groups. Mice in treatment group were injected with 1M of respective CBNK cells, one day post tumor injection. Bioluminescence images (FIG. 13A) of mice in each groups, and quantification (FIG. 13B) of luciferase signal shows that CD70 CAR CBNK cells generated from m14-CD70 clone was better at reducing Molm14 tumor burden when compared to non transduced (NT) CBNK cells and when compared to CD70 CAR T cells generated from ARGX-110 (FIGS. 14A-14B).

### Example 5—Functional Performance of huCD70CAR T Cells Against Raji and Mec-1 Cells

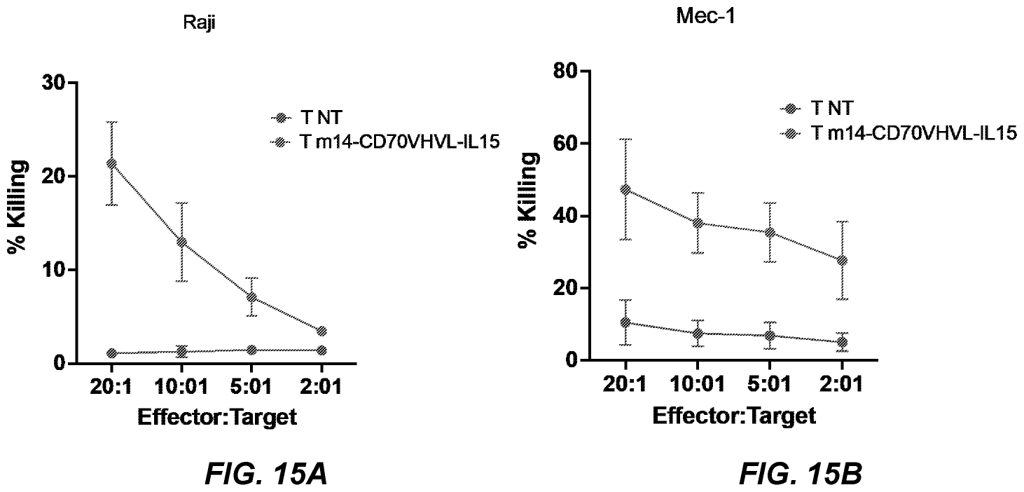

Cytotoxic function T cells transduced with the m14-CD70VHVL-IL15 construct corresponding to SEQ ID NOs:42 and 104 shown above in Table 4 was evaluated against Raji and Mec-1 cells using chromium release assays. Compared to non-transduced (NT) cells, T cells transduced with the m14-CD70VHVL-IL15 construct showed increased cytotoxicity of Raji (FIG. 15A) and Mec-1 (FIG. 15B) cells (which have high CD70 expression on their cell surface), as shown by chromium release assay, suggesting that T-cells transduced with the CD70 CAR have greater killing activity against cancer cells with high CD70 expression compared to NT T cells. Various effector to target ratio were used in this assay, as shown in X-axis of both graphs.

Cytotoxic function T cells transduced with the m14-CD70VHVL-IL15 construct corresponding to SEQ ID NOs:42 and 104 shown above in Table 4 was evaluated against the SKOV3 CD70-expressing ovarian cancer cell line using the Xcelligence assay (Agilent). Compared to non-transduced (NT) cells, T cells transduced with the m14-CD70VHVL-IL15 construct showed increased cytotoxicity of SKOV3 cells. SKOV3 cells, which have high CD70 expression, were grown in 96 well RTCA E-Plates overnight, and m14-CD70VHVL-IL15 CAR T cells were added the next day at 2:1 effector to target (E:T) ratio. Cytotoxicity was measured continuously by the Xcelligence machine and represented as normalized cell index. Experiments were performed with CAR T cells generated from two different donors.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references and those cited elsewhere herein, to the extent that they provide exemplary procedural or other details supplementary to those set forth herein, are specifically incorporated herein by reference.
1. Borst, J., et al. (2005). “CD27 and CD70 in T cell and B cell activation.” Curr Opin Immunol 17(3): 275-281.
2. Jacobs, J., et al. (2015). “CD70: An emerging target in cancer immunotherapy.” Pharmacol Ther 155: 1-10.
3. Oflazoglu, E., et al. (2008). “Potent anticarcinoma activity of the humanized anti-CD70 antibody h1F6 conjugated to the tubulin inhibitor auristatin via an uncleavable linker.” Clin Cancer Res 14(19): 6171-6180.
4. Owonikoko, T. K., et al. (2016). “First-in-human multicenter phase I study of BMS-936561 (MDX-1203), an antibody-drug conjugate targeting CD70.” Cancer Chemother Pharmacol 77(1): 155-162.
5. Riether, C., et al. (2017). “CD70/CD27 signaling promotes blast stemness and is a viable therapeutic target in acute myeloid leukemia.” J Exp Med 214(2): 359-380.
6. Ryan, M. C., et al. (2010). “Targeting pancreatic and ovarian carcinomas using the auristatin-based anti-CD70 antibody-drug conjugate SGN-75.” Br J Cancer 103(5): 676-684.
7. Shaffer, D. R., et al. (2011). “T cells redirected against CD70 for the immunotherapy of CD70-positive malignancies.” Blood 117(16): 4304-4314.
8. Wajant, H. (2016). “Therapeutic targeting of CD70 and CD27.” Expert Opin Ther Targets 20(8): 959-973.
9. Goodwin R G, Alderson M R, Smith C A, Armitage R J, VandenBos T, Jerzy R, Tough T W, Schoenborn M A, Davis-Smith T, Hennen K (1993) Molecular and biological characterization of a ligand for CD27 defines a new family of cytokines with homology to tumor necrosis factor. Cell 73: 447-456
10. van Gisbergen K P, van Olffen R W, van Beek J, et al. Protective CD8 T cell memory is impaired during chronic CD70-driven costimulation. J Immunol 2009; 182(9): 5352-62.
11. O'Neill R E, Du W, Mohammadpour H, et al. T Cell-Derived CD70 Delivers an Immune Checkpoint Function in Inflammatory T Cell Responses. J Immunol 2017; 199(10): 3700-10.
12. Claus C, Riether C, Schurch C, Matter M S, Hilmenyuk T, Ochsenbein A F. CD27 signaling increases the frequency of regulatory T cells and promotes tumor growth. Cancer Res 2012; 72(14): 3664-76.
13. Grewal I S. CD70 as a therapeutic target in human malignancies. Expert Opin Ther Targets 2008; 12(3): 341-51.
14. Maude S L, Laetsch T W, Buechner J, et al. Tisagenlecleucel in Children and Young Adults with B-Cell Lymphoblastic Leukemia. N Engl J Med 2018; 378(5): 439-48.
15. Neelapu S S, Locke F L, Bartlett N L, et al. Axicabtagene Ciloleucel CAR T-Cell Therapy in Refractory Large B-Cell Lymphoma. N Engl J Med 2017; 377(26): 2531-44.
16. Schuster S J, Bishop M R, Tam C S, et al. Tisagenlecleucel in Adult Relapsed or Refractory Diffuse Large B-Cell Lymphoma. N Engl J Med 2019; 380(1): 45-56.
17. Daher M, Rezvani K. Next generation natural killer cells for cancer immunotherapy: the promise of genetic engineering. Curr Opin Immunol 2018; 51: 146-53.
18. Liu E, Tong Y, Dotti G, et al. Cord blood NK cells engineered to express IL-15 and a CD19-targeted CAR show long-term persistence and potent antitumor activity. Leukemia 2018; 32(2): 520-31.
19. Liu E, Marin D, Banerjee P, et al. Use of CAR-Transduced Natural Killer Cells in CD19-Positive Lymphoid Tumors. N Engl J Med 2020; 382(6): 545-53.
20. Silence K, Dreier T, Moshir M, et al. ARGX-110, a highly potent antibody targeting CD70, eliminates tumors via both enhanced ADCC and immune checkpoint blockade. MAbs 2014; 6(2): 523-32.

What is claimed is:

1. An anti-CD70 antibody comprising:
(a) a heavy chain variable region (V_{H}) comprising:
(i) a CDR-H1 comprising an amino acid sequence having at least 80% sequence identity to SEQ ID NO:45;
(ii) a CDR-H2 comprising an amino acid sequence having at least 80% sequence identity to SEQ ID NO:46; and
(iii) a CDR-H3 comprising an amino acid sequence having at least 80% sequence identity to SEQ ID NO:47; and
(b) a light chain variable region (V_{L}) comprising:
(i) a CDR-L1 comprising an amino acid sequence having at least 80% sequence identity to SEQ ID NO:49;
(ii) a CDR-L2 comprising an amino acid sequence having at least 80% sequence identity to SEQ ID NO:50; and
(iii) a CDR-L3 comprising an amino acid sequence having at least 80% sequence identity to SEQ ID NO:51.

2. The anti-CD70 antibody of claim 1, wherein the CDR-H1 comprises SEQ ID NO:45.

3. The anti-CD70 antibody of claim 1 or 2, wherein the CDR-H2 comprises SEQ ID NO:46.

4. The anti-CD70 antibody of any of claims 1-3, wherein the CDR-H3 comprises SEQ ID NO:47.

5. The anti-CD70 antibody of any of claims 1-4, wherein the CDR-L1 comprises SEQ ID NO:49.

6. The anti-CD70 antibody of any of claims 1-5, wherein the CDR-L2 comprises SEQ ID NO:50.

7. The anti-CD70 antibody of any of claims 1-6, wherein the CDR-L3 comprises SEQ ID NO:51.

8. The anti-CD70 antibody of any of claims 1-7, wherein the V_{H} comprises an amino acid sequence having at least 85% identity to SEQ ID NO:44.

9. The anti-CD70 antibody of any of claims 1-8, wherein the V_{H} comprises an amino acid sequence having at least 90% identity to SEQ ID NO:44.

10. The anti-CD70 antibody of any of claims 1-9, wherein the V_{H} comprises an amino acid sequence having at least 95% identity to SEQ ID NO:44.

11. The anti-CD70 antibody of any of claims 1-10, wherein the V_{H} comprises SEQ ID NO:44.

12. The anti-CD70 antibody of any of claims 1-11, wherein the V_{L} comprises an amino acid sequence having at least 85% identity to SEQ ID NO:48.

13. The anti-CD70 antibody of any of claims 1-12, wherein the V_{L} comprises an amino acid sequence having at least 90% identity to SEQ ID NO:48.

14. The anti-CD70 antibody of any of claims 1-13, wherein the V_{L} comprises an amino acid sequence having at least 95% identity to SEQ ID NO:48.

15. The anti-CD70 antibody of any of claims 1-14, wherein the V_{L} comprises SEQ ID NO:48.

16. An anti-CD70 antibody comprising:
(a) a V_{H} comprising:
(i) a CDR-H1 comprising an amino acid sequence having at least 80% sequence identity to SEQ ID NO:53;
(ii) a CDR-H2 comprising an amino acid sequence having at least 80% sequence identity to SEQ ID NO:54; and
(iii) a CDR-H3 comprising an amino acid sequence having at least 80% sequence identity to SEQ ID NO:55; and
(b) a V_{L} comprising:
(i) a CDR-L1 comprising an amino acid sequence having at least 80% sequence identity to SEQ ID NO:57;
(ii) a CDR-L2 comprising an amino acid sequence having at least 80% sequence identity to SEQ ID NO:58; and
(iii) a CDR-L3 comprising an amino acid sequence having at least 80% sequence identity to SEQ ID NO:59.

17. The anti-CD70 antibody of claim 16, wherein the CDR-H1 comprises SEQ ID NO:53.

18. The anti-CD70 antibody of claim 16 or 17, wherein the CDR-H2 comprises SEQ ID NO:54.

19. The anti-CD70 antibody of any of claims 16-18, wherein the CDR-H3 comprises SEQ ID NO:55.

20. The anti-CD70 antibody of any of claims 16-19, wherein the CDR-L1 comprises SEQ ID NO:57.

21. The anti-CD70 antibody of any of claims 16-20, wherein the CDR-L2 comprises SEQ ID NO:58.

22. The anti-CD70 antibody of any of claims 16-21, wherein the CDR-L3 comprises SEQ ID NO:59.

23. The anti-CD70 antibody of any of claims 16-22, wherein the V_{H} comprises an amino acid sequence having at least 85% identity to SEQ ID NO:52.

24. The anti-CD70 antibody of any of claims 16-23, wherein the V_{H} comprises an amino acid sequence having at least 90% identity to SEQ ID NO:52.

25. The anti-CD70 antibody of any of claims 16-24, wherein the V_{H} comprises an amino acid sequence having at least 95% identity to SEQ ID NO:52.

26. The anti-CD70 antibody of any of claims 16-25, wherein the V_{H} comprises SEQ ID NO:52.

27. The anti-CD70 antibody of any of claims 16-26, wherein the V_{L} comprises an amino acid sequence having at least 85% identity to SEQ ID NO:56.

28. The anti-CD70 antibody of any of claims 16-27, wherein the V_{L} comprises an amino acid sequence having at least 90% identity to SEQ ID NO:56.

29. The anti-CD70 antibody of any of claims 16-28, wherein the V_{L} comprises an amino acid sequence having at least 95% identity to SEQ ID NO:56.

30. The anti-CD70 antibody of any of claims 16-29, wherein the V_{L} comprises SEQ ID NO:56.

31. An anti-CD70 antibody comprising:
(a) a V_{H} comprising:
(i) a CDR-H1 comprising an amino acid sequence having at least 80% sequence identity to SEQ ID NO:61;
(ii) a CDR-H2 comprising an amino acid sequence having at least 80% sequence identity to SEQ ID NO:62; and
(iii) a CDR-H3 comprising an amino acid sequence having at least 80% sequence identity to SEQ ID NO:63; and
(b) a V_{L} comprising:
(i) a CDR-L1 comprising an amino acid sequence having at least 80% sequence identity to SEQ ID NO:65;
(ii) a CDR-L2 comprising an amino acid sequence having at least 80% sequence identity to SEQ ID NO:66; and
(iii) a CDR-L3 comprising an amino acid sequence having at least 80% sequence identity to SEQ ID NO:67.

32. The anti-CD70 antibody of claim 31, wherein the CDR-H1 comprises SEQ ID NO:61.

33. The anti-CD70 antibody of claim 31 or 32, wherein the CDR-H2 comprises SEQ ID NO:62.

34. The anti-CD70 antibody of any of claims 31-33, wherein the CDR-H3 comprises SEQ ID NO:63

35. The anti-CD70 antibody of any of claims 31-34, wherein the CDR-L1 comprises SEQ ID NO:65.

36. The anti-CD70 antibody of any of claims 31-35, wherein the CDR-L2 comprises SEQ ID NO:66.

37. The anti-CD70 antibody of any of claims 31-36, wherein the CDR-L3 comprises SEQ ID NO:67.

38. The anti-CD70 antibody of any of claims 31-37, wherein the V_{H} comprises an amino acid sequence having at least 85% identity to SEQ ID NO:60.

39. The anti-CD70 antibody of any of claims 31-38, wherein the V_{H} comprises an amino acid sequence having at least 90% identity to SEQ ID NO:60.

40. The anti-CD70 antibody of any of claims 31-39, wherein the V_{H} comprises an amino acid sequence having at least 95% identity to SEQ ID NO:60.

41. The anti-CD70 antibody of any of claims 31-40, wherein the V_{H} comprises SEQ ID NO:60.

42. The anti-CD70 antibody of any of claims 31-41, wherein the V_{L} comprises an amino acid sequence having at least 85% identity to SEQ ID NO:64.

43. The anti-CD70 antibody of any of claims 31-42, wherein the V_{L} comprises an amino acid sequence having at least 90% identity to SEQ ID NO:64.

44. The anti-CD70 antibody of any of claims 31-43, wherein the V_{L} comprises an amino acid sequence having at least 95% identity to SEQ ID NO:64.

45. The anti-CD70 antibody of any of claims 31-44, wherein the V_{L} comprises SEQ ID NO:64.

46. A polynucleotide encoding the antibody of any of claims 1-45.

47. A vector comprising the polynucleotide of claim 46.

48. A method for generating the antibody of any of claims 1-45, comprising (a) providing a polynucleotide encoding for the antibody to a cell and (b) subjecting the cell to conditions sufficient to express the antibody from the polynucleotide.

49. A pharmaceutical composition comprising:
(a) the antibody of any of claims 1-45, the polynucleotide of claim 46, or the vector of claim 47; and
(b) a pharmaceutically acceptable excipient.

50. The pharmaceutical composition of claim 49, further comprising an additional therapeutic.

51. The pharmaceutical composition of claim 50, wherein the additional therapeutic is a chemotherapeutic.

52. A method for treating a subject for cancer, the method comprising administering to the subject a therapeutically effective amount of a composition comprising the antibody of any of claims 1-45, the polynucleotide of claim 46, or the vector of claim 47.

53. The method of claim 52, wherein the subject has a CD70⁺ cancer.

54. The method of claim 52 or 53, wherein the subject has lymphoma, leukemia, glioblastoma, melanoma, non-small cell lung cancer, renal cell carcinoma, pancreatic cancer, ovarian cancer, or breast cancer.

55. The method of any of claims 52-54, further comprising administering to the subject an additional therapy.

56. The method of claim 55, wherein the additional therapy is radiotherapy, chemotherapy, or immunotherapy.

57. A polynucleotide encoding a CD70-specific engineered receptor, the receptor comprising:
(a) an antigen binding region comprising:
(i) a V_{H} comprising:
(1) a CDR-H1 comprising an amino acid sequence having at least 80% sequence identity to SEQ ID NO:45;
(2) a CDR-H2 comprising an amino acid sequence having at least 80% sequence identity to SEQ ID NO:46; and
(3) a CDR-H3 comprising an amino acid sequence having at least 80% sequence identity to SEQ ID NO:47; and
(ii) a V_{L} comprising:
(1) a CDR-L1 comprising an amino acid sequence having at least 80% sequence identity to SEQ ID NO:49;
(2) a CDR-L2 comprising an amino acid sequence having at least 80% sequence identity to SEQ ID NO:50; and
(3) a CDR-L3 comprising an amino acid sequence having at least 80% sequence identity to SEQ ID NO:51; and
(b) a transmembrane domain; and
(c) an intracellular domain.

58. The polynucleotide of claim 57, wherein the CDR-H1 comprises SEQ ID NO:45.

59. The polynucleotide of claim 57 or 58, wherein the CDR-H2 comprises SEQ ID NO:46.

60. The polynucleotide of any of claims 57-59, wherein the CDR-H3 comprises SEQ ID NO:47.

61. The polynucleotide of any of claims 57-60, wherein the CDR-L1 comprises SEQ ID NO:49.

62. The polynucleotide of any of claims 57-61, wherein the CDR-L2 comprises SEQ ID NO:50.

63. The polynucleotide of any of claims 57-62, wherein the CDR-L3 comprises SEQ ID NO:51.

64. The polynucleotide of any of claims 57-63, wherein the V_{H} comprises an amino acid sequence having at least 85% identity to SEQ ID NO:44.

65. The polynucleotide of any of claims 57-64, wherein the V_{H} comprises an amino acid sequence having at least 90% identity to SEQ ID NO:44.

66. The polynucleotide of any of claims 57-65, wherein the V_{H} comprises an amino acid sequence having at least 95% identity to SEQ ID NO:44.

67. The polynucleotide of any of claims 57-66, wherein the V_{H} comprises SEQ ID NO:44.

68. The polynucleotide of any of claims 57-67, wherein the V_{L} comprises an amino acid sequence having at least 85% identity to SEQ ID NO:48.

69. The polynucleotide of any of claims 57-68, wherein the V_{L} comprises an amino acid sequence having at least 90% identity to SEQ ID NO:48.

70. The polynucleotide of any of claims 57-69, wherein the V_{L} comprises an amino acid sequence having at least 95% identity to SEQ ID NO:48.

71. The polynucleotide of any of claims 57-70, wherein the V_{L} comprises SEQ ID NO:48.

72. The polynucleotide of any of claims 57-71, wherein the antigen binding region comprises a linker.

73. The polynucleotide of claim 72, wherein the linker comprises SEQ ID NO:74.

74. The polynucleotide of any of claims 57-73, wherein the region of the polynucleotide encoding the V_{H} is upstream of the region of the polynucleotide encoding the V_{L} in a 5′ to 3′ direction.

75. The polynucleotide of claim 74, wherein the antigen binding region comprises SEQ ID NO:68.

76. The polynucleotide of any of claims 57-73, wherein the region of the polynucleotide encoding the V_{L} is upstream of the region of the polynucleotide encoding the V_{H} in a 5′ to 3′ direction.

77. The polynucleotide of claim 76, wherein the antigen binding region comprises SEQ ID NO:69.

78. A polynucleotide encoding a CD70-specific engineered receptor, the receptor comprising:
(a) an antigen binding region comprising:
(i) a V_{H} comprising:
(1) a CDR-H1 comprising an amino acid sequence having at least 80% sequence identity to SEQ ID NO:53;
(2) a CDR-H2 comprising an amino acid sequence having at least 80% sequence identity to SEQ ID NO:54; and
(3) a CDR-H3 comprising an amino acid sequence having at least 80% sequence identity to SEQ ID NO:55; and
(ii) a V_{L} comprising:
(1) a CDR-L1 comprising an amino acid sequence having at least 80% sequence identity to SEQ ID NO:57;
(2) a CDR-L2 comprising an amino acid sequence having at least 80% sequence identity to SEQ ID NO:58 and
(3) a CDR-L3 comprising an amino acid sequence having at least 80% sequence identity to SEQ ID NO:59; and
(b) a transmembrane domain; and
(c) an intracellular domain.

79. The polynucleotide of claim 78, wherein the CDR-H1 comprises SEQ ID NO:53.

80. The polynucleotide of claim 78 or 79, wherein the CDR-H2 comprises SEQ ID NO:54.

81. The polynucleotide of any of claims 78-80, wherein the CDR-H3 comprises SEQ ID NO:55.

82. The polynucleotide of any of claims 78-81, wherein the CDR-L1 comprises SEQ ID NO:57.

83. The polynucleotide of any of claims 78-82, wherein the CDR-L2 comprises SEQ ID NO:58.

84. The polynucleotide of any of claims 78-83, wherein the CDR-L3 comprises SEQ ID NO:59.

85. The polynucleotide of any of claims 78-84, wherein the V_{H} comprises an amino acid sequence having at least 85% identity to SEQ ID NO:52.

86. The polynucleotide of any of claims 78-85, wherein the V_{H} comprises an amino acid sequence having at least 90% identity to SEQ ID NO:52.

87. The polynucleotide of any of claims 78-86, wherein the V_{H} comprises an amino acid sequence having at least 95% identity to SEQ ID NO:52.

88. The polynucleotide of any of claims 78-87, wherein the V_{H} comprises SEQ ID NO:52.

89. The polynucleotide of any of claims 78-88, wherein the V_{L} comprises an amino acid sequence having at least 85% identity to SEQ ID NO:56.

90. The polynucleotide of any of claims 78-89, wherein the V_{L} comprises an amino acid sequence having at least 90% identity to SEQ ID NO:56.

91. The polynucleotide of any of claims 78-90, wherein the V_{L} comprises an amino acid sequence having at least 95% identity to SEQ ID NO:56.

92. The polynucleotide of any of claims 78-91, wherein the V_{L} comprises SEQ ID NO:56.

93. The polynucleotide of any of claims 78-92, wherein the antigen binding region comprises a linker.

94. The polynucleotide of claim 93, wherein the linker comprises SEQ ID NO:74.

95. The polynucleotide of any of claims 78-94, wherein the region of the polynucleotide encoding the V_{H} is upstream of the region of the polynucleotide encoding the V_{L} in a 5′ to 3′ direction.

96. The polynucleotide of claim 95, wherein the antigen binding region comprises SEQ ID NO:70.

97. The polynucleotide of any of claims 78-94, wherein the region of the polynucleotide encoding the V_{L} is upstream of the region of the polynucleotide encoding the V_{H} in a 5′ to 3′ direction.

98. The polynucleotide of claim 97, wherein the antigen binding region comprises SEQ ID NO:71.

99. A polynucleotide encoding a CD70-specific engineered receptor, the receptor comprising:
(a) an antigen binding region comprising:
(i) a V_{H} comprising:
(1) a CDR-H1 comprising an amino acid sequence having at least 80% sequence identity to SEQ ID NO:61;
(2) a CDR-H2 comprising an amino acid sequence having at least 80% sequence identity to SEQ ID NO:62 and
(3) a CDR-H3 comprising an amino acid sequence having at least 80% sequence identity to SEQ ID NO:63; and
(ii) a V_{L} comprising:
(1) a CDR-L1 comprising an amino acid sequence having at least 80% sequence identity to SEQ ID NO:65;
(2) a CDR-L2 comprising an amino acid sequence having at least 80% sequence identity to SEQ ID NO:66; and
(3) a CDR-L3 comprising an amino acid sequence having at least 80% sequence identity to SEQ ID NO:67; and
(b) a transmembrane domain; and
(c) an intracellular domain.

100. The polynucleotide of claim 99, wherein the CDR-H1 comprises SEQ ID NO:61.

101. The polynucleotide of claim 99 or 100, wherein the CDR-H2 comprises SEQ ID NO:62.

102. The polynucleotide of any of claims 99-101, wherein the CDR-H3 comprises SEQ ID NO:63.

103. The polynucleotide of any of claims 99-102, wherein the CDR-L1 comprises SEQ ID NO:65.

104. The polynucleotide of any of claims 99-103, wherein the CDR-L2 comprises SEQ ID NO:66.

105. The polynucleotide of any of claims 99-104, wherein the CDR-L3 comprises SEQ ID NO:67.

106. The polynucleotide of any of claims 99-105, wherein the V_{H} comprises an amino acid sequence having at least 85% identity to SEQ ID NO:60.

107. The polynucleotide of any of claims 99-106, wherein the V_{H} comprises an amino acid sequence having at least 90% identity to SEQ ID NO:60.

108. The polynucleotide of any of claims 99-107, wherein the V_{H} comprises an amino acid sequence having at least 95% identity to SEQ ID NO:60.

109. The polynucleotide of any of claims 99-108, wherein the V_{H} comprises SEQ ID NO:60.

110. The polynucleotide of any of claims 99-109, wherein the V_{L} comprises an amino acid sequence having at least 85% identity to SEQ ID NO:64.

111. The polynucleotide of any of claims 99-110, wherein the V_{L} comprises an amino acid sequence having at least 90% identity to SEQ ID NO:64.

112. The polynucleotide of any of claims 99-111, wherein the V_{L} comprises an amino acid sequence having at least 95% identity to SEQ ID NO:64.

113. The polynucleotide of any of claims 99-112, wherein the V_{L} comprises SEQ ID NO:64.

114. The polynucleotide of any of claims 99-113, wherein the antigen binding region comprises a linker.

115. The polynucleotide of claim 114, wherein the linker comprises SEQ ID NO:74.

116. The polynucleotide of any of claims 99-115, wherein the region of the polynucleotide encoding the V_{H} is upstream of the region of the polynucleotide encoding the V_{L} in a 5′ to 3′ direction.

117. The polynucleotide of claim 116, wherein the antigen binding region comprises SEQ ID NO:72.

118. The polynucleotide of any of claims 99-115, wherein the region of the polynucleotide encoding the V_{L} is upstream of the region of the polynucleotide encoding the V_{H} in a 5′ to 3′ direction.

119. The polynucleotide of claim 118, wherein the antigen binding region comprises SEQ ID NO:73.

120. The polynucleotide of any of claims 57-113, wherein the transmembrane domain is a transmembrane domain from CD28, the alpha chain of the T-cell receptor, beta chain of the T-cell receptor, zeta chain of the T-cell receptor, CD3 zeta, CD3 epsilon, CD3 gamma, CD3 delta, CD45, CD4, CD5, CD8, CD9, CD16, CD22, CD33, CD37, CD64, CD80, CD86, CD 134, CD137, CD154, ICOS/CD278, GITR/CD357, NKG2D, DAP10, or DAP12.

121. The polynucleotide of claim 120, wherein the transmembrane domain is a CD28 transmembrane domain.

122. The polynucleotide of claim 120 or 121, wherein the transmembrane domain comprises SEQ ID NO:75.

123. The polynucleotide of any of claims 57-122, wherein the intracellular domain is an intracellular domain from CD3 zeta, CD27, CD28, 4-1BB, DAP12, NKG2D, OX-40 (CD134), DAP10, CD40L, 2B4, DNAM, CS1, CD48, NKp30, NKp44, NKp46, or NKp80.

124. The polynucleotide of claim 123, wherein the intracellular domain is a CD28 intracellular domain.

125. The polynucleotide of claim 123, wherein the intracellular domain is a CD3 zeta intracellular domain.

126. The polynucleotide of claim 125, wherein the intracellular domain comprises SEQ ID NO:81 or SEQ ID NO:82.

127. The polynucleotide of any of claims 57-126, wherein the engineered receptor comprises two or more intracellular domains.

128. The polynucleotide of claim 127, wherein the two or more intracellular domains comprise a CD3 zeta intracellular domain and an additional intracellular domain selected from a CD28, DAP10, DAP12, 4-1BB, NKG2D, and 2B4 intracellular domain.

129. The polynucleotide of claim 128, wherein the two or more intracellular domains comprise a CD3 zeta intracellular domain and a CD28 intracellular domain.

130. The polynucleotide of any of claims 57-129, further comprising a signal peptide.

131. The polynucleotide of claim 130, wherein the signal peptide is a signal peptide from CD8, CD27, granulocyte-macrophage colony-stimulating factor receptor (GMSCF-R), Ig heavy chain, a killer cell immunoglobulin-like receptor (KIR), CD3, or CD4.

132. The polynucleotide of claim 131, wherein the signal peptide is a CD8 signal peptide.

133. The polynucleotide of any of claims 57-132, further comprising a hinge between the antigen binding domain and the transmembrane domain.

134. The polynucleotide of claim 133, wherein the hinge is an IgG hinge, a CD28 hinge, or a CD8a hinge.

135. The polynucleotide of claim 133 or 134, wherein the hinge is IgG1 hinge, IgG2 hinge, IgG3 hinge, or IgG4 hinge.

136. The polynucleotide of claim 135, wherein the hinge is an IgG1 hinge.

137. The polynucleotide of any of claims 57-136, wherein the polynucleotide further encodes an additional polypeptide.

138. The polynucleotide of claim 137, wherein the additional polypeptide is a therapeutic protein or a protein that enhances cell activity, expansion, and/or persistence.

139. The polynucleotide of claim 137 or 138, wherein the additional polypeptide is a suicide gene, a cytokine, or a human or viral protein that enhances proliferation, expansion and/or metabolic fitness.

140. The polynucleotide of claim 139, wherein the additional polypeptide is a cytokine.

141. The polynucleotide of claim 140, wherein the cytokine is IL-15, IL-2, IL-12, IL-18, IL-21, IL-23, or IL-7.

142. The polynucleotide of claim 141, wherein the cytokine is IL-15.

143. The polynucleotide of any of claims 57-142, wherein the CD70-specific engineered receptor is a chimeric antigen receptor (CAR).

144. The polynucleotide of any of claims 57-142, wherein the CD70-specific engineered receptor is a T cell receptor.

145. A vector comprising the polynucleotide of any one of claims 57-144.

146. The vector of claim 145, wherein the vector is a viral vector.

147. The vector of claim 146, wherein the viral vector is an adenoviral vector, adeno-associated viral vector, lentiviral vector, or retroviral vector.

148. The vector of claim 145, wherein the vector is a non-viral vector.

149. The vector of claim 148, wherein the non-viral vector is a plasmid.

150. An immune cell comprising the polynucleotide of any one of claims 57-144 or the vector of any one of claims 145-149.

151. The immune cell of claim 150, wherein the immune cell is a natural killer (NK) cell, T cell, gamma delta T cell, alpha beta T cell, invariant NKT (iNKT) cell, B cell, macrophage, mesenchymal stromal cell, or dendritic cell.

152. The immune cell of claim 151, wherein the immune cell is an NK cell.

153. The immune cell of claim 152, wherein the NK cell is derived from cord blood, peripheral blood, induced pluripotent stem cells, hematopoietic stem cells, bone marrow, or from a cell line.

154. The immune cell of claim 153, wherein the NK cell is derived from a cell line, wherein the NK cell line is NK-92.

155. The immune cell of claim 153, wherein the NK cell is derived from a cord blood mononuclear cell.

156. The immune cell of any of claims 150-155, wherein the NK cell is a CD56⁺ NK cell.

157. The immune cell of any of claims 150-156, wherein the NK cell expresses a recombinant cytokine.

158. The immune cell of claim 157, wherein the cytokine is IL-15, IL-2, IL-12, IL-18, IL-21, IL-7, or IL-23.

159. The immune cell of claim 158, wherein the cytokine is IL-15.

160. A population of immune cells comprising the immune cell of any one of claims 150-159.

161. A method of killing CD70-positive cells in an individual, comprising administering to the individual an effective amount of cells harboring the polynucleotide of any of claims 57-144.

162. A method for treating a subject for cancer, the method comprising administering to the subject a therapeutically effective amount of the immune cell of any of claims 150-159 or the population of immune cells of claim 160.

163. The method of claim 162, wherein the subject has a CD70⁺ cancer.

164. The method of claim 162 or 163, wherein the subject has lymphoma, leukemia, glioblastoma, melanoma, non-small cell lung cancer, renal cell carcinoma, pancreatic cancer, ovarian cancer, or breast cancer.

165. The method of any of claims 162-164, further comprising administering to the subject an additional therapy.

166. The method of claim 165, wherein the additional therapy is radiotherapy, chemotherapy, or immunotherapy.

167. A pharmaceutical composition comprising:
(a) the immune cell of any of claims 150-159 or the population of immune cells of claim 160; and
(b) a pharmaceutically acceptable excipient.

168. The pharmaceutical composition of claim 167, further comprising an additional therapeutic.

169. The pharmaceutical composition of claim 168, wherein the additional therapeutic is a chemotherapeutic.

170. An immune cell engager comprising:
(a) a CD70-binding region comprising:
(i) a V_{H} comprising:
(1) a CDR-H1 comprising an amino acid sequence having at least 80% sequence identity to SEQ ID NO:45;
(2) a CDR-H2 comprising an amino acid sequence having at least 80% sequence identity to SEQ ID NO:46; and
(3) a CDR-H3 comprising an amino acid sequence having at least 80% sequence identity to SEQ ID NO:47; and
(ii) a V_{L} comprising:
(1) a CDR-L1 comprising an amino acid sequence having at least 80% sequence identity to SEQ ID NO:49;
(2) a CDR-L2 comprising an amino acid sequence having at least 80% sequence identity to SEQ ID NO:50; and
(3) a CDR-L3 comprising an amino acid sequence having at least 80% sequence identity to SEQ ID NO:51; and
(b) an immune cell binding region.

171. The immune cell engager of claim 170, wherein the CDR-H1 comprises SEQ ID NO:45.

172. The immune cell engager of claim 170 or 171, wherein the CDR-H2 comprises SEQ ID NO:46.

173. The immune cell engager of any of claims 170-172, wherein the CDR-H3 comprises SEQ ID NO:47.

174. The immune cell engager of any of claims 170-173, wherein the CDR-L1 comprises SEQ ID NO:49.

175. The immune cell engager of any of claims 170-174, wherein the CDR-L2 comprises SEQ ID NO:50.

176. The immune cell engager of any of claims 170-175, wherein the CDR-L3 comprises SEQ ID NO:51.

177. The immune cell engager of any of claims 170-176, wherein the V_{H} comprises an amino acid sequence having at least 85% identity to SEQ ID NO:44.

178. The immune cell engager of any of claims 170-177, wherein the V_{H} comprises an amino acid sequence having at least 90% identity to SEQ ID NO:44.

179. The immune cell engager of any of claims 170-178, wherein the V_{H} comprises an amino acid sequence having at least 95% identity to SEQ ID NO:44.

180. The immune cell engager of any of claims 170-179, wherein the V_{H} comprises SEQ ID NO:44.

181. The immune cell engager of any of claims 170-180, wherein the V_{L} comprises an amino acid sequence having at least 85% identity to SEQ ID NO:48.

182. The immune cell engager of any of claims 170-181, wherein the V_{L} comprises an amino acid sequence having at least 90% identity to SEQ ID NO:48.

183. The immune cell engager of any of claims 170-182, wherein the V_{L} comprises an amino acid sequence having at least 95% identity to SEQ ID NO:48.

184. The immune cell engager of any of claims 170-183, wherein the V_{L} comprises SEQ ID NO:48.

185. The immune cell engager of any of claims 170-184, wherein the CD70-binding region comprises a linker.

186. The immune cell engager of claim 185, wherein the linker comprises SEQ ID NO:74.

187. The immune cell engager of any of claims 170-186, wherein the V_{H} of the CD70-binding region is upstream of the V_{L} of the CD70-binding region in a 5′ to 3′ direction.

188. The immune cell engager of claim 187, wherein the CD70-binding region comprises SEQ ID NO:68.

189. The immune cell engager of any of claims 170-186, wherein the V_{L} of the CD70-binding region is upstream of the V_{H} of the CD70-binding region in a 5′ to 3′ direction.

190. The immune cell engager of claim 189, wherein the CD70-binding region comprises SEQ ID NO:69.

191. An immune cell engager comprising:
(a) a CD70-binding region comprising:
(i) a V_{H} comprising:
(1) a CDR-H1 comprising an amino acid sequence having at least 80% sequence identity to SEQ ID NO:53;
(2) a CDR-H2 comprising an amino acid sequence having at least 80% sequence identity to SEQ ID NO:54; and
(3) a CDR-H3 comprising an amino acid sequence having at least 80% sequence identity to SEQ ID NO:55; and
(ii) a V_{L} comprising:
(1) a CDR-L1 comprising an amino acid sequence having at least 80% sequence identity to SEQ ID NO:57;
(2) a CDR-L2 comprising an amino acid sequence having at least 80% sequence identity to SEQ ID NO:58; and
(3) a CDR-L3 comprising an amino acid sequence having at least 80% sequence identity to SEQ ID NO:59; and
(b) an immune cell binding region.

192. The immune cell engager of claim 191, wherein the CDR-H1 comprises SEQ ID NO:53.

193. The immune cell engager of claim 191 or 192, wherein the CDR-H2 comprises SEQ ID NO:54.

194. The immune cell engager of any of claims 191-193, wherein the CDR-H3 comprises SEQ ID NO:55.

195. The immune cell engager of any of claims 191-194, wherein the CDR-L1 comprises SEQ ID NO:57.

196. The immune cell engager of any of claims 191-195, wherein the CDR-L2 comprises SEQ ID NO:58.

197. The immune cell engager of any of claims 191-196, wherein the CDR-L3 comprises SEQ ID NO:59.

198. The immune cell engager of any of claims 191-197, wherein the V_{H} comprises an amino acid sequence having at least 85% identity to SEQ ID NO:52.

199. The immune cell engager of any of claims 191-198, wherein the V_{H} comprises an amino acid sequence having at least 90% identity to SEQ ID NO:52.

200. The immune cell engager of any of claims 191-199, wherein the V_{H} comprises an amino acid sequence having at least 95% identity to SEQ ID NO:52.

201. The immune cell engager of any of claims 191-200, wherein the V_{H} comprises SEQ ID NO:52.

202. The immune cell engager of any of claims 191-193, wherein the V_{L} comprises an amino acid sequence having at least 85% identity to SEQ ID NO:56.

203. The immune cell engager of any of claims 191-201, wherein the V_{L} comprises an amino acid sequence having at least 90% identity to SEQ ID NO:56.

204. The immune cell engager of any of claims 191-202, wherein the V_{L} comprises an amino acid sequence having at least 95% identity to SEQ ID NO:56.

205. The immune cell engager of any of claims 191-203, wherein the V_{L} comprises SEQ ID NO:56.

206. The immune cell engager of any of claims 191-205, wherein the CD70-binding region comprises a linker.

207. The immune cell engager of claim 206, wherein the linker comprises SEQ ID NO:74.

208. The immune cell engager of any of claims 191-207, wherein the V_{H} of the CD70-binding region is upstream of the V_{L} of the CD70-binding region in a 5′ to 3′ direction.

209. The immune cell engager of claim 208, wherein the CD70-binding region comprises SEQ ID NO:70.

210. The immune cell engager of any of claims 191-207, wherein the V_{L} of the CD70-binding region is upstream of the V_{H} of the CD70-binding region in a 5′ to 3′ direction.

211. The immune cell engager of claim 210, wherein the CD70-binding region comprises SEQ ID NO:71.

212. An immune cell engager comprising:
(a) a CD70-binding region comprising:
(i) a V_{H} comprising:
(1) a CDR-H1 comprising an amino acid sequence having at least 80% sequence identity to SEQ ID NO:61;
(2) a CDR-H2 comprising an amino acid sequence having at least 80% sequence identity to SEQ ID NO:62; and
(3) a CDR-H3 comprising an amino acid sequence having at least 80% sequence identity to SEQ ID NO:63; and
(ii) a V_{L} comprising:
(1) a CDR-L1 comprising an amino acid sequence having at least 80% sequence identity to SEQ ID NO:65
(2) a CDR-L2 comprising an amino acid sequence having at least 80% sequence identity to SEQ ID NO:66; and
(3) a CDR-L3 comprising an amino acid sequence having at least 80% sequence identity to SEQ ID NO:67; and
(b) an immune cell binding region.

213. The immune cell engager of claim 212, wherein the CDR-H1 comprises SEQ ID NO:61.

214. The immune cell engager of claim 212 or 213, wherein the CDR-H2 comprises SEQ ID NO:62.

215. The immune cell engager of any of claims 212-214, wherein the CDR-H3 comprises SEQ ID NO:63.

216. The immune cell engager of any of claims 212-215, wherein the CDR-L1 comprises SEQ ID NO:65.

217. The immune cell engager of any of claims 212-216, wherein the CDR-L2 comprises SEQ ID NO:66.

218. The immune cell engager of any of claims 212-217, wherein the CDR-L3 comprises SEQ ID NO:67.

219. The immune cell engager of any of claims 212-218, wherein the V_{H} comprises an amino acid sequence having at least 85% identity to SEQ ID NO:60.

220. The immune cell engager of any of claims 212-219, wherein the V_{H} comprises an amino acid sequence having at least 90% identity to SEQ ID NO:60.

221. The immune cell engager of any of claims 212-220, wherein the V_{H} comprises an amino acid sequence having at least 95% identity to SEQ ID NO:60.

222. The immune cell engager of any of claims 212-221, wherein the V_{H} comprises SEQ ID NO:60.

223. The immune cell engager of any of claims 212-222, wherein the V_{L} comprises an amino acid sequence having at least 85% identity to SEQ ID NO:64.

224. The immune cell engager of any of claims 212-223, wherein the V_{L} comprises an amino acid sequence having at least 90% identity to SEQ ID NO:64.

225. The immune cell engager of any of claims 212-224, wherein the V_{L} comprises an amino acid sequence having at least 95% identity to SEQ ID NO:64.

226. The immune cell engager of any of claims 212-225, wherein the V_{L} comprises SEQ ID NO:64.

227. The immune cell engager of any of claims 212-226, wherein the CD70-binding region comprises a linker.

228. The immune cell engager of claim 227, wherein the linker comprises SEQ ID NO:74.

229. The immune cell engager of any of claims 212-228, wherein the V_{H} of the CD70-binding region is upstream of the V_{L} of the CD70-binding region in a 5′ to 3′ direction.

230. The immune cell engager of claim 229, wherein the CD70-binding region comprises SEQ ID NO:72.

231. The immune cell engager of any of claims 212-228, wherein the V_{L} of the CD70-binding region is upstream of the V_{H} of the CD70-binding region in a 5′ to 3′ direction.

232. The immune cell engager of claim 231, wherein the CD70-binding region comprises SEQ ID NO:73.

233. The immune cell engager of any of claims 170-226, wherein the immune cell binding region specifically binds to a protein expressed on a surface of an immune cell.

234. The immune cell engager of claim 233, wherein the immune cell is an NK cell, T cell, gamma delta T cell, alpha beta T cell, iNKT cell, B cell, macrophage, mesenchymal stromal cell, or dendritic cell.

235. The immune cell engager of claim 234, wherein the immune cell is a T cell.

236. The immune cell engager of claim 235, wherein the immune cell binding region specifically binds to CD3, the T cell receptor (TCR), CD28, Ox40, 4-1 BB, CD2, CD5, CD95, CD27, IL-7R, ICOS, IL2Rβ, CD45, CD48, and CD137.

237. The immune cell engager of claim 236, wherein the immune cell binding region specifically binds to CD3.

238. The immune cell engager of claim 237, wherein the immune cell binding region comprises an scFv from an anti-CD3 antibody.

239. The immune cell engager of claim 234, wherein the immune cell is an NK cell.

240. The immune cell engager of claim 239, wherein the immune cell binding region specifically binds to CD16A, NKp46, or NKG2D.

241. The immune cell engager of claim 240, wherein the immune cell binding region specifically binds to CD16A.

242. The immune cell engager of claim 241, wherein the immune cell binding region comprises an scFv from an anti-CD16A antibody.

243. A polynucleotide encoding the immune cell engager of any of claims 170-242.

244. A vector comprising the immune cell engager of claim 243.

245. A method for generating the immune cell engager of any of claims 170-242, comprising (a) providing a polynucleotide encoding for the immune cell engager to a cell and (b) subjecting the cell to conditions sufficient to express the immune cell engager from the polynucleotide.

246. A pharmaceutical composition comprising:
(a) the immune cell engager of any of claims 170-242, the polynucleotide of claim 243, or the vector of claim 244; and
(b) a pharmaceutically acceptable excipient.

247. The pharmaceutical composition of claim 246, further comprising an additional therapeutic.

248. The pharmaceutical composition of claim 247, wherein the additional therapeutic is a chemotherapeutic.

249. A method for treating a subject for cancer, the method comprising administering to the subject a therapeutically effective amount of a composition comprising the immune cell engager of any of claims 170-242, the polynucleotide of claim 243, or the vector of claim 244.

250. The method of claim 249, wherein the subject has a CD70⁺ cancer.

251. The method of claim 249 or 250, wherein the subject has lymphoma, leukemia, multiple myeloma, glioblastoma, mesothelioma, head and neck cancers, osteosarcoma, melanoma, non-small cell lung cancer, renal cell carcinoma, pancreatic cancer, ovarian cancer, germ cell tumors, or breast cancer.

252. The method of any of claims 249-251, further comprising administering to the subject an additional therapy.

253. The method of claim 252, wherein the additional therapy is radiotherapy, chemotherapy, or immunotherapy.
